SEQUENCE LISTING (227 sequences)

SEQ ID NO: 1 - protein, 41 aa, Bacillus anthracis
MSNNNYSNGLNPDESLSASAFDPNLVGPTLPPIPPFTLPTG

SEQ ID NO: 2 - protein, 332 aa, Bacillus anthracis
MSNNNYSNGLNPDESLSASAFDPNLVGPTLPPIPPFTLPTGPTGPFTTGPTGPTGPTGPTGPTGPTGPTGPTGDTGTTGPTGPTGPTGPTGPTGPTGPTGPTGPTGFTPTGPTGPTGPTGDTGTTGPTGPTGPTGPTGPTGDTGTTGPTGPTGPTGPTGPTGPTGPTFTGPTGPTGPTGATGLTGPTGPTGPSGLGLPAGLYAFNSGGISLDLGINDPVPFNTVGSQFFTGTAISQLDADTFVISETGFYKITVIANTATASVLGGLTIQVNGVPVPGTGSSLISLGAPFTIVIQAITQITTTPSLVEVIVTGLGLSLALGTSASIIIEKVA

SEQ ID NO: 3 - protein, 33 aa, Bacillus anthracis
MSEKYIILHGTALEPNLIGPTLPPIPPFTFPNG

SEQ ID NO: 4 - protein, 209 aa, Bacillus anthracis
MSEKYIILHGTALEPNLIGPTLPPIPPFTFPNGPTGITGPTGATGFTGIGITGPTGVTGPTGIGITGPTGATGLGILPVFGTITTDVGIGFSVIVNTNINFTLPGPVSGTTLNPVDNSIIINTTGVYSVSFSIVFVIQAISSSILNLTINDSIQFAIESRIGGGPGVRATSARTDLLSLNQGDVLRVRIREATGDIIYSNASLVVSKVD

SEQ ID NO: 5 - protein, 44 aa, Bacillus anthracis
MVKVVEGNGGKSKIKSPLNSNFKILSDLVGPTFPPVPTGMTGIT

SEQ ID NO: 6 - protein, 647 aa, Bacillus anthracis
VVKVVEGNGGKSKIKSPLNSNFKILSDLVGPTFPPVPTGMTGITGSTGATGNTGPTGETGATGSAGITGSTGPTGNTGGTGSTGPTGNTGATGSTGVTGSTGVTGSTGVTGSTGVTGSTGPTGETGGTGSTGVTGSTGATGSTGVTGNTGPTGSTGATGNTGSIGETGGTGSMGPTGETGVTGSTGGTGSTGVTGNTGPTGSTGVTGSTGVTGSTGPTGSTGVTGSTGPTGSTGVTGSTGVTGNMGPTGSTGVTGNTGSTGTTGATGETGPMGSTGATGTTGPTGETGETGETGGTGSTGPTGNTGATGSTGVTGSTGVTGSTGVTGETGPTGSTGATGNTGPTGETGGTGSTGATGSTGVTGNTGPTGSTGVTGNTGATGETGPTGNTGATGNTGPTGETGVTGSTGPTGETGVTGSTGPTGNTGATGETGATGSTGVTGNTGSTGETGPTGSTGPTGSTGATGVTGNTGPTGSTGATGATGSTGPTGSTGTTGNTGVTGDTGPTGATGVSTTATYAFANNTSGSVISVLLGGTNIPLPNNQNIGPGITVSGGNTVFTVANAGNYYIAYTINLTAGLLVSSRITVNGSPLAGTINSPTVATGSFSATIIASLPAGAAVSLQLFGVVALATLSTATPGATLTIIRLS

SEQ ID NO: 7 - protein, 34 aa, Bacillus anthracis
MKQNDKLWLDKGIIGPENIGPTFPVLPPIHIPTG

SEQ ID NO: 8 - protein, 366 aa, Bacillus anthracis
MKQNDKLWLDKGIIGPENIGPTFPVLPPIHIPTGITGATGATGITGATGPTGTTGATGATGITGVTGATGITGVTGATGITGVTGATGITGVTGPTGITGATGPTGITGATGPAGITGVTGPTGITGATGPTGTTGVTGPTGDTGLAGATGPTGATGLAGATGPTGDTGATGPTGATGLAGATGPTGATGLTGATGATGATGGGAIIPFASGTTPALLVNAVLANTGTLLGFGFSQPGIAPGVGGTLTILPGVVGDYAFVAPRDGIITSLAGFFSATAALAPLTPVQIQMQIFIAPAASNTFTPVAPPLLLTPALPAIAIGTTATGIQAYNVPVVAGDKILVYVSLTGASPIAAVAGFVSAGLNIV

SEQ ID NO: 9 - protein, 30 aa, Bacillus anthracis
MDEFLSSAALNPGSVGPTLPPMQPFQFRTG

SEQ ID NO: 10 - protein, 77 aa, Bacillus anthracis
MDEFLSSAALNPGSVGPTLPPMQPFQFRTGPTGSTGAKGAIGNTEPYWHTGPPGIVLLTYDFKSLIISFAFRILPIS

SEQ ID NO: 11 - protein, 39 aa, Bacillus weihenstephensis
MFDKNEIQKINGILQANALNPNLIGPTLPPIPPFTLPTG

SEQ ID NO: 12 - protein, 299 aa, Bacillus weihenstephensis
MFDKNEIQKINGILQANALNPNLIGPTLPPIPPFTLPTGPTGVTGPTGVTGPTGVTGPTGVTGPTGVTGPTGVTGPTGVTGPTGVTGPTGVTGPTGVTGPTGVTGPTGVTGPTGVTGPTGVTGPTGETGPTGGTEGCLCDCCVLPMQSVLQQLIGETVILGTIADTPNTPPLFFLFTITSVNDFLVTVTDGTTTFVVNISDVTGVGFLPPGPPITLLPPTDVGCECECRERPIRQLLDAFIGSTVSLLASNGSIAADFSVEQTGLGIVLGTLPINPTTTVRFAISTCKITAVNITPITM

SEQ ID NO: 13 - protein, 39 aa, Bacillus weihenstephensis
MFDKNEMKKTNEVLQANALDPNIIGPTLPPIPPFTLPTG

SEQ ID NO: 14 - protein, 289 aa, Bacillus weihenstephensis
MFDKNEMKKTNEVLQANALDPNIIGPTLPPIPPFTLPTGPTGPTGPTGPTGPTGPTGPTGPTGPTGPTGPTGPTGPTGLTGPTGPTGLTGPTGLTGPTGPTGLTGQTGSTGPTGATEGCLCDCCVFPMQEVLRQLVGQTVILATIADAPNVAPRFFLFNITSVNDFLVTVTDPVSNTTFVVNISDVIGVGFSLTVPPLTLLPPADLGCECDCRERPIRELLDTLIGSTVNLLVSNGSIATGFNVEQTALGIVIGTLPIPINPPPPTLFRFAISTCKITAVDITPTPTAT

SEQ ID NO: 15 - protein, 49 aa, Bacillus cereus
MSRKDKFNRSRMSRKDRFNSPKIKSEISISPDLVGPTFPPIPSFTLPTG

SEQ ID NO: 16 - protein, 189 aa, Bacillus cereus
MSRKDKFNRSRMSRKDRFNSPKIKSEISISPDLVGPTFPPIPSFTLPTGITGPTFNINFRAEKNVAQSFTPPADIQVSYGNIIFNNGGGYSSVTNTFTAPINGIYLFSASIGFNPTLGTTSTLRITIRKNLVSVASQTGTITTGGTPQLEITTIIDLLASQTIDIQFSAAESGTLTVGSSNFFSGALLP

SEQ ID NO: 17 - protein, 33 aa, Bacillus cereus
MNEEYSILHGPALEPNLIGPTLPSIPPFTFPTG

SEQ ID NO: 18 - protein, 84 aa, Bacillus cereus
MNEEYSILHGPALEPNLIGPTLPSIPPFTFPTGPTGITGPTGATGFTGIGITGPTGVTGPTGIGITGPTGATGPTGIGITGPTG

SEQ ID NO: 19 - protein, 39 aa, Bacillus cereus
MKNRDNNRKQNSLSSNFRIPPELIGPTFPPVPTGFTGIG

SEQ ID NO: 20 - protein, 1056 aa, Bacillus cereus
MKNRDNNRKQNSLSSNFRIPPELIGPTFPPVPTGFTGIGITGPTGPQGPTGPQGPRGLQGPMGEMGPTGPQGVQGIQGSVGPIGATGPEGQQGPQGLRGPQGETGATGPGGVQGLQGPIGPTGATGAQGIQGIQGLQGPIGATGPEGSQGIQGVQGLPGATGPQGIQGAQGIQGTPGPSGNTGATGATGATGQGITGPTGITGPTGITGPSGGPPGPTGPTGATGPGGGPSGSTGATGATGNTGATGSTGVTGATGSTGPTGSTGAQGLQGIQGIQGPIGPTGPEGSQGIQGIPGPTGVTGEQGIQGVQGIQGATGATGDQGPQGIQGVIGPQGVTGATGDQGPQGIQGVPGPSGETGPQGVQGIQGPMGDIGPTGPEGPEGLQGPQGIQGVPGPVGATGPEGPQGIQGIQGPVGATGPQGPQGIQGIQGVQGITGATGVQGATGIQGIQGEIGATGPEGPQGVQGAQGAIGPTGPMGPQGVQGVQGIQGATGAQGVQGPQGIQGIQGPTGATGDMGATGATGEGTTGPTGVTGPTGVTGPSGGPAGPTGPTGPSGPAGVTGPSGGPPGPTGATGATGVTGDTGATGSTGVTGATGETGATGVTGLQGPQGIQGVQGEIGPTGPQGVQGPQGIQGVTGATGDQGPQGIQGPQGDIGPTGPQGIQGPQGSQGIQGATGGTGAQGPQGIQGPQGDIGLTGSQGPTGIQGIQGEIGPTGPEGPEGLQGPQGIQGIQGPVGATGPEGPQGIQGIQGVQGATGPQGPQGIQGIQGVQGITGATGAQGATGIQGIQGEIGATGPEGPQGVQGIQGAIGPTGPMGAQGVQGIQGIQGATGAQGVQGPQGIQGVQGPTGATGETGATGATGEGTTGPTGVTGPTGVTGPSGGPAGPTGPTGPSGPAGVTGPSGGPPGPTGATGATGVTGDTGATGSTGVTGATGATGATGVTGLQGPQGIQGVQGEIGPTGPQGIQGPQGIQGVTGATGAQGPQGIQGPQGDIGPTGSQGIQGPQGPQGIQGATGATGAQGPQGIQGPQGEIGPTGPQGPQGIQGPQGIQGPTG

SEQ ID NO: 21 - protein, 39 aa, Bacillus weihenstephensis
MSDKHQMKKISEVLQAHALDPNLIGPPLPPITPFTFPTG

SEQ ID NO: 22 - protein, 365 aa, Bacillus weihenstephensis
MSDKHQMKKISEVLQAHALDPNLIGPPLPPITPFTFPTGSTGPTGSTGSTGPTGSTGNTGPTGPTGPPVGTNLDTIYVTNDISNNVSAIDGNTNTVLTTIPVGTNPVGVGVNSSTNLIYVVNNGSDNISVINGSTNTVVATIPVGTQPFGVGVNPSTNLIYVANRTSNNVSVIKGGTNTVLTTIPVGTNPVGVGVNSSTNLIYVTNEIPNSVSVIKGGTNTVVATIPVGLFPFGVGVNSLTNLIYVVNNSPHNVSVIDGNTNTVLTTISVGTSPVGVGVNLSTNLIYVANEVPNNISVINGNTNTVLTTIPVGTTPFEVGVNSSTNLIYVSNLNSNNVSVINGSANTVIATVPVGSVPRGIGVKP

SEQ ID NO: 23 - protein, 30 aa, Bacillus weihenstephensis
MDEFLSFAALNPGSIGPTLPPVPPFQFPTG

SEQ ID NO: 24 - protein, 160 aa, Bacillus weihenstephensis
MDEFLSFAALNPGSIGPTLPPVPPFQFPTGPTGSTGSTGPTGSTGSTGPTGFNLPAGPASITLTSNETTACVSTQGNNTLFFSGQVLVNGSPTPGVVVSFSFSNPSLAFMVPLAVITNASGNFTAVFLAANGPGTVTVTASLLDSPGTMASVTITIVNCP

SEQ ID NO: 25 - protein, 30 aa, Bacillus weihenstephensis
MDEFLSSTALNPCSIGPTLPPMQPFQFPTG

SEQ ID NO: 26 - protein, 69 aa, Bacillus weihenstephensis
MDEFLSSTALNPCSIGPTLPPMQPFQFPTGPTGSTGTTGPTGSIGPTGNTGLTGNTGPTGITGPTGDTG

SEQ ID NO: 27 - protein, 36 aa, Bacillus weihenstephensis
MKERDRQNSLNSNFRISPNLIGPTFPPVPTGFTGIG

SEQ ID NO: 28 - protein, 934 aa, Bacillus weihenstephensis
MKERDRQNSLNSNFRISPNLIGPTFPPVPTGFTGIGITGPTGPQGPTGPQGPRGFQGPMGEMGPTGPQGVQGIQGPAGQMGATGPEGQQGPQGLRGPQGETGATGPQGVQGLQGPIGPTGATGAQGIQGIQGLQGPIGATGPEGPQGIQGVQGVPGATGSQGIQGAQGIQGPQGPSGNTGATGVTGQGISGPTGITGPTGITGPSGGPPGPTGATGATGPGGGPSGSTGATGATGNTGVTGSAGVTGNTGSTGSTGETGAQGLQGIQGVQGPIGPTGPEGPQGIQGIPGPTGVTGEQGIQGVQGIQGITGATGDQGPQGIQGAIGPQGITGATGDQGPQGIQGVPGPTGDTGSQGVQGIQGPMGDIGPTGPEGPEGLQGPQGIQGVPGPAGATGPEGPQGIQGIQGPIGVTGPEGPQGIQGIQGIQGITGATGAQGATGVQGVQGNIGATGPEGPQGVQGTQGDIGPTGPMGPQGVQGIQGIQGPTGAQGVQGPQGIQGIQGPTGVTGDTGTTGATGEGTTGATGVTGPSGVTGPSGGPAGPTGPTGPSGPTGLTGPSGGPPGPTGATGVTGGVGDTGATGSTGVTGATGVTGATGATGLQGPQGIQGVQGDIGPTGPQGVQGPQGIQGITGATGDQGPQGIQGPQGIQGPTGPQGIQGGQGPQGIQGATGATGAQGPQGIQGIQGVQGPTGPQGPTGIQGVQGEIGPTGPQGVQGLQGPQGPTGDTGPTGPQGPQGIQGPTGATGATGSQGIQGPTGATGATGSQGIQGPTGATGATGATGATGATGATGATGVTGVSTTATYSFANNTSGSAISVLLGGTNIPLPNNQNIGPGITVSGGNTVFTVTNAGNYYIAYTINITAALLVSSRITVNGSPLAGTINSPAVATGSFNATIISNLAAGSAISLQLFGLLAVATLSTTTPGATLTIIRLS

SEQ ID NO: 29 - protein, 39 aa, Bacillus mycoides
VFDKNEIQKINGILQANALNPNLIGPTLPPIPPFTLPTG

SEQ ID NO: 30 - protein, 287 aa, Bacillus mycoides
VFDKNEIQKINGILQANALNPNLIGPTLPPIPPFTLPTGPTGGTGPTGVTGPTGVTGPTGVTGPTGVTGPTGVTGPTGVTGPTGVTGPTGVTGPTGVTGPTGVTGPTGVTGPTGVTGPTGGTEGCLCDCCVLPMQSVLQQLIGETVILGTIADTPNTPPLFFLFTITSVNDFLVTVTDGTTTFVVNISDVTGVGFLPPGPPITLLPPTDVGCECECRERPIRQLLDAFIGSTVSLLASNGSIAADFSVEQTGLGIVLGTLPINPTTTVRFAISTCKITAVNITPITM

SEQ ID NO: 31 - protein, 30 aa, Bacillus mycoides
MDEFLYFAALNPGSIGPTLPPVQPFQFPTG

SEQ ID NO: 32 - protein, 190 aa, Bacillus mycoides
MDEFLYFAALNPGSIGPTLPPVQPFQFPTGPTGSTGATGSTGSTGSTGPTGSTGSTGSTGSTGPTGPTGPTGSTGPTGPTGFNLPAGPASITLTSNETTACVSTQGNNTLFFSGQVLVNGSPTPGVVVSFSFSNPSLAFMVPLAVITNASGNFTAVFLAANGPGTVTVTASLLDSPGTMASVTITIVNCP

SEQ ID NO: 33 - protein, 21 aa, Bacillus mycoides
MDSKNIGPTFPPLPSINFPTG

SEQ ID NO: 34 - protein, 335 aa, Bacillus mycoides
MDSKNIGPTFPPLPSINFPTGVTGETGATGETGATGATGETGATGETGETGATGATGATGATGETGATGATGATGAAGATGETGATGETGATGETGATGETGATGVTGETGATGETGAAGETGITGVTGPTGETGATGETGATGATGITGATGITGVAGATGETGAAGETGPTGATGAIGAIGATGATGITGVTGATGETGAAGATGITGVTGATGETGAAGATGITGATGITGVAGATGITGPTGIPGTIPTTNLLYFTFSDGEKLIYTNADGIAQYGTTQILSPSEVSYINLFINGILQPQPFYEVTAGQLTLLDDEPPSQGSSIILQFIIIN

SEQ ID NO: 35 - protein, 22 aa, Bacillus thuringiensis
MIGPENIGPTFPILPPIYIPTG

SEQ ID NO: 36 - protein, 234 aa, Bacillus thuringiensis
MIGPENIGPTFPILPPIYIPTGETGPTGITGATGETGPTGITGPTGITGATGETGSTGITGATGETGSTGITGPIGITGATGETGPIGITGATGETGPTGITGSTGITGLTGVTGLTGETGPIGITGPTGITGPTGVTGATGPTGGIGPITTTNLLYYTFADGEKLIYTDTDGIPQYGTTNILSPSEVSYINLFVNGILQPQPLYEVSTGKLTLLDTQPPSQGSSIILQFIIIN

SEQ ID NO: 37 - DNA, 299 bp, Bacillus cereus
tttcttaatcctttaccctttacttttgtaaaagttgatacacttccatccggctctgtaatttctaattcatcaataaatggtcttcgcaaaaagcctgtaattttatcataaacaattaaacgagtgagcctaaaagcagctaacgcgaaaataaaaaataaaagccagcttgtaaacagcataattccaccttcccttatcctctttcgcctatttaaaaaaaggtcttgagattgtgaccaaatctcctcaactccaatatcttattaatgtaaatacaaacaagaagataagga

SEQ ID NO: 38 - DNA, 58 bp, Bacillus cereus
accaaatctcctcaactccaatatcttattaatgtaaatacaaacaagaagataagga

SEQ ID NO: 39 - DNA, 300 bp, Bacillus anthracis
accacctaccgacgatccaatctgtacattcctagctgtaccaaatgcaagattaatatcgactaacacttgtcttactgttgatttaagttgcttctgtgcgattcaatgcttgcgtgatgttacgatttaaaactaaataatgagctaagcatggattgggtggcagaattatctgccacccaatccatgcttaacgagtattattatgtaaatttcttaaaattgggaacttgtctagaacatagaacctgtccttttcattaactgaaagtagaaacagataaaggagtgaaaaac

SEQ ID NO: 40 - DNA, 58 bp, Bacillus anthracis
acatagaacctgtccttttcattaactgaaagtagaaacagataaaggagtgaaaaac

SEQ ID NO: 41 - DNA, 300 bp, Bacillus anthracis
tagaagaagaacgccgactactttatgtcgcaattacacgggcgaaagaagaactttacatttcctctccgcaattttttagaggaaaaaaattagatatatctcgttttttatacactgtgcgaaaagatttacctgaaaagacatccactaaataaggatgtctttttttatattgtattatgtacatccctactatataaattccctgcttttatcgtaagaattaacgtaatatcaaccatatcccgttcatattgtagtagtgtatgtcagaactcacgagaaggagtgaacata

SEQ ID NO: 42 - DNA, 63 bp, Bacillus anthracis
tcaaccatatcccgttcatattgtagtagtgtatgtcagaactcacgagaaggagtgaacata

SEQ ID NO: 43 - protein, 35 aa, Bacillus cereus
MSNNNIPSPFFFNNFNPELIGPTFPPIPPLTLPTG

SEQ ID NO: 44 - protein, 222 aa, Bacillus cereus
MSNNNIPSPFFFNNFNPELIGPTFPPIPPLTLPTGPTGSTGATGATGPTGATGPTGATGPTGATGATGSTGATGPTGATGTFSSANASIVTPAPQTVNNLAPIQFTAPVLISKNVTFNGIDTFTIQIPGNYFFIGAVMTSNNQAGPVAVGVGFNGIPVPSLDGANYGTPTGQEVVCFGFSGQIPAGTTINLYNISDKTISIGGATAAGSSIVAARLSFFRIS

SEQ ID NO: 45 - protein, 41 aa, Bacillus cereus
MFSEKKRKDLIPDNFLSAPALDPNLIGPTFPPIPSFTLPTG

SEQ ID NO: 46 - protein, 293 aa, Bacillus cereus
MFSEKKRKDLIPDNFLSAPALDPNLIGPTFPPIPSFTLPTGSTGPTGPTGDTGPTGPTATICIRTDPDNGCSVAEGSGTVASGFASHAEACNTQAIGDCSHAEGQFATASGTASHAEGFQTTASGFASHTEGSGTTADANFSHTEGINTIVDVLHPGSHIMGKNGTTRSSFSWHLANGLAVGPSLNSAVIEGVTGNLYLDGVVISPNAADYAEMFETIDGNLIDVGYFVTLYGEKIRKANANDDYILGVVSATPAMIADASDLRWHNLFVRDEWGRTQYHEVVVPEKKMAMEE

SEQ ID NO: 47 - protein, 49 aa, Bacillus cereus
MTRKDKFNRSRISRRDRFNSPKIKSEILISPDLVGPTFPPIPSFTLPTG

SEQ ID NO: 48 - protein, 83 aa, Bacillus cereus
MTRKDKFNRSRISRRDRFNSPKIKSEILISPDLVGPTFPPIPSFTLPTGVTGPTGNTGPTGITGPTGDTGPTGDTGPTGITGP

SEQ ID NO: 49 - protein, 38 aa, Bacillus cereus
MSRKDRFNSPKIKSEISISPDLVGPTFPPIPSFTLPTG

SEQ ID NO: 50 - protein, 163 aa, Bacillus cereus
MSRKDRFNSPKIKSEISISPDLVGPTFPPIPSFTLPTGITGPTGNTGPTGDTGPTGPTFNINFRAEKNGAQSFTPPADIQVSYGNIIFNNGGGYSSVTNTFTAPINGIYLFSANIGFNPTLGTTSTLRITIRKNLVSVASQTIDIQFSAAESGTLTVGSSNFF

SEQ ID NO: 51 - protein, 39 aa, Bacillus cereus
MKERDNKGKQHSLNSNFRIPPELIGPTFPPVPTGFTGIG

SEQ ID NO: 52 - protein, 323 aa, Bacillus cereus
MKERDNKGKQHSLNSNFRIPPELIGPTFPPVPTGFTGIGITGPTGPQGPTGPQGPRGFQGPMGEMGPTGPQGVQGIQGPAGQMGATGPEGQQGPEGLRGPVGATGATGLQGVQGIQGPIGSTGATGAQGIQGIQGLQGPIGATGPEGPQGIQGVQGLPGATGPQGVQGVQGVIGPQGPSGSTGGTGATGQGVTGPTGITGSTGVTGPSGGPPGPTGPTGATGPGGGPSGSTGVTGSTGNTGATGSPGVTGATGPTGSTGATGIQGSQGIQGIQGIQGPLGPTGPEGPQGIQGIPGPTGITGEQGIQGVQGIQGITGATGDQGT

SEQ ID NO: 53 - protein, 39 aa, Bacillus cereus
MRERDNKRQQHSLNPNFRISPELIGPTFPPVPTGFTGIG

SEQ ID NO: 54 - protein, 436 aa, Bacillus cereus
MRERDNKRQQHSLNPNFRISPELIGPTFPPVPTGFTGIGITGPTGPQGPTGPQGPRGFQGPMGEMGPTGPQGVQGIQGPVGPIGATGPEGQQGPQGLRGPQGETGATGPGGVQGLQGPIGPTGATGAQGVQGIQGLQGPIGATGPEGPQGIQGVQGLPGATGSQGIQGVQGIQGPQGPSGNTGATGATGQGITGPTGITGPTGITGPSGGPPGPTGPTGATGPGGGPSGSTGATGATGNTGATGNTGITGATGSTGPTGSTGAQGLQGIQGIQGPIGPTGPEGPQGIQGIPGPTGVTGEQGIQGVQGIQGITGATGDQGPQGIQGVIGAQGVTGATGDQGPQGIQGVPGPSGATGPQGVQGIQGPMGDIGPTGPEGPEGLQGPQGIQGVPGPVGATGPEGPQGIQGIQGVQGATGPQGPQGIQGIQGVQGITGATG

SEQ ID NO: 55 - protein, 36 aa, Bacillus thuringiensis
MKNRDNKGKQQSNFRIPPELIGPTFPPVPTGFTGIG

SEQ ID NO: 56 - protein, 470 aa, Bacillus thuringiensis
MKNRDNKGKQQSNFRIPPELIGPTFPPVPTGFTGIGITGPTGPQGPTGPQGPRGFQGPMGEMGPTGPQGVQGIQGPVGPIGATGPEGQQGAQGLRGPQGETGATGPQGVQGLQGPIGPTGATGAQGIQGIQGLQGPIGATGPEGPQGIQGVQGLPGATGPQGIQGAQGIQGTQGPSGNTGATGATGQGLTGPTGITGPTGITGPSGGPPGPTGPTGATGPGGGPSGSTGATGATGDTGATGSTGVTGATGAQGPQGVQGIQGPTGATGATGATGPQGIQGPQGIQGPTGATGATGSQGPTGNTGPTGSQGIQGPTGPTGAGATGATGATGATGVSTTATYAFANNTSGSIISVLLGGTNIPLPNNQNIGPGITVSGGNTVFTVANAGNYYIAYTINLTAGLLVSSRITVNGSPLAGTINSPAVAAGSFSATIIANLPAGAAVSLQLFGVIALATLSTATPGATLTIIRLS

SEQ ID NO: 57 - protein, 136 aa, Bacillus mycoides
MKFSKKSTVDSSIVGKRVVSKVNILRFYDARSCQDKDVDGFVDVGELFTIFRKLNMEGSVQFKAHNSIGKTYYITINEVYVFVTVLLQYSTLIGGSYVFDKNEIQKINGILQANALNPNLIGPTLPPIPPFTLPTG

SEQ ID NO: 58 - protein, 384 aa, Bacillus mycoides
MKFSKKSTVDSSIVGKRVVSKVNILRFYDARSCQDKDVDGFVDVGELFTIFRKLNMEGSVQFKAHNSIGKTYYITINEVYVFVTVLLQYSTLIGGSYVFDKNEIQKINGILQANALNPNLIGPTLPPIPPFTLPTGPTGGTGPTGVTGPTGVTGPTGVTGPTGVTGPTGVTGPTGVTGPTGVTGPTGVTGPTGVTGPTGVTGPTGVTGPTGVTGPTGGTEGCLCDCCVLPMQSVLQQLIGETVILGTIADTPNTPPLFFLFTITSVNDFLVTVTDGTTTFVVNISDVTGVGFLPPGPPITLLPPTDVGCECECRERPIRQLLDAFIGSTVSLLASNGSIAADFSVEQTGLGIVLGTLPINPTTTVRFAISTCKITAVNITPITM

SEQ ID NO: 59 - protein, 36 aa, Bacillus cereus
MKERDKQNSLNSNFRISPNLIGPTFPPVPTGFTGIG

SEQ ID NO: 60 - protein, 1321 aa, Bacillus cereus
MKERDKQNSLNSNFRISPNLIGPTFPPVPTGFTGIGITGPTGPQGPTGPQGPRGLQGPMGEMGPTGPQGVQGIQGPVGSIGATGPEGQQGPQGLRGPQGETGATGPQGVQGLQGPAGPTGATGAQGIQGIQGLQGPIGATGPEGPQGIQGVQGLPGATGPQGIQGAQGMQGLQGPSGNTGATGATGQGITGPTGVTGPTGITGPSGGPPGPTGPTGATGPGGGPSGSTGATGATGNTGATGSTGVTGSTGVTGATGSTGPTGSTGAQGLQGIQGIQGPIGPTGPEGPQGIQGIPGPTGVTGEQGIQGVQGIQGATGATGDQGPQGIQGAIGPQGATGATGDQGPQGIQGVPGPSGATGPQGVQGLQGPMGDIGPTGPEGPEGLQGPQGIQGVPGPVGATGPEGPQGIQGIQGPVGATGPQGPQGIQGIQGVQGITGATGVQGATGIQGIQGEIGATGPEGPQGVQGAQGGIGPTGPMGPQGVQGVQGIQGATGAQGVQGPQGIQGIQGIQGIQGPTGATGDTGATGATGEGTTGPTGVTGPTGPSGGPAGPTGPTGPSGPAGVTGPSGGPPGPTGATGATGVTGDTGATGSTGVTGATGETGATGVTGLQGPQGIQGVQGEIGPTGPQGVQGPQGIQGVTGATGDQGPQGVQGPQGDIGPTGPQGIQGPQGSQGIQGATGGTGAQGPQGIQGPQGDVGPTGPQGPTGIQGIQGEIGPTGPEGPEGLQGPQGIQGVQGPVGATGPEGPQGIQGIQGVQGATGSQGPQGIQGIQGVQGITGATGAQGATGIQGIQGEIGATGPEGPQGVQGVQGEIGPTGPMGPQGVQGVQGIQGATGAQGVQGPQGIQGIQGPTGATGETGATGATGEGTTGPTGVTGPTGVTGPSGGPAGPTGPTGPSGPAGVTGPSGGPPGPTGATGATGATGVTGDTGATGSTGVTGATGETGATGVTGLQGPQGIQGVQGEIGPTGPQGIQGPQGIQGVTGATGAQGPQGIQGPQGDIGPTGPQGIQGPQGPQGIQGATGATGAQGPQGIQGPQGEIGPTGPQGPQGIQGPQGIQGPTGATGATGATGLQGIQGPQGIQGPQGIQGPTGATGATGATGLQGIQGPQGIQGPQGIQGPTGATGATGATGLQGIQGPQGIQGPQGIQGPTGATGATGATGSQGPTGDTGPTGAGATGATGATGVSTTATYAFANNTSGTAISVLLGGTNIPLPNNQNIGPGITVSGGNTVFTVASAGNYYIAYTINLTAGLLVSSRITVNGSPLAGTINAPTVATGSFSATIIANLPAGAAISLQLFGLVAIATLSTTTPGATLTIIRLS

SEQ ID NO: 61 - protein, 39 aa, Bacillus cereus
MMENKKGSKHNEFLSAKAFNPNLVGPTLPPVPSFTLPTG

SEQ ID NO: 62 - protein, 309 aa, Bacillus cereus
MMENKKGSKHNEFLSAKAFNPNLVGPTLPPVPSFTLPTGPTGATGATGVTGATGATGATGATGVTGATGATGVTGATGATGVTGATGATGATGATGVTGATGATGVTGATGATGVTGATGATGVTGVTGATGATGATGVTGPTGATGATGATGVTGPTGATGATGATGGLAVASASAMTSTAQTVDNLVAVQFTAPVLELDSVIFNGTDTFTVLVPGNYYCIGSLMPAETQTGPFAVGIGLNGIPVPALDGANYAQSAGQEVVGFGLTGQIPAGTTISLFNLSGHTISIGGTISGATSVAARLLLFRIS

SEQ ID NO: 63 - protein, 41 aa, Bacillus weihenstephanensis
MSNNNYSDGLNPDEFLSASAFDPNLVGPTLPPIPPFTLPTG

SEQ ID NO: 64 - protein, 292 aa, Bacillus weihenstephanensis
MSNNNYSDGLNPDEFLSASAFDPNLVGPTLPPIPPFTLPTGPTGPTGPTGPTGPTVPTGPTGPTGPTGPTGPTGDTGTTGATGDTGATGDTGATGPTGPTGDTGATGPTGPTGDTGATGPTGPTGDTGATGPTGPTGDTGATGATGPTGPTGPTGPSGLGLPAGLYAFNSATISLALGINDPVPFNTVGSQFGTAISQLDADTFIISETGFYKITVIAYTAAVSILGSLAIQVNGVNIPGAGTSLISLGAPLVIQAITQITITPSMVEAVVTGLGLSLALGTSASIIIEKIA

SEQ ID NO: 65 - protein, 30 aa, Bacillus thuringiensis
MDEFLSSAAINPNLVGPTLPPVPPFTLPTG

SEQ ID NO: 66 - protein, 233 aa, Bacillus thuringiensis
MDEFLSSAAINPNLVGPTLPPVPPFTLPTGPTGSTGPTGTTGPTGPTGTTGTTGTTGPTGPTGTTGPTGPTGTTGTTGPTGTTGTLSVAYGHFWQTDIITVPFESPFSFDQAGPMVGGISLLNPTTISITQPGDYRVSFISSINLTVALVFPYSPTISILLNNSLIPNFKATFGLLIQDLEDVDCDQLTGETILSIPANSTLQLINNSFVGNRDIRTCDNGINALELTIIKLN

SEQ ID NO: 67 - protein, 33 aa, Bacillus cereus
MFDKNKILQANAFNSNLIGPTLPPIPPFTLPTG

SEQ ID NO: 68 - protein, 295 aa, Bacillus cereus
MFDKNKILQANAFNSNLIGPTLPPIPPFTLPTGPTGGTGPTGVTGPTGVTGPIGVTGPTGVTGPTGVTGPTGITGPTGVTGPTGVTGPTGVTGPTGVTGPTGVTGPTGVTGPTGVTGPTGVTGPTGVTGPTGSTESCLCDCCVLPMQNVLQQLIGETVLLGTIADAPNTPPLFFLFTITSVNDFLVTVTDGSTSYVVNISDVTGVGFLPPGPSITLLPPVDIGCECDCRERPIRELLDTLIGSTVNLLASTGSIAADFNVEQTGLGIVLGTLPINPTTIVRFAISTCKITAVNIL

SEQ ID NO: 69 - protein, 44 aa, Bacillus cereus
MSDENEKKYSNELAQADFISAAAFDPSLVGPTLPPTPPFTLPTG

SEQ ID NO: 70 - protein, 639 aa, Bacillus cereus
MSDENEKKYSNELAQADFISAAAFDPSLVGPTLPPTPPFTLPTGPTGATGPTGATGPTGATGSTGVTGPTGVTGPTGATGPTGATGSTGVTGPTGATGPSGATGSTGTTGPTGDTGPTGITGPTGVTGPTGATGPTGATGPTGATGSTGVTGPTGVTGPTGATGPTGATGSTGVTGPTGITGPTGATGTTGSTGPTGVTGPTGATGPTGATGPTGATGSTGVTGPTGITGPTGATGTTGSTGPTGVTGPTGATGPTGATGPTGATGSTGVTGPTGVTGPTGATGPTGATGSTGVTGPTGATGPTGATGSTGVTGPTGVTGPTGATGSTGATGPTGATGSTGVTGPTGATGPTGATGPTGATGSTGVTGPTGITGPTGATGTTGSTGPTGVTGPTGVTGPTGVTGPTGATGPTGATGSTGVTGPTGITGPTGATGTTGSTGPTGVTGPTGVTGPTGATGPTGVTGPTGATGPTGATGATATTSTKAILFGGTNAGFQRIAGSPGADSQTLPYVTAGAGSVVAFSASINVNNLGTGVYLLRVCDNVPTNLASPGAGQIVSTITLTLTANITGTIVFSIKPTDIGAQPVKVFNPNPVVAPATVTWTSTIPGNPVARTDAISLFITPGITQSAVYSVFISTAV

SEQ ID NO: 71 - protein, 38 aa, Bacillus cereus; identical to SEQ ID NO: 49

SEQ ID NO: 72 - protein, 163 aa, Bacillus cereus
MSRKDRFNSPKIKSEISISPDLVGPTFPPIPSFTLPTGITGPTGNTGATGDTGPTGPTFNINFRAEKNGAQSFTPPADIQVSYGNIIFNNGGGYSSVTNTFTAPINGIYLFSANIGFNPTLGTTSTLRITIRKNLVSVASQTIDIQFSAAESGTLTVGSSNFF

SEQ ID NO: 73 - protein, 30 aa, Bacillus cereus
MDEFLSSAALNPGSVGPTLPPMQPFQFSTG

SEQ ID NO: 74 - protein, 77 aa, Bacillus cereus
MDEFLSSAALNPGSVGPTLPPMQPFQFSTGPTGSTGATGATGNTEPYWHTGPPGIVLLTYDFKSLIISFAFQILPIS

SEQ ID NO: 75 - protein, 48 aa, Bacillus weihenstephanensis
MFLGGGYMERKNKWYGLNSNVNLSASSFDPNLVGPTLPPISPISVPTG

SEQ ID NO: 76 - protein, 247 aa, Bacillus weihenstephanensis
MFLGGGYMERKNKWYGLNSNVNLSASSFDPNLVGPTLPPISPISVPTGPTGETGITGPTGPTGPTGPTGVTGITGPTGPTGATGITGPTGPTGETGITGPTGPGPTVSLKFLYVANFNENTVEIYDIFNPIFPVRIGEFNGGNLANPAGLAITGTTLYVTNNGDNTVEIYDILNPIAPVHVGEFNGGNLSEPDGLAITGTTLYVANFNDNTVEIYDILNPIAPVRVGEFNAGNLSSPAGLIIFSLFG

SEQ ID NO: 77 - protein, 30 aa, Bacillus cereus
MDELLSSTLINPDLLGPTLPAIPPFTLPTG

SEQ ID NO: 78 - protein, 536 aa, Bacillus cereus
MDELLSSTLINPDLLGPTLPAIPPFTLPTGPTGSTGPTGPTGSTGPTGPTGSTGPTGPTGSTGLTGLTGPTGPTGPTGPTGPTGPTGSTGLTGPTGPNSDTGPTGPTGPTGPSDGPTGPTGATGPTGPPDGPTGDTGPTGSTGPTGDTGPTGSTGPTGDTGPTGSTGPTGDTGPTGSTGPTGDTGPTGSTGPTGDTGPTGSTGPTGPGCIEPLPTFTQIVYVNKAGNDATADGSECAPFLTVTAAMASITDAIAPFPDPLNITKRYAISIGPGNYIEPLIHLKANVQLVGTSTLLTRLQIPFDINDPSWFDLNFSQDPRSGFVNLTLLSGPLDFNFQTAQSVSGKLFFVSVNITPTPIFTALSTSVNQVNIRDSMLSGGYTQNGINMAMFASFVSSGNITINSQATTDTQVNLVGGGINGNVIINVLPGHIPIDPLNLTSFAITENIFNPSPNSGNLFVNGANNVITRVRATVDSLPIRSRINLIGTSTSLIRVDDAFDLAYTPINPANWAPLPPTTVQEALDRIAALMAITIGTP

SEQ ID NO: 79 - protein, 39 aa, Bacillus cereus; identical to SEQ ID NO: 19

SEQ ID NO: 80 - protein, 1309 aa, Bacillus cereus
MKNRDNNRKQNSLSSNFRIPPELIGPTFPPVPTGFTGIGITGPTGPQGPTGPQGPRGLQGPMGEMGPTGPQGVQGIQGSVGPIGATGPEGQQGPQGLRGPQGETGATGPGGVQGLQGPIGPTGATGAQGIQGIQGLQGPIGATGPEGSQGIQGVQGLPGATGPQGIQGAQGIQGTPGPSGNTGATGATGATGQGITGPTGITGPTGITGPSGGPPGPTGPTGATGPGGGPSGSTGATGATGNTGATGSTGVTGATGSTGPTGSTGAQGLQGIQGIQGPIGPTGPEGSQGIQGIPGPTGVTGEQGIQGVQGIQGATGATGDQGPQGIQGVIGPQGVTGATGDQGPQGIQGVPGPSGETGPQGVQGIQGPMGDIGPTGPEGPEGLQGPQGIQGVPGPVGATGPEGPQGIQGIQGPVGATGPQGPQGIQGIQGVQGITGATGVQGATGIQGIQGEIGATGPEGPQGVQGAQGAIGPTGPMGPQGVQGVQGIQGATGAQGVQGPQGIQGIQGPTGATGDMGATGATGEGTTGPTGVTGPTGVTGPSGGPAGPTGPTGPSGPAGVTGPSGGPPGPTGATGATGVTGDTGATGSTGVTGATGETGATGVTGLQGPQGIQGVQGEIGPTGPQGVQGPQGIQGVTGATGDQGPQGIQGPQGDIGPTGPQGIQGPQGSQGIQGATGGTGAQGPQGIQGPQGDIGPTGSQGPTGIQGIQGEIGPTGPRRPEGCRGRKRIQGVQGPVGATGPEGPQGIQGIQGVQGATGPQGPQGIQGIQGVQGITGATGAQGATGIQGIQGEIGATGPEGPQGVQGIQGAIGPTGPMGAQGVQGIQGIQGATGAQGVQGPQGIQGVQGPTGATGDTGATGATGEGTTGPTGVTGPTGVTGPSGGPAGPTGPTGPSGPAGVTGPSGGPPGPTGATGATGVTGDTGATGSTGVTGATGATGVTGLQGPQGIQGVQGEIGPTGPQGIQGPQGIQGVTGATGAQGPQGIQGPQGDIGPTGSQGIQGPQGPQGIQGATGATGAQGPQGIQGPQGEIGPTGPQGPQGIQGPQGIQGPTGATGATGATGPQGIQGPQGIQGPQGIQGPTGVTGATGATGPQGIQGPQGIQGPQGIQGPTGATGATGATGPQGIQGPQGIQGPQGIQGPTGATGATGSQGPTGDTGPTGAGATGATGATGVSTTATYAFANNTSGTAISVLLGGTNVPLPNNQNIGPGITVSGGNTVFTVANAGNYYIAYTINLTAGLLVSSRITVNGSPLAGTINAPTVATGSFSATIIANLPAGAAVSLQLFGVVAVATLSTATPGATLTIIRLS

SEQ ID NO: 81 - protein, 44 aa, Bacillus cereus
MVKVVEGNSGKSKIKSSLNSNFKLSSGLVGPTFPPVPTGMTGIT

SEQ ID NO: 82 - protein, 815 aa, Bacillus cereus
MVKVVEGNSGKSKIKSSLNSNFKLSSGLVGPTFPPVPTGMTGITGSTGATGNTGPTGETGATGSTGVTGNTGPTGETGVTGSTGPTGETGSTGNTGATGETGPTGSTGATGSTGVTGSTGPTGNTGPTGETGVTGSTGPTGNTGATGETGPTGSTGPTGETGVTGSTGVTGPTGATGNTGATGSTGVTGNTGPTGETGVTGSTGPTGNTGATGNTGPTGETGSTGVTGSTGATGSTGVTGPTGVTGPTGETGVTGSTGPTGNTGATGNTGPTGETGVTGSTGPTGNTGATGNTGATGETGSTGVTGSTGVTGSTGATGSTGVTGNTGATGSTGATGNTGPTGATGSTGVTGNTGPTGETGPTGSTGPTGNTGATGNTGATGATGATGATGSTGPTGNTGATGNTGPTGVTGSTGPTGSTGETGETGPTGETGVTGSTGPTGPTGATGNTGPTGETGATGSTGETGETGPTGETGVTGSTGPTGNTGATGNTGPTGETGVTGSTGPTGNTGATGNTGPTGETGETGVTGSTGVTGNTGATGSTGATGNTGPTGETGATGPTGATGVTGSTGPTGNTGPTGETGSTGSTGATGSTGVTGNTGATGETGPTGSTGATGNTGATGETGPTGATGVTGPTGSTGVTGSTGPTGSTGATGATGSTGPTGSTGTTGDTGPTGATGVSTTATYAFANNTSGSVISVLLGGTNIPLPNNQNIGPGITVSGGNTVFTVANAGNYYIAYTINLTAGLLVSSRITVNGSPLAGTINSPTVATGSFNATIIASLPAGAAVSLQLFGVVALATLSTATPGATLTIIRLS

SEQ ID NO: 83 - protein, 40 aa, Bacillus anthracis
MEGNGGKSKIKSPLNSNFKILSDLVGPTFPPVPTGMTGIT

SEQ ID NO: 84 - protein, 469 aa, Bacillus anthracis
MEGNGGKSKIKSPLNSNFKILSDLVGPTFPPVPTGMTGITGSTGATGNTGPTGETGATGSAGITGSTGPTGNTGGTGSTGSTGNTGATGSTGVTGSTGVTGSTGVTGSTGVTGSTGPTGETGGTGSTGVTGSTGATGSTGVTGSTGVTGETGPTGSTGATGNTGPTGETGGTGSTGATGSTGVTGNTGPTGSTGVTGNTGATGETGPTGNTGATGNTGPTGETGVTGSTGPTGETGVTGSTGPTGNTGATGETGATGSTGVTGNTGSTGETGPTGSTGPTGSTGATGVTGNTGPTGSTGATGATGSTGPTGSTGTTGNTGVTGDTGPTGATGVSTTATYAFANNTSGSVISVLLGGTNIPLPNNQNIGPGITVSGGNTVFTVANAGNYYIAYTINLTAGLLVSSRITVNGSPLAGTINSPTVATGSFSATIIASLPAGAAVSLQLFGVVALATLSTATPGATLTIIRLS

SEQ ID NO: 85 - protein, 34 aa, Bacillus thuringiensis; identical to SEQ ID NO: 7

SEQ ID NO: 86 - protein, 285 aa, Bacillus thuringiensis
MKQNDKLWLDKGIIGPENIGPTFPVLPPIHIPTGITGATGATGITGATGPTGTTGATGATGITGVTGATGITGVTGATGITGATGPTGITGATGPAGITGATGPAGITGATGPAGITGATGPTGITGATGPTGITGATGPTGITGATGPAGITGATGPTGTTGVTGATGITGVTGATGITGATGPTGTTGVTGPTGVIGPITTTNLLFYTFADGEKLIYTDSDGLAQYGTTHILSPDEVSYINLFINGILQPQPLYQVSTGQLTLLDNQPPSQGSSIILQFIIIN

SEQ ID NO: 87 - protein, 34 aa, Bacillus cereus
MNSNEKLSLNKGMVRPENIGPTFPVLPPIYIPTG

SEQ ID NO: 88 - protein, 258 aa, Bacillus cereus
MNSNEKLSLNKGMVRPENIGPTFPVLPPIYIPTGATGATGATGATGATGATGATGATGATGATGATGATGATGATGVTGATGATGATGVTGATGATGVTGATGVTGATGATGVTGATGVTGATGATGATGVTGATGATGATGATGVTGATGVTGVTGVTGATGATGPTGVIGPITTTNLLFYTFSDGEKLIYTDSDGLAQYGTTHILSPDEVSYINLFINGILQPQPLYQVSTGQLTLLDNQPPSQGSSIILQFIIIN

SEQ ID NO: 89 - protein, 34 aa, Bacillus cereus
MKRNDNLSLNKGMIGPENIGPTFPILPPIYIPTG

SEQ ID NO: 90 - protein, 279 aa, Bacillus cereus
MKRNDNLSLNKGMIGPENIGPTFPILPPIYIPTGATGPTGITGPTGETGPTGITGPTGVTGPTGITGPTGATGPTGITGPTGATGPTGITGPTGATGPTGETGPTGITGPTGATGPTGITGPTGATGPTGETGPTGETGPTGVTGPTGITGPTGATGPTGITGPTGATGPTGETGPTGITGPTGATGPTGGIGPITTTNLLYYTFADGEKLIYTDADGIPQYGTTNILSPSEVSYINLFVNGILQPQPLYEVSTGKLTLLDTQPPSQGSSIILQFIIIN

SEQ ID NO: 91 - protein, 99 aa, Bacillus cereus
MDSFVDVGEIFTIFRKLNMEGSLQFKVHNSMGKTYYITINEVYVYVTVLLQYSTLIGGSYVFDKNEIQKINGILQANALNPNLIGPTLPPIPPFTLPTG

SEQ ID NO: 92 - protein, 145 aa, Bacillus cereus
MDSFVDVGEIFTIFRKLNMEGSLQFKVHNSMGKTYYITINEVYVYVTVLLQYSTLIGGSYVFDKNEIQKINGILQANALNPNLIGPTLPPIPPFTLPTGPTGVTGPTGGTGPTGVTGPTGVTGPTGVTGVTGPTGVTGPTGVTGP

SEQ ID NO: 93 - protein, 136 aa, Bacillus weihenstephanensis
MKFSKKSTVDSSIVGKRVVSKVNILRFYDARSWQDKDVDGFVDVGELFTIFRKLNMEGSVQFKAHNSIGKTYYITINEVYVFVTVLLQYSTLIGGSYVFDKNEIQKINGILQANALNPNLIGPTLPPIPPFTLPTG

SEQ ID NO: 94 - protein, 142 aa, Bacillus weihenstephanensis
MKFSKKSTVDSSIVGKRVVSKVNILRFYDARSWQDKDVDGFVDVGELFTIFRKLNMEGSVQFKAHNSIGKTYYITINEVYVFVTVLLQYSTLIGGSYVFDKNEIQKINGILQANALNPNLIGPTLPPIPPFTLPTGPTGGTG

SEQ ID NO: 95 - protein, 196 aa, Bacillus anthracis
MSNNNYSNGLNPDESLSASAFDPNLVGPTLPPIPPFTLPTGPTGPFTTGPTGPTGPTGPTGPTGPTGPTGPTGDTGTTGPTGPTGPTGPTGPTGPTGPTGPTGPTGFTPTGPTGPTGPTGDTGTTGPTGPTGPTGPTGPTGDTGTTGPTGPTGPTGPTGPTGPTGPTFTGPTGPTGPTGATGLTGPTGPTGPSGLG

SEQ ID NO: 96 - protein, 17 aa, Bacillus anthracis
MAFDPNLVGPTLPPIPP

SEQ ID NO: 97 - protein, 17 aa, Bacillus anthracis
MALEPNLIGPTLPPIPP

SEQ ID NO: 98 - protein, 17 aa, Bacillus weihenstephanensis
MALNPNLIGPTLPPIPP

SEQ ID NO: 99 - protein, 17 aa, Bacillus weihenstephanensis
MALDPNIIGPTLPPIPP

SEQ ID NO: 100 - protein, 17 aa, Bacillus cereus
MALEPNLIGPTLPSIPP

SEQ ID NO: 101 - protein, 17 aa, Bacillus weihenstephanensis
MALDPNLIGPPLPPITP

SEQ ID NO: 102 - protein, 17 aa, Bacillus weihenstephanensis
MALNPGSIGPTLPPVPP

SEQ ID NO: 103 - protein, 17 aa, Bacillus weihenstephanensis
MALNPCSIGPTLPPMQP

SEQ ID NO: 104 - protein, 17 aa, Bacillus mycoides
MALNPGSIGPTLPPVQP

SEQ ID NO: 105 - protein, 17 aa, Bacillus anthracis
MALNPGSVGPTLPPMQP

SEQ ID NO: 106 - protein, 17 aa, Bacillus cereus
MALDPNLIGPTFPPIPS

SEQ ID NO: 107 - protein, 18 aa, Bacillus thuringiensis
MAAINPNLVGPTLPPVPP

SEQ ID NO: 108 - protein, 799 aa, Bacillus mycoides
MKRKTPFKVFSSLAITTMLGCTFALGTSVAYAETTSQSKGSISTTPIDNNLIQEERLAEALKERGTIDQSASKEETQKAVEQYIEKKKGDQPNKEILPDDPAKEASDFVKKVKEKKMEEKEKVKKSVENASSEQTPSQNKKQLNGKVPTSPAKQAPYNGAVRTDKVLVLLVEFSDYKHNNIEQSPGYMYANDFSREHYQKMLFGNEPFTLFDGSKVKTFKQYYEEQSGGSYTTDGYVTEWLTVPGKAADYGADGKTGHDNKGPKGARDLVKEALKAAAEKGLDLSQFDQFDRYDTNGDGNQNEPDGVIDHLMVIHAGVGQEAGGGKLGDDAIWSHRSKLAQDPVAIEGTKSKVSYWDGKVAAHDYTIEPEDGAVGVFAHEFGHDLGLPDEYDTNYTGAGSPVEAWSLMSGGSWTGRIAGTEPTSFSPQNKDFLQKNMDGNWAKIVEVDYDKIKRGVGFPTYIDQSVTKSNRPGLVRVNLPEKSVETIKTGFGKHAYYSTRGDDMHTTLETPLFDLTKAANAKFDYKANYELEAECDFIEVHAVTEDGTKTLIDKLGDKVVKGDQDTTEGKWIDKSYDLSQFKGKKVKLQFDYITDPALTYKGFAMDNVNVTVDGKVVFSDDAEGQAKMKLNGFVVSDGTEKKPHYYYLEWRNYAGSDEGLKVGRGPVYNTGLVVWYADDSFKDNWVGRHPGEGFLGVVDSHPEAVVGNLNGKPVYGNTGLQIADAAFSLDQTPAWNVNSFTRGQFNYPGLPGVATFDDSKVYSNTQIPDAGRKVPQLGLKFQVVGQADDKSAGAIWIRR

SEQ ID NO: 109 - protein, 152 aa, Bacillus anthracis
MSCNENKHHGSSHCVVDVVKFINELQDCSTTTCGSGCEIPFLGAHNTASVANTRPFILYTKAGAPFEAFAPSANLTSCRSPIFRVESVDDDSCAVLRVLSVVLGDSSPVPPTDDPICTFLAVPNARLVSTSTCITVDLSCFCAIQCLRDVTI

SEQ ID NO: 110 - protein, 167 aa, Bacillus anthracis
MFSSDCEFTKIDCEAKPASTLPAFGFAFNASAPQFASLFTPLLLPSVSPNPNITVPVINDTVSVGDGIRILRAGIYQISYTLTISLDNSPVAPEAGRFFLSLGTPANIIPGSGTAVRSNVIGTGEVDVSSGVILINLNPGDLIRIVPVELIGTVDIRAAALTVAQIS

SEQ ID NO: 111 - protein, 156 aa, Bacillus anthracis
MSCNCNEDHHHHDCDFNCVSNVVRFIHELQECATTTCGSGCEVPFLGAHNSASVANTRPFILYTKAGAPFEAFAPSANLTSCRSPIFRVESIDDDDCAVLRVLSVVLGDTSPVPPTDDPICTFLAVPNARLISTNTCLTVDLSCFCAIQCLRDVTI

SEQ ID NO: 112 - protein, 182 aa, Bacillus anthracis
MEVGGTSVKNKNKSSTVGKPLLYIAQVSLELAAPKTKRIILTNFENEDRKEESNRNENVVSSAVEEVIEQEEQQQEQEQEQEEQVEEKTEEEEQVQEQQEPVRTVPYNKSFKDMNNEEKIHFLLNRPHYIPKVRCRIKTATISYVGSIISYRNGIVAIMPPNSMRDIRLSIEEIKSIDMAGF

SEQ ID NO: 113 - protein, 174 aa, Bacillus anthracis
MKERSENMRSSSRKLTNFNCRAQAPSTLPALGFAFNATSPQFATLFTPLLLPSTGPNPNITVPVINDTISTGTGIRIQVAGIYQISYTLTISLDNVPVTPEAARFFLTLNSSTNIIAGSGTAVRSNIIGTGEVDVSSGVILINLNPGDLIQIVPVEVIGTVDIRSAALTVAQIR

SEQ ID NO: 114 - protein, 796 aa, Bacillus thuringiensis
MSKKPFKVLSSIALTAVLGLSFGAGTQSAYAETPVNKTATSPVDDHLIPEERLADALKKRGVIDSKASETETKKAVEKYVENKKGENPGKEAANGDQLTKDASDFLKKVKDAKADTKEKLNQPATGTPAATGPVKGGLNGKVPTSPAKQKDYNGEVRKDKVLVLLVEYADFKHNNIDKEPGYMYSNDFNKEHYEKMLFGNEPFTLDDGSKIETFKQYYEEQSGGSYTVDGTVTKWLTVPGKAADYGADAPGGGHDNKGPKGPRDLVKDALKAAVDSGIDLSEFDQFDQYDVNGDGNKNQPDGLIDHLMIIHAGVGQEAGGGKLGDDAIWSHRWTVGPKPFPIEGTQAKVPYWGGKMAAFDYTIEPEDGAVGVFAHEYGHDLGLPDEYDTQYSGQGEPIEAWSIMSGGSWAGKIAGTTPTSFSPQNKEFFQKTIGGNWANIVEVDYEKLNKGIGLATYLDQSVTKSARPGMIRVNLPDKDVKTIEPAFGKQYYYSTKGDDLHTKMETPLFDLTNATSAKFDFKSLYEIEAGYDFLEVHAVTEDGKQTLIERLGEKANSGNADSTNGKWIDKSYDLSQFKGKKVKLTFDYITDGGLALNGFALDNASLTVDGKVVFSDDAEGTPQLKLDGFVVSNGTEKKKHNYYVEWRNYAGADNALKFARGPVFNTGMVVWYADSAYTDNWVGVHPGHGFLGVVDSHPEAIVGTLNGKPTVKSSTRFQIADAAFSFDKTPAWKVVSPTRGTFTYDGLAGVPKFDDSKTYINQQIPDAGRILPKLGLKFEVVGQADDNSAGAVRLYR

SEQ ID NO: 115 - protein, 430 aa, Bacillus cereus
MKHNDCFDHNNCNPIVFSADCCKNPQSVPITREQLSQLITLLNSLVSAISAFFANPSNANRLVLLDLFNQFLIFLNSLLPSPEVNFLKQLTQSIIVLLQSPAPNLGQLSTLLQQFYSALAQFFFALDLIPISCNSNVDSATLQLLFNLLIQLINATPGATGPTGPTGPTGPTGPAGTGAGPTGATGATGATGPTGATGPAGTGGATGATGATGVTGATGATGATGPTGPTGATGPTGATGATGATGPTGATGPTGATGLTGATGAAGGGAIIPFASGTTPSALVNALVANTGTLLGFGFSQPGVALTGGTSITLALGVGDYAFVAPRAGTITSLAGFFSATAALAPISPVQVQIQILTAPAASNTFTVQGAPLLLTPAFAAIAIGSTASGIIAEAIPVAAGDKILLYVSLTAASPIAAVAGFVSAGINIV

SEQ ID NO: 116 - protein, 437 aa, Bacillus cereus
MKHNDCFGHNNCNNPIVFTPDCCNNPQTVPITSEQLGRLITLLNSLIAAIAAFFANPSDANRLALLNLFTQLLNLLNELAPSPEGNFLKQLIQSIINLLQSPNPNLGQLLSLLQQFYSALAPFFFSLILDPASLQLLLNLLAQLIGVTPGGGATGPTGPTGPGGGATGPTGPTGPGGGATGPTGATGPTGDTGLAGATGATGPTGDTGVAGPAGPTGPTGDTGLAGATGPTGPTGDTGLAGATGPTGATGLAGATGPTGATGLTGATGATGAAGGGAIIPFASGTTPAALVNALIANTGTLLGFGFSQPGIGLAGGTSITLALGVGDYAFVAPRDGVITSLAGFFSATAALSPLSPVQVQIQILTAPAASNTFTVQGAPLLLTPAFAAIAIGSTASGIIPEAIPVVAGDKILLYVSLTAASPIAAVAGFVSAGINIV

SEQ ID NO: 117 - protein, 119 aa, Bacillus anthracis
MLFTSWLLFFIFALAAFRLTRLIVYDKITGFLRRPFIDELEITEPDGSVSTFTKVKGKGLRKWIGELLSCYWCTGVWVSAFLLVLYNWIPIVAEPLLALLAIAGAAAIIETITGYFMGE

SEQ ID NO: 118 - protein, 61 aa, Bacillus anthracis
MFAVSNNPRQNSYDLQQWYHMQQQHQAQQQAYQEQLQQQGFVKKKGCNCGKKKSTIKHYEE

SEQ ID NO: 119 - protein, 481 aa, Bacillus anthracis
MSRYDDSQNKFSKPCFPSSAGRIPNTPSIPVTKAQLRTFRAIIIDLTKIIPKLFANPSPQNIEDLIDTLNLLSKFICSLDAASSLKAQGLAIIKNLITILKNPTFVASAVFIELQNLINYLLSITKLFRIDPCTLQELLKLIAALQTALVNSASFIQGPTGPTGPTGPTGPAGATGATGPQGVQGPAGATGATGPQGVQGPAGATGATGPQGAQGPAGATGATGPQGAQGPAGATGATGPQGIQGPAGATGATGPQGVQGPTGATGIGVTGPTGPSGGPAGATGPQGPQGNTGATGPQGIQGPAGATGATGPQGAQGPAGATGATGPQGVQGPTGATGIGVTGPTGPSGPSFPVATIVVTNNIQQTVLQFNNFIFNTAINVNNIIFNGTDTVTVINAGIYVISVSISTTAPGCAPLGVGISINGAVATDNFSSNLIGDSLSFTTIETLTAGANISVQSTLNEITIPATGNTNIRLTVFRIA

SEQ ID NO: 120 - protein, 275 aa, Bacillus thuringiensis
MKMKRGITTLLSVAVLSTSLVACSGITEKTVAKEEKVKLTDQQLMADLWYQTAGEMKALYYQGYNIGQLKLDAVLAKGTEKKPAIVLDLDETVLDNSPHQAMSVKTGKGYPYKWDDWINKAEAEALPGAIDFLKYTESKGVDIYYISNRKTNQLDATIKNLERVGAPQATKEHILLQDPKEKGKEKRRELVSQTHDIVLFFGDNLSDFTGFDGKSVKDRNQAVADSKAQFGEKFIIFPNPMYGDWEGALYDYDFKKSDAEKDKIRRDNLKSFDTK

SEQ ID NO: 121 - protein, 795 aa, Bacillus thuringiensis
MKKKKKLKPLAVLTTAAVLSSTFAFGGHAAYAETPTSSLPIDEHLIPEERLAEALKQRGVIDQSASQAETSKAVEKYVEKKKGENPGKEILTGDSLTQEASDFMKKVKDAKMRENEQAQQPEVGPVAGQGAALNPGKLNGKVPTTSAKQEEYNGAVRKDKVLVLLVEFSDFKHNNIDQEPGYMYSKDFNREHYQKMLFGDEPFTLFDGSKINTFKQYYEEQSGGSYTVDGTVTEWLTVPGKASDYGADAGTGHDNKGPLGPKDLVKEALKAAVAKGINLADFDQYDQYDQNGNGNKNEPDGIIDHLMVVHAGVGQEAGGGKLKDDAIWSHRSKLGSKPYAIDGTKSSVSNWGGKMAAYDYTIEPEDGAVGVFAHEYGHDLGLPDEYDTKYSGQGEPVESWSIMSGGSWAGKIAGTEPTSFSPQNKEFFQKNMKGNWANILEVDYDKLSKGIGVATYVDQSTTKSKRPGIVRVNLPDKDIKNIESAFGKKFYYSTKGNDIHTTLETPVFDLTNAKDAKFDYKAFYELEAKYDFLDVYAIAEDGTKTRIDRMGEKDIKGGADTTDGKWVDKSYDLSQFKGKKVKLQFEYLTDIAVAYKGFALDNAALTVDGKVVFSDDAEGQPAMTLKGFTVSNGFEQKKHNYYVEWRNYAGSDTALQYARGPVFNTGMVVWYADQSFTDNWVGVHPGEGFLGVVDSHPEAIVGTLNGQPTVKSSTRYQIADAAFSFDQTPAWKVNSPTRGIFDYKGLPGVAKFDDSKQYINSVIPDAGRKLPKLGLKFEVVGQAEDKSAGAVWLHR

SEQ ID NO: 122 - protein, 798 aa, Bacillus mycoides
KRKTPFKVFSSLAITTMLGCTFALGTSVAYAETTSQSKGSISTTPIDNNLIQEERLAEALKERGTIDQSASKEETQKAVEQYIEKKKGDQPNKEILPDDPAKEASDFVKKVKEKKMEEKEKVKKSVENASSEQTPSQNKKQLNGKVPTSPAKQAPYNGAVRTDKVLVLLVEFSDYKHNNIEQSPGYMYANDFSREHYQKMLFGNEPFTLFDGSKVKTFKQYYEEQSGGSYTTDGYVTEWLTVPGKAADYGADGKTGHDNKGPKGARDLVKEALKAAAEKGLDLSQFDQFDRYDTNGDGNQNEPDGVIDHLMVIHAGVGQEAGGGKLGDDAIWSHRSKLAQDPVAIEGTKSKVSYWDGKVAAHDYTIEPEDGAVGVFAHEFGHDLGLPDEYDTNYTGAGSPVEAWSLMSGGSWTGRIAGTEPTSFSPQNKDFLQKNMDGNWAKIVEVDYDKIKRGVGFPTYIDQSVTKSNRPGLVRVNLPEKSVETIKTGFGKHAYYSTRGDDMHTTLETPLFDLTKAANAKFDYKANYELEAECDFIEVHAVTEDGTKTLIDKLGDKVVKGDQDTTEGKWIDKSYDLSQFKGKKVKLQFDYITDPALTYKGFAMDNVNVTVDGKVVFSDDAEGQAKMKLNGFVVSDGTEKKPHYYYLEWRNYAGSDEGLKVGRGPVYNTGLVVWYADDSFKDNWVGRHPGEGFLGVVDSHPEAVVGNLNGKPVYGNTGLQIADAAFSLDQTPAWNVNSFTRGQFNYPGLPGVATFDDSKVYSNTQIPDAGRKVPQLGLKFQVVGQADDKSAGAIWIRR

SEQ ID NO: 123 - DNA, 300 bp, Bacillus cereus
taactcaatcttaagagaaattgaggagcgcgcaccacttcgtcgtacaacaacgcaagaagaagttggggatacagcagtattcttattcagtgatttagcacgcggcgtaacaggagaaaacattcacgttgattcagggtatcatatcttaggataaatataatattaattttaaaggacaatctctacatgttgagattgtcctttttatttgttcttagaaagaacgatttttaacgaaagttcttaccacgttatgaatataagtataatagtacacgatttattcagctacgt

SEQ ID NO: 124 - DNA, 172 bp, Bacillus cereus
acgttgattcagggtatcatatcttaggataaatataatattaattttaaaggacaatctctacatgttgagattgtcctttttatttgttcttagaaagaacgatttttaacgaaagttcttaccacgttatgaatataagtataatagtacacgatttattcagctacgt

SEQ ID NO: 125 - DNA, 291 bp, Bacillus cereus
cataaaaatctacttttcttgtcaaagagtatgcttatatgcgtgctctttttatttggttttctttcatttctaaataacattttcaactctattcatactattctttcaactttaggttacaaactatttctgtaagcgtagtgtttcttttgtactataggcagttagttttatccataacagtacacctctgcactattcactataaattttcatatattatattgtgcttgtccaaaacatgtggttattactcacgcgatctaaatgaaagaaaggagtgaaaat

SEQ ID NO: 126 - DNA, 94 bp, Bacillus cereus
actattcactataaattttcatatattatattgtgcttgtccaaaacatgtggttattactcacgcgatctaaatgaaagaaaggagtgaaaat

SEQ ID NO: 127 - DNA, 300 bp, Bacillus thuringiensis
aatacatgataatgaaatccgattttgtgttttatatagtgaattatcaaatattgtgtagatgaaacaaagataaaatccccattaaactccctctatggaaattataaattgttcgataaaaactttcaatattttcagaaaacattgttgaattgtgatatattcgtatgctaactatgaaatttttacaaatatattaaaaacattacataatatgactaaatattgaaaaaatattgaatttttaataaaatttaatttgtaatacatattatttattaggggaggaaataaggg

SEQ ID NO: 128 - DNA, 48 bp, Bacillus thuringiensis
aaaatttaatttgtaatacatattatttattaggggaggaaataaggg

SEQ ID NO: 129 - DNA, 300 bp, Bacillus mycoides
aattgtgcatattgtcttttaaattttctatctaagttatttaatatataataaataactcttttttgtgagtttttttgatacgaggtaaataatcagtacagggtctgaccagaggactggagggcatgattctataagggaatatttactattccatgattatagaactatgtcttttttattgtatatagaaggggggataggtctatattatagaacttatatatattgtgcattccatattatcaattatctaaattttaagtcttgttacaattaataagggaggaaatagta

SEQ ID NO: 130 - DNA, 80 bp, Bacillus mycoides
acttatatatattgtgcattccatattatcaattatctaaattttaagtcttgttacaattaataagggaggaaatagta

SEQ ID NO: 131 - DNA, 141 bp, Bacillus thuringiensis
aatgacgttttcaagtttgattatcattcatgtttcctattttaagagaaacatataactcaactacttttttcaatggcatcttttatagtacttagaataggaaaacactcaactataagaaaagtaaggaggaaataa

SEQ ID NO: 132 - DNA, 79 bp, Bacillus thuringiensis
actacttttttcaatggcatcttttatagtacttagaataggaaaacactcaactataagaaaagtaaggaggaaataa

SEQ ID NO: 133 - DNA, 300 bp, Bacillus cereus
atatgctaatgcttagtttttatactcaagttaaaatgtgcttttggacctaagagataaacgtggaaaaataaaataaactcttaagtttaggtgtttaatctaagcagtcaattattaaaaacatataattaatatgtgagtcatgaacataattaaataatgttttcaagtttaattatcgttcatgtttcctattttaagcagaacaaataactcaattacttttttcgattggatcttttttaactcttataataggaaaacactcaactataaaaataagtaaggaggaaataa

SEQ ID NO: 134 - DNA, 167 bp, Bacillus cereus
aatatgtgagtcatgaacataattaaataatgttttcaagtttaattatcgttcatgtttcctattttaagcagaacaaataactcaattacttttttcgattggatcttttttaactcttataataggaaaacactcaactataaaaataagtaaggaggaaataa

SEQ ID NO: 135 - DNA, 300 bp, Bacillus thuringiensis
tataaaataaaagggcgtgtatttgctactgatgcagtattgtgtgcgcctaaaaatggaatttcacaaccagatccacatgttgttgtagaacaatcttgtaattcattgatgaattttacaacgtcaactacacaatgagaagagccatggtgtttattttcgttacaactcattaatgtcactccttatcttcttgtttgtatttacattaataagatattggagttgaggagatttggtcacaatctcaagaccttttttttaaataggcgaaagaggataagggaaggtggaatt

SEQ ID NO: 136 - DNA, 106 bp, Bacillus thuringiensis
tcttgtttgtatttacattaataagatattggagttgaggagatttggtcacaatctcaagaccttttttttaaataggcgaaagaggataagggaaggtggaatt

SEQ ID NO: 137 - DNA, 300 bp, Bacillus thuringiensis
atcaacttttacaaaagtaaagggtaaaggattaagaaagtggattggcgaattattaagctgttattggtgtacaggtgtatgggttagtgcttttttattagttttatataattggattccgatcgttgcagagccgttacttgcattattagctattgcaggagcagcagcaatcattgaaacgattacaggatattttatgggagaataatatattttcataatacgagaaaaagcggagtttaaaagaatgagggaacggaaataaagagttgttcatatagtaaatagacagaa

SEQ ID NO: 138 - DNA, 39 bp, Bacillus thuringiensis
acggaaataaagagttgttcatatagtaaatagacagaa

SEQ ID NO: 139 - DNA, 300 bp, Bacillus anthracis
tgaagtatctagagctaatttacgcaaaggaatctcaggacaacactttcgcaacacctatattttaaatttaataaaaaaagagactccggagtcagaaattataaagctagctgggttcaaatcaaaaatttcactaaaacgatattatcaatacgcagaaaatggaaaaaacgccttatcataaggcgttttttccattttttcttcaaacaaacgattttactatgaccatttaactaatttttgcatctactatgatgagtttcattcacattctcattagaaaggagagattta

SEQ ID NO: 140 - DNA, 70 bp, Bacillus anthracis
accatttaactaatttttgcatctactatgatgagtttcattcacattctcattagaaaggagagattta

SEQ ID NO: 141 - DNA, 300 bp, Bacillus cereus
gactatgtttattcaggataaaatatagcactacactctctcctcttattatgtagcatctctctaatccatcatttgtttcatttagttaaaattgtaaataaaatcacatgatttgtcaattataattgtcatttcgacaattaaacttgtcaaaataattctcatcattttttctcatctttctaatataggacatactactatatatacaaaagacaatatgcaaatgttcatacaaaaaatattatttttcgatatataatattaactgattttctaacatcaaggagggtacat

SEQ ID NO: 142 - DNA, 84 bp, Bacillus cereus
agacaatatgcaaatgttcatacaaaaaatattatttttcgatatataatattaactgattttctaacatcaaggagggtacat

SEQ ID NO: 143 - DNA, 300 bp, Bacillus thuringiensis
atagtgagtaatatggtaatccatagattaaatagtatagaaaatatttaattcttatttttattaaaaaagcatgaatcccagatttactgggttttgattgtaactaagaacatataaaagttcactgttatttataggagagtctgtttgtttttatatcttatgtatttcaccctgcataaaaaaatatttctcaacattttatttgttgaaaaatattgaatattcgtattataacgaatattatgttgttatcggcaaaaaacgataatttgcagacactggggaggaaataca

SEQ ID NO: 144 - DNA, 139 bp, Bacillus thuringiensis
tcttatgtatttcaccctgcataaaaaaatatttctcaacattttatttgttgaaaaatattgaatattcgtattataacgaatattatgttgttatcggcaaaaaacgataatttgcagacactggggaggaaataca

SEQ ID NO: 145 - DNA, 300 bp, Bacillus cereus
cttcgtcagcaataagtgtgagcggagaattggttgatcttggctttacaattggagcattgacgaaagactctttaacgtggtcgcataacggagtagaatatatgctcgtgtctaaaggtttagagccgaaggagctattaatggttgctcgttcagttacagagaagcaagtgaagtaaacttcttagacgtggtgatatatgtgcaccacgtcttttcttagtttgaagggtggatttcataaaagaagcatataaaagaataagcttcgcatatcgtgtataaggaagtgtattt

SEQ ID NO: 146 - DNA, 44 bp, Bacillus cereus
ataaaagaataagcttcgcatatcgtgtataaggaagtgtattt

SEQ ID NO: 147 - DNA, 300 bp, Bacillus thuringiensis
catttcaaataatgaacgcttcgattgaatcggagctattttcaaatcaatttcagtatattgatccagcatttgaatagaagtatcaacagcaactttaagttgatgcaatgcagattgtacaaacattgtaattctcctcttctccgtatataatagtttcttgagggtattatatcatgctcaaaattccgaaaattctagtagtttgactagcatattgaaaagtattatattgtaaaaggtcatatgaaacgtgaaatagaatggaatgcaattattgagttaggagttagacca

SEQ ID NO: 148 - DNA, 70 bp, Bacillus thuringiensis
ttatattgtaaaaggtcatatgaaacgtgaaatagaatggaatgcaattattgagttaggagttagacca

SEQ ID NO: 149 - DNA, 300 bp, Bacillus cereus
atcgatggaacctgtatcaaccactataatttcatccacaattttttcaactgagtctaaacaacgggctattgtcttctcctcatctcgaacaatcatacataaactaattgtaattccttgcttgttcaacataatcaccctcttccaaatcaatcatatgttatacatatactaaactttccatttttttaaattgttcaagtagtttaagatttcttttcaataattcaaatgtccgtgtcattttctttcggttttgcatctactatataatgaacgctttatggaggtgaattt

SEQ ID NO: 150 - DNA, 150 bp, Bacillus cereus
aatcaatcatatgttatacatatactaaactttccatttttttaaattgttcaagtagtttaagatttcttttcaataattcaaatgtccgtgtcattttctttcggttttgcatctactatataatgaacgctttatggaggtgaattt

SEQ ID NO: 151 - DNA, 300 bp, Bacillus thuringiensis
gacctgtaagtctgtagggaagaataatttcaagagccagtgataatagatttttttgttttttcattcttatcttgaatataaatcacctcatcttttaattagaacgtaaccaatttagtattttgaaatagagctatcattttataatatgaatactactagttatagaaacggcaaaaagtttaatatatgtaaaaatcatttggatatgaaaaaagtagccatagattttttcgaaatgataaatgttttattttgttaattaggaaacaaaaatgtggaatgagggggatttaa

SEQ ID NO: 152 - DNA, 91 bp, Bacillus thuringiensis
atatgaaaaaagtagccatagattttttcgaaatgataaatgttttattttgttaattaggaaacaaaaatgtggaatgagggggatttaa

SEQ ID NO: 153 - DNA, 300 bp, Bacillus anthracis
ttttcatctgctacatcgtgaagtaatgctgccatttcaattataaaacgatttcctccttcttgctcggataaagaaatcgccagtttatgtacacgctcaatatgataccaatcatgcccactggcatctttttctaaaatatgttttacaaaagtaattgttttttctatcttttcttgttttgtcattttatcttcacccagttacttattgtaacacgcccgcattttttcatcacatattttcttgtccgcccatacactaggtggtaggcatcatcatgaaggaggaatagat

SEQ ID NO: 154 - DNA, 61 bp, Bacillus anthracis
acatattttcttgtccgcccatacactaggtggtaggcatcatcatgaaggaggaatagat

SEQ ID NO: 155 - DNA, 300 bp, Bacillus anthracis
ggtgacgacaacatatacaagaggcactcctgctggtactgtaacaggaacaaatatggggcaaagtgtaaatacatcgggtatagcacaagctgtcccgaatacagataatatggattcaacggcgggactcccttaagaaattaggggagtctttatttggaaaaagagcttatgttacataaaaacaggagtaattgttttaaaagtagtattggtgacgttgttagaaaatacaatttaagtagaaggtgcgtttttatatgaaatatattttatagctgtactttacctttcaag

SEQ ID NO: 156 - DNA, 213 bp, Bacillus anthracis
acaagctgtcccgaatacagataatatggattcaacggcgggactcccttaagaaattaggggagtctttatttggaaaaagagcttatgttacataaaaacaggagtaattgttttaaaagtagtattggtgacgttgttagaaaatacaatttaagtagaaggtgcgtttttatatgaaatatattttatagctgtactttacctttcaag

SEQ ID NO: 157 - DNA, 300 bp, Bacillus anthracis
atttatttcattcaatttttcctatttagtacctaccgcactcacaaaaagcacctctcattaatttatattatagtcattgaaatctaatttaatgaaatcatcatactatatgttttataagaagtaaaggtaccatacttaattaatacatatctatacacttcaatatcacagcatgcagttgaattatatccaactttcatttcaaattaaataagtgcctccgctattgtgaatgtcatttactctccctactacatttaataattatgacaagcaatcataggaggttactac

SEQ ID NO: 158 - DNA, 180 bp, Bacillus anthracis
taagaagtaaaggtaccatacttaattaatacatatctatacacttcaatatcacagcatgcagttgaattatatccaactttcatttcaaattaaataagtgcctccgctattgtgaatgtcatttactctccctactacatttaataattatgacaagcaatcataggaggttactac

SEQ ID NO: 159 - DNA, 300 bp, Bacillus cereus
agttgtacaagaatttaaatcttcacaaacatatgtaaatgacttactacagctagttgcaagtacgatttctaacaacgtaacagatgaaatattaatttcaactaatggcgatgtattgaagggtgaaacgggcgcagcggtagaaagtaaaaaaggaaattgtggttgttaaagagatgtcgaaatgacatctctttttttagtggattaaacgtaagttcttctcaaaaaaagaatgacacattccgctattgtcacgcatatgattaagtgaatagtgattgaggagggttacga

SEQ ID NO: 160 - DNA, 57 bp, Bacillus cereus
acattccgctattgtcacgcatatgattaagtgaatagtgattgaggagggttacga

SEQ ID NO: 161 - DNA, 300 bp, Bacillus cereus
aacgttattagcgtagacaaacaagtaacggcagaagcagttcttgcattaaatcgtatgttagagcgtgtgtaaagcaacggtattcccgttgctttttttcatacatataatcataacgagaacgaaatgggcatacattgttttgaagaaatcattgtggttctttatgcttattccacttcgaatgatattgaaaatcgaagaagtgataaaagtaaaaagaagttaatgttatttagaaagagttacttcatgagatttgttacttatagataagttatacaggagggggaaaat

SEQ ID NO: 162 - DNA, 47 bp, Bacillus cereus
tcatgagatttgttacttatagataagttatacaggagggggaaaat

SEQ ID NO: 163 - DNA, 300 bp, Bacillus thuringiensis
aagccgcggtcaatgctgtatatgcaaataagattgcagctttacctgaagaagagcgtgatagcttcattgctgaaaaacgagaagagtataagaaagatattgatatttaccatttagcatcagagatggtcattgatggtattgttcatccaaacaatttaagagaagagttaaaaggacgattcgaaatgtatatgagtaaatatcaagtatttacggatcgtaaacatcctgtttatccagtttaaaagccctatttagggctttcttgctcaaaaagttaaggaggggaaaaca

SEQ ID NO: 164 - DNA, 92 bp, Bacillus thuringiensis
tcaagtatttacggatcgtaaacatcctgtttatccagtttaaaagccctatttagggctttcttgctcaaaaagttaaggaggggaaaaca

SEQ ID NO: 165 - DNA, 300 bp, Bacillus cereus
aggatttcagtgggacgcctcctctcttcttacattaaattaatcatactataaaatgaaagaaatgaaatgaaaaatagcggaaaaatcagaaattttttctggtagtatacaatatgttacaataagctttgtcaatgaaagaaggaattccgtgcaatgcacgggagaggttcgcgaactccctctataaaaaactatggaaacaacaatatctttaggtattgttttgtttttttattgtgacagttcaagaacgttctttcttcttattcgtagtagagaaggagaatgagtgaa

SEQ ID NO: 166 - DNA, 104 bp, Bacillus cereus
actatggaaacaacaatatctttaggtattgttttgtttttttattgtgacagttcaagaacgttctttcttcttattcgtagtagagaaggagaatgagtgaa

SEQ ID NO: 167 - DNA, 300 bp, Bacillus cereus
ttttgcacaacgccgtaaaactttaatgaataatttatcaaataatttaaatggtttcccgaaagataaagagctgttggatcgaattttaacagaagtaggaattgatccaaaacgaagaggcgaaacgctatctatcgaagagtttgcgacattaagtaatgcattagttcttcataagttatcataagaatacaaaagggacagttcaatttgaactgtcccttttgtcacctttctcctcctaaattcatactttaaaaacaggtaagatggcctaacgagtttggaggtaggaga

SEQ ID NO: 168 - DNA, 63 bp, Bacillus cereus
tctcctcctaaattcatactttaaaaacaggtaagatggcctaacgagtttggaggtaggaga

SEQ ID NO: 169 - DNA, 300 bp, Bacillus thuringiensis
ggaaacagaagtcatcccatttgaaaatgcagcaggtcgtattatagctgatttcgttatggtttatccgccagggattccaatctttactccgggggaaattattacacaagacaacttagagtatattcgtaaaaacttagaagcaggtttacctgtacaaggtcctgaagatatgacattacaaacattacgcgtgatcaaagagtacaagcctatcagttgataggctttttttcaccctttttcccttttctcatacgatattatgtaatgtaacgtataggtggggatactact

SEQ ID NO: 170 - DNA, 61 bp, Bacillus thuringiensis
accctttttcccttttctcatacgatattatgtaatgtaacgtataggtggggatactact

SEQ ID NO: 171 - DNA, 300 bp, Bacillus cereus
attgtggacccttagctcagctggttagagcagacggctcataaccgtccggtcgtaggttcgagtcctacagggtccatatccatttcacatgtttattatgtcggcaggaagcttccttgtagaagggagctttttttatgaaatatatgagcattttaattgaaatgaagtgggaattttgctactttaatgatagcaagacaatgtgatttatttgtttgcaccctatggcaattagggtagaatgaagttgtatgtcacttaagtggcaatacataaactgggaggaatataaca

SEQ ID NO: 172 - DNA, 38 bp, Bacillus cereus
acttaagtggcaatacataaactgggaggaatataaca

SEQ ID NO: 173 - DNA, 300 bp, Bacillus cereus
aatataacagaaaattctgatgttttttcaaatcctataataaggagtgttccgtatgatgcctttatattttccggaagataaaacagaatatattattccagggattgtttgtgttctatttatcatcggtgcgattgctacgtggcgtatgttcattcgtgtatcaaaacgagaagcagagcgattacagaaagttgaagaaaagctgttagctgaaaagaaacagtaactcatttttgtatgtttccctctatgctcggacaatctaagggcagaatgtattttggagggaatgaa

SEQ ID NO: 174 - DNA, 228 bp, Bacillus cereus
tccggaagataaaacagaatatattattccagggattgtttgtgttctatttatcatcggtgcgattgctacgtggcgtatgttcattcgtgtatcaaaacgagaagcagagcgattacagaaagttgaagaaaagctgttagctgaaaagaaacagtaactcatttttgtatgtttccctctatgctcggacaatctaagggcagaatgtattttggagggaatgaa

SEQ ID NO: 175 - DNA, 166 bp, Bacillus anthracis
taatcaccctcttccaaatcaatcatatgttatacatatactaaactttccatttttttaaattgttcaagtagtttaagatttcttttcaataattcaaatgtccgtgtcattttctttcggttttgcatctactatataatgaacgctttatggaggtgaattt

SEQ ID NO: 176 - DNA, 173 bp, Bacillus anthracis
aattacataacaagaactacattagggagcaagcagtctagcgaaagctaactgcttttttattaaataactattttattaaatttcatatatacaatcgcttgtccatttcatttggctctacccacgcatttactattagtaatatgaatttttcagaggtggattttatt

SEQ ID NO: 177 - DNA, 121 bp, Bacillus weihenstephanensis
ctatgatttaagatacacaatagcaaaagagaaacatattatataacgataaatgaaacttatgtatatgtatggtaactgtatatattactacaatacagtatactcataggaggtaggt

SEQ ID NO: 178 - DNA, 373 bp, Bacillus weihenstephanensis
ggtaggtagatttgaaatatgatgaagaaaaggaataactaaaaggagtcgatatccgactccttttagttataaataatgtggaattagagtataattttatataggtatattgtattagatgaacgctttatcctttaattgtgattaatgatggattgtaagagaaggggcttacagtcctttttttatggtgttctataagcctttttaaaaggggtaccaccccacacccaaaaacagggggggttataactacatattggatgttttgtaacgtacaagaatcggtattaattaccctgtaaataagttatgtgtatataaggtaactttatatattctcctacaataaaataaaggaggtaataaa

SEQ ID NO: 179 - DNA, 225 bp, Bacillus thuringiensis
aacccttaatgcattggttaaacattgtaaagtctaaagcatggataatgggcgagaagtaagtagattgttaacaccctgggtcaaaaattgatatttagtaaaattagttgcactttgtgcattttttcataagatgagtcatatgttttaaattgtagtaatgaaaaacagtattatatcataatgaattggtatcttaataaaagagatggaggtaactta

SEQ ID NO: 180 - DNA, 115 bp, Bacillus thuringiensis
taattccaccttcccttatcctctttcgcctatttaaaaaaaggtcttgagattgtgaccaaatctcctcaactccaatatcttattaatgtaaatacaaacaagaagataagga

SEQ ID NO: 181 - DNA, 144 bp, Bacillus thuringiensis
aggatgtctttttttatattgtattatgtacatccctactatataaattccctgcttttatcgtaagaattaacgtaatatcaaccatatcccgttcatattgtagtagtgtatgtcagaactcacgagaaggagtgaacataa

SEQ ID NO: 182 - DNA, 125 bp, Bacillus thuringiensis
ttaatgtcactccttatcttcttgtttgtatttacattaataagatattggagttgaggagatttggtcacaatctcaagaccttttttttaaataggcgaaagaggataagggaaggtggaatt

SEQ ID NO: 183 - DNA, 87 bp, Bacillus thuringiensis
atatattttcataatacgagaaaaagcggagtttaaaagaatgagggaacggaaataaagagttgttcatatagtaaatagacagaa

SEQ ID NO: 184 - DNA, 168 bp, Bacillus thuringiensis
aaactaaataatgagctaagcatggattgggtggcagaattatctgccacccaatccatgcttaacgagtattattatgtaaatttcttaaaattgggaacttgtctagaacatagaacctgtccttttcattaactgaaagtagaaacagataaaggagtgaaaaac

SEQ ID NO: 185 - DNA, 111 bp, Bacillus thuringiensis
attcactacaacggggatgagtttgatgcggatacatatgagaagtaccggaaagtgtttgtagaacattacaaagatatattatctccatcataaaggagagatgcaaag

SEQ ID NO: 186 - DNA, 273 bp, Bacillus anthracis
cgcgcaccacttcgtcgtacaacaacgcaagaagaagttggggatacagcagtattcttattcagtgatttagcacgcggcgtaacaggagaaaacattcacgttgattcagggtatcatatcttaggataaatataatattaattttaaaggacaatctctacatgttgagattgtcctttttatttgttcttagaaagaacgatttttaacgaaagttcttaccacgttatgaatataagtataatagtacacgatttattcagctacgta

SEQ ID NO: 187 - DNA, 240 bp, Bacillus anthracis
tatatcatatgtaaaattagttcttattcccacatatcatatagaatcgccatattatacatgcagaaaactaagtatggtattattcttaaattgtttagcaccttctaatattacagatagaatccgtcattttcaacagtgaacatggatttcttctgaacacaactctttttctttccttatttccaaaaagaaaagcagcccattttaaaatacggctgcttgtaatgtacatta

SEQ ID NO: 188 - DNA, 267 bp, Bacillus thuringiensis
tatcacataactctttatttttaatatttcgacataaagtgaaactttaatcagtgggggctttgttcatccccccactgattattaattgaaccaagggataaaaagatagagggtctgaccagaaaactggagggcatgattctataacaaaaagcttaatgtttatagaattatgtctttttatatagggagggtagtaaacagagatttggacaaaaatgcaccgatttatctgaattttaagttttataaaggggagaaatg

SEQ ID NO: 189 - DNA, 124 bp, Bacillus thuringiensis
attttttacttagcagtaaaactgatatcagttttactgctttttcatttttaaattcaatcattaaatcttccttttctacatagtcataatgttgtatgacattccgtaggaggcacttata

SEQ ID NO: 190 - DNA, 170 bp, Bacillus thuringiensis
acataaattcacctccataaagcgttcattatatagtagatgcaaaaccgaaagaaaatgacacggacatttgaattattgaaaagaaatcttaaactacttgaacaatttaaaaaaatggaaagtttagtatatgtataacatatgattgatttggaagagggtgatta

SEQ ID NO: 191 - DNA, 212 bp, Bacillus thuringiensis
ttctattttccaacataacatgctacgattaaatggttttttgcaaatgccttcttgggaagaaggattagagcgtttttttatagaaaccaaaagtcattaacaattttaagttaatgacttttttgtttgcctttaagaggttttatgttactataattatagtatcaggtactaataacaagtataagtatttctgggaggatatatca

SEQ ID NO: 192 - protein, 180 aa, Bacillus cereus
MSEFREIITKAVVGKGRKYTKSTHTCESNNEPTSILGCWVINHSYEARKNGKHVEIEGFYDVNTWYSFDGNTKTEVVTERVNYTDEVSIGYRDKNFSGDDLEIIARVIQPPNCLEALVSPNGNKIVVTVEREFVTEVVGETKICVSVNPEGCVESDEDFQIDDDEFEELDPNFIVDAEEE

SEQ ID NO: 193 - protein, 155 aa, Bacillus thuringiensis
MTLMSCNENKHHGSSHCVVDVVKFINELQDCSTTTCGSGCEIPFLGAHNTASVANTRPFILYTKAGAPFEAFAPSANLTSCRSPIFRVESVDDDSCAVLRVLSVVLGDSSPVPPTDDPICTFLAVPNARLVSTSTCITVDLSCFCAIQCLRDVTI

SEQ ID NO: 194 - protein, 366 aa, Bacillus anthracis; identical to SEQ ID NO: 8

SEQ ID NO: 195 - protein, 347 aa, Bacillus anthracis
MQNDKLWLDKGIIGPENIGPTFPVLPPIHIPTGITGATGATGITGATGPTGTTGATGATGITGVTGATGITGVTGATGITGVTGATGITGVTGATGITGVTGPTGITGATGPTGTTGVTGPTGDTGLAGATGPTGATGLAGATGPTGDTGATGPTGATGLAGATGPTGATGLTGATGATGATGGGAIIPFASGTTPALLVNAVLANTGTLLGFGFSQPGIAPGVGGTLTILPGVVGDYAFVAPRDGIITSLAGFFSATAALAPLTPVQIQMQIFIAPAASNTFTPVAPPLLLTPALPAIAIGTTATGIQAYNVPVVAGDKILVYVSLTGASPIAAVAGFVSAGLNIV

SEQ ID NO: 196 - protein, 61 aa, Bacillus cereus; identical to SEQ ID NO: 118

SEQ ID NO: 197 - protein, 66 aa, Bacillus cereus
LTVEEMFAVSNNPRQNSYDLQQWYHMQQQHQAQQQAYQEQLQQQGFVKKKGCNCGKKKSTIKHYEE

SEQ ID NO: 198 - protein, 156 aa, Bacillus cereus; identical to SEQ ID NO: 111

SEQ ID NO: 199 - protein, 182 aa, Bacillus anthracis; identical to SEQ ID NO: 112

SEQ ID NO: 200 - DNA, 9 bp, Artificial Sequence
  Consensus sequence
  misc_feature (5)..(7): n is a, c, g, or t
  misc_feature (9)..(9): n is a, c, g, or t
catannntn

SEQ ID NO: 201 - protein, 17 aa, Bacillus anthracis
MKILSDLVGPTFPPVPT

SEQ ID NO: 202 - protein, 17 aa, Bacillus anthracis
MIIGPENIGPTFPVLPP

SEQ ID NO: 203 - protein, 156 aa, Bacillus anthracis
MSCNCNEDHHEHDCDFNCVSNVVRFIHELQDCATTTCGSGCEVPFLGAHNTASVANTRPFILYTKAGAPFEAFAPSASLTSCRSPIFRVESIDDDDCAVLRVLTVVLGDNSPVPPTDDPICTFLAVPNARLVSTSTCITVDLSCFCAIQCLRDVTI

SEQ ID NO: 204 - protein, 296 aa, Bacillus thuringiensis
MSNNNYSNGLNPDESLSASAFDPNLVGPTLPPIPPFTLPTGPTGPTGPTGPTGPTGGGTGITGPTGPTGDTGATGPTGPTGDTGATGLTGPTGDTGATGPTGPTGDTGATGPTGPTGDTGATGLTGPTGDTGATGPTGPTGATGPTGATGPTGATGPTGPSGLGLPAGLYAFNSGGISLDLGINDPVPFNTVGSQFGTAISQLDADTFVISETGFYKITVIAYTATVSLLGGLTIQVNGVPVPGTGSSLISLGAPIVIQAITQITTTPSLVEVIVTGLGLSLALGTSASIIIEKVA

SEQ ID NO: 205 - protein, 166 aa, Bacillus thuringiensis
MSNNNYSNGLNPDESLSASAFDPNLVGPTLPPIPPFTLPTGPTGPTGPTGPTGPTGGGTGITGPTGPTGDTGATGPTGPTGDTGATGLTGPTGDTGATGPTGPTGDTGATGPTGPTGDTGATGLTGPTGDTGATGPTGPTGATGPTGATGPTGATGPTGPSGLGLP

SEQ ID NO: 206 - protein, 322 aa, Bacillus anthracis
MSNNNYSNGLNPDESLSASAFDPNLVGPTLPPIPPFTLPTGPTGPTGPTGPTGPTGPTGPTGPTGPTGPTGDTGTTGPTGPTGPTGPTGPTGPTGPTGPTGPTGPTGPTGPTGPTGDTGTTGPTGPTGPTGPTGPTGDTGTTGPTGPTGPTGPTGPTGPTGPTGPTGPTGPTGATGLTGPTGPTGPSGLGLPAGLYAFNSGGISLDLGINDPVPFNTVGSQFGTAISQLDADTFVISETGFYKITVIANTATASVLGGLTIQVNGVPVPGTGSSLISLGAPIVIQAITQITTTPSLVEVIVTGLGLSLALGTSASIIIEKVA

SEQ ID NO: 207 - protein, 196 aa, Bacillus anthracis
MSNNNYSNGLNPDESLSASAFDPNLVGPTLPPIPPFTLPTGPTGPTGPTGPTGPTGPTGPTGPTGPTGPTGDTGTTGPTGPTGPTGPTGPTGPTGPTGPTGPTGPTGPTGPTGPTGDTGTTGPTGPTGPTGPTGPTGDTGTTGPTGPTGPTGPTGPTGPTGPTGPTGPTGPTGATGLTGPTGPTGPSGLGLPAGLY

SEQ ID NO: 208 - protein, 8 aa, Artificial Sequence
  Synthetic construct
AAAAADLE

SEQ ID NO: 209 - protein, 8 aa, Artificial Sequence
  Synthetic construct
AAAAAALE

SEQ ID NO: 210 - protein, 367 aa, Aspergillus niger
MHSYQLLGLAAVGSLVSAAPAPSRVSEFAKKASTCTFTSASEASESISSCSDVVLSSIEVPAGETLDLSDAADGSTITFEGTTSFGYKEWKGPLIRFGGKDLTVTMADGAVIDGDGSRWWDSKGTNGGKTKPKFMYIHDVEDSTFKGINIKNTPVQAISVQATNVHLNDFTIDNSDGDDNGGHNTDGFDISESTGVYISGATVKNQDDCIAINSGESISFTGGTCSGGHGLSIGSVGGRDDNTVKNVTISDSTVSNSANGVRIKTIYKETGDVSEITYSNIQLSGITDYGIVIEQDYENGSPTGTPSTGIPITDVTVDGVTGTLEDDATQVYILCGDGSCSDWTWSGVDLSGGKTSDKCENVPSGAS

SEQ ID NO: 211 - protein, 543 aa, Bacillus simplex
MAIGNEVNVFDFGAKGDGIADDTIAIQAAIDYCSVFGRPTRVLLPIGNYKVTSPIILPTSRQGLEIAGYGNRATVINPYHEGNVIETSSQGSKIRDLMIYTTGSFSKKYTGIYATNVHSVSIDNVHIMQPKNGIILEGGVYYMNARDILILNYFNKGIYLKKSIDGATPNANTFEIKMVTSDNSANPGTIGVHIEGGTLNEFSGGQISHSDIAFYLENASRNYFRNIWVEHTKNAVKAISGINFMDCHGLIGNILSGGAIIKTSYGEVSFYSFLPQEQPNITKSLKGLFFFNEGEGNLVVDKSGNGKHLQLTGTPEWTDEGKWGKSVRFNNQIGNRILAPADVLDWSKPYTFGILAKIEDMGNNPYPQFGLFIDGGEKYVAFNLHSSYLQIVNHNNSSDTNPHSEQSVSFGTGSFNDFQWIFIYVDPVKNYVEVIDPTRDKPVGVITTIPIPIKDSQSISKLYLSGRSIAVSNTTIGVMSMSVFYQRKLSKKEVLEIVNSVERPFIEMNEKVVKSTTLISPNGLSYKISVDETGQLVITRMLK

SEQ ID NO: 212 - protein, 541 aa, Bacillus spp.
MPNGNGVNVLDFGAIGDGVADDTRAIQEAINYCSVAGRPTKVLIPRGDYKVTSPIVLTSSRQGLEIVGEGNRASVISPNHSGNVIEISSQGSKIRDLMIYTTGNFSKKYTGIYVLNVHSVSIDNVHIMYPKNGIILEGDVYYLNARDILILNYINKGIYLKRSINGAMPNSNTFEIKIVTSDNGATPGTIGVHIEGGILNEFRGGQISHSDIAFYLENANRNYFRNIWVEHTKNAVKAISGINFMDCHGLLGNILSEGAIIKTSYGEVSFYSFLPQESPNITKCLKGLYFFNEGEGNLVIDKSGNQKHLILTGTPEWTDDGKWGKAVRFNNQIGNRILAPADLLDWSQPYTFGILAKIENRGNNQYPQFGLFIDGGEKYVAFILHSSYLQIVNHNNSLGTNLHSNQTVGYGAGSFDDFQWVFIYVDPLNNYVEVIDPTRDKPVGVNTTIPIPIKDSQTISKLYLSGRSIAVSNMTIGVMSMSVFFQRKLLKKEVLEIVNSVERPFIEMNDKIVKSTTLISPNGLSYKISVDETGQLVIIRV

SEQ ID NO: 213 - protein, 221 aa, Bacillus subtilis
MKKIVSILFMFGLVMGFSQFQPSTVFAADKVVHETIIVPKNTTYDGKGQRFVAGKELGDGSQSENQDPVFRVEDGATLKNVVLGAPAADGVHTYGNVNIQNVKWEDVGEDALTVKKEGKVTIDGGSAQKASDKIFQINKASTFTVKNFTADNGGKFIRQLGGSTFHVDVIIDKCTITNMKEAIFRTDSKTSTVRMTNTRYSNVGQKWIGVQHIYENNNTQF

SEQ ID NO: 214 - protein, 345 aa, Bacillus safensis
MLTLKRKVPMLAGVLAVGLVTSLFAPNGEAKAAAKFPESPSIMANFNQQGFSTLNGGTTGGEGGKTVTVKTGNELLAALKNKGTNEKLKIVVDGTITPSNTSANKIDVKDTNNVSIVGKGTNGEFNGIGIKVWRANNIIIRNLKIHHSKIGDKDAIGIEGGSKNIWVDHNELYNTLNSGKDDYDGLFDVKNDSDYITFSWNYVHDSWKTMLMGSSDNDNYNRKITFHNNRFENLNSRVPSMRFGEGHVYNNYYKGILTTAINSRMGAKMRIEHNVFENTNNAIGSWDSSQVGTWHVINNSYINSTGSLPTSSTGTYNPPYNYSLLNVNNVKSEVISNAGVGKVNP

SEQ ID NO: 215 - protein, 354 aa, Bacillus pumilus
MEMLTLKRKVPMLAGVLAVGLVTSLFAPNGEAKAAAKYPESPSIMANFNQQGFSTLNGGATGGEGGKTVTVKTGSELLAALKNKGTNEKLKIVVDGTITPSNTSANKIDVKDTNNVSIVGKGTRGEFNGVGIKVWRANNIIIRNLKIHHSKIGDKDAIGIEGGSKNIWVDHNELYNTLNSGKDDYDGLFDVKNDSDYITFSWNYVHDSWKTMLMGSSDNDNYNRKITFHNNRFENLNSRVPSMRFGEGHVYYNYYKGILATAINSRMGAKMRIEHNVFENTNNAIGSWYSRQVGTWHVINNSDINSTGSLPTSSTGTYNPPYNYSVLDVNDVKSEVISNAGVEKEKIKLEFSEG

SEQ ID NO: 216 - protein, 341 aa, Bacillus licheniformis
MKKLISIIFIFVLGVVGSLTAAVSAEAASALNSGKVNPLADFSLKGFAALNGGTTGGEGGQTVTVTTGDQLIAALKNKNANTPLKIYVNGTITTSNTSASKIDVKDVSNVSIVGSGTKGELKGIGIKIWRANNIIIRNLKIHEVASGDKDAIGIEGPSKNIWVDHNELYHSLNVDKDYYDGLFDVKRDAEYITFSWNYVHDGWKSMLMGSSDSDNYNRTITFHHNWFENLNSRVPSFRFGEGHIYNNYFNKIIDSGINSRMGARIRIENNLFENAKDPIVSWYSSSPGYWHVSNNKFVNSRGSMPTTSTTTYNPPYSYSLDNVDNVKSIVKQNAGVGKINP

SEQ ID NO: 217 - protein, 421 aa, Bacillus amyloliquefaciens
MKKMLFMLAVCLCMIPADVYAADLGRQTLGTNDGWGAASGGTIGGAKASSSNVYTVSNRQQLVSALGGSANSTPKIIYIQGTINMNTDDRNKTLGFDDYKDPAYDPNAYLKAYDPDKWGKKAPSGTQEDARQRSQKNQKARVVVDIPSNTTIIGSGSNAKVTGGNFNIKNGVDNVIIRNIEFQDAYDYFPQWDPTDGSSGNWNSEYDNITINGATHIWIDHCTFNDGSNPDSGFPYYYGRKYQHHDGQTDIANGANYITLSYNKYHDHDKGSVIGNSDSKTSDEGKLKVTIHHNYYQNIVQRAPRVRYGQVHIYNNFYAGSKSAAYPFSYAWGAGHASKIYAQNNVFEVPGLAADKVISVFSGGKALHEDGTLLNGASINASAANGLNQSVGWTPSLHGSIGSSSNVKSDVISKAGSGILK

SEQ ID NO: 218 - protein, 436 aa, Bacillus licheniformis
MSLQKIKEEIVKKLKVPVFPNRSFDVTSFGADENGKNDSTEAIQKAIDQAHQAGGGRVTVPEGVFLSGALRLKSNVDLHIAKGAVIKFSQNPEDYLPVVLTRFEGVELYNYSPLIYAYEADNIAITGKGTLDGQGDDEHWWPWKRGTNGQPSQEKDRNALFEMAERGIPVTERQFGKGHYLRPNFIQPYRCKHILIQGVTVLNSPMWQVHPVLCENVTVDGIKVIGHGPNTDGVNPESCKNVVIKGCHFDNGDDCIAVKSGRNADGRRINIPSENIVIEHNEMKDGHGGVTIGSEISGGVKNVIAEGNLMDSPNLDRALRIKTNSVRGGVLENIYFHKNTVKSLKREVIAIDMEYEEGDAGDFKPVVRTVDVKQLKSMGGQYGIRVLAYDHSPVTGLKVADSEIDGVDVPMELKHVKDPVFSNLYINGKRYDSHKA

SEQ ID NO: 219 - protein, 471 aa, Bacillus safensis
MSKKRDLMYITAGTVLTGVGIARLAKQVKKNKKANGIDQVLKQIKAPQFQDRELNVVHYGADAEGQELSTNAIQSAIDDAHRLKGGRVLIPAGTFMTGALEMKSNVELHLHEKAYVTFSQDPKDYLPLVLTRYEGVELYNYSPLIYAHHAENIAITGAGTLDGRGDEHHWWPWKYGTNGQPSQDRDRQLLFEMAEKRIPVEERVFGEGHYLRSSFIQPYQCQHVLIEGVTVKDSPMWQIHPVLSDNVIVRGVKIIGHGPNTDGVNPESCRNVLIEDCYFDNGDDCIAIKSGRNEDGRRLGIPSENIVIRRNEMRDGHGGVTIGSEISGGVRHVYAEDNVMDSPNLDRALRIKTNSVRGGTIEHIYFKNNTVKSLKHEVVCIDMMYEEGDAGPHRPVVRHIEVEGLKSSGGRYGVKIAAYAHSPVTHFKMKDCVIDDVTYPLSLEHAVSPAFQKVVINGEQINSLKSEKQSF

SEQ ID NO: 220 - protein, 463 aa, Bacillus altitudinis
MSKKRNLMYVTAGTVLTGIGLARLAKQGKKDKETNGIDHVLKQIKAPKFPDREFNVIHYGADNKGIELSTNAIQSAIDDAHRLKGGRVLIPEGTFLTGALELKSNVELHLHENAYVTFSQDTRDYLPLVLTRYEGIELYNYSPLIYAHHAENIAITGAGTLDGRGDEHHWWPWKYGTNGQPSQDRDRQLLFEMAEKRIPVEERVFGEGHYLRSSFIQPYNCQNILIEGVTVKDSPMWQVHPVLSENVIVRGVNIIGHGPNTDGVNPESCRHVLIEDCYFDNGDDCIAIKSGRNEDGRRIGVPSENIVIRRNTMRDGHGGVTIGSEISGGVKYVYAEDNVMDSPNLDRALRIKTNSVRGGTIEHIYFKNNLVKSLKHEVVCIDMMYEEGDAGPHRPVVRHIEVEGLKSSGGRYGVKIAAYSHSPVTHFKMKGCVIDDVTYPLSLEHAVSPSFQKVVINGEHINQ

SEQ ID NO: 221 - protein, 463 aa, Bacillus pumilus
MSKKRDLMYITAGTVLTGIGLTRLARQGKKDKEANGIDQVLKQIKAPLFPDREFNVVHYGADAKGTELSTDAIQSAIDDAHRLKGGRVLIPEGTFVTGALELKSHVELHLHEKAYVAFSQNRKDYLPLVLTRYEGVELYNYSPLIYAHHAENIAITGAGTLDGRGDEHHWWPWKYGTNGQPSQDRDRQLLFDMAEKRIPVEERVFGEGHYLRSSFIQPYQCQQVLIEGVTVKDSPMWQIHPVLSENVIVRGVHIIGHGPNTDGVNPESCRNVLIEDCYFDNGDDCIAIKSGRNEDGRRIGIPSENIVIRRNKMRDGHGGVTIGSEISGGVRYVYAENNVMDSPNLDRALRIKTNSVRGGTIEHIYFKNNTVKSLKHEVVCIDMMYEEGDAGPHRPVVRHIEVEGLKSSGGRYGVKIAAYSHSPVTHFNMKNCVIDHVTYPLSLEHAVAPSFQKVVINGEQINK

SEQ ID NO: 222 - protein, 194 aa, Bacillus subtilis
ADKVVHETIIVPKNTTYDGKGQRFVAGKELGDGSQSENQDPVFRVEDGATLKNVVLGAPAADGVHTYGNVNIQNVKWEDVGEDALTVKKEGKVTIDGGSAQKASDKIFQINKASTFTVKNFTADNGGKFIRQLGGSTFHVDVIIDKCTITNMKEAIFRTDSKTSTVRMTNTRYSNVGQKWIGVQHIYENNNTQF

SEQ ID NO: 223 - protein, 302 aa, Bacillus safensis
ANFNQQGFSTLNGGTTGGEGGKTVTVKTGNELLAALKNKGTNEKLKIVVDGTITPSNTSANKIDVKDTNNVSIVGKGTNGEFNGIGIKVWRANNIIIRNLKIHHSKIGDKDAIGIEGGSKNIWVDHNELYNTLNSGKDDYDGLFDVKNDSDYITFSWNYVHDSWKTMLMGSSDNDNYNRKITFHNNRFENLNSRVPSMRFGEGHVYNNYYKGILTTAINSRMGAKMRIEHNVFENTNNAIGSWDSSQVGTWHVINNSYINSTGSLPTSSTGTYNPPYNYSLLNVNNVKSEVISNAGVGKVNP

SEQ ID NO: 224 - protein, 320 aa, Bacillus pumilus
AAKYPESPSIMANFNQQGFSTLNGGATGGEGGKTVTVKTGSELLAALKNKGTNEKLKIVVDGTITPSNTSANKIDVKDTNNVSIVGKGTRGEFNGVGIKVWRANNIIIRNLKIHHSKIGDKDAIGIEGGSKNIWVDHNELYNTLNSGKDDYDGLFDVKNDSDYITFSWNYVHDSWKTMLMGSSDNDNYNRKITFHNNRFENLNSRVPSMRFGEGHVYYNYYKGILATAINSRMGAKMRIEHNVFENTNNAIGSWYSRQVGTWHVINNSDINSTGSLPTSSTGTYNPPYNYSVLDVNDVKSEVISNAGVEKEKIKLEFSEG

SEQ ID NO: 225 - protein, 302 aa, Bacillus licheniformis
ADFSLKGFAALNGGTTGGEGGQTVTVTTGDQLIAALKNKNANTPLKIYVNGTITTSNTSASKIDVKDVSNVSIVGSGTKGELKGIGIKIWRANNIIIRNLKIHEVASGDKDAIGIEGPSKNIWVDHNELYHSLNVDKDYYDGLFDVKRDAEYITFSWNYVHDGWKSMLMGSSDSDNYNRTITFHHNWFENLNSRVPSFRFGEGHIYNNYFNKIIDSGINSRMGARIRIENNLFENAKDPIVSWYSSSPGYWHVSNNKFVNSRGSMPTTSTTTYNPPYSYSLDNVDNVKSIVKQNAGVGKINP

SEQ ID NO: 226 - protein, 400 aa, Bacillus amyloliquefaciens
ADLGRQTLGTNDGWGAASGGTIGGAKASSSNVYTVSNRQQLVSALGGSANSTPKIIYIQGTINMNTDDRNKTLGFDDYKDPAYDPNAYLKAYDPDKWGKKAPSGTQEDARQRSQKNQKARVVVDIPSNTTIIGSGSNAKVTGGNFNIKNGVDNVIIRNIEFQDAYDYFPQWDPTDGSSGNWNSEYDNITINGATHIWIDHCTFNDGSNPDSGFPYYYGRKYQHHDGQTDIANGANYITLSYNKYHDHDKGSVIGNSDSKTSDEGKLKVTIHHNYYQNIVQRAPRVRYGQVHIYNNFYAGSKSAAYPFSYAWGAGHASKIYAQNNVFEVPGLAADKVISVFSGGKALHEDGTLLNGASINASAANGLNQSVGWTPSLHGSIGSSSNVKSDVISKAGSGILK

SEQ ID NO: 227 - protein, 368 aa, Aspergillus niger
MHSYQLLGLAAVGSLVSAAPAPSRVSEFAKKASTCTFTSASEASESISSCSDVVLSSIEVPAGETLDLSDAADGSTITFEGTTSFGYKEWKGPLIRFGGKDLTVTMADGAVIDGDGSRWWDSKGTNGGKTKPKFMYIHDVEDSTFKGINIKNTPVQAISVQATNVHLNDFTIDNSDGDDNGGHNTDGFDISESTGVYISGATVKNQDDCIAINSGESISFTGGTCSGGHGLSIGSVGGRDDNTVKNVTISDSTVSNSANGVRIKTIYKETGDVSEITYSNIQLSGITDYGIVIEQDYENGSPTGTPSTGIPITDVTVDGVTGTLEDDATQVYILCGDGSCSDWTWSGVDLSGGKTSDKCENVPSGASC

(19) United States
(12) Patent Application Publication
(10) Pub. No.: US 2022/0169999 A1
(43) Pub. Date: Jun. 2, 2022

(54) FUSION PROTEINS, RECOMBINANT BACTERIA, AND EXOSPORIUM FRAGEMENTS FOR PLANT HEALTH

(71) Applicants: Bayer CropScience LP, St. Louis, MO (US); Spogen Biotech Inc., St. Louis, MO (US)

(72) Inventors: Jörg AUGUSTIN, Chesterfield, MO (US); Damian CURTIS, Davis, CA (US); Elizabeth M. HENRY, Sacramento, CA (US); Sara K. HOTTON, Davis, CA (US); Anne LAMSA, Sacramento, CA (US); Lakshmi Praba MANAVALAN, Davis, CA (US); Varghese P. THOMAS, Davis, CA (US); Brian THOMPSON, Creve Coeur, MO (US)

(21) Appl. No.: 17/439,990

(22) PCT Filed: Mar. 18, 2020
(86) PCT No.: PCT/US2020/023260

§ 371 Date: Sep. 16, 2021

Related U.S. Application Data
(60) Provisional application No. 62/820,789, filed on Mar. 19, 2019.

(51) Int. Cl. C12N 9/24; C07K 14/32; C12N 9/88; A01N 63/22; A01N 63/50; A01P 21/00

(52) U.S. Cl. CPC C12N 9/2402; C07K 14/32; C12N 9/88; A01N 63/22; C07K 2319/00; C12Y 402/02002; A01N 63/50; A01P 21/00; C12Y 302/01015

(57) ABSTRACT

The present invention relates to a fusion protein having a targeting sequence, exosporium protein, or exosporium protein fragment that targets the fusion protein to the exosporium of a recombinant Bacillus cereus family member and a pectinase enzyme, wherein the pectinase is a pectate lyase from Bacillus spp. having any of SEQ ID NOs: 213-217 and 222-226 or a polygalaturonase from Aspergillus niger or certain Bacillus species that can have any of SEQ ID NOs: 210-212, 218-221, and 227. The present invention also provides a recombinant Bacillus cereus family member that expresses such fusion protein and exosporium fragments derived from such recombinant Bacillus cereus family member. Methods of using such recombinant Bacillus cereus family members or exosporium fragments derived therefrom for plant growth promotion are also provided.

### CROSS-REFERENCE TO RELATED APPLICATIONS

This application claims priority to U.S. Provisional Patent Application No. 62/820,789, which was filed on Mar. 19, 2019, the entire contents of which are incorporated herein by reference in its entirety.

### FIELD OF THE INVENTION

The present invention relates to fusion proteins containing a targeting sequence, exosporium protein, or exosporium protein fragment that targets the fusion protein to the exosporium of a recombinant Bacillus cereus family member. The fusion proteins further comprise a pectinase, including a pectate lyase or a polygalacturonase. The invention further relates to recombinant Bacillus cereus family members that express the fusion proteins, exosporium fragments derived from the recombinant Bacillus cereus family members, and formulations containing the recombinant Bacillus cereus family members or exosporium fragments. Plant seeds treated with the recombinant Bacillus cereus family members, exosporium fragments, or formulations are also provided. The invention further relates to methods for stimulating plant growth and/or promoting plant health using the recombinant Bacillus cereus family members, exosporium fragments, or formulations.

### REFERENCE TO SEQUENCE LISTING SUBMITTED ELECTRONICALLY

The official copy of the sequence listing is submitted electronically via EFS-Web as an ASCII-formatted sequence listing with a file named “BCS199003WO_ST25.txt” created on Mar. 13, 2020, and having a size of 321 kilobytes, and is filed concurrently with the specification. The sequence listing contained in this ASCII-formatted document is part of the specification and is herein incorporated by reference in its entirety.

### BACKGROUND OF THE INVENTION

Within the zone surrounding a plant's roots is a region called the rhizosphere. In the rhizosphere, bacteria, fungi, and other organisms compete for nutrients and for binding to the root structures of the plant. Both detrimental and beneficial bacteria and fungi can occupy the rhizosphere. The bacteria, fungi, and the root system of the plant can all be influenced by the actions of peptides, enzymes, and other proteins in the rhizosphere. Augmentation of soil or treatment of plants with certain of these peptides, enzymes, or other proteins would have beneficial effects on the overall populations of beneficial soil bacteria and fungi, create a healthier overall soil environment for plant growth, improve plant growth, and provide for the protection of plants against certain bacterial and fungal pathogens. However, previous attempts to introduce peptides, enzymes, and other proteins into soil to induce such beneficial effects on plants have been hampered by the low survival of enzymes, proteins, and peptides in soil. Additionally, the prevalence of proteases naturally present in the soil can lead to degradation of the proteins in the soil. The environment around the roots of a plant (the rhizosphere) is a unique mixture of bacteria, fungi, nutrients, and roots that has different qualities than that of native soil. The symbiotic relationship between these organisms is unique, and could be altered for the better with inclusion of exogenous proteins. The high concentration of fungi and bacteria in the rhizosphere causes even greater degradation of proteins due to abnormally high levels of proteases and other elements detrimental to proteins in the soil. In addition, enzymes and other proteins introduced into soil can dissipate away from plant roots quickly.

Thus, there exists a need in the art for a method for effectively delivering peptides, enzymes, and other proteins to plants (e.g., to plant root systems) and for extending the period of time during which such molecules remain active. Furthermore, there exists a need in the art for a method of selectively targeting such peptides, enzymes, and proteins to the rhizosphere and to plant leaves and plant roots in particular.

### BRIEF SUMMARY OF THE INVENTION

A fusion protein is provided. The fusion protein comprises a targeting sequence, exosporium protein, or exosporium protein fragment that targets the fusion protein to the exosporium of a recombinant Bacillus cereus family member. The fusion protein also comprises a pectinase, such as a pecate lyase or a polygalacturonase.

The pectate lyase is a pectate lyase from Bacillus subtilis, Bacillus pumilus, Bacillus safensis, Bacillus licheniformis or Bacillus amyloliquefaciens or a pectate lyase comprising an amino acid sequence having at least 60%, at least 65%, at least 70%, at least 75%, at least 80%, at least 85%, at least 90%, at least 95%, at least 98%, at least 99%, or 100% identity to any one of SEQ ID NOs: 213-217 and 222-226.

The polygalacturonase is an endopolygalacturonase from Apergillus niger ATCC 9029; a Bacillus simplex endopolygalacturonase; a Bacillus safensis polygalacturonase; a Bacillus altitudinis polygalacturonase; a Bacillus licheniformis polygalacturonase; a Bacillus pumilus polygalacturonase or a polygalacturonase comprising an amino acid sequence having at least 80%, at least 85%, at least 90%, at least 95%, at least 98%, at least 99%, or 100% identity to SEQ ID NOs: 210-227; or a polygalacturonase comprising an amino acid sequence having at least 60%, at least 65%, at least 70%, at least 75%, at least 80%, at least 85%, at least 90%, at least 95%, at least 98%, at least 99%, or 100% identity to any one of SEQ ID NOs: 218-221, or a polygalacturonase comprising an amino acid sequence having at least 70%, at least 75%, at least 80%, at least 85%, at least 90%, at least 95%, at least 98%, at least 99%, or 100% identity to SEQ ID NOs: 211 or 212.

In one embodiment, the polygalacturonase comprising an amino acid sequence having at least 60%, at least 65%, at least 70%, at least 75%, at least 80%, at least 85%, at least 90%, at least 95%, at least 98%, at least 99%, or 100% identity to any one of SEQ ID NOs: 218-221 is from Bacillus and also contains catalytic residues that are conserved with those of the polygalacturonase I of Aspergillus niger EPG I, which are amino acid residues Asp186, Asp207, Asp208, and H229 of SEQ ID NOs: 210 and 227.

A recombinant Bacillus cereus family member is provided. The recombinant Bacillus cereus family member expresses a fusion protein. The fusion protein can be any of the fusion proteins described herein.

A whole broth culture of the recombinant Bacillus cereus family member is provided. A fermentation product of the recombinant Bacillus cereus family member is provided.

Exosporium fragments are provided. The exosporium fragments are derived from a recombinant Bacillus cereus family member, including a whole broth or fermentation product of a recombinant Bacillus cereus family member. The recombinant Bacillus cereus family member can be any of the recombinant Bacillus cereus family members described herein. The exosporium fragments can comprise any of the fusion proteins described herein.

A formulation is provided. The formulation comprises any of the recombinant Bacillus cereus family members described herein, including a fermentation product of any of the recombinant Bacillus cereus family members described herein. The formulation further comprises an agriculturally acceptable carrier.

Another formulation is provided. The formulation comprises exosporium fragments derived from any of the recombinant Bacillus cereus family members described herein, including a whole broth of a recombinant Bacillus cereus family member described herein. The formulation further comprises an agriculturally acceptable carrier.

Yet another formulation is provided. The formulation comprises a recombinant Bacillus cereus family member that expresses a fusion protein. Alternatively, or in addition, the formulation comprises exosporium fragments derived from a Bacillus cereus family member that expresses a fusion protein. The fusion protein comprises a targeting sequence, exosporium protein, or exosporium protein fragment that targets the fusion protein to the exosporium of a recombinant Bacillus cereus family member. The fusion protein also comprises a pectinase, such as any one of the pectate lyases or polygalacturonases disclosed herein. The formulation further comprises a second enzyme.

A treated plant seed is provided. The plant seed can be treated with any of the recombinant Bacillus cereus family members described herein. The recombinant Bacillus cereus family member can express any of the fusion proteins described herein.

Another treated plant seed is provided. The plant seed can be treated with any of the exosporium fragments described herein. The exosporium fragments can be derived from any of the Bacillus cereus family members described herein. The exosporium fragments can comprise any of the fusion proteins described herein.

Yet another treated plant seed is provided. The plant seed can be treated with any of the formulations described herein.

A method for stimulating plant growth and/or promoting plant health is provided. The method comprises applying a recombinant Bacillus cereus family member to a plant growth medium, a plant, a plant seed, or an area surrounding a plant or a plant seed. The recombinant Bacillus cereus family member can comprise any of the recombinant Bacillus cereus family members described herein. The recombinant Bacillus cereus family member can express any of the fusion proteins described herein.

Another method for stimulating plant growth and/or promoting plant health is provided. The method comprises applying exosporium fragments to a plant growth medium, a plant, a plant seed, or an area surrounding a plant or a plant seed. The exosporium fragments can comprise exosporium fragments derived from any of the recombinant Bacillus cereus family members described herein. The exosporium fragments can comprise any of the fusion proteins described herein.

Yet another method for stimulating plant growth and/or promoting plant health is provided. The method comprises applying a formulation to a plant growth medium, a plant, a plant seed, or an area surrounding a plant or a plant seed. The formulation can comprise any of the formulations described herein.

Another method for stimulating plant growth and/or promoting plant health is provided. The method comprises applying a recombinant Bacillus cereus family member expressing a fusion protein to a plant growth medium, a plant, a plant seed, or an area surrounding a plant or a plant seed. The fusion protein comprises a targeting sequence, exosporium protein, or exosporium protein fragment that targets the fusion protein to the exosporium of a recombinant Bacillus cereus family member. The fusion protein further comprises a pectinase, such as any one of the pectate lyases or polygalacturonases disclosed herein.

Yet another method for stimulating plant growth and/or promoting plant health is provided. The method comprises applying exosporium fragments derived from spores of a recombinant Bacillus cereus family member expressing a fusion protein to a plant growth medium, a plant, a plant seed, or an area surrounding a plant or a plant seed. The fusion protein comprises a targeting sequence, exosporium protein, or exosporium protein fragment that targets the fusion protein to the exosporium of a recombinant Bacillus cereus family member. The fusion protein further comprises a pectinase, such as any one of the pectate lyases or polygalacturonases disclosed herein.

A further method for stimulating plant growth and/or promoting plant health is provided. The method comprises applying a recombinant Bacillus cereus family member expressing a fusion protein to a plant growth medium, a plant, a plant seed, or an area surrounding a plant or a plant seed. The fusion protein comprises a targeting sequence, exosporium protein, or exosporium protein fragment that targets the fusion protein to the exosporium of a recombinant Bacillus cereus family member. The fusion protein further comprises a pectinase, such as any one of the pectate lyases or polygalacturonases disclosed herein. The method further comprises applying a second enzyme to the plant growth medium, the plant, the plant seed, or the area surrounding the plant or the plant seed.

Another method for stimulating plant growth and/or promoting plant health is provided. The method comprises applying exosporium fragments derived from spores of a recombinant Bacillus cereus expressing a fusion protein to a plant growth medium, a plant, a plant seed, or an area surrounding a plant or a plant seed. The fusion protein comprises a targeting sequence, exosporium protein, or exosporium protein fragment that targets the fusion protein to the exosporium of a recombinant Bacillus cereus family member. The fusion protein further comprises a pectinase, such as any one of the pectate lyases or polygalacturonases disclosed herein. The method further comprises applying a second enzyme to the plant growth medium, the plant, the plant seed, or the area surrounding the plant or the plant seed.

Other objects and features will be in part apparent and in part pointed out hereinafter.

### Definitions

When the articles “a”, “an”, “one”, “the”, and “said” are used herein, they mean “at least one” or “one or more” unless otherwise indicated.

The term “Bacillus cereus family member” as used herein refers to any Bacillus species that is capable of producing an exosporium. Thus, the Bacillus cereus family of bacteria includes the species Bacillus anthracia, Bacillus cereus, Bacillus thuringiensis, Bacillus mycoides, Bacillus pseudomycoides, Bacillus samanii, Bacillus gaemokensis, Bacillus weihenstephensis, and Bacillus toyoiensis. Bacillus cereus family members are also referred to in the art as “Bacillus cereus senso lato.”

The terms “comprising,” “including” and “having” are intended to be inclusive and mean that there may be additional elements other than the listed elements.

The term “free enzyme” as used herein refers to an enzyme preparation that is substantially free of intact cells. The term “free enzyme” includes, but is not limited to, crude cell extracts containing an enzyme, partially purified, substantially purified, or purified enzyme. Free enzymes can optionally be immobilized on a chemical matrix or support to allow for controlled release of the enzyme. The term “immobilizing” as used herein in reference to immobilizing an enzyme on a matrix or support refers to the binding of the enzyme to the matrix or support such that the enzyme is maintained on the matrix or support or released from the support over a controlled period of time, instead of dissipating into the environment in an uncontrolled manner Illustrative matrices and supports include, but are not limited to, charcoal, biochar, nanocarbon, agarose, an alginate, cellulose, a cellulose derivative, silica, plastic, stainless steel, glass, polystyrene, a ceramic, dolomite, a clay, diatomaceous earth, talc, a polymer, a gum, a water-dispersable material, and combinations of any thereof.

The term “foliar” used herein with respect to the application of enzymes or recombinant microorganisms to plants means that the enzyme or recombinant microorganism is applied to one or more aerial portions of the plant, including stems, leaves, fruits, flowers, or other exposed aerial portions of the plant.

The term “fusion protein” as used herein refers to a protein having a polypeptide sequence that comprises sequences derived from two or more separate proteins. A fusion protein can be generated by joining together a nucleic acid molecule that encodes all or part of a first polypeptide with a nucleic acid molecule that encodes all or part of a second polypeptide to create a nucleic acid sequence which, when expressed, yields a single polypeptide having functional properties derived from each of the original proteins.

The term “germination rate” as used herein refers to the number of seeds that germinate during a particular time period. For example, a germination rate of 85% indicates that 85 out of 100 seeds germinate during a given time period.

The term “inactivate” or “inactivation” as used herein in reference to the inactivation of spores of a recombinant Bacillus cereus family member means that the spores are unable to germinate, or that the spores can germinate, but are damaged such that germination does not result in a living bacterium. The terms “partially inactivate” or “partial inactivation” mean that a percentage of the spores are inactivated, but that some spores retain the ability to germinate and return to a live, replicating state. The term “genetic inactivation” refers to inactivation of spores a recombinant Bacillus cereus family member by a mutation of the spore's DNA that results in complete or partial inactivation of the spore. The terms “physical inactivation” and “chemical inactivation” refer to inactivation of spores using any physical or chemical means, e.g., by heat treatment, gamma irradiation, x-ray irradiation, UV-A irradiation, UV-B irradiation, or treatment with a solvent such as glutaraldehyde, formaldehyde, hydrogen peroxide, acetic acid, bleach, chloroform, phenol, or any combination thereof.

The terms “native sequence”, “native amino acid sequence”, “wild-type sequence”, and “wild-type amino acid sequence” are used interchangeably herein to refer to an amino acid sequence as it exists in a naturally occurring protein.

A “plant growth medium” includes any material that is capable of supporting the growth of a plant.

The terms “promoting plant growth” and “stimulating plant growth” are used interchangeably herein, and refer to the ability to enhance or increase at least one of the plant's height, weight, leaf size, root size, fruit size, shoot size or stem size, and/or the ability to increase protein yield from the plant and/or to increase crop yield and/or to improve plant vigor. For example, this may relate to increased length and/or fresh and/or dry weights of roots and/or shoots of treated plants or crops compared to untreated plants or crops.

Increased yield of a plant, in particular of an agricultural, silvicultural and/or ornamental plant, means that the yield of a product of the respective plant is increased by a measurable amount over the yield of the same product of the plant produced under the same conditions, but without the application of the compositions disclosed herein.

Improved plant vigor includes the following: (a) improved vitality of the plant, (b) improved quality of the plant and/or of the plant products, e.g., enhanced protein content, (c) improved visual appearance, (d) delay of senescence, (e) enhanced root growth and/or more developed root system (e.g., determined by the dry mass of the root), (f) enhanced nodulation, in particular rhizobial nodulation, (g) longer panicles, (h) bigger leaf blade, (i) less dead basal leaves, (j) increased chlorophyll content, (k) prolonged photosynthetically active period, (l) increased or improved plant stand density, (m) less plant verse (lodging), (n) increased plant weight, (o) increased plant height, (p) tillering increase, (q) stronger and/or more productive tillers, (r) less non-productive tillers, (s) enhanced photosynthetic activity and/or enhanced pigment content and thus greener leaf color, (t) earlier and/or improved germination, (u) improved and/or more uniform and/or earlier emergence, (v) increased shoot growth, (w) earlier flowering, (x) earlier fruiting, (y) earlier grain maturity, (z) less fertilizers needed, (aa) less seeds needed.

The term “recombinant” as used in reference to the bacteria described herein encompasses bacteria having any genetic modification as compared to wild-type bacteria of the same type, including bacteria that have been modified to delete of a gene or a portion of a gene (e.g., bacteria that have a “knock-out” of a gene), as well as bacteria that have been modified to express an exogenous peptide or protein.

The term “rhizosphere” is used interchangeably with “root zone” to denote that segment of the soil that surrounds the roots of a plant and is influenced by them.

The term “synergistically effective amount” as used herein refers an amount of a first substance (e.g., a first enzyme) that when used in combination with a second substance (e.g., a second enzyme) produces a biological effect that is greater than the sum of the biological effects of each of the respective first and second substances when used alone.

The term “targeting sequence” as used herein refers to a polypeptide sequence that, when present as part of a longer polypeptide or a protein, results in the localization of the longer polypeptide or the protein to a specific subcellular location. The targeting sequences described herein result in localization of proteins to the exosporium of a Bacillus cereus family member.

### BRIEF DESCRIPTION OF THE DRAWINGS

FIGS. 1A and 1B depict alignments of the amino acid sequence of an amino-terminal portion of Bacillus anthracis Sterne strain BclA and with the corresponding region from various exosporium proteins from Bacillus cereus family members.

### DETAILED DESCRIPTION OF THE INVENTION

### I. Fusion Proteins for Expression in Bacillus Cereus Family Members

The present invention relates to fusion proteins comprising a targeting sequence, exosporium protein, or exosporium protein fragment that targets the fusion protein to the exosporium of a recombinant Bacillus cereus family member. The fusion protein further comprises a pectinase, such as any one of the pectate lyases or polygalacturonases disclosed herein. When expressed in Bacillus cereus family member bacteria, these fusion proteins are targeted to the exosporium layer of the spore and are physically oriented such that the pectinase is displayed on the outside of the spore.

This Bacillus exosporium display (BEMD) system can be used to deliver the pectinase, such as any one of the pectate lyases or polygalacturonases disclosed herein, to plants (e.g., to plant foliage, fruits, flowers, stems, or roots) or to a plant growth medium such as soil. Enzymes and proteins delivered to the soil or another plant growth medium in this manner persist and exhibit activity in the soil for extended periods of time. Introduction of recombinant Bacillus cereus family member bacteria expressing the fusion proteins described herein into soil or the rhizosphere of a plant leads to a beneficial enhancement of plant growth in many different soil conditions. The use of the BEMD to create these enzymes allows them to continue to exert their beneficial results to the plant and the rhizosphere over the first months of a plant's life.

In addition, as is described further hereinbelow, the BEMD system can be modified such that the exosporium of the recombinant Bacillus cereus family member can be removed from the spore, generating exosporium fragments containing the fusion proteins. The exosporium fragments can also be used to deliver the pectinase, such as any one of the pectate lyases or polygalacturonases disclosed herein, to plants in a cell-free preparation.

A. Targeting Sequences, Exosporium Proteins, and Exosporium Protein Fragments for Targeting Pectinases to the Exosporium of a Bacillus cereus Family Member

For ease of reference, descriptions of the amino acid sequences for the targeting sequences, exosporium proteins, and exosporium protein fragments that can be used for targeting of enzymes or proteins (e.g., pectinase, such as any one of the pectate lyases or polygalacturonases disclosed herein) to the exosporium of a Bacillus cereus family members, are provided in Table 1 together with their SEQ ID NOs.

TABLE 1
Peptide and Protein Sequences Used for Targeting of Proteins or Peptides of
Interest to the Exosporium of Bacillus cereus Family Members

| Protein, Protein Fragment, or Targeting Sequence | SEQ ID NO: |
|---|---|
| AA 1-41 of BclA (B. anthracis Sterne) | 1 |
| Full length BclA (B. anthracis Sterne) | 2 |
| AA 1-33 of BetA/BAS3290 (B. anthracis Sterne) | 3 |
| Full length BetA/BAS3290 (B. anthracis Sterne) | 4 |
| Met + AA 2-43 of BAS4623 (B. anthracis Sterne) | 5 |
| Full length BAS4623 (B. anthracis Sterne) | 6 |
| AA 1-34 of BclB (B. anthracis Sterne) | 7 |
| Full length BclB (B. anthracis Sterne) | 8 |
| AA 1-30 of BAS1882 (B. anthracis Sterne) | 9 |
| Full length BAS1882 (B. anthracis Sterne) | 10 |
| AA 1-39 of gene 2280 (B. weihenstephensis KBAB4) | 11 |
| Full length KBAB4 gene 2280 (B. weihenstephensis KBAB4) | 12 |
| AA 1-39 of gene 3572 (B. weihenstephensis KBAB4) | 13 |
| Full Length KBAB4 gene 3572 (B. weihenstephensis KBAB4) | 14 |
| AA 1-49 of Exosporium Leader Peptide (B. cereus VD200) | 15 |
| Full Length Exosporium Leader Peptide (B. cereus VD200) | 16 |
| AA 1-33 of Exosporium Leader Peptide (B. cereus VD166) | 17 |
| Full Length Exosporium Leader Peptide (B. cereus VD166) | 18 |
| AA 1-39 of hypothetical protein IKG_04663 (B. cereus VD200) | 19 |
| Hypothetical protein IKG_04663, partial (B. cereus VD200) | 20 |
| AA 1-39 of YVTN β-propeller protein (B. weihenstephensis KBAB4) | 21 |
| Full length YVTN β-propeller protein KBAB4 (B. weihenstephensis KBAB4) | 22 |
| AA 1-30 of hypothetical protein bcerkbab4_2363 | 23 |
| (B. weihenstephensis KBAB4) | |
| Full length hypothetical protein bcerkbab4_2363 KBAB4 | 24 |
| (B. weihenstephensis KBAB4) | |
| AA 1-30 of hypothetical protein bcerkbab4_2131 | 25 |
| (B. weihenstephensis KBAB4) | |
| Full length hypothetical protein bcerkbab4_2131 | 26 |
| (B. weihenstephensis KBAB4) | |
| AA 1-36 of triple helix repeat containing collagen | 27 |
| (B. weihenstephensis KBAB4) | |
| Full length triple helix repeat-containing collagen KBAB4 | 28 |
| AA 1-39 of hypothetical protein bmyco0001_21660 (B. mycoides 2048) | 29 |
| Full length hypothetical protein bmyco0001_21660 (B. mycoides 2048) | 30 |
| AA 1-30 of hypothetical protein bmyc0001_22540 (B. mycoides 2048) | 31 |
| Full length hypothetical protein bmyc0001_22540 (B. mycoides 2048) | 32 |
| AA 1-21 of hypothetical protein bmyc0001_21510 (B. mycoides 2048) | 33 |
| Full length hypothetical protein bmyc0001_21510 (B. mycoides 2048) | 34 |
| AA 1-22 of collagen triple helix repeat protein (B. thuringiensis 35646) | 35 |
| Full length collagen triple helix repeat protein (B. thuringiensis 35646) | 36 |
| AA 1-35 of hypothetical protein WP_69652 (B. cereus) | 43 |
| Full length hypothetical protein WP_69652 (B. cereus) | 44 |
| AA 1-41 of exosporium leader WP016117717 (B. cereus) | 45 |
| Full length exosporium leader WP016117717 (B. cereus) | 46 |
| AA 1-49 of exosporium peptide WP002105192 (B. cereus) | 47 |
| Full length exosporium peptide WP002105192 (B. cereus) | 48 |
| AA 1-38 of hypothetical protein WP87353 (B. cereus) | 49 |
| Full length hypothetical protein WP87353 (B. cereus) | 50 |
| AA 1-39 of exosporium peptide 02112369 (B. cereus) | 51 |
| Full length exosporium peptide 02112369 (B. cereus) | 52 |
| AA 1-39 of exosporium protein WP016099770 (B. cereus) | 53 |
| Full length exosporium protein WP016099770 (SEQ ID NO: 54) | 54 |
| AA 1-36 of hypothetical protein YP006612525 (B. thuringiensis) | 55 |
| Full length hypothetical protein YP006612525 (B. thuringiensis) | 56 |
| AA 1-136 of hypothetical protein TIGR03720 (B. mycoides) | 57* |
| Full length hypothetical protein TIGR03720 (B. mycoides) | 58* |
| AA 1-36 of collagen triple helix repeat domain protein | 59 |
| (B. cereus ATCC 10987) | |
| Full length collagen triple helix repeat domain protein | 60 |
| (B. cereus ATCC 10987) | |
| AA 1-39 of collagen-like protein (B. cereus E33L) | 61 |
| Full length collagen-like protein (B. cereus E33L) | 62 |
| AA 1-41 of triple helix repeat-containing collagen | 63 |
| (B. weihenstephanensis KBAB4) | |
| Full length triple helix repeat-containing collagen | 64 |
| (B. weihenstephanensis KBAB4) | |
| AA 1-30 of hypothetical protein BALH_2230 | 65 |
| (B. thuringiensis str. Al Hakam) | |
| Full length hypothetical protein BALH_2230 | 66 |
| (B. thuringiensis str. Al Hakam) | |
| AA 1-33 of triple helix repeat-containing collagen (B. cereus ATCC 14579) | 67 |
| Full length triple helix repeat-containing collagen (B. cereus ATCC 14579) | 68 |
| AA 1-44 of collagen triple helix repeat (B. cereus) | 69 |
| Full length collagen triple helix repeat (B. cereus) | 70 |
| AA 1-38 of triple helix repeat-containing collagen (B. cereus ATCC 14579) | 71 |
| Full length triple helix repeat-containing collagen (B. cereus ATCC 14579) | 72 |
| AA 1-30 of hypothetical protein BCZK1835 (B. cereus E33L) | 73 |
| Full length hypothetical protein BCZK1835 (B. cereus E33L) | 74 |
| AA 1-48 of triple helix repeat-containing collagen | 75 |
| (B. weihenstephanensis KBAB4) | |
| Full length triple helix repeat-containing collagen | 76 |
| (B. weihenstephanensis KBAB4) | |
| AA 1-30 of triple helix repeat-containing collagen (B. cereus ATCC 14579) | 77 |
| Full length triple helix repeat-containing collagen (B. cereus ATCC 14579) | 78 |
| AA 1-39 of hypothetical protein BC4725 (B. cereus ATCC 14579) | 79 |
| Full length hypothetical protein BC4725 (B. cereus ATCC 14579) | 80 |
| AA 1-44 of hypothetical protein BCZK4476 (B. cereus E33L) | 81 |
| Full length hypothetical protein BCZK4476 (B. cereus E33L) | 82 |
| AA 1-40 of triple helix repeat-containing collagen | 83 |
| (B. anthracis str. ‘Ames Ancestor’) | |
| Full length triple helix repeat-containing collagen | 84 |
| (B. anthracis str. ‘Ames Ancestor’) | |
| AA 1-34 of BclA protein (B. thuringiensis serovar konkukian str. 97-27) | 85 |
| Full length BclA protein (B. thuringiensis serovar konkukian str. 97-27) | 86 |
| AA 1-34 of conserved hypothetical protein (B. cereus ATCC 10987) | 87 |
| Full length conserved hypothetical protein (B. cereus ATCC 10987) | 88 |
| AA 1-34 of triple helix repeat-containing collagen (B. cereus ATCC 14579) | 89 |
| Full length triple helix repeat-containing collagen (B. cereus ATCC 14579) | 90 |
| AA 1-99 of exosporium leader peptide partial sequence (B. cereus) | 91 |
| Exosporium leader peptide partial sequence (B. cereus) | 92 |
| AA 1-136 of hypothetical protein ER45_27600, partial sequence | 93 |
| (B. weihenstephanensis) | |
| Hypothetical protein ER45_27600, partial sequence (B. weihenstephanensis) | 94 |
| AA 1-196 of BclA (B. anthracis Sterne) | 95 |
| Met + AA 20-35 of BclA (B. anthracis Sterne) | 96 |
| Met + AA 12-27 of BetA/BAS3290 (B. anthracis Sterne) | 97 |
| Met + AA 18-33 of gene 2280 (B. weihenstephensis KBAB4) | 98 |
| Met + AA 18-33 of gene 3572 (B. weihenstephensis KBAB4) | 99 |
| Met + AA 12-27 of Exosporium Leader Peptide (B. cereus VD166) | 100 |
| Met + AA 18-33 of YVTN β-propeller protein | 101 |
| (B. weihenstephensis KBAB4) | |
| Met + AA 9-24 of hypothetical protein bcerkbab4_2363 | 102 |
| (B. weihenstephensis KBAB4) | |
| Met + AA 9-24 of hypothetical protein bcerkbab4_2131 | 103 |
| (B. weihenstephensis KBAB4) | |
| Met + AA 9-24 of hypothetical protein bmyc0001_22540 (B. mycoides 2048) | 104 |
| Met + AA 9-24 of BAS1882 (B. anthracis Sterne) | 105 |
| Met + AA 20-35 of exosporium leader WP016117717 (B. cereus) | 106 |
| Met + AA 9-24 of hypothetical protein BALH_2230 | 107 |
| (B. thuringiensis str. Al Hakam) | |
| Full length InhA (B. mycoides) | 108 |
| Full length BAS1141 (ExsY) (B. anthracis Sterne) | 109 |
| Full length BAS1144 (BxpB/ExsFA) (B. anthracis Sterne) | 110 |
| Full length BAS1145 (CotY)(B. anthracis Sterne) | 111 |
| Full length BAS1140 (B. anthracis Sterne) | 112 |
| Full length ExsFB (B. anthracis H9401) | 113 |
| Full length InhA1 (B. thuringiensis HD74) | 114 |
| Full length ExsJ (B. cereus ATCC 10876) | 115 |
| Full length ExsH (B. cereus) | 116 |
| Full length YjcA (B. anthracis Ames) | 117 |
| Full length YjcB (B. anthracis) | 118 |
| Full length BclC (B. anthracis Sterne) | 119 |
| Full length acid phosphatase | 120 |
| (Bacillus thuringiensis serovar konkukian str. 97-27) | |
| Full length InhA2 (B. thuringiensis HD74) | 121 |
| Full length InhA3 (B. mycoides) | 122 |
| Met + AA 23-38 of BAS4623 (B. anthracis Sterne) | 201 |
| Met + AA 13-28 of BclB (B. anthracis Sterne) | 202 |
| Cot Y variant (Bacillus anthracis) | 203 |
| BclA (Bacillus thuringiensis) | 204 |
| Amino acids 1-166 of BclA (Bacillus thuringiensis) | 205 |
| BclA (Bacillus anthracis) | 206 |
| AA 1-196 of BclA (Bacillus anthracis) | 207 |

AA = amino acids
*B. mycoides hypothetical protein TIGR03720 has 100% sequence identity with B. mycoides hypothetical protein WP003189234. Thus, SEQ ID NOs: 57 and 58 also represent amino acids 1-136 of B. mycoides hypothetical protein WP003189234 and full length B. mycoides hypothetical protein WP003189234, respectively.

Bacillus is a genus of rod-shaped bacteria. The Bacillus cereus family of bacteria includes any Bacillus species that is capable of producing an exosporium. Thus, the Bacillus cereus family of bacteria includes the species Bacillus anthracia, Bacillus cereus, Bacillus thuringiensis, Bacillus mycoides, Bacillus pseudomycoides, Bacillus samanii, Bacillus gaemokensis, Bacillus weihenstephensis, and Bacillus toyoiensis. Under stressful environmental conditions, Bacillus cereus family bacteria undergo sporulation and form oval endospores that can stay dormant for extended periods of time. The outermost layer of the endospores is known as the exosporium and comprises a basal layer surrounded by an external nap of hair-like projections. Filaments on the hair-like nap are predominantly formed by the collagen-like glycoprotein BclA, while the basal layer is comprised of a number of different proteins. Another collagen-related protein, BclB, is also present in the exosporium and exposed on endospores of Bacillus cereus family members. BclA, the major constituent of the surface nap, has been shown to be attached to the exosporium with its amino-terminus (N-terminus) positioned at the basal layer and its carboxy-terminus (C-terminus) extending outward from the spore.

The scientific literature describes the Bacillus cereus “family” or “group” as a subgroup within the genus Bacillus. See Priest et al., “Population Structure and Evolution of the Bacillus cereus Group,” J. Bacteriology, 2004, vol. 186. no. 23, pp. 7959-7970; Peng et al., “The Regulation of Exosporium-Related Genes in Bacillus thuringiensis,” Nature Scientific Reports, 2016, vol. 6, no. 19005, pp. 1-12. Peng et al. states:
Spores of the B. cereus group are complex, multilayered structures. The nucleoid containing core is enclosed within a peptidoglycan cortex, which is surrounded by the spore coat. Spores of all the B. cereus group species are encircled by an additional loose-fitting layer called the exosporium, which is not present on other species such as Bacillus subtilis, for which the coat constitutes the outermost layer of the mature spore. The exosporium is a balloon-like layer that acts as the outer permeability barrier of the spore and contributes to spore survival and virulence.

It was previously discovered that certain sequences from the N-terminal regions of BclA and BclB could be used to target a peptide or protein to the exosporium of a Bacillus cereus family member endospore (see U.S. Patent Application Publication Nos. 2010/0233124 and 2011/0281316, and Thompson et al., “Targeting of the BclA and BclB Proteins to the Bacillus anthracis Spore Surface,” Molecular Microbiology 70(2):421-34 (2008)). It was also found that the BetA/BAS3290 protein of Bacillus anthracis localized to the exosporium. Further targeting sequences, as well as exosporium proteins and fragments of exosporium proteins, that can be incorporated into a fusion protein and used to target a peptide or protein of interest to the exosporium of a recombinant Bacillus cereus family member are described in U.S. Patent Application Publication Nos. 2016/0031948 and 2016/0108096, which are incorporated by reference herein in their entirety.

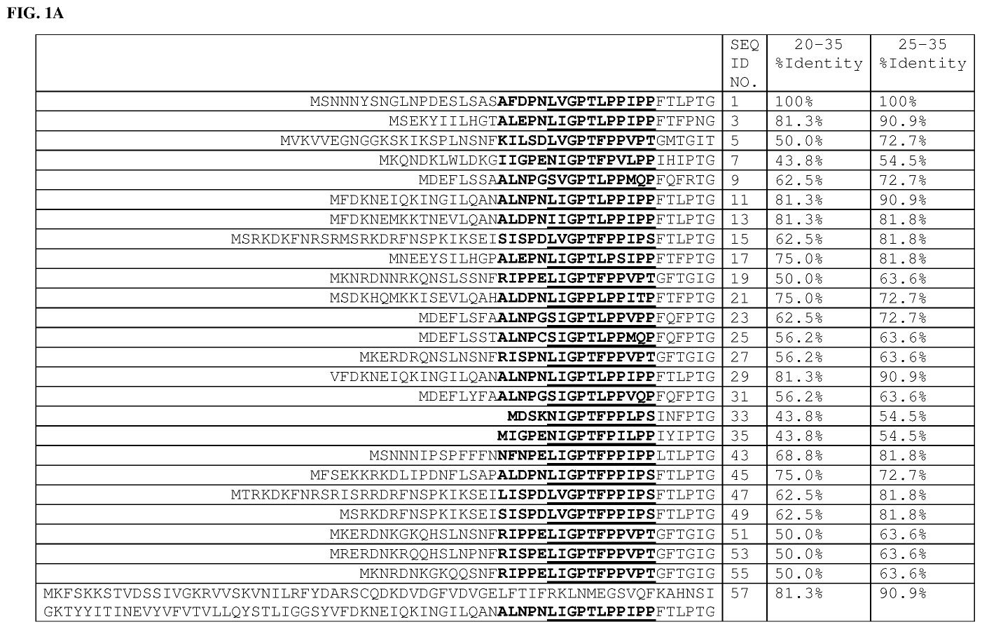

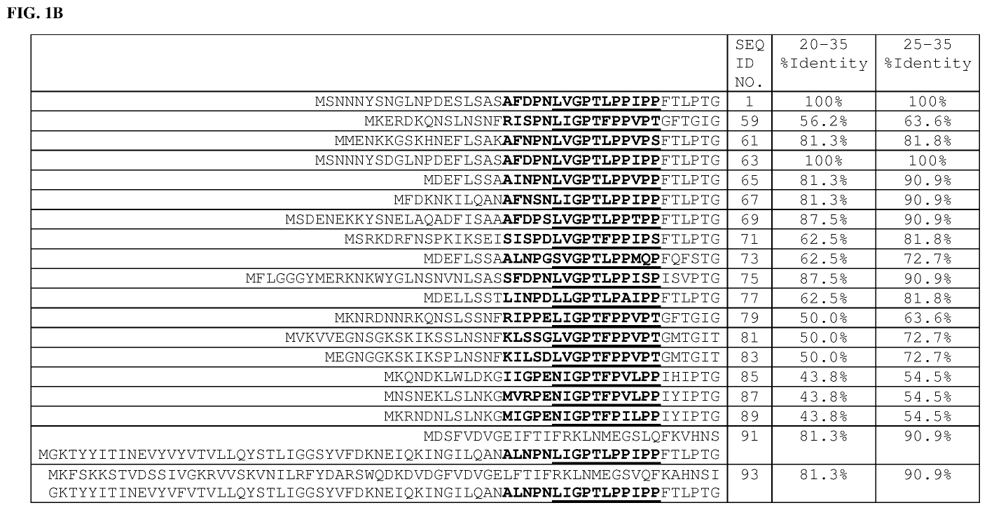

In particular, amino acids 20-35 of BclA from Bacillus anthracis Sterne strain have been found to be sufficient for targeting to the exosporium. A sequence alignment of amino acids 1-41 of BclA (SEQ ID NO: 1) with the corresponding N-terminal regions of several other Bacillus cereus family exosporium proteins and Bacillus cereus family proteins having related sequences is shown in FIGS. 1A and 1B. As can be seen from FIGS. 1A and 1B, there is a region of high homology among all of the proteins in the region corresponding to amino acids 20-41 of BclA. However, in these sequences, the amino acids corresponding to amino acids 36-41 of BclA contain secondary structure and are not necessary for fusion protein localization to the exosporium. The conserved targeting sequence region of BclA (amino acids 20-35 of SEQ ID NO: 1) is shown in bold in FIGS. 1A and 1B. A more highly conserved region spanning amino acids 25-35 of BclA within the targeting sequence is underlined in the sequences in FIGS. 1A and 1B, and is the recognition sequence for ExsFA/BxpB/ExsFB and homologs, which direct and assemble the described proteins on the surface of the exosporium. The amino acid sequences of SEQ ID NOs: 3, 5, and 7 in FIG. 1A are amino acids 1-33 of Bacillus anthracis Sterne strain BetA/BAS3290, a methionine followed by amino acids 2-43 of Bacillus anthracis Sterne strain BAS4623, and amino acids 1-34 of Bacillus anthracis Sterne strain BclB, respectively. (For BAS4623, it was found that replacing the valine present at position 1 in the native protein with a methionine resulted in better expression.) As can be seen from FIG. 1A, each of these sequences contains a conserved region corresponding to amino acids 20-35 of BclA (SEQ ID NO: 1; shown in bold), and a more highly conserved region corresponding to amino acids 25-35 of BclA (underlined).

Additional proteins from Bacillus cereus family members also contain the conserved targeting region. In particular, in FIGS. 1A and 1B, SEQ ID NO: 9 is amino acids 1-30 of Bacillus anthracis Sterne strain BAS1882, SEQ ID NO: 11 is amino acids 1-39 of the Bacillus weihenstephensis KBAB4 2280 gene product, SEQ ID NO: 13 is amino acids 1-39 of the Bacillus weihenstephensis KBAB4 3572 gene product, SEQ ID NO: 15 is amino acids 1-49 of Bacillus cereus VD200 exosporium leader peptide, SEQ ID NO: 17 is amino acids 1-33 of Bacillus cereus VD166 exosporium leader peptide, SEQ ID NO: 19 is amino acids 1-39 of Bacillus cereus VD200 hypothetical protein IKG_04663, SEQ ID NO: 21 is amino acids 1-39 of Bacillus weihenstephensis KBAB4 YVTN β-propeller protein, SEQ ID NO: 23 is amino acids 1-30 of Bacillus weihenstephensis KBAB4 hypothetical protein bcerkbab4_2363, SEQ ID NO: 25 is amino acids 1-30 of Bacillus weihenstephensis KBAB4 hypothetical protein bcerkbab4_2131, SEQ ID NO: 27 is amino acids 1-36 of Bacillus weihenstephensis KBAB4 triple helix repeat containing collagen, SEQ ID NO: 29 is amino acids 1-39 of Bacillus mycoides 2048 hypothetical protein bmyco0001_21660, SEQ ID NO: 31 is amino acids 1-30 of Bacillus mycoides 2048 hypothetical protein bmyc0001_22540, SEQ ID NO: 33 is amino acids 1-21 of Bacillus mycoides 2048 hypothetical protein bmyc0001_21510, SEQ ID NO: 35 is amino acids 1-22 of Bacillus thuringiensis 35646 collagen triple helix repeat protein, SEQ ID NO: 43 is amino acids 1-35 of Bacillus cereus hypothetical protein WP_69652, SEQ ID NO: 45 is amino acids 1-41 of Bacillus cereus exosporium leader WP016117717, SEQ ID NO: 47 is amino acids 1-49 of Bacillus cereus exosporium peptide WP002105192, SEQ ID NO: 49 is amino acids 1-38 of Bacillus cereus hypothetical protein WP87353, SEQ ID NO: 51 is amino acids 1-39 of Bacillus cereus exosporium peptide 02112369, SEQ ID NO: 53 is amino acids 1-39 of Bacillus cereus exosporium protein WP016099770, SEQ ID NO: 55 is amino acids 1-36 of Bacillus thuringiensis hypothetical protein YP006612525, SEQ ID NO: 57 is amino acids 1-136 of Bacillus mycoides hypothetical protein TIGR03720, SEQ ID NO: 59 is amino acids 1-36 of B. cereus ATCC 10987 collagen triple helix repeat domain protein, SEQ ID NO: 61 is amino acids 1-39 of B. cereus E33L collagen-like protein, SEQ ID NO: 63 is amino acids 1-41 of B. weihenstephanensis KBAB4 triple helix repeat-containing collagen, SEQ ID NO: 65 is amino acids 1-30 of B. thuringiensis str. Al Hakam hypothetical protein BALH_2230, SEQ ID NO: 67 is amino acids 1-33 of B. cereus ATCC 14579 triple helix repeat-containing collagen, SEQ ID NO: 69 is amino acids 1-44 of B. cereus collagen triple helix repeat, SEQ ID NO: 71 is amino acids 1-38 of B. cereus ATCC 14579 triple helix repeat-containing collagen, SEQ ID NO: 73 is amino acids 1-30 of B. cereus E33L hypothetical protein BCZK1835, SEQ ID NO: 75 is amino acids 1-48 of B. weihenstephanensis KBAB4 triple helix repeat-containing collagen, SEQ ID NO: 77 is amino acids 1-30 of B. cereus ATCC 14579 triple helix repeat-containing collagen, SEQ ID NO: 79 is amino acids 1-39 of B. cereus ATCC 14579 hypothetical protein BC4725, SEQ ID NO: 81 is amino acids 1-44 of B. cereus E33L hypothetical protein BCZK4476, SEQ ID NO: 83 is amino acids 1-40 of B. anthracis str. ‘Ames Ancestor’ triple helix repeat-containing collagen, SEQ ID NO: 85 is amino acids 1-34 of B. thuringiensis serovar konkukian str. 97-27 BclA protein, SEQ ID NO: 87 is amino acids 1-34 of B. cereus ATCC 10987 conserved hypothetical protein, SEQ ID NO: 89 is amino acids 1-34 of B. cereus ATCC 14579 triple helix repeat-containing collagen, SEQ ID NO: 91 is amino acids 1-99 of B. cereus exosporium leader peptide partial sequence, and SEQ ID NO: 93 is amino acids 1-136 of B. weihenstephanensis hypothetical protein ER45_27600. As shown in FIGS. 1A and 1B, each of the N-terminal regions of these proteins contains a region that is conserved with amino acids 20-35 of BclA (SEQ ID NO: 1), and a more highly conserved region corresponding to amino acids 25-35 of BclA.

Amino acids 1-41 of BclA from B. thuringiensis (SEQ ID NO: 204) and amino acids 1-41 of BclA from B. anthracis (SEQ ID NO: 206) are identical to SEQ ID NO: 2 and are thus not depicted in FIG. 1.

Any portion of BclA which includes amino acids 20-35 can be used as to target a fusion protein to the exosporium. In addition, full-length exosporium proteins or exosporium protein fragments can be used for targeting the fusion proteins to the exosporium. Thus, full-length BclA or a fragment of BclA that includes amino acids 20-35 can be used for targeting to the exosporium. For example, full length BclA (SEQ ID NO: 2, 204, or 206) or a midsized fragment of BclA that lacks the carboxy-terminus such as SEQ ID NO: 95 or 207 (amino acids 1-196 of BclA) or 205 (amino acids 1-166 of BclA) can be used to target the fusion proteins to the exosporium. Midsized fragments such as the fragments of SEQ ID NO: 95, 205, and 207 have less secondary structure than full length BclA and have been found to be suitable for use as a targeting sequence. The targeting sequence can also comprise much shorter portions of BclA which include amino acids 20-35, such as SEQ ID NO: 1 (amino acids 1-41 of BclA), amino acids 1-35 of SEQ ID NO: 1, amino acids 20-35 of SEQ ID NO: 1, or SEQ ID NO: 96 (a methionine residue linked to amino acids 20-35 of BclA). Even shorter fragments of BclA which include only some of amino acids 20-35 also exhibit the ability to target fusion proteins to the exosporium. For example, the targeting sequence can comprise amino acids 22-31 of SEQ ID NO: 1, amino acids 22-33 of SEQ ID NO: 1, or amino acids 20-31 of SEQ ID NO: 1.

Alternatively, any portion of BetA/BAS3290, BAS4623, BclB, BAS1882, the KBAB4 2280 gene product, the KBAB4 3572 gene product, B. cereus VD200 exosporium leader peptide, B. cereus VD166 exosporium leader peptide, B. cereus VD200 hypothetical protein IKG_04663, B. weihenstephensis KBAB4 YVTN β-propeller protein, B. weihenstephensis KBAB4 hypothetical protein bcerkbab4_2363, B. weihenstephensis KBAB4 hypothetical protein bcerkbab4_2131, B. weihenstephensis KBAB4 triple helix repeat containing collagen, B. mycoides 2048 hypothetical protein bmyco0001_21660, B. mycoides 2048 hypothetical protein bmyc0001_22540, B. mycoides 2048 hypothetical protein bmyc0001_21510, B. thuringiensis 35646 collagen triple helix repeat protein, B. cereus hypothetical protein WP_69652, B. cereus exosporium leader WP016117717, B. cereus exosporium peptide WP002105192, B. cereus hypothetical protein WP87353, B. cereus exosporium peptide 02112369, B. cereus exosporium protein WP016099770, B. thuringiensis hypothetical protein YP006612525, B. mycoides hypothetical protein TIGR03720, B. cereus ATCC 10987 collagen triple helix repeat domain protein, B. cereus E33L collagen-like protein, B. weihenstephanensis KBAB4 triple helix repeat-containing collagen, B. thuringiensis str. Al Hakam hypothetical protein BALH_2230, B. cereus ATCC 14579 triple helix repeat-containing collagen, B. cereus collagen triple helix repeat, B. cereus ATCC 14579 triple helix repeat-containing collagen, B. cereus E33L hypothetical protein BCZK1835, B. weihenstephanensis KBAB4 triple helix repeat-containing collagen, B. cereus ATCC 14579 triple helix repeat-containing collagen, B. cereus ATCC 14579 hypothetical protein BC4725, B. cereus E33L hypothetical protein BCZK4476, B. anthracia str. ‘Ames Ancestor’ triple helix repeat-containing collagen, B. thuringiensis serovar konkukian str. 97-27 BclA protein, B. cereus ATCC 10987 conserved hypothetical protein, B. cereus ATCC 14579 triple helix repeat-containing collagen, B. cereus exosporium leader peptide partial sequence, or B. weihenstephanensis hypothetical protein ER45_27600 which includes the amino acids corresponding to amino acids 20-35 of BclA can serve as the targeting sequence.

As can be seen from FIG. 1A, amino acids 12-27 of BetA/BAS3290, amino acids 23-38 of BAS4623, amino acids 13-28 of BclB, amino acids 9-24 of BAS1882, amino acids 18-33 of KBAB4 2280 gene product, amino acids 18-33 of KBAB4 3572 gene product, amino acids 28-43 of B. cereus VD200 exosporium leader peptide, amino acids 12-27 of B. cereus VD166 exosporium leader peptide, amino acids 18-33 of B. cereus VD200 hypothetical protein IKG_04663, amino acids 18-33 B. weihenstephensis KBAB4 YVTN β-propeller protein, amino acids 9-24 of B. weihenstephensis KBAB4 hypothetical protein bcerkbab4_2363, amino acids 9-24 of B. weihenstephensis KBAB4 hypothetical protein bcerkbab4_2131, amino acids 15-30 of B. weihenstephensis KBAB4 triple helix repeat containing collagen, amino acids 18-33 of B. mycoides 2048 hypothetical protein bmyco0001_21660, amino acids 9-24 of B. mycoides 2048 hypothetical protein bmyc0001_22540, amino acids 1-15 of B. mycoides 2048 hypothetical protein bmyc0001_21510, amino acids 1-16 of B. thuringiensis 35646 collagen triple helix repeat protein, amino acids 14-29 of B. cereus hypothetical protein WP_69652, amino acids 20-35 of B. cereus exosporium leader WP016117717, amino acids 28-43 of B. cereus exosporium peptide WP002105192, amino acids 17-32 of B. cereus hypothetical protein WP87353, amino acids 18-33 of B. cereus exosporium peptide 02112369, amino acids 18-33 of B. cereus exosporium protein WP016099770, amino acids 15-30 of B. thuringiensis hypothetical protein YP006612525, and amino acids 115-130 of B. mycoides hypothetical protein TIGR03720 correspond to amino acids 20-35 of BclA. As can be seen from FIG. 1B of the '661 Publication, amino acids 15-30 of B. cereus ATCC 10987 collagen triple helix repeat domain protein, amino acids 18-33 of B. cereus E33L collagen-like protein, amino acids 20-35 of B. weihenstephanensis KBAB4 triple helix repeat-containing collagen, amino acids 9-24 of B. thuringiensis str. Al Hakam hypothetical protein BALH_2230, amino acids 12-27 of B. cereus ATCC 14579 triple helix repeat-containing collagen, amino acids 23-38 of B. cereus collagen triple helix repeat, amino acids 17-32 of B. cereus ATCC 14579 triple helix repeat-containing collagen, amino acids 9-24 of B. cereus E33L hypothetical protein BCZK1835, amino acids 27-42 of B. weihenstephanensis KBAB4 triple helix repeat-containing collagen, amino acids 9-24 of B. cereus ATCC 14579 triple helix repeat-containing collagen, amino acids 18-33 of B. cereus ATCC 14579 hypothetical protein BC4725, amino acids 23-38 of B. cereus E33L hypothetical protein BCZK4476, amino acids 19-34 B. anthracia str. ‘Ames Ancestor’ triple helix repeat-containing collagen, amino acids 13-28 of B. thuringiensis serovar konkukian str. 97-27 BclA protein, amino acids 13-28 of B. cereus ATCC 10987 conserved hypothetical protein, amino acids 13-28 of B. cereus ATCC 14579 triple helix repeat-containing collagen, amino acids 78-93 of B. cereus exosporium leader peptide partial sequence, and amino acids 115-130 of B. weihenstephanensis hypothetical protein ER45_27600 correspond to amino acids 20-35 of BclA. Thus, any portion of these proteins that includes the above-listed corresponding amino acids can serve as the targeting sequence.

Furthermore, any amino acid sequence comprising amino acids 20-35 of BclA, or any of the above-listed corresponding amino acids, can serve as the targeting sequence.

The targeting sequence can comprise amino acids 1-35 of SEQ ID NO: 1, amino acids 20-35 of SEQ ID NO: 1, SEQ ID NO: 1, SEQ ID NO: 96, amino acids 22-31 of SEQ ID NO: 1, amino acids 22-33 of SEQ ID NO: 1, or amino acids 20-31 of SEQ ID NO: 1. Alternatively, the targeting sequence can consist of amino acids 1-35 of SEQ ID NO: 1, amino acids 20-35 of SEQ ID NO: 1, SEQ ID NO: 1, or SEQ ID NO: 96. Alternatively, the targeting sequence can consist of amino acids 22-31 of SEQ ID NO: 1, amino acids 22-33 of SEQ ID NO: 1, or amino acids 20-31 of SEQ ID NO: 1. Alternatively, the exosporium protein can comprise full length BclA (SEQ ID NO: 2), or the exosporium protein fragment can comprise a midsized fragment of BclA that lacks the carboxy-terminus, such as SEQ ID NO: 95 (amino acids 1-196 of BclA). Alternatively, the exosporium protein fragment can consist of SEQ ID NO: 95.

The targeting sequence can comprise amino acids 2-35 of SEQ ID NO: 1; amino acids 5-35 of SEQ ID NO: 1; amino acids 8-35 of SEQ ID NO: 1; amino acids 10-35 of SEQ ID NO: 1; or amino acids 15-35 of SEQ ID NO: 1.

The targeting sequence can comprise amino acids 1-27 of SEQ ID NO: 3, amino acids 12-27 of SEQ ID NO: 3, or SEQ ID NO: 3, or the exosporium protein can comprise full length BetA/BAS3290 (SEQ ID NO: 4). It has also been found that a methionine residue linked to amino acids 12-27 of BetA/BAS3290 can be used as a targeting sequence. Thus, the targeting sequence can comprise SEQ ID NO: 97. Alternatively, the targeting sequence can comprise amino acids 14-23 of SEQ ID NO: 3, amino acids 14-25 of SEQ ID NO: 3, or amino acids 12-23 of SEQ ID NO: 3.

The targeting sequence can comprise amino acids 2-27 of SEQ ID NO: 3; amino acids 5-27 of SEQ ID NO: 3; amino acids 8-27 of SEQ ID NO: 3; or amino acids 10-27 of SEQ ID NO: 3.

The targeting sequence can comprise amino acids 1-38 of SEQ ID NO: 5, amino acids 23-38 of SEQ ID NO: 5, SEQ ID NO: 5, or SEQ ID NO: 201 (a methionine residue linked to amino acids 23-38 of BAS4623) or the exosporium protein can comprise full length BAS4623 (SEQ ID NO: 6).

The targeting sequence can comprise amino acids 2-38 of SEQ ID NO: 5; amino acids 5-38 of SEQ ID NO: 5; amino acids 8-38 of SEQ ID NO: 5; amino acids 10-38 of SEQ ID NO: 5; amino acids 15-38 of SEQ ID NO: 5; or amino acids 20-38 of SEQ ID NO: 5.

Alternatively, the targeting sequence can comprise amino acids 1-28 of SEQ ID NO: 7, amino acids 13-28 of SEQ ID NO: 7, SEQ ID NO: 7, or SEQ ID NO: 202 (a methionine residue linked to amino acids 13-28 of BclB) or the exosporium protein can comprise full length BclB (SEQ ID NO: 8).

The targeting sequence can comprise amino acids 2-28 of SEQ ID NO: 7; amino acids 5-28 of SEQ ID NO: 7; amino acids 8-28 of SEQ ID NO: 7; or amino acids 10-28 of SEQ ID NO: 7.

The targeting sequence can comprise amino acids 1-24 of SEQ ID NO: 9, amino acids 9-24 of SEQ ID NO: 9, or SEQ ID NO: 9, or the exosporium protein can comprise full length BAS1882 (SEQ ID NO: 10). A methionine residue linked to amino acids 9-24 of BAS1882 can be used as a targeting sequence. Thus, the targeting sequence can comprise SEQ ID NO: 105.

The targeting sequence can comprise amino acids 2-24 of SEQ ID NO: 9; amino acids 5-24 of SEQ ID NO: 9; or amino acids 8-24 of SEQ ID NO: 9.

The targeting sequence can comprise amino acids 1-33 of SEQ ID NO: 11, amino acids 18-33 of SEQ ID NO: 11, or SEQ ID NO: 11, or the exosporium protein can comprise the full length B. weihenstephensis KBAB4 2280 gene product (SEQ ID NO: 12). A methionine residue linked to amino acids 18-33 of the B. weihenstephensis KBAB4 2280 gene product can be used as a targeting sequence. Thus, the targeting sequence can comprise SEQ ID NO: 98.

The targeting sequence can comprise amino acids 2-33 of SEQ ID NO: 11; amino acids 5-33 of SEQ ID NO: 11; amino acids 8-33 of SEQ ID NO: 11; amino acids 10-33 of SEQ ID NO: 11; or amino acids 15-33 of SEQ ID NO: 11.

The targeting sequence can also comprise amino acids 1-33 of SEQ ID NO: 13, amino acids 18-33 of SEQ ID NO: 13, or SEQ ID NO: 13, or the exosporium protein can comprise the full length B. weihenstephensis KBAB4 3572 gene product (SEQ ID NO: 14). A methionine residue linked to amino acids 18-33 of the B. weihenstephensis KBAB4 3572 gene product can be used as a targeting sequence. Thus, the targeting sequence can comprise SEQ ID NO: 99.

The targeting sequence can comprise amino acids 2-33 of SEQ ID NO: 13; amino acids 5-33 of SEQ ID NO: 13; amino acids 8-33 of SEQ ID NO: 13; amino acids 10-33 of SEQ ID NO: 13; or amino acids 15-33 of SEQ ID NO: 13.

Alternatively, the targeting sequence can comprise amino acids 1-43 of SEQ ID NO: 15, amino acids 28-43 of SEQ ID NO: 15, or SEQ ID NO: 15, or the exosporium protein can comprise full length B. cereus VD200 exosporium leader peptide (SEQ ID NO: 16).

The targeting sequence can comprise amino acids 2-43 of SEQ ID NO: 15; amino acids 5-43 of SEQ ID NO: 15; amino acids 8-43 of SEQ ID NO: 15; amino acids 10-43 of SEQ ID NO: 15; amino acids 15-43 of SEQ ID NO: 15; amino acids 20-43 of SEQ ID NO: 15; or amino acids 25-43 of SEQ ID NO: 15.

The targeting sequence can also comprise amino acids 1-27 of SEQ ID NO: 17, amino acids 12-27 of SEQ ID NO: 17, or SEQ ID NO: 17, or the exosporium protein can comprise full-length B. cereus VD166 exosporium leader peptide (SEQ ID NO: 18). A methionine residue linked to amino acids 12-27 of the B. cereus VD166 exosporium leader peptide can be used as a targeting sequence. Thus, the targeting sequence can comprise SEQ ID NO: 100.

The targeting sequence can comprise amino acids 2-27 of SEQ ID NO: 17; amino acids 5-27 of SEQ ID NO: 17; amino acids 8-27 of SEQ ID NO: 17; or amino acids 10-27 of SEQ ID NO: 17.

The targeting sequence can also comprise amino acids 1-33 of SEQ ID NO: 19, amino acids 18-33 of SEQ ID NO: 19, or SEQ ID NO: 19, or the exosporium protein can comprise full length B. cereus VD200 hypothetical protein IKG_04663 (SEQ ID NO: 20).

The targeting sequence can comprise amino acids 2-33 of SEQ ID NO: 19; amino acids 5-33 of SEQ ID NO: 19; amino acids 8-33 of SEQ ID NO: 19; amino acids 10-33 of SEQ ID NO: 19; or amino acids 15-33 of SEQ ID NO: 19.

Alternatively, the targeting sequence comprises amino acids 1-33 of SEQ ID NO: 21, amino acids 18-33 of SEQ ID NO: 21, or SEQ ID NO: 21, or the exosporium protein can comprise full length B. weihenstephensis KBAB4 YVTN β-propeller protein (SEQ ID NO: 22). A methionine residue linked to amino acids 18-33 of the B. weihenstephensis KBAB4 YVTN β-propeller protein can be used as a targeting sequence. Thus, the targeting sequence can comprise SEQ ID NO: 101.

The targeting sequence can comprise amino acids 2-33 of SEQ ID NO: 21; amino acids 5-33 of SEQ ID NO: 21; amino acids 8-33 of SEQ ID NO: 21; amino acids 10-33 of SEQ ID NO: 21; or amino acids 15-33 of SEQ ID NO: 21.

The targeting sequence can also comprise amino acids 1-24 of SEQ ID NO: 23, amino acids 9-24 of SEQ ID NO: 23, or SEQ ID NO: 23, or the exosporium protein can comprise full length B. weihenstephensis KBAB4 hypothetical protein bcerkbab4_2363 (SEQ ID NO: 24). A methionine residue linked to amino acids 9-24 of B. weihenstephensis KBAB4 hypothetical protein bcerkbab4_2363 can be used as a targeting sequence. Thus, the targeting sequence can comprise SEQ ID NO: 102.

The targeting sequence can comprise amino acids 2-24 of SEQ ID NO: 23; amino acids 5-24 of SEQ ID NO: 23; or amino acids 8-24 of SEQ ID NO: 23.

The targeting sequence comprise amino acids 1-24 of SEQ ID NO: 25, amino acids 9-24 of SEQ ID NO: 25, or SEQ ID NO: 25, or the exosporium protein can comprise full length B. weihenstephensis KBAB4 hypothetical protein bcerkbab4_2131 (SEQ ID NO: 26). A methionine residue linked to amino acids 9-24 of B. weihenstephensis KBAB4 hypothetical protein bcerkbab4_2131 can be used as a targeting sequence. Thus, the targeting sequence can comprise SEQ ID NO: 103.

The targeting sequence can comprise amino acids 2-24 of SEQ ID NO: 25; amino acids 5-24 of SEQ ID NO: 25; or amino acids 8-24 of SEQ ID NO: 25.

Alternatively, the targeting sequence comprises amino acids 1-30 of SEQ ID NO: 27, amino acids 15-30 of SEQ ID NO: 27, or SEQ ID NO: 27, or the exosporium protein can comprise full length B. weihenstephensis KBAB4 triple helix repeat containing collagen (SEQ ID NO: 28).

The targeting sequence can comprise amino acids 2-30 of SEQ ID NO: 27; amino acids 5-30 of SEQ ID NO: 27; amino acids 8-30 of SEQ ID NO: 27; or amino acids 10-30 of SEQ ID NO: 27.

The targeting sequence can also comprise amino acids 1-33 of SEQ ID NO: 29, amino acids 18-33 of SEQ ID NO: 29, or SEQ ID NO: 29, or the exosporium protein can comprise full length B. mycoides 2048 hypothetical protein bmyco0001_21660 (SEQ ID NO: 30).

The targeting sequence can comprise amino acids 2-33 of SEQ ID NO: 29; amino acids 5-33 of SEQ ID NO: 29; amino acids 8-33 of SEQ ID NO: 29; amino acids 10-33 of SEQ ID NO: 29; or amino acids 15-33 of SEQ ID NO: 29.

The targeting sequence can also comprise amino acids 1-24 of SEQ ID NO: 31, amino acids 9-24 of SEQ ID NO: 31, or SEQ ID NO: 31, or the exosporium protein can comprise full length B. mycoides 2048 hypothetical protein bmyc0001_22540 (SEQ ID NO: 32). A methionine residue linked to amino acids 9-24 of B. mycoides 2048 hypothetical protein bmyc0001_22540 can be used as a targeting sequence. Thus, the targeting sequence can comprise SEQ ID NO: 104.

The targeting sequence can comprise amino acids 2-24 of SEQ ID NO: 31; amino acids 5-24 of SEQ ID NO: 31; or amino acids 8-24 of SEQ ID NO: 31.

Alternatively, the targeting sequence comprises amino acids 1-15 of SEQ ID NO: 33, SEQ ID NO: 33, or the exosporium protein comprises full length B. mycoides 2048 hypothetical protein bmyc0001_21510 (SEQ ID NO: 34).

The targeting sequence can also comprise amino acids 1-16 of SEQ ID NO: 35, SEQ ID NO: 35, or the exosporium protein can comprise full length B. thuringiensis 35646 collagen triple helix repeat protein (SEQ ID NO: 36).

The targeting sequence can comprise amino acids 1-29 of SEQ ID NO: 43, amino acids 14-29 of SEQ ID NO: 43, or SEQ ID NO: 43, or the exosporium protein can comprise full length B. cereus hypothetical protein WP_69652 (SEQ ID NO: 44).

The targeting sequence can comprise amino acids 2-29 of SEQ ID NO: 43; amino acids 5-29 of SEQ ID NO: 43; amino acids 8-29 of SEQ ID NO: 43; or amino acids 10-29 of SEQ ID NO: 43.

Alternatively, the targeting sequence can comprise amino acids 1-35 of SEQ ID NO: 45, amino acids 20-35 of SEQ ID NO: 45, or SEQ ID NO: 45, or the exosporium protein can comprise full length B. cereus exosporium leader WP016117717 (SEQ ID NO: 46). A methionine residue linked to amino acids 20-35 of B. cereus exosporium leader WP016117717 can be used as a targeting sequence. Thus, the targeting sequence can comprise SEQ ID NO: 106.

The targeting sequence can comprise amino acids 2-35 of SEQ ID NO: 45; amino acids 5-35 of SEQ ID NO: 45; amino acids 8-35 of SEQ ID NO: 45; amino acids 10-35 of SEQ ID NO: 45; or amino acids 15-35 of SEQ ID NO: 45.

The targeting sequence can comprise amino acids 1-43 of SEQ ID NO: 47, amino acids 28-43 of SEQ ID NO: 47, or SEQ ID NO: 47, or the exosporium protein can comprise full length B. cereus exosporium peptide WP002105192 (SEQ ID NO: 48).

The targeting sequence can comprise amino acids 2-43 of SEQ ID NO: 47; amino acids 5-43 of SEQ ID NO: 47; amino acids 8-43 of SEQ ID NO: 47; amino acids 10-43 of SEQ ID NO: 47; amino acids 15-43 of SEQ ID NO: 47; amino acids 20-43 of SEQ ID NO: 47; or amino acids 25-43 of SEQ ID NO: 47.

The targeting sequence can comprise amino acids 1-32 of SEQ ID NO: 49, amino acids 17-32 of SEQ ID NO: 49, or SEQ ID NO: 49, or the exosporium protein can comprise full length B. cereus hypothetical protein WP87353 (SEQ ID NO: 50).

The targeting sequence can comprise amino acids 2-32 of SEQ ID NO: 49; amino acids 5-32 of SEQ ID NO: 49; amino acids 8-32 of SEQ ID NO: 49; amino acids 10-32 of SEQ ID NO: 49; or amino acids 15-32 of SEQ ID NO: 49.

Alternatively, the targeting sequence can comprise amino acids 1-33 of SEQ ID NO: 51, amino acids 18-33 of SEQ ID NO: 51, or SEQ ID NO: 51, or the exosporium protein can comprise full length B. cereus exosporium peptide 02112369 (SEQ ID NO: 52).

The targeting sequence can comprise amino acids 2-33 of SEQ ID NO: 51; amino acids 5-33 of SEQ ID NO: 51; amino acids 8-33 of SEQ ID NO: 51; amino acids 10-33 of SEQ ID NO: 51; or amino acids 15-33 of SEQ ID NO: 51.

The targeting sequence can comprise amino acids 1-33 of SEQ ID NO: 53, amino acids 18-33 of SEQ ID NO: 53, or SEQ ID NO: 53, or the exosporium protein can comprise full length B. cereus exosporium protein WP016099770 (SEQ ID NO: 54).

The targeting sequence can comprise amino acids 2-33 of SEQ ID NO: 53; amino acids 5-33 of SEQ ID NO: 53; amino acids 8-33 of SEQ ID NO: 53; amino acids 10-33 of SEQ ID NO: 53; or amino acids 15-33 of SEQ ID NO: 53.

Alternatively, the targeting sequence can comprise acids 1-30 of SEQ ID NO: 55, amino acids 15-30 of SEQ ID NO: 55, or SEQ ID NO: 55, or the exosporium protein can comprise full length B. thuringiensis hypothetical protein YP006612525 (SEQ ID NO: 56).

The targeting sequence can comprise amino acids 2-30 of SEQ ID NO: 55; amino acids 5-30 of SEQ ID NO: 55; amino acids 8-30 of SEQ ID NO: 55; or amino acids 10-30 of SEQ ID NO: 55.

The targeting sequence can comprise amino acids 1-130 of SEQ ID NO: 57, amino acids 115-130 of SEQ ID NO: 57, or SEQ ID NO: 57, or the exosporium protein can comprise full length B. mycoides hypothetical protein TIGR03720 (SEQ ID NO: 58).

The targeting sequence can comprise amino acids 2-130 of SEQ ID NO: 57; amino acids 5-130 of SEQ ID NO: 57; amino acids 10-130 of SEQ ID NO: 57; amino acids 20-130 of SEQ ID NO: 57; amino acids 30-130 of SEQ ID NO: 57; amino acids 40-130 of SEQ ID NO: 57; amino acids 50-130 of SEQ ID NO: 57; amino acids 60-130 of SEQ ID NO: 57; amino acids 70-130 of SEQ ID NO: 57; amino acids 80-130 of SEQ ID NO: 57; amino acids 90-130 of SEQ ID NO: 57; amino acids 100-130 of SEQ ID NO: 57; or amino acids 110-130 of SEQ ID NO: 57.

The targeting sequence can comprise amino acids 1-30 of SEQ ID NO: 59; or SEQ ID NO: 59; or the exosporium protein can comprise full length B. cereus ATCC 10987 collagen triple helix repeat domain protein (SEQ ID NO: 60).

The targeting sequence can comprise amino acids 2-30 of SEQ ID NO: 59; amino acids 4-30 of SEQ ID NO: 59; or amino acids 6-30 of SEQ ID NO: 59.

The targeting sequence can comprise amino acids 1-33 of SEQ ID NO: 61; amino acids 18-33 of SEQ ID NO: 61; or SEQ ID NO: 61; or the exosporium protein can comprise full length B. cereus E33L collagen-like protein (SEQ ID NO: 62).

The targeting sequence can comprise amino acids 2-33 of SEQ ID NO: 61; amino acids 5-33 of SEQ ID NO: 61; amino acids 10-33 of SEQ ID NO: 61; or amino acids 15-33 of SEQ ID NO: 61.

The targeting sequence can comprise amino acids 1-35 of SEQ ID NO: 63; or SEQ ID NO: 63; or the exosporium protein can comprise full length B. weihenstephanensis KBAB4 triple helix repeat-containing collagen (SEQ ID NO: 64).

The targeting sequence can comprise amino acids 2-35 of SEQ ID NO: 63; amino acids 5-35 of SEQ ID NO: 63; amino acids 8-35 of SEQ ID NO: 63; amino acids 10-35 of SEQ ID NO: 63; or amino acids 15-35 of SEQ ID NO: 63.

The targeting sequence can comprise amino acids 1-24 of SEQ ID NO: 65; acids 9-24 of SEQ ID NO: 65; SEQ ID NO: 65; or SEQ ID NO: 107; or the exosporium protein can comprise full length B. thuringiensis str. Al Hakam hypothetical protein BALH_2230 (SEQ ID NO: 66).

The targeting sequence can comprise amino acids 2-24 of SEQ ID NO: 65; or amino acids 5-24 of SEQ ID NO: 65.

The targeting sequence can comprise acids 1-27 of SEQ ID NO: 67; amino acids 12-27 of SEQ ID NO: 67; or SEQ ID NO: 67; or the exosporium protein can comprise full length B. cereus ATCC 14579 triple helix repeat-containing collagen (SEQ ID NO: 68).

The targeting sequence can comprise amino acids 2-27 of SEQ ID NO: 67; amino acids 5-27 of SEQ ID NO: 67; or amino acids 10-27 of SEQ ID NO: 67.

The targeting sequence can comprise amino acids 1-38 of SEQ ID NO: 69; amino acids 23-38 of SEQ ID NO: 69; or SEQ ID NO: 69; or the exosporium protein can comprise full length B. cereus collagen triple helix repeat (SEQ ID NO: 70).

The targeting sequence can comprise amino acids 2-38 of SEQ ID NO: 69; amino acids 5-38 of SEQ ID NO: 69; amino acids 10-38 of SEQ ID NO: 69; or amino acids 15-38 of SEQ ID NO: 69.

The exosporium protein can comprise full length B. cereus ATCC 14579 triple helix repeat-containing collagen (SEQ ID NO: 72).

The targeting sequence can comprise SEQ ID NO: 73, or the exosporium protein can comprise full length B. cereus E33L hypothetical protein BCZK1835 (SEQ ID NO: 74).

The targeting sequence can comprise amino acids 1-42 of SEQ ID NO: 75; amino acids 27-42 of SEQ ID NO: 75; or SEQ ID NO: 75; or the exosporium protein can comprise full length B. weihenstephanensis KBAB4 triple helix repeat-containing collagen (SEQ ID NO: 76).

The targeting sequence can comprise amino acids 2-42 of SEQ ID NO: 75; amino acids 5-42 of SEQ ID NO: 75; amino acids 10-42 of SEQ ID NO: 75; amino acids 15-42 of SEQ ID NO: 75; amino acids 20-42 of SEQ ID NO: 75; or amino acids 25-42 of SEQ ID NO: 75.

The targeting sequence can comprise amino acids 1-24 of SEQ ID NO: 77; amino acids 9-24 of SEQ ID NO: 77; or SEQ ID NO: 77; or the exosporium protein can comprise full length B. cereus ATCC 14579 triple helix repeat-containing collagen (SEQ ID NO: 78).

The targeting sequence can comprise amino acids 2-24 of SEQ ID NO: 77; or amino acids 5-24 of SEQ ID NO: 77.

The exosporium protein can comprise full length B. cereus ATCC 14579 hypothetical protein BC4725 (SEQ ID NO: 80).

The targeting sequence can comprise amino acids 1-38 of SEQ ID NO: 81; amino acids 23-38 of SEQ ID NO: 81; or SEQ ID NO: 81; or the exosporium protein can comprise full length B. cereus E33L hypothetical protein BCZK4476 (SEQ ID NO: 82).

The targeting sequence can comprise amino acids 2-38 of SEQ ID NO: 81; acids 5-38 of SEQ ID NO: 81; amino acids 10-38 of SEQ ID NO: 81; amino acids 15-38 of SEQ ID NO: 81; or amino acids 20-38 of SEQ ID NO: 81.

The targeting sequence can comprise amino acids 1-34 of SEQ ID NO: 83; or SEQ ID NO: 83; or the exosporium protein can comprise full length B. anthracia str. ‘Ames Ancestor’ triple helix repeat-containing collagen (SEQ ID NO: 84).

The exosporium protein can comprise full length B. thuringiensis serovar konkukian str. 97-27 BclA protein (SEQ ID NO: 86).

The targeting sequence can comprise amino acids 1-28 of SEQ ID NO: 87; amino acids 13-28 of SEQ ID NO: 87; or SEQ ID NO: 87; or the exosporium protein can comprise full length B. cereus ATCC 10987 conserved hypothetical protein (SEQ ID NO: 88).

The targeting sequence can comprise amino acids 2-28 of SEQ ID NO: 87; amino acids 5-28 of SEQ ID NO: 87; or amino acids 10-28 of SEQ ID NO: 87.

The targeting sequence can comprise amino acids 1-28 of SEQ ID NO: 89; or SEQ ID NO: 89; or the exosporium protein can comprise full length B. cereus ATCC 14579 triple helix repeat-containing collagen (SEQ ID NO: 90).

The targeting sequence can comprise amino acids 2-28 of SEQ ID NO: 89; amino acids 5-28 of SEQ ID NO: 89; or amino acids 10-28 of SEQ ID NO: 89.

The targeting sequence can comprise amino acids 1-93 of SEQ ID NO: 91; or SEQ ID NO: 91; or the exosporium protein can comprise B. cereus exosporium leader peptide partial sequence (SEQ ID NO: 92).

The targeting sequence can comprise amino acids 2-93 of SEQ ID NO: 91; amino acids 10-93 of SEQ ID NO: 91; amino acids 20-93 of SEQ ID NO: 91; amino acids 30-93 of SEQ ID NO: 91; amino acids 40-93 of SEQ ID NO: 91; amino acids 50-93 of SEQ ID NO: 91; or amino acids 60-93 of SEQ ID NO: 91.

The targeting sequence can comprise amino acids 1-130 of SEQ ID NO: 93; or SEQ ID NO: 93; or the exosporium protein can comprise B. weihenstephanensis) hypothetical protein ER45_27600, partial sequence (SEQ ID NO: 94).

The targeting sequence can comprise amino acids 2-130 of SEQ ID NO: 93; amino acids 10-130 of SEQ ID NO: 93; amino acids 20-130 of SEQ ID NO: 93; or amino acids 30-130 of SEQ ID NO: 93.

The targeting sequence can comprise amino acids 1-35 of SEQ ID NO: 204, amino acids 20-35 of SEQ ID NO: 204, SEQ ID NO: 204, or SEQ ID NO: 205.

The targeting sequence can comprise amino acids 1-35 of SEQ ID NO: 206, amino acids 20-35 of SEQ ID NO: 206, SEQ ID NO: 206, or SEQ ID NO: 207.

Furthermore, it has been found that sequences shorter than amino acids 20-35 of BclA can be used to target a fusion protein to the exosporium of a recombinant Bacillus cereus family member. In particular, amino acids 20-33 of BclA, amino acids 20-31 of BclA, amino acids 21-33 of BclA, or amino acids 23-31 of BclA can be used to target a fusion protein to the exosporium of a recombinant Bacillus cereus family member. Thus, the targeting sequence can consist of amino acids 20-33 of SEQ ID NO: 1, amino acids 20-31 of SEQ ID NO: 1, amino acids 21-33 of SEQ ID NO: 1, or amino acids 23-31 of SEQ ID NO: 1. The corresponding regions of any of the SEQ ID NOs. shown in FIGS. 1A and 1B can also be used to target a fusion protein to the exosporium of a recombinant Bacillus cereus family member. By “corresponding regions,” it is meant that when the sequences are aligned with SEQ ID NO: 1, as shown in FIGS. 1A and 1B, the regions of the other amino acid sequences that align with the amino acids of SEQ ID NO: are the “corresponding regions” of those sequences. Thus, for example, amino acids 12-25 of SEQ ID NO: 3, amino acids 23-36 of SEQ ID NO: 5, amino acids 13-26 of SEQ ID NO: 7, etc., can be used to target a fusion protein to the exosporium of a recombinant Bacillus cereus family member, since these regions align with amino acids 20-33 of SEQ ID NO: 1 as shown in FIG. 1A.

Even shorter regions within amino acids 20-35 of BclA can also be used for targeting a fusion protein to the exosporium of a recombinant Bacillus cereus family member. In particular, any amino acid sequence that includes amino acids 25-30 of SEQ ID NO: 1 or the corresponding amino acids from any of the sequences shown in FIGS. 1A and 1B can be used. A skilled person will recognize that starting with amino acids 25-30 of SEQ ID NO: 1 or the corresponding region of any of the sequences shown in FIGS. 1A and 1B, additional amino acids can be added to the amino-terminus, the carboxy terminus, or both the amino- and carboxy termini to create a targeting sequence that will be effective for targeting a fusion protein to the exosporium of a recombinant Bacillus cereus family member.

In addition, it can readily be seen from the sequence alignment in FIGS. 1A and 1B of the '661 Publication that while amino acids 20-35 of BclA are conserved, and amino acids 25-35 are more conserved, some degree of variation can occur in this region without affecting the ability of the targeting sequence to target a protein to the exosporium. FIGS. 1A and 1B of the '661 Publication list the percent identity of each of the corresponding amino acids of each sequence to amino acids 20-35 of BclA (“20-35% Identity”) and to amino acids 25-35 of BclA (“25-35% Identity”). Thus, for example, as compared to amino acids 20-35 of BclA, the corresponding amino acids of BetA/BAS3290 are about 81.3% identical, the corresponding amino acids of BAS4623 are about 50.0% identical, the corresponding amino acids of BclB are about 43.8% identical, the corresponding amino acids of BAS1882 are about 62.5% identical, the corresponding amino acids of the KBAB4 2280 gene product are about 81.3% identical, and the corresponding amino acids of the KBAB4 3572 gene product are about 81.3% identical. The sequence identities over this region for the remaining sequences are listed in FIGS. 1A and 1B.

With respect to amino acids 25-35 of BclA, the corresponding amino acids of BetA/BAS3290 are about 90.9% identical, the corresponding amino acids of BAS4623 are about 72.7% identical, the corresponding amino acids of BclB are about 54.5% identical, the corresponding amino acids of BAS1882 are about 72.7% identical, the corresponding amino acids of the KBAB4 2280 gene product are about 90.9% identical, and the corresponding amino acids of the KBAB4 3572 gene product are about 81.8% identical. The sequence identities over this region for the remaining sequences are listed in FIGS. 1A and 1B.

Thus, the targeting sequence can comprise an amino acid sequence having at least about 43% identity with amino acids 20-35 of SEQ ID NO: 1, wherein the identity with amino acids 25-35 is at least about 54%. Alternatively, the targeting sequence consists of an amino acid sequence consisting of 16 amino acids and having at least about 43% identity with amino acids 20-35 of SEQ ID NO: 1, wherein the identity with amino acids 25-35 is at least about 54%.

The targeting sequence can also comprise an amino acid sequence having at least about 50% identity with amino acids 20-35 of SEQ ID NO: 1, wherein the identity with amino acids 25-35 is at least about 63%. Alternatively, the targeting sequence consists of an amino acid sequence consisting of 16 amino acids and having at least about 50% identity with amino acids 20-35 of SEQ ID NO: 1, wherein the identity with amino acids 25-35 is at least about 63%.

The targeting sequence can also comprise an amino acid sequence having at least about 50% identity with amino acids 20-35 of SEQ ID NO: 1, wherein the identity with amino acids 25-35 is at least about 72%. Alternatively, the targeting sequence consists of an amino acid sequence consisting of 16 amino acids and having at least about 50% identity with amino acids 20-35 of SEQ ID NO: 1, wherein the identity with amino acids 25-35 is at least about 72%.

The targeting sequence can also comprise an amino acid sequence having at least about 56% identity with amino acids 20-35 of SEQ ID NO: 1, wherein the identity with amino acids 25-35 is at least about 63%. Alternatively, the targeting sequence consists of an amino acid sequence consisting of 16 amino acids and having at least about 56% identity with amino acids 20-35 of SEQ ID NO: 1, wherein the identity with amino acids 25-35 is at least about 63%.

The targeting sequence can comprise an amino sequence having at least about 62% identity with amino acids 20-35 of SEQ ID NO: 1, wherein the identity with amino acids 25-35 is at least about 72%. Alternatively, the targeting sequence can consist of an amino acid sequence consisting of 16 amino acids and having at least about 62% identity with amino acids 20-35 of SEQ ID NO: 1, wherein the identity with amino acids 25-35 of SEQ ID NO: 1 is at least about 72%.

The targeting sequence can comprise an amino acid sequence having at least 68% identity with amino acids 20-35 of SEQ ID NO: 1, wherein the identity with amino acids 25-35 is at least about 81%. Alternatively, the targeting sequence consists of an amino acid sequence consisting of 16 amino acids and having at least 68% identity with amino acids 20-35 of SEQ ID NO: 1, wherein the identity with amino acids 25-35 is at least about 81%.

The targeting sequence can also comprises an amino sequence having at least about 75% identity with amino acids 20-35 of SEQ ID NO: 1, wherein the identity with amino acids 25-35 is at least about 72%. Alternatively, the targeting sequence consists of an amino acid sequence consisting of 16 amino acids and having at least about 75% identity with amino acids 20-35 of SEQ ID NO: 1, wherein the identity with amino acids 25-35 of SEQ ID NO: 1 is at least about 72%.

The targeting sequence can also comprise an amino sequence having at least about 75% identity with amino acids 20-35 of SEQ ID NO: 1, wherein the identity with amino acids 25-35 is at least about 81%. Alternatively, the targeting sequence consists of an amino acid sequence consisting of 16 amino acids and having at least about 75% identity with amino acids 20-35 of SEQ ID NO: 1, wherein the identity with amino acids 25-35 of SEQ ID NO: 1 is at least about 81%.

The targeting sequence can also comprise an amino acid sequence having at least about 81% identity with amino acids 20-35 of SEQ ID NO: 1, wherein the identity with amino acids 25-35 is at least about 81%. Alternatively, the targeting sequence consists of an amino acid sequence consisting of 16 amino acids and having at least about 81% identity with amino acids 20-35 of SEQ ID NO: 1, wherein the identity with amino acids 25-35 is at least about 81%.

The targeting sequence can comprise an amino acid sequence having at least about 81% identity with amino acids 20-35 of SEQ ID NO: 1, wherein the identity with amino acids 25-35 is at least about 90%. Alternatively, the targeting sequence consists of an amino acid sequence consisting of 16 amino acids and having at least about 81% identity with amino acids 20-35 of SEQ ID NO: 1, wherein the identity with amino acids 25-35 is at least about 90%.

The skilled person will recognize that variants of the above sequences can also be used as targeting sequences, so long as the targeting sequence comprises amino acids 20-35 of BclA, the corresponding amino acids of BetA/BAS3290, BAS4263, BclB, BAS1882, the KBAB4 2280 gene product, or the KBAB 3572 gene product, or a sequence comprising any of the above noted sequence identities to amino acids 20-35 and 25-35 of BclA is present.

Certain Bacillus cereus family exosporium proteins which lack regions having homology to amino acids 25-35 of BclA can also be used to target a peptide or protein to the exosporium of a Bacillus cereus family member. In particular, the fusion proteins can comprise an exosporium protein comprising SEQ ID NO: 108 (B. mycoides InhA), an exosporium protein comprising SEQ ID NO: 109 (B. anthracis Sterne BAS1141 (ExsY)), an exosporium protein comprising SEQ ID NO: 110 (B. anthracis Sterne BAS1144 (BxpB/ExsFA)), an exosporium protein comprising SEQ ID NO: 111 (B. anthracis Sterne BAS1145 (CotY)), an exosporium protein comprising SEQ ID NO: 112 (B. anthracis Sterne BAS1140), an exosporium protein comprising SEQ ID NO: 113 (B. anthracis H9401 ExsFB), an exosporium protein comprising SEQ ID NO: 114 (B. thuringiensis HD74 InhA1), an exosporium protein comprising SEQ ID NO: 115 (B. cereus ATCC 10876 ExsJ), an exosporium protein comprising SEQ ID NO: 116 (B. cereus ExsH), an exosporium protein comprising SEQ ID NO: 117 (B. anthracis Ames YjcA), an exosporium protein comprising SEQ ID NO: 118 (B. anthracis YjcB), an exosporium protein comprising SEQ ID NO: 119 (B. anthracis Sterne BclC), an exosporium protein comprising SEQ ID NO: 120 (Bacillus thuringiensis serovar konkukian str. 97-27 acid phosphatase), an exosporium protein comprising SEQ ID NO: 121 (B. thuringiensis HD74 InhA2), an exosporium protein comprising SEQ ID NO: 122 (B. mycoides InhA3), or an exosporium protein comprising SEQ ID NO: 203 (B. anthracis CotY variant). Inclusion of an exosporium protein comprising any of SEQ ID NOs: 108-122 or 203 in the fusion proteins described herein will result in targeting to the exosporium of a B. cereus family member.

Moreover, exosporium proteins having a high degree of sequence identity with any of the full-length exosporium proteins or the exosporium protein fragments described above can also be used to target a peptide or protein to the exosporium of a Bacillus cereus family member. Thus, the fusion protein can comprise an exosporium protein or exosporium protein fragment comprising an amino acid sequence having at least 85% identity with any one of SEQ ID NOs: 2, 4, 6, 8, 10, 12, 14, 16, 18, 20, 22, 24, 26, 28, 30, 32, 34, 36, 44, 46, 48, 50, 52, 54, 56, 58, 60, 62, 64, 66, 68, 70, 72, 74, 76, 78, 80, 82, 84, 86, 88, 90, 92, 94, 95, 108, 109, 110, 111, 112, 113, 114, 115, 116, 117, 118, 119, 120, 121, 122, and 203.

Alternatively, the fusion protein can comprise an exosporium protein having at least 90% identity with any one of SEQ ID NOs: 2, 4, 6, 8, 10, 12, 14, 16, 18, 20, 22, 24, 26, 28, 30, 32, 34, 36, 44, 46, 48, 50, 52, 54, 56, 58, 60, 62, 64, 66, 68, 70, 72, 74, 76, 78, 80, 82, 84, 86, 88, 90, 92, 94, 95, 108, 109, 110, 111, 112, 113, 114, 115, 116, 117, 118, 119, 120, 121, 122, and 203.

The fusion protein can comprise an exosporium protein having at least 95% identity with any one of SEQ ID NOs: 2, 4, 6, 8, 10, 12, 14, 16, 18, 20, 22, 24, 26, 28, 30, 32, 34, 36, 44, 46, 48, 50, 52, 54, 56, 58, 60, 62, 64, 66, 68, 70, 72, 74, 76, 78, 80, 82, 84, 86, 88, 90, 92, 94, 95, 108, 109, 110, 111, 112, 113, 114, 115, 116, 117, 118, 119, 120, 121, 122, and 203.

The fusion protein can comprise an exosporium protein having at least 98% identity with any one of SEQ ID NOs: 2, 4, 6, 8, 10, 12, 14, 16, 18, 20, 22, 24, 26, 28, 30, 32, 34, 36, 44, 46, 48, 50, 52, 54, 56, 58, 60, 62, 64, 66, 68, 70, 72, 74, 76, 78, 80, 82, 84, 86, 88, 90, 92, 94, 95, 108, 109, 110, 111, 112, 113, 114, 115, 116, 117, 118, 119, 120, 121, 122, and 203.

The fusion protein can comprise an exosporium protein having at least 99% identity with any one of SEQ ID NOs: 2, 4, 6, 8, 10, 12, 14, 16, 18, 20, 22, 24, 26, 28, 30, 32, 34, 36, 44, 46, 48, 50, 52, 54, 56, 58, 60, 62, 64, 66, 68, 70, 72, 74, 76, 78, 80, 82, 84, 86, 88, 90, 92, 94, 95, 108, 109, 110, 111, 112, 113, 114, 115, 116, 117, 118, 119, 120, 121, 122, and 203.

The fusion protein can comprise an exosporium protein having 100% identity with any one of SEQ ID NOs: 2, 4, 6, 8, 10, 12, 14, 16, 18, 20, 22, 24, 26, 28, 30, 32, 34, 36, 44, 46, 48, 50, 52, 54, 56, 58, 60, 62, 64, 66, 68, 70, 72, 74, 76, 78, 80, 82, 84, 86, 88, 90, 92, 94, 95, 108, 109, 110, 111, 112, 113, 114, 115, 116, 117, 118, 119, 120, 121, 122, and 203.

The targeting sequence, exosporium protein or exosporium protein fragment of the present invention may also be described in terms of a motif that provides the targeting function. FIGS. 1A and 1B show a sequence alignment of the amino-terminal region of BclA (SEQ ID NO: 1) with the corresponding amino-terminal regions of a number of other Bacillus cereus family member exosporium proteins. As can be seen from FIG. 1, there is a conserved motif at amino acids 20-35 of BclA (shown in bold in FIG. 1), with a more highly conserved motif at amino acids 25-35 of BclA (shown in bold and underlined in FIG. 1). This more highly conserved region is the recognition sequence for ExsFA/BxpB/ExsFB and homologs, which direct and assemble the described exosporium proteins on the surface of the exosporium.

Furthermore, while amino acids 20-35 of BclA are conserved, and amino acids 25-35 are more conserved, some degree of variation can occur in this region without affecting the ability of the targeting sequence to target a protein to the exosporium. FIG. 1 lists the percent identity of the corresponding amino acids of each sequence to amino acids 20-35 of BclA (“20-35% Identity”) and to amino acids 25-35 of BclA (“25-35% Identity”). Sequences having a targeting sequence identity as low as 43.8% with amino acids 20-35 of BclA (SEQ ID NO: 1), wherein the identity with amino acids 25-35 of BclA is 54.5%, retain the ability to target fusion proteins to the exosporium. Data are provided in Table 58 in Example 59 of PCT Publication No. WO 2016/044661, which is incorporated herein by reference in its entirety. Table 58 shows the enzyme levels of phosphatidylcholine-specific phospholipase C gene (PC-PLC) and lipase on Bacillus cereus family member spores expressing fusion proteins containing these enzymes and various targeting sequences. The relevant portion of Table 58 of PCT Publication No. WO 2016/044661 is reproduced below, with two additional columns added to show the percent identity of each of the targeting sequences with amino acids 20-35 and 25-35 of BclA (SEQ ID NO: 1):

| | Sequence | Sequence | | |
|---|---|---|---|---|
| | Identity | Identity | PC-PLC | Lipase |
| Targeting | to AA 20-35 | to AA 25-35 | Enzyme | Enzyme |
| Sequence | of BclA | of BclA | Levels | Levels |
| Control (H₂O) | N/A | N/A | 0.0 | 0.0 |
| AA 20-35 of | 100% | 100% | .787 | .436 |
| SEQ ID NO: 1 | | | | |
| AA 23-38 of | 50.0% | 72.7% | .688 | .602 |
| SEQ ID NO: 5 | | | | |
| AA 28-43 of | 68.8% | 81.8% | .372 | .228 |
| SEQ ID NO: 15 | | | | |
| AA 9-24 of | 56.3% | 63.6% | .247 | .359 |
| SEQ ID NO: 25 | | | | |

These data show that targeting of a protein of interest (e.g., an enzyme) to the exosporium proteins can be achieved using targeting sequences having 50-68.8% identity to amino acids 20-35 of BclA (SEQ ID NO: 1), wherein the identity to amino acids 25-35 of BclA is 63.6% to 81.8%. Such motif is present in a targeting sequence, exosporium protein, or exosporium protein fragment that targets the fusion protein to the exosporium of the recombinant Bacillus bacterium and comprises the sequence X₁-X₂-X₃-X₄-X₅-X₆-X₇-X₈-X₉-X₁₀-X₁₁-X₁₂-X₁₃-X₁₄-X₁₅-X₁₆, wherein:

X₁ is any amino acid or absent;

X₂ is phenylalanine (F), leucine (L), isoleucine (I), or methionine (M);

X₃ is any amino acid;

X₄ is proline (P) or serine (S);

X₅ is any amino acid;

X₆ is leucine (L), asparagine (N), serine (S), or isoleucine (I);

X₇ is valine (V) or isoleucine (I);

X₈ is glycine (G);

X₉ is proline (P);

X₁₀ is threonine (T) or proline (P);

X₁₁ is leucine (L) or phenylalanine (F);

X₁₂ is proline (P);

X₁₃ is any amino acid;

X₁₄ is any amino acid;

X₁₅ is proline (P), glutamine (Q), or threonine (T); and

X₁₆ is proline (P), threonine (T), or serine (S).

Any of the targeting sequences, exosporuim proteins, or exosporium protein fragments can be used to target any protein or peptide of interest, including the pectinases described herein, to the exosporium of a recombinant Bacillus cereus family member.

For example, any of the targeting sequences, exosporium proteins, or exosporium protein fragments (e.g., any of SEQ ID NOs: 203-207) can be used to target a protein or peptide of interest (e.g., any of SEQ ID NOs: 210-227) to the exosporium of a recombinant Bacillus cereus family member.

During sporulation of a recombinant Bacillus cereus family member expressing any of the fusion proteins described herein, the targeting motif, exosporium protein, or exosporium protein fragment is recognized by the spore exosporium assembly machinery and directed to the exosporium, resulting in display of the protein or peptide of interest portion of the fusion protein (e.g., the pectinase) on the outside of the spore.

The use of different targeting sequences allows for control of the expression level of the fusion protein on the surface of the Bacillus cereus family member spore. Use of certain of the targeting sequences described herein will result in a higher level of expression of the fusion protein, whereas use of others of the targeting sequences will result in lower levels of expression of the fusion protein on the surface of the spore.

In any of the fusion proteins described herein, the targeting sequence, exosporium protein, or exosporium protein fragment can comprise the amino acid sequence GXT at its carboxy terminus, wherein X is any amino acid.

In any of the fusion proteins described herein, the targeting sequence, exosporium protein, or exosporium protein fragment, can comprise an alanine residue at the position of the targeting sequence that corresponds to amino acid 20 of SEQ ID NO: 1.

In any of the fusion proteins described herein, the targeting sequence, exosporium protein, or exosporium protein fragment can further comprise a methionine, serine, or threonine residue at the amino acid position immediately preceding the first amino acid of the targeting sequence, exosporium protein, or exosporium protein fragment or at the position of the targeting sequence that corresponds to amino acid 20 of SEQ ID NO: 1.

### B. Pectinases—Pectin Lyase, Pectate Lyase and Endopolygalacturonase

The fusion proteins can comprise a pectin lyase, pectate lyase, or a polygalacturonase. Pectinases act on pectin and related polysaccharides to release small sugars. Specifically, pectin lyases (EC 4.2.2.10), also referred to as pectolyases, pectate lyases (EC 4.2.2.2) and polygalacturonases, including endopolygalacturonases (EC 3.2.1.15) and exopolygalacturonases (EC 3.2.1.67), are enzyme classes that catalyze the cleavage of internal α-1,4-bonds in galacturonan main chains. Galacturonans are found within the plant cell walls. However, they are primarily known as pectin, which is the main constituent of the middle lamella. Galacturonans mainly consist of α-1,4-linked galacturonic acid monomers, which can be esterified and extensively decorated. The small sugars that are released from polysaccharides through the action of pectinases can be taken up by a plant as a carbon source and can also feed the inherent microbes that surround the plant. Research provides insight into the activity of endopolygalacturonase I, including endopolygalacturonase I and endopolygalacturonase II, both from Aspergillus niger. See, for example, van Pouderoyen, et al., “Structural Insights in to the Processivity of Endopolygalacturonase I from Aspergillus niger,” FEBS Letters 554: 462-466 (2003). See also Santen, et al., “1.681.68-Å Crystal Structure of Endopolygalacturonase II from Aspergillus niger and Identification of Active Site Residues by Site-directed Mutagenesis,” The Journal of Biological Chemistry 274: 30474-30480 (1999).

Pectate lyase amino acid sequences are provided in Table 2 below, together with their SEQ ID NOs. The first SEQ ID NO in the second column corresponds to the sequence of the enzyme with its signal peptide. The second SEQ ID NO in the second column corresponds to the sequence of the enzyme without its signal peptide.

TABLE 2
Amino Acid Sequence for Pectate Lyases

| Enzyme | SEQ ID NO: |
|---|---|
| PECTATE LYASE C | 213, 222 |
| Bacillus subtilis 168 | |
| PECTATE LYASE 103 | 214, 223 |
| Bacillus safensis KSM-P103 | |
| PECTATE LYASE 22 | 215, 224 |
| Bacillus pumilus BS22 | |
| PECTATE LYASE A | 216, 225 |
| Bacillus licheniformis A4 | |
| PECTATE LYASE | 217, 226 |
| Bacillus amyloliquefaciens subsp. plantarum UCMB5113 | |

Polygalacturonase amino acid sequences are provided in Table 3 below, together with their SEQ ID NOs. SEQ ID NO: 227 is the same as SEQ ID NO: 210 with the addition of a cysteine residue at the carboxy terminus of SEQ ID NO: 210.

TABLE 3
Amino Acid Sequence for Polygalacturonases

| Enzyme | SEQ ID NO: |
|---|---|
| ENDOPOLYGALACTURONASE I (PGI) | 210, 227 |
| Aspergillus niger | |
| ENDOPOLYGALACTURONASE | 211 |
| Bacillus simplex 30N-5 | |
| ENDOPOLYGALACTURONASE | 212 |
| Bacillus spp. Strain 1 | |
| EXO-POLYGALACTURONASE | 218 |
| Bacillus licheniformis A4 | |
| POLYGALACTURONASE | 219 |
| Bacillus safensis | |
| POLYGALACTURONASE | 220 |
| Bacillus altitudinis | |
| POLYGALACTURONASE | 221 |
| Bacillus pumilus | |

### Signal Peptides

When a fusion protein comprises an enzyme whose native sequence includes a signal peptide, the enzyme can be used without the signal peptide. Alternatively, the native signal peptide (or another signal peptide) can optionally be included at the amino terminus of the enzyme, immediately preceding the first amino acid of the enzyme.

In addition, a signal peptide can optionally be included at the amino terminus of the enzymes whose native sequences do not include a signal peptide.

C. Fusion Proteins for Expression in Recombinant Bacillus cereus Family Members

Fusion proteins comprising a targeting sequence, exosporium protein, or exosporium protein fragment that targets the fusion protein to the exosporium of a recombinant Bacillus cereus family member are provided. The fusion proteins further comprise a pectinase, such as any one of the pectate lyases or polygalacturonases disclosed herein.

In any of the fusion proteins described herein, the fusion protein can comprise: (1) a targeting sequence comprising an amino acid sequence having at least about 43% identity with amino acids 20-35 of SEQ ID NO: 1, wherein the identity with amino acids 25-35 is at least about 54%; (2) a targeting sequence comprising amino acids 1-35 of SEQ ID NO: 1; (3) a targeting sequence comprising amino acids 20-35 of SEQ ID NO: 1; (4) a targeting sequence comprising SEQ ID NO: 1; (5) an exosporium protein comprising an amino acid sequence having at least 85% identity with SEQ ID NO: 2; (6) a targeting sequence comprising amino acids 2-35 of SEQ ID NO: 1; (7) a targeting sequence comprising amino acids 5-35 of SEQ ID NO: 1; (8) a targeting sequence comprising amino acids 8-35 of SEQ ID NO: 1; (9) a targeting sequence comprising amino acids 10-35 of SEQ ID NO: 1; (10) a targeting sequence comprising amino acids 15-35 of SEQ ID NO: 1; (11) a targeting sequence comprising amino acids 1-27 of SEQ ID NO: 3; (12) a targeting sequence comprising amino acids 12-27 of SEQ ID NO: 3; (13) a targeting sequence comprising SEQ ID NO: 3; (14) an exosporium protein comprising an amino acid sequence having at least 85% identity with SEQ ID NO: 4; (15) a targeting sequence comprising amino acids 2-27 of SEQ ID NO: 3; (16) a targeting sequence comprising amino acids 5-27 of SEQ ID NO: 3; (17) a targeting sequence comprising amino acids 8-27 of SEQ ID NO: 3; (18) a targeting sequence comprising amino acids 10-27 of SEQ ID NO: 3; (19) a targeting sequence comprising amino acids 1-38 of SEQ ID NO: 5; (20) a targeting sequence comprising amino acids 23-38 of SEQ ID NO: 5; (21) a targeting sequence comprising SEQ ID NO: 5; (22) an exosporium protein comprising an amino acid sequence having at least 85% identity with SEQ ID NO: 6; (23) a targeting sequence comprising amino acids 2-38 of SEQ ID NO: 5; (24) a targeting sequence comprising amino acids 5-38 of SEQ ID NO: 5; (25) a targeting sequence comprising amino acids 8-38 of SEQ ID NO: 5; (26) a targeting sequence comprising amino acids 10-38 of SEQ ID NO: 5; (27) a targeting sequence comprising amino acids 15-38 of SEQ ID NO: 5; (28) a targeting sequence comprising amino acids 20-38 of SEQ ID NO: 5; (29) a targeting sequence comprising amino acids 1-28 of SEQ ID NO: 7; (30) a targeting sequence comprising amino acids 13-28 of SEQ ID NO: 7; (31) a targeting sequence comprising SEQ ID NO: 7; (32) an exosporium protein comprising an amino acid sequence having at least 85% identity with SEQ ID NO: 8; (33) a targeting sequence comprising amino acids 2-28 of SEQ ID NO: 7; (34) a targeting sequence comprising amino acids 5-28 of SEQ ID NO: 7; (35) a targeting sequence comprising amino acids 8-28 of SEQ ID NO: 7; (36) a targeting sequence comprising amino acids 10-28 of SEQ ID NO: 7; (37) a targeting sequence comprising amino acids 1-24 of SEQ ID NO: 9; (38) a targeting sequence comprising amino acids 9-24 of SEQ ID NO: 9; (39) a targeting sequence comprising SEQ ID NO: 9; (40) an exosporium protein comprising an amino acid sequence having at least 85% identity with SEQ ID NO: 10; (41) a targeting sequence comprising amino acids 2-24 of SEQ ID NO: 9; (42) a targeting sequence comprising amino acids 5-24 of SEQ ID NO: 9; (43) a targeting sequence comprising amino acids 8-24 of SEQ ID NO: 9; (44) a targeting sequence comprising amino acids 1-33 of SEQ ID NO: 11; (45) a targeting sequence comprising amino acids 18-33 of SEQ ID NO: 11; (46) a targeting sequence comprising SEQ ID NO: 11; (47) an exosporium protein comprising an amino acid sequence having at least 85% identity with SEQ ID NO: 12; (48) a targeting sequence comprising amino acids 2-33 of SEQ ID NO: 11; (49) a targeting sequence comprising amino acids 5-33 of SEQ ID NO: 11; (50) a targeting sequence comprising amino acids 8-33 of SEQ ID NO: 11; (51) a targeting sequence comprising amino acids 10-33 of SEQ ID NO: 11; (52) a targeting sequence comprising amino acids 15-33 of SEQ ID NO: 11; (53) a targeting sequence comprising amino acids 1-33 of SEQ ID NO: 13; (54) a targeting sequence comprising amino acids 18-33 of SEQ ID NO: 13; (55) a targeting sequence comprising SEQ ID NO: 13; (56) an exosporium protein comprising an amino acid sequence having at least 85% identity with SEQ ID NO: 14; (57) a targeting sequence comprising amino acids 2-33 of SEQ ID NO: 13; (58) a targeting sequence comprising amino acids 5-33 of SEQ ID NO: 13; (59) a targeting sequence comprising amino acids 8-33 of SEQ ID NO: 13; (60) a targeting sequence comprising amino acids 10-33 of SEQ ID NO: 13; (61) a targeting sequence comprising amino acids 15-33 of SEQ ID NO: 13; (62) a targeting sequence comprising amino acids 1-43 of SEQ ID NO: 15; (63) a targeting sequence comprising amino acids 28-43 of SEQ ID NO: 15; (64) a targeting sequence comprising SEQ ID NO: 15; (65) an exosporium protein comprising an amino acid sequence having at least 85% identity with SEQ ID NO: 16; (66) a targeting sequence comprising amino acids 2-43 of SEQ ID NO: 15; (67) a targeting sequence comprising amino acids 5-43 of SEQ ID NO: 15; (68) a targeting sequence comprising amino acids 8-43 of SEQ ID NO: 15; (69) a targeting sequence comprising amino acids 10-43 of SEQ ID NO: 15; (70) a targeting sequence comprising amino acids 15-43 of SEQ ID NO: 15; (71) a targeting sequence comprising amino acids 20-43 of SEQ ID NO: 15; (72) a targeting sequence comprising amino acids 25-43 of SEQ ID NO: 15; (73) a targeting sequence comprising amino acids 1-27 of SEQ ID NO: 17; (74) a targeting sequence comprising amino acids 12-27 of SEQ ID NO: 17; (75) a targeting sequence comprising SEQ ID NO: 17; (76) an exosporium protein comprising an amino acid sequence having at least 85% identity with SEQ ID NO: 18; (77) a targeting sequence comprising amino acids 2-27 of SEQ ID NO: 17; (78) a targeting sequence comprising amino acids 5-27 of SEQ ID NO: 17; (79) a targeting sequence comprising amino acids 8-27 of SEQ ID NO: 17; (80) a targeting sequence comprising amino acids 10-27 of SEQ ID NO: 17; (81) a targeting sequence comprising amino acids 1-33 of SEQ ID NO: 19; (82) a targeting sequence comprising amino acids 18-33 of SEQ ID NO: 19; (83) a targeting sequence comprising SEQ ID NO: 19; (84) an exosporium protein comprising an amino acid sequence having at least 85% identity with SEQ ID NO: 20; (85) a targeting sequence comprising amino acids 2-33 of SEQ ID NO: 19; (86) a targeting sequence comprising amino acids 5-33 of SEQ ID NO: 19; (87) a targeting sequence comprising amino acids 8-33 of SEQ ID NO: 19; (88) a targeting sequence comprising amino acids 10-33 of SEQ ID NO: 19; (89) a targeting sequence comprising amino acids 15-33 of SEQ ID NO: 19; (90) a targeting sequence comprising amino acids 1-33 of SEQ ID NO: 21; (91) a targeting sequence comprising amino acids 18-33 of SEQ ID NO: 21; (92) a targeting sequence comprising SEQ ID NO: 21; (93) an exosporium protein comprising an amino acid sequence having at least 85% identity with SEQ ID NO: 22; (94) a targeting sequence comprising amino acids 2-33 of SEQ ID NO: 21; (95) a targeting sequence comprising amino acids 5-33 of SEQ ID NO: 21; (96) a targeting sequence comprising amino acids 8-33 of SEQ ID NO: 21; (97) a targeting sequence comprising amino acids 10-33 of SEQ ID NO: 21; (98) a targeting sequence comprising amino acids 15-33 of SEQ ID NO: 21; (99) a targeting sequence comprising amino acids 1-24 of SEQ ID NO: 23; (100) a targeting sequence comprising amino acids 9-24 of SEQ ID NO: 23; (101) a targeting sequence comprising SEQ ID NO: 23; (102) an exosporium protein comprising an amino acid sequence having at least 85% identity with SEQ ID NO: 24; (103) a targeting sequence comprising amino acids 2-24 of SEQ ID NO: 23; (104) a targeting sequence comprising amino acids 5-24 of SEQ ID NO: 23; (105) a targeting sequence comprising amino acids 8-24 of SEQ ID NO: 23; (106) a targeting sequence comprising amino acids 1-24 of SEQ ID NO: 25; (107) a targeting sequence comprising amino acids 9-24 of SEQ ID NO: 25; (108) a targeting sequence comprising SEQ ID NO: 25; (109) an exosporium protein comprising an amino acid sequence having at least 85% identity with SEQ ID NO: 26; (110) a targeting sequence comprising amino acids 2-24 of SEQ ID NO: 25; (111) a targeting sequence comprising amino acids 5-24 of SEQ ID NO: 25; (112) a targeting sequence comprising amino acids 8-24 of SEQ ID NO: 25; (113) a targeting sequence comprising amino acids 1-30 of SEQ ID NO: 27; (114) a targeting sequence comprising amino acids 15-30 of SEQ ID NO: 27; (115) a targeting sequence comprising SEQ ID NO: 27; (116) an exosporium protein comprising an amino acid sequence having at least 85% identity with SEQ ID NO: 28; (117) a targeting sequence comprising amino acids 2-30 of SEQ ID NO: 27; (118) a targeting sequence comprising amino acids 5-30 of SEQ ID NO: 27; (119) a targeting sequence comprising amino acids 8-30 of SEQ ID NO: 27; (120) a targeting sequence comprising amino acids 10-30 of SEQ ID NO: 27; (121) a targeting sequence comprising amino acids 1-33 of SEQ ID NO: 29; (122) a targeting sequence comprising amino acids 18-33 of SEQ ID NO: 29; (123) a targeting sequence comprising SEQ ID NO: 29; (124) an exosporium protein comprising an amino acid sequence having at least 85% identity with SEQ ID NO: 30; (125) a targeting sequence comprising amino acids 2-33 of SEQ ID NO: 29; (126) a targeting sequence comprising amino acids 5-33 of SEQ ID NO: 29; (127) a targeting sequence comprising amino acids 8-33 of SEQ ID NO: 29; (128) a targeting sequence comprising amino acids 10-33 of SEQ ID NO: 29; (129) a targeting sequence comprising amino acids 15-33 of SEQ ID NO: 29; (130) a targeting sequence comprising amino acids 1-24 of SEQ ID NO: 31; (131) a targeting sequence comprising amino acids 9-24 of SEQ ID NO: 31; (132) a targeting sequence comprising SEQ ID NO: 31; (133) an exosporium protein comprising an amino acid sequence having at least 85% identity with SEQ ID NO: 32; (134) a targeting sequence comprising amino acids 2-24 of SEQ ID NO: 31; (135) a targeting sequence comprising amino acids 5-24 of SEQ ID NO: 31; (136) a targeting sequence comprising amino acids 8-24 of SEQ ID NO: 31; (137) a targeting sequence comprising amino acids 1-15 of SEQ ID NO: 33; (138) a targeting sequence comprising SEQ ID NO: 33; (139) an exosporium protein comprising an amino acid sequence having at least 85% identity with SEQ ID NO: 34; (140) a targeting sequence comprising amino acids 1-16 of SEQ ID NO: 35; (141) a targeting sequence comprising SEQ ID NO: 35; (142) an exosporium protein comprising an amino acid sequence having at least 85% identity with SEQ ID NO: 36; (143) a targeting sequence comprising amino acids 1-29 of SEQ ID NO: 43; (144) a targeting sequence comprising amino acids 14-29 of SEQ ID NO: 43; (145) a targeting sequence comprising SEQ ID NO: 43; (146) an exosporium protein comprising an amino acid sequence having at least 85% identity with SEQ ID NO: 44; (147) a targeting sequence comprising amino acids 2-29 of SEQ ID NO: 43; (148) a targeting sequence comprising amino acids 5-29 of SEQ ID NO: 43; (149) a targeting sequence comprising amino acids 8-29 of SEQ ID NO: 43; (150) a targeting sequence comprising amino acids 10-29 of SEQ ID NO: 43; (151) a targeting sequence comprising amino acids 1-35 of SEQ ID NO: 45; (152) a targeting sequence comprising amino acids 20-35 of SEQ ID NO: 45; (153) a targeting sequence comprising SEQ ID NO: 45; (154) an exosporium protein comprising an amino acid sequence having at least 85% identity with SEQ ID NO: 46; (155) a targeting sequence comprising amino acids 2-35 of SEQ ID NO: 45; (156) a targeting sequence comprising amino acids 5-35 of SEQ ID NO: 45; (157) a targeting sequence comprising amino acids 8-35 of SEQ ID NO: 45; (158) a targeting sequence comprising amino acids 10-35 of SEQ ID NO: 45; (159) a targeting sequence comprising amino acids 15-35 of SEQ ID NO: 45; (160) a targeting sequence comprising amino acids 1-43 of SEQ ID NO: 47; (161) a targeting sequence comprising amino acids 28-43 of SEQ ID NO: 47; (162) a targeting sequence comprising SEQ ID NO: 47; (163) an exosporium protein comprising an amino acid sequence having at least 85% identity with SEQ ID NO: 48; (164) a targeting sequence comprising amino acids 2-43 of SEQ ID NO: 47; (165) a targeting sequence comprising amino acids 5-43 of SEQ ID NO: 47; (166) a targeting sequence comprising amino acids 8-43 of SEQ ID NO: 47; (167) a targeting sequence comprising amino acids 10-43 of SEQ ID NO: 47; (168) a targeting sequence comprising amino acids 15-43 of SEQ ID NO: 47; (169) a targeting sequence comprising amino acids 20-43 of SEQ ID NO: 47; (170) a targeting sequence comprising amino acids 25-43 of SEQ ID NO: 47; (171) a targeting sequence comprising amino acids 1-32 of SEQ ID NO: 49; (172) a targeting sequence comprising amino acids 17-32 of SEQ ID NO: 49; (173) a targeting sequence comprising SEQ ID NO: 49; (174) an exosporium protein comprising an amino acid sequence having at least 85% identity with SEQ ID NO: 50; (175) a targeting sequence comprising amino acids 2-32 of SEQ ID NO: 49; (176) a targeting sequence comprising amino acids 5-32 of SEQ ID NO: 49; (177) a targeting sequence comprising amino acids 8-32 of SEQ ID NO: 49; (178) a targeting sequence comprising amino acids 10-32 of SEQ ID NO: 49; (179) a targeting sequence comprising amino acids 15-32 of SEQ ID NO: 49; (180) a targeting sequence comprising amino acids 1-33 of SEQ ID NO: 51; (181) a targeting sequence comprising amino acids 18-33 of SEQ ID NO: 51; (182) a targeting sequence comprising SEQ ID NO: 51; (183) an exosporium protein comprising an amino acid sequence having at least 85% identity with SEQ ID NO: 52; (184) a targeting sequence comprising amino acids 2-33 of SEQ ID NO: 51; (185) a targeting sequence comprising amino acids 5-33 of SEQ ID NO: 51; (186) a targeting sequence comprising amino acids 8-33 of SEQ ID NO: 51; (187) a targeting sequence comprising amino acids 10-33 of SEQ ID NO: 51; (188) a targeting sequence comprising amino acids 15-33 of SEQ ID NO: 51; (189) a targeting sequence comprising amino acids 1-33 of SEQ ID NO: 53; (190) a targeting sequence comprising amino acids 18-33 of SEQ ID NO: 53; (191) a targeting sequence comprising SEQ ID NO: 53; (192) an exosporium protein comprising an amino acid sequence having at least 85% identity with SEQ ID NO: 54; (193) a targeting sequence comprising amino acids 2-33 of SEQ ID NO: 53; (194) a targeting sequence comprising amino acids 5-33 of SEQ ID NO: 53; (195) a targeting sequence comprising amino acids 8-33 of SEQ ID NO: 53; (196) a targeting sequence comprising amino acids 10-33 of SEQ ID NO: 53; (197) a targeting sequence comprising amino acids 15-33 of SEQ ID NO: 53; (198) a targeting sequence comprising amino acids 1-30 of SEQ ID NO: 55; (199) a targeting sequence comprising amino acids 15-30 of SEQ ID NO: 55; (200) a targeting sequence comprising SEQ ID NO: 55; (201) an exosporium protein comprising an amino acid sequence having at least 85% identity with SEQ ID NO: 56; (202) a targeting sequence comprising amino acids 2-30 of SEQ ID NO: 55; (203) a targeting sequence comprising amino acids 5-30 of SEQ ID NO: 55; (204) a targeting sequence comprising amino acids 8-30 of SEQ ID NO: 55; (205) a targeting sequence comprising amino acids 10-30 of SEQ ID NO: 55; (206) a targeting sequence comprising amino acids 1-130 of SEQ ID NO: 57; (207) a targeting sequence comprising amino acids 115-130 of SEQ ID NO: 57; (208) a targeting sequence comprising SEQ ID NO: 57; (209) an exosporium protein comprising an amino acid sequence having at least 85% identity with SEQ ID NO: 58; (210) a targeting sequence comprising amino acids 2-130 of SEQ ID NO: 57; (211) a targeting sequence comprising amino acids 5-130 of SEQ ID NO: 57; (212) a targeting sequence comprising amino acids 10-130 of SEQ ID NO: 57; (213) a targeting sequence comprising amino acids 20-130 of SEQ ID NO: 57; (214) a targeting sequence comprising amino acids 30-130 of SEQ ID NO: 57; (215) a targeting sequence comprising amino acids 40-130 of SEQ ID NO: 57; (216) a targeting sequence comprising amino acids 50-130 of SEQ ID NO: 57; (217) a targeting sequence comprising amino acids 60-130 of SEQ ID NO: 57; (218) a targeting sequence comprising amino acids 70-130 of SEQ ID NO: 57; (219) a targeting sequence comprising amino acids 80-130 of SEQ ID NO: 57; (220) a targeting sequence comprising amino acids 90-130 of SEQ ID NO: 57; (221) a targeting sequence comprising amino acids 100-130 of SEQ ID NO: 57; (222) a targeting sequence comprising amino acids 110-130 of SEQ ID NO: 57; (223) an exosporium protein fragment comprising an amino acid sequence having at least 85% identity with SEQ ID NO: 95; (224) a targeting sequence comprising SEQ ID NO: 96; (225) a targeting sequence comprising SEQ ID NO: 97; (226) a targeting sequence comprising SEQ ID NO: 98; (227) a targeting sequence comprising SEQ ID NO: 99; (228) a targeting sequence comprising SEQ ID NO: 100; (229) a targeting sequence comprising SEQ ID NO: 101; (230) a targeting sequence comprising SEQ ID NO: 102; (231) a targeting sequence comprising SEQ ID NO: 103; (232) a targeting sequence comprising SEQ ID NO: 104; (233) a targeting sequence comprising SEQ ID NO: 105; (234) a targeting sequence comprising SEQ ID NO: 106; (235) an exosporium protein comprising an amino acid sequence having at least 85% identity with SEQ ID NO: 108; (236) an exosporium protein comprising an amino acid sequence having at least 85% identity with SEQ ID NO: 109; (237) an exosporium protein comprising an amino acid sequence having at least 85% identity with SEQ ID NO: 110; (238) an exosporium protein comprising an amino acid sequence having at least 85% identity with SEQ ID NO: 111; (239) an exosporium protein comprising an amino acid sequence having at least 85% identity with SEQ ID NO: 112; (240) an exosporium protein comprising an amino acid sequence having at least 85% identity with SEQ ID NO: 113; (241) an exosporium protein comprising an amino acid sequence having at least 85% identity with SEQ ID NO: 114; (242) an exosporium protein comprising an amino acid sequence having at least 85% identity with SEQ ID NO: 115; (243) an exosporium protein comprising an amino acid sequence having at least 85% identity with SEQ ID NO: 116; (244) an exosporium protein comprising an amino acid sequence having at least 85% identity with SEQ ID NO: 117; (245) an exosporium protein comprising an amino acid sequence having at least 85% identity with SEQ ID NO: 118; (246) an exosporium protein comprising an amino acid sequence having at least 85% identity with SEQ ID NO: 119; (247) an exosporium protein comprising an amino acid sequence having at least 85% identity with SEQ ID NO: 120; (248) an exosporium protein comprising an amino acid sequence having at least 85% identity with SEQ ID NO: 121; (249) a targeting sequence comprising amino acids 22-31 of SEQ ID NO: 1; (250) a targeting sequence comprising amino acids 22-33 of SEQ ID NO: 1; (251) a targeting sequence comprising amino acids 20-31 of SEQ ID NO: 1; (252) a targeting sequence comprising amino acids 14-23 of SEQ ID NO: 3; (253) a targeting sequence comprising amino acids 14-25 of SEQ ID NO: 3; (254) a targeting sequence comprising amino acids 12-23 of SEQ ID NO: 3; (255) a targeting sequence comprising amino acids 1-30 of SEQ ID NO: 59; (256) a targeting sequence comprising SEQ ID NO: 59; (257) an exosporium protein comprising an amino acid sequence having at least 85% identity with SEQ ID NO: 60; (258) a targeting sequence comprising amino acids 2-30 of SEQ ID NO: 59; (259) a targeting sequence comprising amino acids 4-30 of SEQ ID NO: 59; (260) a targeting sequence comprising amino acids 6-30 of SEQ ID NO: 59; (261) a targeting sequence comprising amino acids 1-33 of SEQ ID NO: 61; (262) a targeting sequence comprising amino acids 18-33 of SEQ ID NO: 61; (263) a targeting sequence comprising SEQ ID NO: 61; (264) an exosporium protein comprising an amino acid sequence having at least 85% sequence identity with SEQ ID NO: 62; (265) a targeting sequence comprising amino acids 2-33 of SEQ ID NO: 61; (266) a targeting sequence comprising amino acids 5-33 of SEQ ID NO: 61; (267) a targeting sequence comprising amino acids 10-33 of SEQ ID NO: 61; (268) a targeting sequence comprising amino acids 15-33 of SEQ ID NO: 61; (269) a targeting sequence comprising amino acids 1-35 of SEQ ID NO: 63; (270) a targeting sequence comprising SEQ ID NO: 63; (271) an exosporium protein comprising an amino acid sequence having at least 85% identity with SEQ ID NO: 64; (272) a targeting sequence comprising amino acids 2-35 of SEQ ID NO: 63; (273) a targeting sequence comprising amino acids 5-35 of SEQ ID NO: 63; (274) a targeting sequence comprising amino acids 8-35 of SEQ ID NO: 63; (275) a targeting sequence comprising amino acids 10-35 of SEQ ID NO: 63; (276) a targeting sequence comprising amino acids 15-35 of SEQ ID NO: 63; (277) a targeting sequence comprising amino acids 1-24 of SEQ ID NO: 65; (278) a targeting sequence comprising amino acids 9-24 of SEQ ID NO: 65; (279) a targeting sequence comprising SEQ ID NO: 65; (280) an exosporium protein comprising an amino acid sequence having at least 85% identity with SEQ ID NO: 66; (281) a targeting sequence comprising SEQ ID NO: 107; (282) a targeting sequence comprising amino acids 2-24 of SEQ ID NO: 65; (283) a targeting sequence comprising amino acids 5-24 of SEQ ID NO: 65; (284) a targeting sequence comprising amino acids 1-27 of SEQ ID NO: 67; (285) a targeting sequence comprising amino acids 12-27 of SEQ ID NO: 67; (286) a targeting sequence comprising SEQ ID NO: 67; (287) an exosporium protein comprising an amino acid sequence having at least 85% identity with SEQ ID NO: 68; (288) an targeting sequence comprising amino acids 2-27 of SEQ ID NO: 67; (289) a targeting sequence comprising amino acids 5-27 of SEQ ID NO: 67; (290) a targeting sequence comprising amino acids 10-27 of SEQ ID NO: 67; (291) a targeting sequence comprising amino acids 1-38 of SEQ ID NO: 69; (292) a targeting sequence comprising amino acids 23-38 of SEQ ID NO: 69; (293) a targeting sequence comprising SEQ ID NO: 69; (294) an exosporium protein comprising an amino acid sequence having at least 85% identity with SEQ ID NO: 70; (295) a targeting sequence comprising amino acids 2-38 of SEQ ID NO: 69; (296) a targeting sequence comprising amino acids 5-38 of SEQ ID NO: 69; (297) a targeting sequence comprising amino acids 10-38 of SEQ ID NO: 69; (298) a targeting sequence comprising amino acids 15-38 of SEQ ID NO: 69; (299) an exosporium protein comprising SEQ ID NO: 72; (300) a targeting sequence comprising SEQ ID NO: 73; (301) an exosporium protein comprising an amino acid sequence having at least 95% identity with SEQ ID NO: 74; (302) a targeting sequence comprising amino acids 1-42 of SEQ ID NO: 75; (303) a targeting sequence comprising amino acids 27-42 of SEQ ID NO: 75; (304) a targeting sequence comprising SEQ ID NO: 75; (305) an exosporium protein comprising an amino acid sequence having at least 85% identity with SEQ ID NO: 76; (306) a targeting sequence comprising amino acids 2-42 of SEQ ID NO: 75; (307) a targeting sequence comprising amino acids 5-42 of SEQ ID NO: 75; (308) a targeting sequence comprising amino acids 10-42 of SEQ ID NO: 75; (309) a targeting sequence comprising amino acids 15-42 of SEQ ID NO: 75; (310) a targeting sequence comprising amino acids 20-42 of SEQ ID NO: 75; (311) a targeting sequence comprising amino acids 25-42 of SEQ ID NO: 75; (312) a targeting sequence comprising amino acids 1-24 of SEQ ID NO: 77; (313) a targeting sequence comprising amino acids 9-24 of SEQ ID NO: 77; (314) a targeting sequence comprising SEQ ID NO: 77; (315) an exosporium protein comprising an amino acid sequence having at least 85% identity with SEQ ID NO: 78; (316) a targeting sequence comprising amino acids 2-24 of SEQ ID NO: 77; (317) a targeting sequence comprising amino acids 5-24 of SEQ ID NO: 77; (318) an exosporium protein comprising an amino acid sequence having at least 85% identity with SEQ ID NO: 80; (319) a targeting sequence comprising amino acids 1-38 of SEQ ID NO: 81; (320) a targeting sequence comprising amino acids 23-38 of SEQ ID NO: 81; (321) a targeting sequence comprising SEQ ID NO: 81; (322) an exosporium protein comprising an amino acid sequence having at least 85% identity with SEQ ID NO: 82; (323) a targeting sequence comprising amino acids 2-38 of SEQ ID NO: 81; (324) a targeting sequence comprising amino acids 5-38 of SEQ ID NO: 81; (325) a targeting sequence comprising amino acids 10-38 of SEQ ID NO: 81; (326) a targeting sequence comprising amino acids 15-38 of SEQ ID NO: 81; (327) a targeting sequence comprising amino acids 20-38 of SEQ ID NO: 81; (328) a targeting sequence comprising amino acids 1-34 of SEQ ID NO: 83; (329) a targeting sequence comprising SEQ ID NO: 83; (330) an exosporium protein comprising an amino acid sequence having at least 85% identity with SEQ ID NO: 84; (331) an exosporium protein comprising an amino acid sequence having at least 85% identity with SEQ ID NO: 86; (332) a targeting sequence comprising amino acids 1-28 of SEQ ID NO: 87; (333) a targeting sequence comprising amino acids 13-28 of SEQ ID NO: 87; (334) a targeting sequence comprising SEQ ID NO: 87; (335) an exosporium protein comprising an amino acid sequence having at least 85% identity with SEQ ID NO: 88; (336) a targeting sequence comprising amino acids 2-28 of SEQ ID NO: 87; (337) a targeting sequence comprising amino acids 5-28 of SEQ ID NO: 87; (338) a targeting sequence comprising amino acids 10-28 of SEQ ID NO: 87; (339) a targeting sequence comprising amino acids 1-28 of SEQ ID NO: 89; (340) a targeting sequence comprising SEQ ID NO: 89; (341) an exosporium protein comprising an amino acid sequence having at least 85% identity with SEQ ID NO: 90; (342) a targeting sequence comprising amino acids 2-28 of SEQ ID NO: 89; (343) a targeting sequence comprising amino acids 5-28 of SEQ ID NO: 89; (344) a targeting sequence comprising amino acids 10-28 of SEQ ID NO: 89; (345) a targeting sequence comprising amino acids 1-93 of SEQ ID NO: 91; (346) a targeting sequence comprising SEQ ID NO: 91; (347) an exosporium protein comprising an amino acid sequence having at least 85% identity with SEQ ID NO: 92; (348) a targeting sequence comprising amino acids 2-93 of SEQ ID NO: 91; (349) a targeting sequence comprising amino acids 10-93 of SEQ ID NO: 91; (350) a targeting sequence comprising amino acids 20-93 of SEQ ID NO: 91; (351) a targeting sequence comprising amino acids 30-93 of SEQ ID NO: 91; (352) a targeting sequence comprising amino acids 40-93 of SEQ ID NO: 91; (353) a targeting sequence comprising amino acids 50-93 of SEQ ID NO: 91; (354) a targeting sequence comprising amino acids 60-93 of SEQ ID NO: 91; (355) a targeting sequence comprising amino acids 1-130 of SEQ ID NO: 93; (356) a targeting sequence comprising SEQ ID NO: 93; (357) an exosporium protein comprising an amino acid sequence having at least 85% identity with SEQ ID NO: 94; (358) a targeting sequence comprising amino acids 2-130 of SEQ ID NO: 93; (359) a targeting sequence comprising amino acids 10-130 of SEQ ID NO: 93; (360) a targeting sequence comprising amino acids 20-130 of SEQ ID NO: 93; (361) a targeting sequence comprising amino acids 30-130 of SEQ ID NO: 93; (362) an exosporium protein comprising an amino acid sequence having at least 85% identity with SEQ ID NO: 122; (363) a targeting sequence consisting of amino acids 20-33 of SEQ ID NO: 1; (364) a targeting sequence consisting of amino acids 21-33 of SEQ ID NO: 1; (365) a targeting sequence consisting of amino acids 23-31 of SEQ ID NO: 1; (366) a targeting sequence consisting of amino acids 1-15 of SEQ ID NO: 96; (367) a targeting sequence consisting of amino acids 1-13 of SEQ ID NO: 96; (368) a targeting sequence consisting of amino acids 12-25 of SEQ ID NO: 3; (369) a targeting sequence consisting of amino acids 13-25 of SEQ ID NO: 3; (370) a targeting sequence consisting of amino acids 15-23 of SEQ ID NO: 3; (371) a targeting sequence consisting of amino acids 1-15 of SEQ ID NO: 97; (372) a targeting sequence consisting of amino acids 1-13 of SEQ ID NO: 98; (373) a targeting sequence consisting of amino acids 23-36 of SEQ ID NO: 5; (374) a targeting sequence consisting of amino acids 23-34 of SEQ ID NO: 5; (375) a targeting sequence consisting of amino acids 24-36 of SEQ ID NO: 5; (376) a targeting sequence consisting of amino acids 26-34 of SEQ ID NO: 5; (377) a targeting sequence consisting of amino acids 13-26 of SEQ ID NO: 7; (378) a targeting sequence consisting of amino acids 13-24 of SEQ ID NO: 7; (379) a targeting sequence consisting of amino acids 14-26 of SEQ ID NO: 7; (380) a targeting sequence consisting of amino acids 16-24 of SEQ ID NO: 7; (381) a targeting sequence consisting of amino acids 9-22 of SEQ ID NO: 9; (382) a targeting sequence consisting of amino acids 9-20 of SEQ ID NO: 9; (383) a targeting sequence consisting of amino acids 10-22 of SEQ ID NO: 9; (384) a targeting sequence consisting of amino acids 12-20 of SEQ ID NO: 9; (385) a targeting sequence consisting of amino acids 1-15 of SEQ ID NO: 105; (386) a targeting sequence consisting of amino acids 1-13 of SEQ ID NO: 105; (387) a targeting sequence consisting of amino acids 18-31 of SEQ ID NO: 11; (388) a targeting sequence consisting of amino acids 18-29 of SEQ ID NO: 11; (389) a targeting sequence consisting of amino acids 19-31 of SEQ ID NO: 11; (390) a targeting sequence consisting of amino acids 1-15 of SEQ ID NO: 98; (391) a targeting sequence consisting of amino acids 1-13 of SEQ ID NO: 98; (392) a targeting sequence consisting of amino acids 18-31 of SEQ ID NO: 13; (393) a targeting sequence consisting of amino acids 18-29 of SEQ ID NO: 13; (394) a targeting sequence consisting of amino acids 19-31 of SEQ ID NO: 13; (395) a targeting sequence consisting of amino acids 21-29 of SEQ ID NO: 13; (396) a targeting sequence consisting of amino acids 1-15 of SEQ ID NO: 99; (397) a targeting sequence consisting of amino acids 1-13 of SEQ ID NO: 99; (398) a targeting sequence consisting of amino acids 28-41 of SEQ ID NO: 15; (399) a targeting sequence consisting of amino acids 28-39 of SEQ ID NO: 15; (400) a targeting sequence consisting of amino acids 29-41 of SEQ ID NO: 15; (401) a targeting sequence consisting of amino acids 31-39 of SEQ ID NO: 15; (402) a targeting sequence consisting of amino acids 12-25 of SEQ ID NO: 17; (403) a targeting sequence consisting of amino acids 13-25 of SEQ ID NO: 17; (404) a targeting sequence consisting of amino acids 1-15 of SEQ ID NO: 100; (405) a targeting sequence consisting of amino acids 18-31 of SEQ ID NO: 19; (406) a targeting sequence consisting of amino acids 18-29 of SEQ ID NO: 19; (407) a targeting sequence consisting of amino acids 19-31 of SEQ ID NO: 19; (408) a targeting sequence consisting of amino acids 21-29 of SEQ ID NO: 19; (409) a targeting sequence consisting of amino acids 18-31 of SEQ ID NO: 21; (410) a targeting sequence consisting of amino acids 18-29 of SEQ ID NO: 21; (411) a targeting sequence consisting of amino acids 19-31 of SEQ ID NO: 21; (412) a targeting sequence consisting of amino acids 21-29 of SEQ ID NO: 21; (413) a targeting sequence consisting of amino acids 1-15 of SEQ ID NO: 101; (414) a targeting sequence consisting of amino acids 1-13 of SEQ ID NO: 101; (415) a targeting sequence consisting of amino acids 9-22 of SEQ ID NO: 23; (416) a targeting sequence consisting of amino acids 9-20 of SEQ ID NO: 23; (417) a targeting sequence consisting of amino acids 10-22 of SEQ ID NO: 23; (418) a targeting sequence consisting of amino acids 12-20 of SEQ ID NO: 23; (419) a targeting sequence consisting of amino acids 1-15 of SEQ ID NO: 102; (420) a targeting sequence consisting of amino acids 1-13 of SEQ ID NO: 102; (421) a targeting sequence consisting of amino acids 9-22 of SEQ ID NO: 25; (422) a targeting sequence consisting of amino acids 9-20 of SEQ ID NO: 25; (423) a targeting sequence consisting of amino acids 10-22 of SEQ ID NO: 25; (424) a targeting sequence consisting of amino acids 12-20 of SEQ ID NO: 25; (425) a targeting sequence consisting of amino acids 1-15 of SEQ ID NO: 103; (426) a targeting sequence consisting of amino acids 1-13 of SEQ ID NO: 103; (427) a targeting sequence consisting of amino acids 15-28 of SEQ ID NO: 27; (428) a targeting sequence consisting of amino acids 15-26 of SEQ ID NO: 27; (429) a targeting sequence consisting of amino acids 16-28 of SEQ ID NO: 27; (430) a targeting sequence consisting of amino acids 18-26 of SEQ ID NO: 27; (431) a targeting sequence consisting of amino acids 1-15 of SEQ ID NO: 104; (432) a targeting sequence consisting of amino acids 1-13 of SEQ ID NO: 104; (433) a targeting sequence consisting of amino acids 1-13 of SEQ ID NO: 33; (434) a targeting sequence consisting of amino acids 1-11 of SEQ ID NO: 33; (435) a targeting sequence consisting of amino acids 3-11 of SEQ ID NO: 33; (436) a targeting sequence consisting of amino acids 1-14 of SEQ ID NO: 35; (437) a targeting sequence consisting of amino acids 1-12 of SEQ ID NO: 35; (438) a targeting sequence consisting of amino acids 2-14 of SEQ ID NO: 35; (439) a targeting sequence consisting of amino acids 14-27 of SEQ ID NO: 43; (440) a targeting sequence consisting of amino acids 14-25 of SEQ ID NO: 43; (441) a targeting sequence consisting of amino acids 15-27 of SEQ ID NO: 43; (442) a targeting sequence consisting of amino acids 20-33 of SEQ ID NO: 45; (443) a targeting sequence consisting of amino acids 20-31 of SEQ ID NO: 45; (444) a targeting sequence consisting of amino acids 21-33 of SEQ ID NO: 45; (445) a targeting sequence consisting of amino acids 1-15 of SEQ ID NO: 106; (446) a targeting sequence consisting of amino acids 1-13 of SEQ ID NO: 106; (447) a targeting sequence consisting of amino acids 28-41 of SEQ ID NO: 47; (448) a targeting sequence consisting of amino acids 28-39 of SEQ ID NO: 47; (449) a targeting sequence consisting of amino acids 18-31 of SEQ ID NO: 53; (450) a targeting sequence consisting of amino acids 18-29 of SEQ ID NO: 53; (451) a targeting sequence consisting of amino acids 19-31 of SEQ ID NO: 53; (452) a targeting sequence comprising amino acids 18-31 of SEQ ID NO: 61; (453) a targeting sequence comprising amino acids 18-29 of SEQ ID NO: 61; (454) a targeting sequence comprising amino acids 19-31 of SEQ ID NO: 61; (455) a targeting sequence comprising amino acids 9-22 of SEQ ID NO: 65; (456) a targeting sequence comprising amino acids 9-20 of SEQ ID NO: 65; (457) a targeting sequence comprising amino acids 10-22 of SEQ ID NO: 65; (458) a targeting sequence comprising amino acids 1-15 of SEQ ID NO: 107; (459) a targeting sequence comprising amino acids 1-13 of SEQ ID NO: 107; (460) a targeting sequence comprising amino acids 12-25 of SEQ ID NO: 67; (461) a targeting sequence comprising amino acids 12-23 of SEQ ID NO: 67; (462) a targeting sequence comprising amino acids 13-25 of SEQ ID NO: 67; (463) a targeting sequence comprising amino acids 15-23 of SEQ ID NO: 67; (464) a targeting sequence comprising amino acids 23-36 of SEQ ID NO: 69; (465) a targeting sequence comprising amino acids 23-34 of SEQ ID NO: 69; (466) a targeting sequence comprising amino acids 24-36 of SEQ ID NO: 69; (467) a targeting sequence comprising amino acids 26-34 of SEQ ID NO: 69; (468) a targeting sequence comprising amino acids 27-40 of SEQ ID NO: 75; (469) a targeting sequence comprising amino acids 27-38 of SEQ ID NO: 75; (470) a targeting sequence comprising amino acids 9-22 of SEQ ID NO: 77; (471) a targeting sequence comprising amino acids 9-20 of SEQ ID NO: 77; (472) a targeting sequence comprising amino acids 10-22 of SEQ ID NO: 77; (473) a targeting sequence comprising amino acids 12-20 of SEQ ID NO: 77; (474) a targeting sequence comprising amino acids 23-36 of SEQ ID NO: 81; (475) a targeting sequence comprising amino acids 23-34 of SEQ ID NO: 81; (476) a targeting sequence comprising amino acids 24-36 of SEQ ID NO: 81; (477) a targeting sequence comprising amino acids 26-34 of SEQ ID NO: 81; (478) a targeting sequence comprising amino acids 13-26 of SEQ ID NO: 87; (479) a targeting sequence comprising amino acids 13-24 of SEQ ID NO: 87; or (480) a targeting sequence comprising amino acids 14-26 of SEQ ID NO: 87; (481) a targeting sequence comprising SEQ ID NO: 201; (482) a targeting sequence comprising SEQ ID NO: 202; (483) an exosporium protein comprising an amino acid sequence having at least 85% identity with SEQ ID NO: 203; (484) an exosporium protein comprising an amino acid sequence having at least 85% identity with SEQ ID NO: 204; (485) an exosporium protein fragment comprising an amino acid sequence having at least 85% identity with SEQ ID NO: 205; (486) an exosporium protein comprising an amino acid sequence having at least 85% identity with SEQ ID NO: 206; or (487) an exosporium protein fragment comprising an amino acid sequence having at least 85% identity with SEQ ID NO: 207.

For example, the targeting sequence can comprise an amino acid sequence having at least about 50% identity with amino acids 20-35 of SEQ ID NO: 1, wherein the identity with amino acids 25-35 is at least about 63%.

Alternatively, the targeting sequence can consist of an amino acid sequence having at least about 50% identity with amino acids 20-35 of SEQ ID NO: 1, wherein the identity with amino acids 25-35 is at least about 63%.

The targeting sequence can comprise an amino acid sequence having at least about 50% identity with amino acids 20-35 of SEQ ID NO: 1, wherein the identity with amino acids 25-35 is at least about 72%.

Alternatively, the targeting sequence can consist of an amino acid sequence having at least about 50% identity with amino acids 20-35 of SEQ ID NO: 1, wherein the identity with amino acids 25-35 is at least about 72%.

The targeting sequence can comprise an amino acid sequence having at least about 56% identity with amino acids 20-35 of SEQ ID NO: 1, wherein the identity with amino acids 25-35 is at least about 63%.

Alternatively, the targeting sequence can consist of an amino acid sequence having at least about 56% identity with amino acids 20-35 of SEQ ID NO: 1, wherein the identity with amino acids 25-35 is at least about 63%.

The targeting sequence can comprise an amino sequence having at least about 62% identity with amino acids 20-35 of SEQ ID NO: 1, wherein the identity with amino acids 25-35 is at least about 72%.

Alternatively, the targeting sequence can consist of an amino sequence having at least about 62% identity with amino acids 20-35 of SEQ ID NO: 1, wherein the identity with amino acids 25-35 is at least about 72%.

The targeting sequence can comprise an amino acid sequence having at least about 68% identity with amino acids 20-35 of SEQ ID NO: 1, wherein the identity with amino acids 25-35 is at least about 81%.

Alternatively, the targeting sequence can consist of an amino acid sequence having at least about 68% identity with amino acids 20-35 of SEQ ID NO: 1, wherein the identity with amino acids 25-35 is at least about 81%.

The targeting sequence can comprise an amino sequence having at least about 75% identity with amino acids 20-35 of SEQ ID NO: 1, wherein the identity with amino acids 25-35 is at least about 72%.

Alternatively, the targeting sequence can consist of an amino sequence having at least about 75% identity with amino acids 20-35 of SEQ ID NO: 1, wherein the identity with amino acids 25-35 is at least about 72%.

The targeting sequence can comprise an amino sequence having at least about 75% identity with amino acids 20-35 of SEQ ID NO: 1, wherein the identity with amino acids 25-35 is at least about 81%.

Alternatively, the targeting sequence can consist of an amino sequence having at least about 75% identity with amino acids 20-35 of SEQ ID NO: 1, wherein the identity with amino acids 25-35 is at least about 81%.

The targeting sequence can comprise an amino acid sequence having at least about 81% identity with amino acids 20-35 of SEQ ID NO: 1, wherein the identity with amino acids 25-35 is at least about 81%.

Alternatively, the targeting sequence can consist of an amino acid sequence having at least about 81% identity with amino acids 20-35 of SEQ ID NO: 1, wherein the identity with amino acids 25-35 is at least about 81%.

The targeting sequence can comprise an amino acid sequence having at least about 81% identity with amino acids 20-35 of SEQ ID NO: 1, wherein the identity with amino acids 25-35 is at least about 90%.

Alternatively, the targeting sequence can consist of an amino acid sequence having at least about 81% identity with amino acids 20-35 of SEQ ID NO: 1, wherein the identity with amino acids 25-35 is at least about 90%.

For example, the targeting sequence can consist of: (a) an amino acid sequence consisting of 16 amino acids and having at least about 43% identity with amino acids 20-35 of SEQ ID NO: 1, wherein the identity with amino acids 25-35 is at least about 54%; (b) amino acids 1-35 of SEQ ID NO: 1; (c) amino acids 20-35 of SEQ ID NO: 1; (d) SEQ ID NO: 1; (e) SEQ ID NO: 96; or (f) SEQ ID NO: 120.

In any of the fusion proteins described herein, the fusion protein can comprise an exosporium protein or an exosporium protein fragment comprising an amino acid sequence having at least 90% identity with SEQ ID NO: 2, 4, 6, 8, 10, 12, 14, 16, 18, 20, 22, 24, 26, 28, 30, 32, 34, 36, 44, 46, 48, 50, 52, 54, 56, 58, 95, 108, 109, 110, 111, 112, 113, 114, 115, 116, 117, 118, 119, 120, and 121.

The fusion protein can comprise an exosporium protein or an exosporium protein fragment comprising an amino acid sequence having at least 95% identity with SEQ ID NO: 2, 4, 6, 8, 10, 12, 14, 16, 18, 20, 22, 24, 26, 28, 30, 32, 34, 36, 44, 46, 48, 50, 52, 54, 56, 58, 95, 108, 109, 110, 111, 112, 113, 114, 115, 116, 117, 118, 119, 120, and 121.

The fusion protein can comprise an exosporium protein or an exosporium protein fragment comprising an amino acid sequence having at least 98% identity with SEQ ID NO: 2, 4, 6, 8, 10, 12, 14, 16, 18, 20, 22, 24, 26, 28, 30, 32, 34, 36, 44, 46, 48, 50, 52, 54, 56, 58, 95, 108, 109, 110, 111, 112, 113, 114, 115, 116, 117, 118, 119, 120, and 121.

The fusion protein can comprise an exosporium protein or an exosporium protein fragment comprising an amino acid sequence having at least 99% identity with SEQ ID NO: 2, 4, 6, 8, 10, 12, 14, 16, 18, 20, 22, 24, 26, 28, 30, 32, 34, 36, 44, 46, 48, 50, 52, 54, 56, 58, 95, 108, 109, 110, 111, 112, 113, 114, 115, 116, 117, 118, 119, 120, and 121.

The fusion protein can comprise an exosporium protein or an exosporium protein fragment comprising an amino acid sequence having 100% identity with SEQ ID NO: 2, 4, 6, 8, 10, 12, 14, 16, 18, 20, 22, 24, 26, 28, 30, 32, 34, 36, 44, 46, 48, 50, 52, 54, 56, 58, 95, 108, 109, 110, 111, 112, 113, 114, 115, 116, 117, 118, 119, 120, and 121.

In any of the fusion proteins described herein, the fusion protein can comprise an exosporium protein comprising an amino acid sequence having at least 90% identity with SEQ ID NO: 60, 62, 64, 66, 68, 70, 76, 78, 80, 82, 84, 86, 88, 90, 92, 94, or 122.

The fusion protein can comprise an exosporium protein comprising an amino acid sequence having at least 95% identity with SEQ ID NO: 60, 62, 64, 66, 68, 70, 76, 78, 80, 82, 84, 86, 88, 90, 92, 94, or 122.

The fusion protein can comprise an exosporium protein comprising an amino acid sequence having at least 98% identity with SEQ ID NO: 60, 62, 64, 66, 68, 70, 76, 78, 80, 82, 84, 86, 88, 90, 92, 94, or 122.

The fusion protein can comprise an exosporium protein comprising an amino acid sequence having at least 99% identity with SEQ ID NO: 60, 62, 64, 66, 68, 70, 76, 78, 80, 82, 84, 86, 88, 90, 92, 94, or 122.

The fusion protein can comprise an exosporium protein comprising an amino acid sequence having 100% identity with SEQ ID NO: 60, 62, 64, 66, 68, 70, 76, 78, 80, 82, 84, 86, 88, 90, 92, 94, or 122.

In any of the fusion proteins described herein, the fusion protein can comprise an exosporium protein or an exosporium protein fragment comprising an amino acid sequence having at least 90% identity with any one of SEQ ID NOs: 203-207.

The fusion protein can comprise an exosporium protein or an exosporium protein fragment comprising an amino acid sequence having at least 95% identity with any one of SEQ ID NOs: 203-207.

The fusion protein can comprise an exosporium protein or an exosporium protein fragment comprising an amino acid sequence having at least 98% identity with any one of SEQ ID NOs: 203-207.

The fusion protein can comprise an exosporium protein or an exosporium protein fragment comprising an amino acid sequence having at least 99% identity with any one of SEQ ID NOs: 203-207.

The fusion protein can comprise an exosporium protein or an exosporium protein fragment comprising an amino acid sequence having 100% identity with any one of SEQ ID NOs: 203-207.

The fusion protein can comprise a targeting sequence, exosporium protein, or exosporium protein fragment that targets the fusion protein to the exosporium of the recombinant Bacillus bacterium, wherein the targeting sequence, exosporium protein, or exosporium protein fragment comprises the sequence X₁-X₂-X₃-X₄-X₅-X₆-X₇-X₈-X₉-X₁₀-X₁₁-X₁₂-X₁₃-X₁₄-X₁₅-X₁₆, wherein:

X₁ is any amino acid or absent;

X₂ is phenylalanine (F), leucine (L), isoleucine (I), or methionine (M);

X₃ is any amino acid;

X₄ is proline (P) or serine (S);

X₅ is any amino acid;

X₆ is leucine (L), asparagine (N), serine (S), or isoleucine (I);

X₇ is valine (V) or isoleucine (I);

X₈ is glycine (G);

X₉ is proline (P);

X₁₀ is threonine (T) or proline (P);

X₁₁ is leucine (L) or phenylalanine (F);

X₁₂ is proline (P);

X₁₃ is any amino acid;

X₁₄ is any amino acid;

X₁₅ is proline (P), glutamine (Q), or threonine (T); and

X₁₆ is proline (P), threonine (T), or serine (S)

In any of the fusion proteins described herein, the targeting sequence, exosporium protein, or exosporium protein fragment can comprise the amino acid sequence GXT at its carboxy terminus, wherein X is any amino acid.

In any of the fusion proteins described herein, the targeting sequence, exosporium protein, or exosporium protein fragment can comprise an alanine residue at the position of the targeting sequence that corresponds to amino acid 20 of SEQ ID NO: 1.

In any of the fusion proteins described herein, the targeting sequence, exosporium protein, or exosporium protein fragment can further comprise a methionine, serine, or threonine residue at the amino acid position immediately preceding the first amino acid of the targeting sequence, exosporium protein, or exosporium protein fragment or at the position of the targeting sequence that corresponds to amino acid 20 of SEQ ID NO: 1.

### Fusion Proteins Comprising Certain Pectinases

Fusion proteins comprising a targeting sequence, exosporium protein, or exosporium protein fragment that targets the fusion protein to the exosporium of a recombinant Bacillus cereus family member and the following pectinases are provided.

For the pectinases described herein, sequence identity is determined by aligning the entire length of the sequences in such a way as to obtain optimal matching so that the minimal number of edit operations (e.g., inserts, deletions and substitutions) are needed in order to transform the one sequence into an exact copy of the other sequence being aligned. The Needleman-Wünsch Global Alignmment of Protein Sequences, which is an algorithm that is available through the U.S National Library of Medicine's National Center for Biotechnology Information (“NCBI”) website, is one example of such analysis.

The pectinase can comprise a pectate lyase from Bacillus spp. In one aspect of this embodiment, the pectate lyase is from Bacillus subtilis (SEQ ID NO: 213 or 222), Bacillus amyloliquefaciens (SEQ ID NO: 217 or 226), Bacillus licheniformis (SEQ ID NO: 216 or 225), Bacillus safensis (SEQ ID NO: 214 or 223) or Bacillus pumilus (SEQ ID NO: 215 or 224).

SEQ ID NO: 214 and SEQ ID NO: 215 have 93% sequence identity. SEQ ID NO: 214 and SEQ ID NO: 216 have 66% sequence identity. SEQ ID NO: 215 and SEQ ID NO: 216 have 62% sequence identity.

Alternatively or in addition, the pectate lyase can comprise an amino acid sequence having at least 70% identity to any one of SEQ ID NOs: 213-217 and 222-226.

For example, the pectate lyase can comprise an amino acid sequence having at least 75% identity to any one of SEQ ID NOs: 213-217 and 222-226.

The pectate lyase can comprise an amino acid sequence having at least 80% identity to any one of SEQ ID NOs: 213-217 and 222-226.

The pectate lyase can comprise an amino acid sequence having at least 85% identity to any one of SEQ ID NOs: 213-217 and 222-226.

The pectate lyase can comprise an amino acid sequence having at least 90% identity to any one of SEQ ID NOs: 213-217 and 222-226.

The pectate lyase can comprise an amino acid sequence having at least 95% identity to any one of SEQ ID NOs: 213-217 and 222-226.

The pectate lyase can comprise an amino acid sequence having at least 98% identity to any one of SEQ ID NOs: 213-217 and 222-226.

The pectate lyase can comprise an amino acid sequence having at least 99% identity to any one of SEQ ID NOs: 213-217 and 222-226.

The pectate lyase can comprise an amino acid sequence having 100% identity to any one of SEQ ID NOs: 213-217 and 222-226.

For example, the enzyme can comprise SEQ ID NOs: 213-217 and 222-226.

Alternatively, the enzyme can consist of SEQ ID NOs: 213-217 and 222-226.

The pectinase can comprise an endopolygalacturonase from Aspergillus niger. In one embodiment, the endoploygalaturonase is from Aspergillus niger ATCC 9029. In one aspect of this embodiment the enzyme comprises SEQ ID NO: 210 or 227. SEQ ID NO: 227 is the same as SEQ ID NO: 210 with the addition of a cysteine residue at the carboxy terminus of SEQ ID NO: 210.

Alternatively or in addition, the endopolygalacturonase can comprise an amino acid sequence having at least 60% identity to SEQ ID NO: 210 or 227.

Alternatively or in addition, the endopolygalacturonase can comprise an amino acid sequence having at least 70% identity to SEQ ID NO: 210 or 227.

For example, the endopolygalacturonase can comprise an amino acid sequence having at least 75% identity to SEQ ID NO: 210 or 227.

The endopolygalacturonase can comprise an amino acid sequence having at least 80% identity to SEQ ID NO: 210 or 227.

The endopolygalacturonase can comprise an amino acid sequence having at least 85% identity to SEQ ID NO: 210 or 227.

The endopolygalacturonase can comprise an amino acid sequence having at least 90% identity to SEQ ID NO: 210 or 227.

The endopolygalacturonase can comprise an amino acid sequence having at least 95% identity to SEQ ID NO: 210 or 227.

The endopolygalacturonase can comprise an amino acid sequence having at least 98% identity to SEQ ID NO: 210 or 227.

The endopolygalacturonase can comprise an amino acid sequence having at least 99% identity to SEQ ID NO: 210 or 227.

The endopolygalacturonase can comprise an amino acid sequence having 100% identity to SEQ ID NO: 210 or 227.

Alternatively, the enzyme can consist of SEQ ID NO: 210 or 227.

In another embodiment the pectinase is an endopolygalacturonase from a Bacillus spp. strain or from Bacillus simplex. In one aspect, the endopolygalacturonase is from Bacillus simplex 30 N-5, as described in Khan, N., et al., “Antifungal Activity of Bacillus Species Against Fusarium and Analyis of the Potential Mechanisms Used in Biocontrol,” Frontiers in Microbiology 9:2363 (2018).

For example, the amino acid sequence of the enzyme can comprise any one of SEQ ID NO: 211-212. SEQ ID NO: 211 is an endopolygalacturonase from Bacillus simplex 30 N-5. SEQ ID NO: 212 is an endopolygalacturonase from another Bacillus spp. strain. SEQ ID NO: 211 and SEQ ID NO:212 have 85% sequence identity.

Alternatively or in addition, the endopolygalacturonase can comprise an amino acid sequence having at least 70% identity to any one of SEQ ID NO: 211-212.

For example, the endopolygalacturonase can comprise an amino acid sequence having at least 75% identity to any one of SEQ ID NO: 211-212.

The endopolygalacturonase can comprise an amino acid sequence having at least 80% identity to any one of SEQ ID NO: 211-212.

The endopolygalacturonase can comprise an amino acid sequence having at least 85% identity to any one of SEQ ID NO: 211-212.

The endopolygalacturonase can comprise an amino acid sequence having at least 90% identity to any one of SEQ ID NO: 211-212.

The endopolygalacturonase can comprise an amino acid sequence having at least 95% identity to any one of SEQ ID NO: 211-212.

The endopolygalacturonase can comprise an amino acid sequence having at least 98% identity to any one of SEQ ID NO: 211-212.

The endopolygalacturonase can comprise an amino acid sequence having at least 99% identity to any one of SEQ ID NO: 211-212.

The endopolygalacturonase can comprise an amino acid sequence having 100% identity to any one of SEQ ID NO: 211-212.

Alternatively, the enzyme can consist of any one of SEQ ID NO: 211-212.

In yet another embodiment, the pectinase is a polygalacturonase from a Bacillus spp. strain in which the catalytic residues of endopolygalacturonase I from Aspergillus niger that are described in van Pouderoyen (2003), above, are conserved. In a particular aspect of this embodiment the polygalaturonase with the conserved catalytic residues is from Bacillus licheniformis, Bacillus safensis, Bacillus altitudinus, or Bacillus pumilus. An exopolygalacturonase from Bacillus licheniformis is described in Evangelista, D., et al. “Biochemical Characterization and Low-Resolution SAXS Structure of an Exo-Polygalacturonase from Bacillus licheniformis,” New Biotechnology 40: 268-274 (2018). In another aspect, such polygalacturonase comprises any one of SEQ ID NOs: 218-221. SEQ ID NO: 218 is an exo-polygalacturonase from Bacillus licheniformis, as described in Evangelista (2018), above. SEQ ID NO: 221 is a polygalacturonase from Bacillus pumilus. SEQ ID NO: 219 is a polygalacturonase from Bacillus safensis. SEQ ID NO: 220 is a polygalacturonase from Bacillus altitudinis. SEQ ID NO: 219 and SEQ ID NO: 220 have 90% sequence identity. SEQ ID NO: 221 has sequence identity of about 90% to each of SEQ ID NO: 219 and SEQ ID NO: 220. SEQ ID NO: 218 has sequence identity of about 60% to each of SEQ ID NO: 219 and SEQ ID NO: 220.

Alternatively or in addition, the polygalacturonase can comprise an amino acid sequence having at least 60% identity to any one of SEQ ID NOs: 218-221. Additionally, such amino acid sequence can comprise the catalytic residues conserved with those of endopolygalacturonase I from Aspergillus niger; namely, Asp186, Asp207, Asp208, and His 229.

For example, the polygalacturonase can comprise an amino acid sequence having at least 75% identity to any one of SEQ ID NOs: 218-221.

The polygalacturonase can comprise an amino acid sequence having at least 80% identity to any one of SEQ ID NOs: 218-221.

The polygalacturonase can comprise an amino acid sequence having at least 85% identity to any one of SEQ ID NOs: 218-221.

The polygalacturonase can comprise an amino acid sequence having at least 90% identity to any one of SEQ ID NOs: 218-221.

The polygalacturonase can comprise an amino acid sequence having at least 95% identity to any one of SEQ ID NOs: 218-221.

The polygalacturonase can comprise an amino acid sequence having at least 98% identity to any one of SEQ ID NOs: 218-221.

The polygalacturonase can comprise an amino acid sequence having at least 99% identity to any one of SEQ ID NOs: 218-221.

The polygalacturonase can comprise an amino acid sequence having 100% identity to any one of SEQ ID NOs: 218-221.

Alternatively, the enzyme can consist of any one of SEQ ID NOs: 218-221.

### Optional Inclusion of Signal Peptides in the Fusion Proteins

In any of the fusion proteins described herein, the pectinase can further comprise a signal peptide.

Where the signal peptide is present, it is preferably present at the amino terminus of the pectinase.

The signal peptide preferably immediately precedes the first amino acid of the pectinase.

Where the fusion protein comprises a signal peptide, the signal peptide can be present at the amino terminus of the pectinase.

### D. Methods for Making the Fusion Proteins

Any of the fusion proteins described herein can be made using standard cloning and molecular biology methods known in the art. For example, a gene encoding a protein or peptide of interest (e.g., a pectinase, including any of the pectate lyases or polygalacturonases described herein) can be amplified by polymerase chain reaction (PCR) and ligated to DNA coding for any of the targeting sequences, exosporium proteins, or exosporium protein fragments described herein, to form a DNA molecule that encodes the fusion protein. The DNA molecule encoding the fusion protein can be cloned into any suitable vector, for example a plasmid vector. The vector suitably comprises a multiple cloning site into which the DNA molecule encoding the fusion protein can be easily inserted. The vector also suitably contains a selectable marker, such as an antibiotic resistance gene, such that bacteria transformed, transfected, or mated with the vector can be readily identified and isolated. Where the vector is a plasmid, the plasmid suitably also comprises an origin of replication. Alternatively, DNA coding for the fusion protein can be integrated into the chromosomal DNA of the B. cereus family member or spore-forming bacterium host.

E. Tags, Markers, and Linkers that can be Included in the Fusion Proteins

Any of the fusion proteins described herein can also comprise additional polypeptide sequences that are not part of the targeting sequence, exosporium protein, exosporium protein fragment, or the pectinase. For example, the fusion protein can include tags or markers to facilitate purification or visualization of the fusion protein (e.g., a polyhistidine tag or a fluorescent protein such as GFP or YFP) or visualization of recombinant Bacillus cereus family member spores expressing the fusion protein.

Expression of fusion proteins on the exosporium of a Bacillus cereus family member using the targeting sequences, exosporium proteins, and exosporium protein fragments described herein is enhanced due to a lack of secondary structure in the amino-termini of these sequences, which allows for native folding of the fused proteins and retention of activity. Proper folding can be further enhanced by the inclusion of a short amino acid linker between the targeting sequence, exosporium protein, exosporium protein fragment, spore coat protein, and the pectinase.

Thus, any of the fusion proteins described herein can comprise an amino acid linker between the targeting sequence, the exosporium protein, or the exosporium protein fragment and the pectinase.

The linker can comprise a polyalanine linker or a polyglycine linker. A linker comprising a mixture of both alanine and glycine residues can also be used. Examples of polyalanine linkers are provided as SEQ ID NOs: 208 and 209.

For example, in a fusion protein where the targeting sequence comprises SEQ ID NO: 1, a fusion protein can have one of the following structures:

No linker: SEQ ID NO: 1-POI

Alanine Linker: SEQ ID NO: 1-Aₙ-POI

Glycine Linker: SEQ ID NO: 1-Gₙ-POI

Mixed Alanine and Glycine Linker: SEQ ID NO: 1-(A/G)ₙ-POI

where Aₙ, Gₙ, and (A/G)ₙ are any number of alanines, any number of glycines, or any number of a mixture of alanines and glycines, respectively. For example, n can be 1 to 25, and is preferably 6 to 10. Where the linker comprises a mixture of alanine and glycine residues, any combination of glycine and alanine residues can be used. In the above structures, “POI” stands for “protein of interest” and represents the pectinase, including any of the pectate lyases or polygalacturonases described herein.

Alternatively or in addition, the linker can comprise a protease recognition site. Inclusion of a protease recognition site allows for targeted removal, upon exposure to a protease that recognizes the protease recognition site, of the pectinase, including any of the pectate lyases or polygalacturonases described herein.

Where the fusion protein comprises both a linker and signal peptide, the linker would typically be amino-terminal to the signal peptide. For example, where the fusion protein comprises SEQ ID NO: 96, a polyalanine linker, a signal sequence, and the endopolygalacturonase of SEQ ID NO: 210, these elements would typically be arranged in the following order within the fusion protein, going from the amino-terminus of the fusion protein to the carboxy-terminus:

SEQ ID NO: 96-Aₙ-signal sequence-SEQ ID NO: 210.

II. Recombinant Bacillus cereus Family Members as Hosts for Expression of the Fusion Proteins and Methods for Fermentation of such Recombinant Bacillus cereus Family Members

The invention further relates to recombinant Bacillus cereus family members that express a fusion protein. The fusion protein can be any of the fusion proteins described in Section I above.

The recombinant Bacillus cereus family member can comprise any Bacillus species that is capable of producing an exosporium. For example, the recombinant Bacillus cereus family member can comprise Bacillus anthracia, Bacillus cereus, Bacillus thuringiensis, Bacillus mycoides, Bacillus pseudomycoides, Bacillus samanii, Bacillus gaemokensis, Bacillus weihenstephensis, Bacillus toyoiensis, or a combination of any thereof. The recombinant Bacillus cereus family member suitably comprises Bacillus thuringiensis or Bacillus mycoides.

To generate a recombinant Bacillus cereus family member expressing a fusion protein, any Bacillus cereus family member can be conjugated, transduced, or transformed with a vector encoding the fusion protein using standard methods known in the art (e.g., by electroporation). The bacteria can then be screened to identify transformants by any method known in the art. For example, where the vector includes an antibiotic resistance gene, the bacteria can be screened for antibiotic resistance. Alternatively, DNA encoding the fusion protein can be integrated into the chromosomal DNA of a B. cereus family member host. The recombinant Bacillus cereus family member can then exposed to conditions which will induce sporulation. Suitable conditions for inducing sporulation are known in the art. For example, the recombinant Bacillus cereus family member can be plated onto agar plates, and incubated at a temperature of about 30° C. for several days (e.g., 3 days).

Thus, the recombinant Bacillus cereus family member can be in the form of a spore.

Inactivated strains, non-toxic strains, or genetically manipulated strains of any of the above species can also suitably be used. For example, a Bacillus thuringiensis that lacks the Cry toxin can be used. Alternatively or in addition, once the recombinant B. cereus family member spores expressing the fusion protein have been generated, they can be inactivated to prevent further germination once in use. Any method for inactivating bacterial spores that is known in the art can be used. Suitable methods include, without limitation, heat treatment, gamma irradiation, x-ray irradiation, UV-A irradiation, UV-B irradiation, chemical treatment (e.g., treatment with glutaraldehyde, formaldehyde, hydrogen peroxide, acetic acid, bleach, or any combination thereof), or a combination thereof. Alternatively, spores derived from nontoxigenic strains, or genetically or physically inactivated strains, can be used.

Thus, the recombinant Bacillus cereus family member can be in the form of a spore, wherein the spore is inactivated.

The recombinant Bacillus cereus family member can coexpress two or more of any of the fusion proteins described herein. For example, the recombinant Bacillus cereus family member can coexpress at least one fusion protein that comprises a pectate lyase together with a fusion protein that comprises a polygalacturonase.

Many Bacillus cereus family member strains have inherent beneficial attributes. For example, some strains have plant-growth promoting effects. Other strains are endophytic. Some strains are both endophytic and have plant-growth promoting effects.

Thus, any of the recombinant Bacillus cereus family members described herein can comprise a plant-growth promoting strain of bacteria, an endophytic strain of bacteria, or a strain of bacteria that is both plant-growth promoting and endophytic.

The plant-growth promoting strain of bacteria can comprise a strain of bacteria that produces an insecticidal toxin (e.g., a Cry toxin), produces a fungicidal compound (e.g., a β-1,3-glucanase, a chitosanase, a lyticase, or a combination of any thereof), produces a nematocidal compound (e.g., a Cry toxin), produces a bacteriocidal compound, is resistant to one or more antibiotics, comprises one or more freely replicating plasmids, binds to plant roots, colonizes plant roots, forms biofilms, solubilizes nutrients, secretes organic acids, or any combination thereof.

The recombinant Bacillus cereus family member can comprises an endophytic strain of bacteria.

The recombinant Bacillus cereus family member can comprise an inactivating mutation in its BclA gene, its CotE gene, or its CotO gene (e.g., a knock-out of the BclA gene, CotE gene, or CotO gene). For example, the recombinant Bacillus cereus family member can comprise an inactivating mutation in its BclA gene (e.g., a knock-out of the BclA gene). It has been found that expression of fusion proteins in a recombinant Bacillus cereus family member having such a mutation results in increased expression levels of the fusion protein.

Compositions of the present invention include cultures, such as whole broth cultures, of the strains described herein. The term culture refers to a population of cells growing in the absence of other species in a predetermined culture media under controlled laboratory or manufacturing conditions. Biologically pure cultures of the recombinant Bacillus cereus family members of the present invention may be obtained according to methods well known in the art.

Conventional large-scale microbial culture processes include submerged fermentation, solid state fermentation, or liquid surface culture. During the fermentation, as nutrients are depleted, cells begin the transition from growth phase to sporulation phase, such that the final product of fermentation is largely spores, metabolites and residual fermentation medium. Sporulation is part of the natural life cycle of Bacillus cereus family members and is generally initiated by the cell in response to stressful environmental conditions, such as nutrient limitation. Fermentation is configured to obtain high levels of colony forming units and to promote sporulation. The bacterial cells, spores and metabolites in culture media resulting from fermentation may be used directly or concentrated by conventional industrial methods, such as centrifugation or filtration such as tangential-flow filtration or depth filtration, and evaporation.

Compositions of the present invention include the products of the microbial culture processes described herein. In embodiments in which submerged fermentation is used as the culture process, the product is referred to as a “fermentation broth” or a “whole broth culture.” Such broth may be concentrated, as described above. The concentrated fermentation broth may be washed, for example, via a diafiltration process, to remove residual fermentation broth and metabolites. The term “broth concentrate,” as used herein, refers to fermentation broth that has been concentrated by conventional industrial methods, as described above, but remains in liquid form. The term “fermentation product,” as used herein, refers to fermentation broth or whole broth culture, broth concentrate and/or dried fermentation broth or broth concentrate.

The fermentation broth or broth concentrate can be dried with or without the addition of carriers using conventional drying processes or methods such as spray drying, freeze drying, tray drying, fluidized-bed drying, drum drying, or evaporation. The term “fermentation product,” as used herein, refers to fermentation broth or whole broth culture, broth concentrate and/or dried fermentation broth or broth concentrate.

The resulting dry products may be further processed, such as by milling or granulation, to achieve a specific particle size or physical format. Carriers, described below, may also be added post-drying.

Cell-free preparations of fermentation broth of the strains of the present invention can be obtained by any means known in the art, such as extraction, centrifugation and/or filtration of fermentation broth. Those of skill in the art will appreciate that so-called cell-free preparations may not be devoid of cells but rather are largely cell-free or essentially cell-free, depending on the technique used (e.g., speed of centrifugation) to remove the cells. The resulting cell-free preparation may be dried and/or formulated with components that aid in its application to plants or to plant growth media. Concentration methods and drying techniques described above for fermentation broth are also applicable to cell-free preparations.

As described further below in Section IV, the recombinant Bacillus cereus family member can comprise a mutation or other modification that allows for collection of exosporium fragments comprising the fusion proteins from spores of the recombinant Bacillus cereus family member.

III. Promoters for Expression of Fusion Proteins in Recombinant Bacillus cereus Family Members

The DNA encoding the fusion proteins used in the recombinant Bacillus cereus family members, exosporium fragments, formulations, plant seeds, and methods, described herein is suitably under the control of a sporulation promoter which will cause expression of the fusion protein on the exosporium of a B. cereus family member endospore (e.g., a native bclA promoter from a B. cereus family member).

Thus, any of the fusion proteins described above in Section I can be expressed in the recombinant Bacillus cereus family member under the control of a sporulation promoter that is native to the targeting sequence, exosporium protein, or exosporium protein fragment of the fusion protein, or a portion of such a promoter.

Any of the fusion proteins can be expressed under the control of a high-expression sporulation promoter.

The high-expression sporulation promoter can comprise a sigma-K sporulation-specific polymerase promoter sequence.

For ease of reference, illustrative nucleotide sequences for promoters that can be used to express any of the fusion proteins in a recombinant Bacillus cereus family member are provided in Table 4 below, together with their SEQ ID NOs. Table 4 also provides illustrative minimal promoter sequences for many of the promoters. In Table 4, sigma-K sporulation-specific polymerase promoter sequences in the promoters are indicated by bold and underlined text. Several of the sequences have multiple sigma K sequences that overlap with one another. The overlaps are indicated by double underlining in the table. The promoter sequences are immediately upstream of the start codon for each of the indicated genes. In other words, in the sequences shown in Table 4 below, the last nucleotide of the promoter sequence immediately precedes the first nucleotide of the start codon for the coding region of the gene encoding the indicated protein.

TABLE 4
Promoter Sequences for Expression of Fusion Proteins in Recombinant
Bacillus cereus Family Members

| Promoter | |
|---|---|
| (SEQ ID NO:) | Promoter Sequence |
| ExsY promoter | TTTCTTAATCCTTTACCCTTTACTTTTGTAAAAGTTGATACACTTCCATCCGGCTCTGTA |
| (B. cereus F837/76) | ATTTCTAATTCATCAATAAATGGTCTTCGCAAAAAGCCTGTAATTTTATCATAAACAAT |
| (SEQ ID NO: 37) | TAAACGAGTGAGCCTAAAAGCAGCTAACGCGAAAATAAAAAATAAAAGCCAGCTTGT |
| | AAACAGCATAATTCCACCTTCCCTTATCCTCTTTCGCCTATTTAAAAAAAGGTCTTGAG |
| | ATTGTGACCAAATCTCCTCAACTCCAATATCTTATTAATGTAAATACAAACAAGAAGA |
| | TAAGGA |
| ExsY minimal promoter | ACCAAATCTCCTCAACTCCAATATCTTATTAATGTAAATACAAACAAGAAGATAAGGA |
| (B. cereus F837/76) | |
| (SEQ ID NO: 38) | |
| ExsFA/BxpB promoter | ACCACCTACCGACGATCCAATCTGTACATTCCTAGCTGTACCAAATGCAAGATTAATAT |
| (B. anthracis Sterne) | CGACTAACACTTGTCTTACTGTTGATTTAAGTTGCTTCTGTGCGATTCAATGCTTGCGTG |
| (SEQ ID NO: 39) | ATGTTACGATTTAAAACTAAATAATGAGCTAAGCATGGATTGGGTGGCAGAATTATCT |
| | GCCACCCAATCCATGCTTAACGAGTATTATTATGTAAATTTCTTAAAATTGGGAACTTG |
| | TCTAGAACATAGAACCTGTCCTTTTCATTAACTGAAAGTAGAAACAGATAAAGGAGT |
| | GAAAAAC |
| ExsFA/BxpB minimal promoter | ACATAGAACCTGTCCTTTTCATTAACTGAAAGTAGAAACAGATAAAGGAGTGAAAAA |
| (B. anthracis Sterne) | C |
| (SEQ ID NO: 40) | |
| CotY/CotZ promoter | TAGAAGAAGAACGCCGACTACTTTATGTCGCAATTACACGGGCGAAAGAAGAACTTTA |
| (B. anthracis Sterne) | CATTTCCTCTCCGCAATTTTTTAGAGGAAAAAAATTAGATATATCTCGTTTTTTATACA |
| (SEQ ID NO: 41) | CTGTGCGAAAAGATTTACCTGAAAAGACATCCACTAAATAAGGATGTCTTTTTTTATAT |
| | TGTATTATGTACATCCCTACTATATAAATTCCCTGCTTTTATCGTAAGAATTAACGTAA |
| | TATCAACCATATCCCGTTCATATTGTAGTAGTGTATGTCAGAACTCACGAGAAGGAGT |
| | GAACATA |
| CotY/CotZ minimal promoter | TCAACCATATCCCGTTCATATTGTAGTAGTGTATGTCAGAACTCACGAGAAGGAGTGA |
| (B. anthracis Sterne) | ACATA |
| (SEQ ID NO: 42) | |
| CotO promoter (B. cereus) | TAACTCAATCTTAAGAGAAATTGAGGAGCGCGCACCACTTCGTCGTACAACAACGCAA |
| (SEQ ID NO: 123) | GAAGAAGTTGGGGATACAGCAGTATTCTTATTCAGTGATTTAGCACGCGGCGTAACAG |
| | GAGAAAACATTCACGTTGATTCAGGGTATCATATCTTAGGATAAATATAATATTAATT |
| | TTAAAGGACAATCTCTACATGTTGAGATTGTCCTTTTTATTTGTTCTTAGAAAGAACGA |
| | TTTTTAACGAAAGTTCTTACCACGTTATGAATATAAGTATAATAGTACACGATTTATTC |
| | AGCTACGT |
| CotO minimal promoter | ACGTTGATTCAGGGTATCATATCTTAGGATAAATATAATATTAATTTTAAAGGACAAT |
| (B. cereus) | CTCTACATGTTGAGATTGTCCTTTTTATTTGTTCTTAGAAAGAACGATTTTTAACGAAA |
| (SEQ ID NO: 124) | GTTCTTACCACGTTATGAATATAAGTATAATAGTACACGATTTATTCAGCTACGT |
| ExsFB promoter | CATAAAAATCTACTTTTCTTGTCAAAGAGTATGCTTATATGCGTGCTCTTTTTATTTGGT |
| (B. cereus F837/76) | TTTCTTTCATTTCTAAATAACATTTTCAACTCTATTCATACTATTCTTTCAACTTTAGGTT |
| (SEQ ID NO: 125) | ACAAACTATTTCTGTAAGCGTAGTGTTTCTTTTGTACTATAGGCAGTTAGTTTTATCCAT |
| | AACAGTACACCTCTGCACTATTCACTATAAATTTTCATATATTATATTGTGCTTGTCCA |
| | AAACATGTGGTTATTACTCACGCGATCTAAATGAAAGAAAGGAGTGAAAAT |
| ExsFB minimal promoter | ACTATTCACTATAAATTTTCATATATTATATTGTGCTTGTCCAAAACATGTGGTTATTA |
| (B. cereus F837/76) | CTCACGCGATCTAAATGAAAGAAAGGAGTGAAAAT |
| (SEQ ID NO: 126) | |
| InhA1 promoter | AATACATGATAATGAAATCCGATTTTGTGTTTTATATAGTGAATTATCAAATATTGTGT |
| (B. thuringiensis serovar | AGATGAAACAAAGATAAAATCCCCATTAAACTCCCTCTATGGAAATTATAAATTGTTC |
| kurstaki str. HD-1) | GATAAAAACTTTCAATATTTTCAGAAAACATTGTTGAATTGTGATATATTCGTATGCTA |
| (SEQ ID NO: 127) | ACTATGAAATTTTTACAAATATATTAAAAACATTACATAATATGACTAAATATTGAAA |
| | AAATATTGAATTTTTAATAAAATTTAATTTGTAATACATATTATTTATTAGGGGAGGA |
| | AATAAGGG |
| InhA1 minimal promoter | AAAATTTAATTTGTAATACATATTATTTATTAGGGGAGGAAATAAGGG |
| (B. thuringiensis serovar | |
| kurstaki str. HD-1) | |
| (SEQ ID NO: 128) | |
| InhA2 promoter | AATTGTGCATATTGTCTTTTAAATTTTCTATCTAAGTTATTTAATATATAATAAATAACT |
| (B. mycoides strain 219298) | CTTTTTTGTGAGTTTTTTTGATACGAGGTAAATAATCAGTACAGGGTCTGACCAGAGGA |
| (SEQ ID NO: 129) | CTGGAGGGCATGATTCTATAAGGGAATATTTACTATTCCATGATTATAGAACTATGTCT |
| | TTTTTATTGTATATAGAAGGGGGGATAGGTCTATATTATAGAACTTATATATATTGTGC |
| | ATTCCATATTATCAATTATCTAAATTTTAAGTCTTGTTACAATTAATAAGGGAGGAAA |
| | TAGTA |
| InhA2 minimal promoter | ACTTATATATATTGTGCATTCCATATTATCAATTATCTAAATTTTAAGTCTTGTTACAA |
| (B. mycoides strain 219298) | TTAATAAGGGAGGAAATAGTA |
| (SEQ ID NO: 130) | |
| ExsJ promoter | AATGACGTTTTCAAGTTTGATTATCATTCATGTTTCCTATTTTAAGAGAAACATATAAC |
| (B. thuringiensis serovar | TCAACTACTTTTTTCAATGGCATCTTTTATAGTACTTAGAATAGGAAAACACTCAACT |
| kurstaki) | |
| (SEQ ID NO: 131) | ATAAGAAAAGTAAGGAGGAAATAA |
| ExsJ minimal promoter | ACTACTTTTTTCAATGGCATCTTTTATAGTACTTAGAATAGGAAAACACTCAACTATA |
| (B. thuringiensis serovar | AGAAAAGTAAGGAGGAAATAA |
| kurstaki) | |
| (SEQ ID NO: 132) | |
| ExsH promoter | ATATGCTAATGCTTAGTTTTTATACTCAAGTTAAAATGTGCTTTTGGACCTAAGAGATA |
| (B. cereus F837/76) | AACGTGGAAAAATAAAATAAACTCTTAAGTTTAGGTGTTTAATCTAAGCAGTCAATTA |
| (SEQ ID NO: 133) | TTAAAAACATATAATTAATATGTGAGTCATGAACATAATTAAATAATGTTTTCAAGTT |
| | TAATTATCGTTCATGTTTCCTATTTTAAGCAGAACAAATAACTCAATTACTTTTTTCGAT |
| | TGGATCTTTTTTAACTCTTATAATAGGAAAACACTCAACTATAAAAATAAGTAAGGAG |
| | GAAATAA |
| ExsH minimal promoter | AATATGTGAGTCATGAACATAATTAAATAATGTTTTCAAGTTTAATTATCGTTCATGTT |
| (B. cereus F837/76) | TCCTATTTTAAGCAGAACAAATAACTCAATTACTTTTTTCGATTGGATCTTTTTTAACTC |
| (SEQ ID NO: 134) | TTATAATAGGAAAACACTCAACTATAAAAATAAGTAAGGAGGAAATAA |
| YjcA promoter | TATAAAATAAAAGGGCGTGTATTTGCTACTGATGCAGTATTGTGTGCGCCTAAAAATG |
| (B. thuringiensis serovar | GAATTTCACAACCAGATCCACATGTTGTTGTAGAACAATCTTGTAATTCATTGATGAAT |
| kurstaki str. HD73) | TTTACAACGTCAACTACACAATGAGAAGAGCCATGGTGTTTATTTTCGTTACAACTCAT |
| (SEQ ID NO: 135) | TAATGTCACTCCTTATCTTCTTGTTTGTATTTACATTAATAAGATATTGGAGTTGAGGA |
| | GATTTGGTCACAATCTCAAGACCTTTTTTTTAAATAGGCGAAAGAGGATAAGGGAAGG |
| | TGGAATT |
| YjcA minimal promoter | TCTTGTTTGTATTTACATTAATAAGATATTGGAGTTGAGGAGATTTGGTCACAATCTCA |
| (B. thuringiensis serovar | AGACCTTTTTTTTAAATAGGCGAAAGAGGATAAGGGAAGGTGGAATT |
| kurstaki str. HD73) | |
| (SEQ ID NO: 136) | |
| YjcB promoter | ATCAACTTTTACAAAAGTAAAGGGTAAAGGATTAAGAAAGTGGATTGGCGAATTATTA |
| (B. thuringiensis serovar | AGCTGTTATTGGTGTACAGGTGTATGGGTTAGTGCTTTTTTATTAGTTTTATATAATTGG |
| kurstaki str. HD73) | ATTCCGATCGTTGCAGAGCCGTTACTTGCATTATTAGCTATTGCAGGAGCAGCAGCAAT |
| (SEQ ID NO: 137) | CATTGAAACGATTACAGGATATTTTATGGGAGAATAATATATTTTCATAATACGAGAA |
| | AAAGCGGAGTTTAAAAGAATGAGGGAACGGAAATAAAGAGTTGTTCATATAGTAAAT |
| | AGACAGAA |
| YjcB minimal promoter | ACGGAAATAAAGAGTTGTTCATATAGTAAATAGACAGAA |
| (B. thuringiensis serovar | |
| kurstaki str. HD73) | |
| (SEQ ID NO: 138) | |
| BclC promoter | TGAAGTATCTAGAGCTAATTTACGCAAAGGAATCTCAGGACAACACTTTCGCAACACC |
| (B. anthracis Sterne) | TATATTTTAAATTTAATAAAAAAAGAGACTCCGGAGTCAGAAATTATAAAGCTAGCTG |
| (SEQ ID NO: 139) | GGTTCAAATCAAAAATTTCACTAAAACGATATTATCAATACGCAGAAAATGGAAAAAA |
| | CGCCTTATCATAAGGCGTTTTTTCCATTTTTTCTTCAAACAAACGATTTTACTATGACCA |
| | TTTAACTAATTTTTGCATCTACTATGATGAGTTTCATTCACATTCTCATTAGAAAGGAG |
| | AGATTTA |
| BclC minimal promoter | ACCATTTAACTAATTTTTGCATCTACTATGATGAGTTTCATTCACATTCTCATTAGAAA |
| (B. anthracis Sterne) | GGAGAGATTTA |
| (SEQ ID NO: 140) | |
| AcpC promoter | GACTATGTTTATTCAGGATAAAATATAGCACTACACTCTCTCCTCTTATTATGTAGCAT |
| (B. cereus F837/76) | CTCTCTAATCCATCATTTGTTTCATTTAGTTAAAATTGTAAATAAAATCACATGATTTGT |
| (SEQ ID NO: 141) | CAATTATAATTGTCATTTCGACAATTAAACTTGTCAAAATAATTCTCATCATTTTTTCTC |
| | ATCTTTCTAATATAGGACATACTACTATATATACAAAAGACAATATGCAAATGTTCAT |
| | ACAAAAAATATTATTTTTCGATATATAATATTAACTGATTTTCTAACATCAAGGAGGG |
| | TACAT |
| AcpC minimal promoter | AGACAATATGCAAATGTTCATACAAAAAATATTATTTTTCGATATATAATATTAACTG |
| (B. cereus F837/76) | ATTTTCTAACATCAAGGAGGGTACAT |
| (SEQ ID NO: 142) | |
| InhA3 promoter | ATAGTGAGTAATATGGTAATCCATAGATTAAATAGTATAGAAAATATTTAATTCTTAT |
| (B. thuringiensis serovar | TTTTATTAAAAAAGCATGAATCCCAGATTTACTGGGTTTTGATTGTAACTAAGAACATA |
| kurstaki str. HD73) | TAAAAGTTCACTGTTATTTATAGGAGAGTCTGTTTGTTTTTATATCTTATGTATTTCAC |
| (SEQ ID NO: 143) | CCTGCATAAAAAAATATTTCTCAACATTTTATTTGTTGAAAAATATTGAATATTCGTAT |
| | TATAACGAATATTATGTTGTTATCGGCAAAAAACGATAATTTGCAGACACTGGGGAGG |
| | AAATACA |
| InhA3 minimal promoter | TCTTATGTATTTCACCCTGCATAAAAAAATATTTCTCAACATTTTATTTGTTGAAAAAT |
| (B. thuringiensis serovar | ATTGAATATTCGTATTATAACGAATATTATGTTGTTATCGGCAAAAAACGATAATTTGC |
| kurstaki str. HD73) | AGACACTGGGGAGGAAATACA |
| (SEQ ID NO: 144) | |
| Alanine racemase 1 promoter | CTTCGTCAGCAATAAGTGTGAGCGGAGAATTGGTTGATCTTGGCTTTACAATTGGAGC |
| (B. cereus F837/76) | ATTGACGAAAGACTCTTTAACGTGGTCGCATAACGGAGTAGAATATATGCTCGTGTCT |
| (SEQ ID NO: 145) | AAAGGTTTAGAGCCGAAGGAGCTATTAATGGTTGCTCGTTCAGTTACAGAGAAGCAAG |
| | TGAAGTAAACTTCTTAGACGTGGTGATATATGTGCACCACGTCTTTTCTTAGTTTGAAG |
| | GGTGGATTTCATAAAAGAAGCATATAAAAGAATAAGCTTCGCATATCGTGTATAAGG |
| | AAGTGTATTT |
| Alanine racemase 1 minimal | ATAAAAGAATAAGCTTCGCATATCGTGTATAAGGAAGTGTATTT |
| promoter (B. cereus F837/76) | |
| (SEQ ID NO: 146) | |
| Alanine racemase 2 promoter | CATTTCAAATAATGAACGCTTCGATTGAATCGGAGCTATTTTCAAATCAATTTCAGTAT |
| (B. thuringiensis serovar | ATTGATCCAGCATTTGAATAGAAGTATCAACAGCAACTTTAAGTTGATGCAATGCAGA |
| kurstaki str. HD73) | TTGTACAAACATTGTAATTCTCCTCTTCTCCGTATATAATAGTTTCTTGAGGGTATTAT |
| (SEQ ID NO: 147) | ATCATGCTCAAAATTCCGAAAATTCTAGTAGTTTGACTAGCATATTGAAAAGTATTAT |
| | ATTGTAAAAGGTCATATGAAACGTGAAATAGAATGGAATGCAATTATTGAGTTAGGA |
| | GTTAGACCA |
| Alanine racemase 2 minimal | TTATATTGTAAAAGGTCATATGAAACGTGAAATAGAATGGAATGCAATTATTGAGTT |
| promoter (B. thuringiensis | AGGAGTTAGACCA |
| serovar kurstaki str. HD73) | |
| (SEQ ID NO: 148) | |
| BclA promoter | ATCGATGGAACCTGTATCAACCACTATAATTTCATCCACAATTTTTTCAACTGAGTCTA |
| (B. cereus F837/76) | AACAACGGGCTATTGTCTTCTCCTCATCTCGAACAATCATACATAAACTAATTGTAAT |
| (SEQ ID NO: 149) | TCCTTGCTTGTTCAACATAATCACCCTCTTCCAAATCAATCATATGTTATACATATACT |
| | AAACTTTCCATTTTTTTAAATTGTTCAAGTAGTTTAAGATTTCTTTTCAATAATTCAAAT |
| | GTCCGTGTCATTTTCTTTCGGTTTTGCATCTACTATATAATGAACGCTTTATGGAGGTG |
| | AATTT |
| BclA minimal promoter | AATCAATCATATGTTATACATATACTAAACTTTCCATTTTTTTAAATTGTTCAAGTAGT |
| (B. cereus F837/76) | TTAAGATTTCTTTTCAATAATTCAAATGTCCGTGTCATTTTCTTTCGGTTTTGCATCTAC |
| (SEQ ID NO: 150) | TATATAATGAACGCTTTATGGAGGTGAATTT |
| BclB promoter | GACCTGTAAGTCTGTAGGGAAGAATAATTTCAAGAGCCAGTGATAATAGATTTTTTTG |
| (B. thuringiensis serovar | TTTTTTCATTCTTATCTTGAATATAAATCACCTCATCTTTTAATTAGAACGTAACCAAT |
| konkukian str. 97-27) | TTAGTATTTTGAAATAGAGCTATCATTTTATAATATGAATACTACTAGTTATAGAAAC |
| (SEQ ID NO: 151) | GGCAAAAAGTTTAATATATGTAAAAATCATTTGGATATGAAAAAAGTAGCCATAGAT |
| | TTTTTCGAAATGATAAATGTTTTATTTTGTTAATTAGGAAACAAAAATGTGGAATGAG |
| | GGGGATTTAA |
| BclB minimal promoter | ATATGAAAAAAGTAGCCATAGATTTTTTCGAAATGATAAATGTTTTATTTTGTTAATTA |
| (B. thuringiensis serovar | GGAAACAAAAATGTGGAATGAGGGGGATTTAA |
| konkukian str. 97-27) | |
| (SEQ ID NO: 152) | |
| BxpA promoter | TTTTCATCTGCTACATCGTGAAGTAATGCTGCCATTTCAATTATAAAACGATTTCCTCC |
| (B. anthracis str. Sterne) | TTCTTGCTCGGATAAAGAAATCGCCAGTTTATGTACACGCTCAATATGATACCAATCA |
| (SEQ ID NO: 153) | TGCCCACTGGCATCTTTTTCTAAAATATGTTTTACAAAAGTAATTGTTTTTTCTATCTTT |
| | TCTTGTTTTGTCATTTTATCTTCACCCAGTTACTTATTGTAACACGCCCGCATTTTTTCA |
| | TCACATATTTTCTTGTCCGCCCATACACTAGGTGGTAGGCATCATCATGAAGGAGGA |
| | ATAGAT |
| BxpA minimal promoter | ACATATTTTCTTGTCCGCCCATACACTAGGTGGTAGGCATCATCATGAAGGAGGAAT |
| (B. anthracis str. Sterne) | AGAT |
| (SEQ ID NO: 154) | |
| BclE promoter | GGTGACGACAACATATACAAGAGGCACTCCTGCTGGTACTGTAACAGGAACAAATAT |
| (B. anthracis Δ Sterne) | GGGGCAAAGTGTAAATACATCGGGTATAGCACAAGCTGTCCCGAATACAGATAATAT |
| (SEQ ID NO: 155) | GGATTCAACGGCGGGACTCCCTTAAGAAATTAGGGGAGTCTTTATTTGGAAAAAGAGC |
| | TTATGTTACATAAAAACAGGAGTAATTGTTTTAAAAGTAGTATTGGTGACGTTGTTAGA |
| | AAATACAATTTAAGTAGAAGGTGCGTTTTTATATGAAATATATTTTATAGCTGTACTTT |
| | ACCTTTCAAG |
| BclE minimal promoter | ACAAGCTGTCCCGAATACAGATAATATGGATTCAACGGCGGGACTCCCTTAAGAAATT |
| (B. anthracis Δ Sterne) | AGGGGAGTCTTTATTTGGAAAAAGAGCTTATGTTACATAAAAACAGGAGTAATTGTTT |
| (SEQ ID NO: 156) | TAAAAGTAGTATTGGTGACGTTGTTAGAAAATACAATTTAAGTAGAAGGTGCGTTTTT |
| | ATATGAAATATATTTTATAGCTGTACTTTACCTTTCAAG |
| BetA promoter | ATTTATTTCATTCAATTTTTCCTATTTAGTACCTACCGCACTCACAAAAAGCACCTCTCA |
| (B. anthracis Sterne) | TTAATTTATATTATAGTCATTGAAATCTAATTTAATGAAATCATCATACTATATGTTTT |
| (SEQ ID NO: 157) | ATAAGAAGTAAAGGTACCATACTTAATTAATACATATCTATACACTTCAATATCACAG |
| | CATGCAGTTGAATTATATCCAACTTTCATTTCAAATTAAATAAGTGCCTCCGCTATTGT |
| | GAATGTCATTTACTCTCCCTACTACATTTAATAATTATGACAAGCAATCATAGGAGGT |
| | TACTAC |
| BetA minimal promoter | TAAGAAGTAAAGGTACCATACTTAATTAATACATATCTATACACTTCAATATCACAGC |
| (B. anthracis Sterne) | ATGCAGTTGAATTATATCCAACTTTCATTTCAAATTAAATAAGTGCCTCCGCTATTGTG |
| (SEQ ID NO: 158) | AATGTCATTTACTCTCCCTACTACATTTAATAATTATGACAAGCAATCATAGGAGGTT |
| | ACTAC |
| CotE promoter | AGTTGTACAAGAATTTAAATCTTCACAAACATATGTAAATGACTTACTACAGCTAGTT |
| (B. cereus AH820) | GCAAGTACGATTTCTAACAACGTAACAGATGAAATATTAATTTCAACTAATGGCGATG |
| (SEQ ID NO: 159) | TATTGAAGGGTGAAACGGGCGCAGCGGTAGAAAGTAAAAAAGGAAATTGTGGTTGTT |
| | AAAGAGATGTCGAAATGACATCTCTTTTTTTAGTGGATTAAACGTAAGTTCTTCTCAAA |
| | AAAAGAATGACACATTCCGCTATTGTCACGCATATGATTAAGTGAATAGTGATTGAGG |
| | AGGGTTACGA |
| CotE minimal promoter | ACATTCCGCTATTGTCACGCATATGATTAAGTGAATAGTGATTGAGGAGGGTTACGA |
| (B. cereus AH820) | |
| (SEQ ID NO: 160) | |
| ExsA promoter | AACGTTATTAGCGTAGACAAACAAGTAACGGCAGAAGCAGTTCTTGCATTAAATCGT |
| (B. cereus strain ATCC 10876) | ATGTTAGAGCGTGTGTAAAGCAACGGTATTCCCGTTGCTTTTTTTCATACATATAATCA |
| (SEQ ID NO: 161) | TAACGAGAACGAAATGGGCATACATTGTTTTGAAGAAATCATTGTGGTTCTTTATGCT |
| | TATTCCACTTCGAATGATATTGAAAATCGAAGAAGTGATAAAAGTAAAAAGAAGTTAA |
| | TGTTATTTAGAAAGAGTTACTTCATGAGATTTGTTACTTATAGATAAGTTATACAGGA |
| | GGGGGAAAAT |
| ExsA minimal promoter | TCATGAGATTTGTTACTTATAGATAAGTTATACAGGAGGGGGAAAAT |
| (B. cereus strain ATCC 10876) | |
| (SEQ ID NO: 162) | |
| ExsK promoter | AAGCCGCGGTCAATGCTGTATATGCAAATAAGATTGCAGCTTTACCTGAAGAAGAGC |
| (B. thuringiensis serovar | GTGATAGCTTCATTGCTGAAAAACGAGAAGAGTATAAGAAAGATATTGATATTTACC |
| konkukian str. 97-27) | ATTTAGCATCAGAGATGGTCATTGATGGTATTGTTCATCCAAACAATTTAAGAGAAGA |
| (SEQ ID NO: 163) | GTTAAAAGGACGATTCGAAATGTATATGAGTAAATATCAAGTATTTACGGATCGTAAA |
| | CATCCTGTTTATCCAGTTTAAAAGCCCTATTTAGGGCTTTCTTGCTCAAAAAGTTAAG |
| | GAGGGGAAAACA |
| ExsK minimal promoter | TCAAGTATTTACGGATCGTAAACATCCTGTTTATCCAGTTTAAAAGCCCTATTTAGGG |
| (B. thuringiensis serovar | CTTTCTTGCTCAAAAAGTTAAGGAGGGGAAAACA |
| konkukian str. 97-27) | |
| (SEQ ID NO: 164) | |
| ExsB promoter | AGGATTTCAGTGGGACGCCTCCTCTCTTCTTACATTAAATTAATCATACTATAAAATGA |
| (B. cereus F837/76) | AAGAAATGAAATGAAAAATAGCGGAAAAATCAGAAATTTTTTCTGGTAGTATACAAT |
| (SEQ ID NO: 165) | ATGTTACAATAAGCTTTGTCAATGAAAGAAGGAATTCCGTGCAATGCACGGGAGAGG |
| | TTCGCGAACTCCCTCTATAAAAAACTATGGAAACAACAATATCTTTAGGTATTGTTTT |
| | GTTTTTTTATTGTGACAGTTCAAGAACGTTCTTTCTTCTTATTCGTAGTAGAGAAGGAG |
| | AATGAGTGAA |
| ExsB minimal promoter | ACTATGGAAACAACAATATCTTTAGGTATTGTTTTGTTTTTTTATTGTGACAGTTCAAG |
| (B. cereus F837/76) | AACGTTCTTTCTTCTTATTCGTAGTAGAGAAGGAGAATGAGTGAA |
| (SEQ ID NO: 166) | |
| YabG promoter | TTTTGCACAACGCCGTAAAACTTTAATGAATAATTTATCAAATAATTTAAATGGTTTC |
| (B. cereus AH820) | CCGAAAGATAAAGAGCTGTTGGATCGAATTTTAACAGAAGTAGGAATTGATCCAAAAC |
| (SEQ ID NO: 167) | GAAGAGGCGAAACGCTATCTATCGAAGAGTTTGCGACATTAAGTAATGCATTAGTTCT |
| | TCATAAGTTATCATAAGAATACAAAAGGGACAGTTCAATTTGAACTGTCCCTTTTGTC |
| | ACCTTTCTCCTCCTAAATTCATACTTTAAAAACAGGTAAGATGGCCTAACGAGTTTGG |
| | AGGTAGGAGA |
| YabG minimal promoter | TCTCCTCCTAAATTCATACTTTAAAAACAGGTAAGATGGCCTAACGAGTTTGGAGGTA |
| (B. cereus AH820) | GGAGA |
| (SEQ ID NO: 168) | |
| Tgl promoter | GGAAACAGAAGTCATCCCATTTGAAAATGCAGCAGGTCGTATTATAGCTGATTTCGTT |
| (B. thuringiensis serovar | ATGGTTTATCCGCCAGGGATTCCAATCTTTACTCCGGGGGAAATTATTACACAAGACA |
| konkukian str. 97-27) | ACTTAGAGTATATTCGTAAAAACTTAGAAGCAGGTTTACCTGTACAAGGTCCTGAAGA |
| (SEQ ID NO: 169) | TATGACATTACAAACATTACGCGTGATCAAAGAGTACAAGCCTATCAGTTGATAGGCT |
| | TTTTTTCACCCTTTTTCCCTTTTCTCATACGATATTATGTAATGTAACGTATAGGTGGGG |
| | ATACTACT |
| Tgl minimal promoter | ACCCTTTTTCCCTTTTCTCATACGATATTATGTAATGTAACGTATAGGTGGGGATACTA |
| (B. thuringiensis serovar | CT |
| konkukian str. 97-27) | |
| (SEQ ID NO: 170) | |
| Superoxide dismutase (SODA1) | ATTGTGGACCCTTAGCTCAGCTGGTTAGAGCAGACGGCTCATAACCGTCCGGTCGTAG |
| promoter (B. cereus F837/76) | GTTCGAGTCCTACAGGGTCCATATCCATTTCACATGTTTATTATGTCGGCAGGAAGCTT |
| (SEQ ID NO: 171) | CCTTGTAGAAGGGAGCTTTTTTTATGAAATATATGAGCATTTTAATTGAAATGAAGTGG |
| | GAATTTTGCTACTTTAATGATAGCAAGACAATGTGATTTATTTGTTTGCACCCTATGGC |
| | AATTAGGGTAGAATGAAGTTGTATGTCACTTAAGTGGCAATACATAAACTGGGAGGA |
| | ATATAACA |
| Superoxide dismutase (SODA1) | ACTTAAGTGGCAATACATAAACTGGGAGGAATATAACA |
| minimal promoter | |
| (B. cereus F837/76) | |
| (SEQ ID NO: 172) | |
| Superoxide dismutase (SODA2) | AATATAACAGAAAATTCTGATGTTTTTTCAAATCCTATAATAAGGAGTGTTCCGTATGA |
| promoter (B. cereus AH820) | TGCCTTTATATTTTCCGGAAGATAAAACAGAATATATTATTCCAGGGATTGTTTGTGT |
| (SEQ ID NO: 173) | TCTATTTATCATCGGTGCGATTGCTACGTGGCGTATGTTCATTCGTGTATCAAAACGAG |
| | AAGCAGAGCGATTACAGAAAGTTGAAGAAAAGCTGTTAGCTGAAAAGAAACAGTAAC |
| | TCATTTTTGTATGTTTCCCTCTATGCTCGGACAATCTAAGGGCAGAATGTATTTTGGAG |
| | GGAATGAA |
| Superoxide dismutase (SODA2) | TCCGGAAGATAAAACAGAATATATTATTCCAGGGATTGTTTGTGTTCTATTTATCATCG |
| minimal promoter | GTGCGATTGCTACGTGGCGTATGTTCATTCGTGTATCAAAACGAGAAGCAGAGCGATT |
| (B. cereus AH820) | ACAGAAAGTTGAAGAAAAGCTGTTAGCTGAAAAGAAACAGTAACTCATTTTTGTATGT |
| (SEQ ID NO: 174) | TTCCCTCTATGCTCGGACAATCTAAGGGCAGAATGTATTTTGGAGGGAATGAA |
| BclA promoter | TAATCACCCTCTTCCAAATCAATCATATGTTATACATATACTAAACTTTCCATTTTTTT |
| (B. anthracis Sterne) | AAATTGTTCAAGTAGTTTAAGATTTCTTTTCAATAATTCAAATGTCCGTGTCATTTTCT |
| (SEQ ID NO: 175) | TTCGGTTTTGCATCTACTATATAATGAACGCTTTATGGAGGTGAATTT |
| BAS1882 promoter | AATTACATAACAAGAACTACATTAGGGAGCAAGCAGTCTAGCGAAAGCTAACTGCTTT |
| (B. anthracis Sterne) | TTTATTAAATAACTATTTTATTAAATTTCATATATACAATCGCTTGTCCATTTCATTTGG |
| (SEQ ID NO: 176) | CTCTACCCACGCATTTACTATTAGTAATATGAATTTTTCAGAGGTGGATTTTATT |
| Gene 3572 promoter | CTATGATTTAAGATACACAATAGCAAAAGAGAAACATATTATATAACGATAAATGAA |
| (B. weihenstephensis | ACTTATGTATATGTATGGTAACTGTATATATTACTACAATACAGTATACTCATAGGAGG |
| KBAB 4) | TAGGT |
| (SEQ ID NO: 177) | |
| YVTN β-propeller protein | GGTAGGTAGATTTGAAATATGATGAAGAAAAGGAATAACTAAAAGGAGTCGATATCC |
| promoter | GACTCCTTTTAGTTATAAATAATGTGGAATTAGAGTATAATTTTATATAGGTATATTGT |
| (B. weihenstephensis KBAB 4) | ATTAGATGAACGCTTTATCCTTTAATTGTGATTAATGATGGATTGTAAGAGAAGGGGCT |
| (SEQ ID NO: 178) | TACAGTCCTTTTTTTATGGTGTTCTATAAGCCTTTTTAAAAGGGGTACCACCCCACACC |
| | CAAAAACAGGGGGGGTTATAACTACATATTGGATGTTTTGTAACGTACAAGAATCGGT |
| | ATTAATTACCCTGTAAATAAGTTATGTGTATATAAGGTAACTTTATATATTCTCCTACA |
| | ATAAAATAAAGGAGGTAATAAA |
| Cry1A promoter | AACCCTTAATGCATTGGTTAAACATTGTAAAGTCTAAAGCATGGATAATGGGCGAGAA |
| (B. thuringiensis HD-73) | GTAAGTAGATTGTTAACACCCTGGGTCAAAAATTGATATTTAGTAAAATTAGTTGCACT |
| (SEQ ID NO: 179) | TTGTGCATTTTTTCATAAGATGAGTCATATGTTTTAAATTGTAGTAATGAAAAACAGT |
| | ATTATATCATAATGAATTGGTATCTTAATAAAAGAGATGGAGGTAACTTA |
| ExsY promoter | TAATTCCACCTTCCCTTATCCTCTTTCGCCTATTTAAAAAAAGGTCTTGAGATTGTGACC |
| (B. thuringiensis serovar | AAATCTCCTCAACTCCAATATCTTATTAATGTAAATACAAACAAGAAGATAAGGA |
| konkukian str. 97-27) | |
| (SEQ ID NO: 180) | |
| CotY promoter | AGGATGTCTTTTTTTATATTGTATTATGTACATCCCTACTATATAAATTCCCTGCTTTTA |
| (B. thuringiensis Al Hakam) | TCGTAAGAATTAACGTAATATCAACCATATCCCGTTCATATTGTAGTAGTGTATGTCA |
| (SEQ ID NO: 181) | GAACTCACGAGAAGGAGTGAACATAA |
| YjcA promoter | TTAATGTCACTCCTTATCTTCTTGTTTGTATTTACATTAATAAGATATTGGAGTTGAGG |
| (B. thuringiensis serovar | AGATTTGGTCACAATCTCAAGACCTTTTTTTTAAATAGGCGAAAGAGGATAAGGGAAG |
| kurstaki str. HD73) | GTGGAATT |
| (SEQ ID NO: 182) | |
| YjcB promoter | ATATATTTTCATAATACGAGAAAAAGCGGAGTTTAAAAGAATGAGGGAACGGAAATA |
| (B. thuringiensis serovar | AAGAGTTGTTCATATAGTAAATAGACAGAA |
| kurstaki str. HD73) | |
| (SEQ ID NO: 183) | |
| ExsFA/BxpB promoter | AAACTAAATAATGAGCTAAGCATGGATTGGGTGGCAGAATTATCTGCCACCCAATCCA |
| (B. thuringiensis Al Hakam) | TGCTTAACGAGTATTATTATGTAAATTTCTTAAAATTGGGAACTTGTCTAGAACATAG |
| (SEQ ID NO: 184) | AACCTGTCCTTTTCATTAACTGAAAGTAGAAACAGATAAAGGAGTGAAAAAC |
| Rhamnose promoter | ATTCACTACAACGGGGATGAGTTTGATGCGGATACATATGAGAAGTACCGGAAAGTG |
| (B. thuringiensis Al Hakam) | TTTGTAGAACATTACAAAGATATATTATCTCCATCATAAAGGAGAGATGCAAAG |
| (SEQ ID NO: 185) | |
| CotO promoter | CGCGCACCACTTCGTCGTACAACAACGCAAGAAGAAGTTGGGGATACAGCAGTATTCT |
| (B. anthracis Sterne) | TATTCAGTGATTTAGCACGCGGCGTAACAGGAGAAAACATTCACGTTGATTCAGGGTA |
| (SEQ ID NO: 186) | TCATATCTTAGGATAAATATAATATTAATTTTAAAGGACAATCTCTACATGTTGAGAT |
| | TGTCCTTTTTATTTGTTCTTAGAAAGAACGATTTTTAACGAAAGTTCTTACCACGTTATG |
| | AATATAAGTATAATAGTACACGATTTATTCAGCTACGTA |
| Sigma K promoter | TATATCATATGTAAAATTAGTTCTTATTCCCACATATCATATAGAATCGCCATATTAT |
| (B. anthracis Sterne) | ACATGCAGAAAACTAAGTATGGTATTATTCTTAAATTGTTTAGCACCTTCTAATATTAC |
| (SEQ ID NO: 187) | AGATAGAATCCGTCATTTTCAACAGTGAACATGGATTTCTTCTGAACACAACTCTTTTT |
| | CTTTCCTTATTTCCAAAAAGAAAAGCAGCCCATTTTAAAATACGGCTGCTTGTAATGTA |
| | CATTA |
| InhA1 promoter | TATCACATAACTCTTTATTTTTAATATTTCGACATAAAGTGAAACTTTAATCAGTGGGG |
| (B. thuringiensis Al Hakam) | GCTTTGTTCATCCCCCCACTGATTATTAATTGAACCAAGGGATAAAAAGATAGAGGGT |
| (SEQ ID NO: 188) | CTGACCAGAAAACTGGAGGGCATGATTCTATAACAAAAAGCTTAATGTTTATAGAATT |
| | ATGTCTTTTTATATAGGGAGGGTAGTAAACAGAGATTTGGACAAAAATGCACCGATTT |
| | ATCTGAATTTTAAGTTTTATAAAGGGGAGAAATG |
| BclA cluster glycosyl | ATTTTTTACTTAGCAGTAAAACTGATATCAGTTTTACTGCTTTTTCATTTTTAAATTCAA |
| transferase operon 1 | TCATTAAATCTTCCTTTTCTACATAGTCATAATGTTGTATGACATTCCGTAGGAGGCAC |
| (B. thuringiensis serovar | TTATA |
| konkukian str. 97-27) | |
| (SEQ ID NO: 189) | |
| BclA cluster glycosyl | ACATAAATTCACCTCCATAAAGCGTTCATTATATAGTAGATGCAAAACCGAAAGAAAA |
| transferase operon 2 | TGACACGGACATTTGAATTATTGAAAAGAAATCTTAAACTACTTGAACAATTTAAAAA |
| (B. thuringiensis serovar | AATGGAAAGTTTAGTATATGTATAACATATGATTGATTTGGAAGAGGGTGATTA |
| kurstaki str. HD73) | |
| (SEQ ID NO: 190) | |
| Glycosyl transferase promoter | TTCTATTTTCCAACATAACATGCTACGATTAAATGGTTTTTTGCAAATGCCTTCTTGGG |
| (B. thuringiensis Al Hakam) | AAGAAGGATTAGAGCGTTTTTTTATAGAAACCAAAAGTCATTAACAATTTTAAGTTAA |
| (SEQ ID NO: 191) | TGACTTTTTTGTTTGCCTTTAAGAGGTTTTATGTTACTATAATTATAGTATCAGGTACTA |
| | ATAACAAGTATAAGTATTTCTGGGAGGATATATCA |

The sigma-K sporulation-specific polymerase promoter sequences in the promoter sequences shown in Table 4 result in high expression levels of the fusion protein during late sporulation. The consensus sequence for the sigma-K sporulation-specific polymerase promoter sequence is CATANNNTN (SEQ ID NO: 200); however, this sequence can comprise up to two mutations and still be functional. The sigma-K sporulation-specific polymerase promoter sequence is generally found upstream of the ribosome binding site (RBS).

Promoters having a high degree of sequence identity to any of the sequences shown above in Table 4 can also be used to express the fusion proteins.

For example, the fusion protein can be expressed under the control of a promoter comprising a nucleic acid sequence having at least 80% identity with a nucleic acid sequence of any one of SEQ ID NOs: 37-42 and 123-191.

The fusion protein can be expressed under the control of a promoter comprising a nucleic acid sequence having at least 85% identity with a nucleic acid sequence of any one of SEQ ID NOs: 37-42 and 123-191.

The fusion protein can be expressed under the control of a promoter comprising a nucleic acid sequence having at least 90% identity with a nucleic acid sequence of any one of SEQ ID NOs: 37-42 and 123-191.

The fusion protein can be expressed under the control of a promoter comprising a nucleic acid sequence having at least 95% identity with a nucleic acid sequence of any one of SEQ ID NOs: 37-42 and 123-191.

The fusion protein can be expressed under the control of a promoter comprising a nucleic acid sequence having at least 98% identity with a nucleic acid sequence of any one of SEQ ID NOs: 37-42 and 123-191.

The fusion protein can be expressed under the control of a promoter comprising a nucleic acid sequence having at least 99% identity with a nucleic acid sequence of any one of SEQ ID NOs: 37-42 and 123-191.

The fusion protein can be expressed under the control of a promoter comprising a nucleic acid sequence having 100% identity with a nucleic acid sequence of any one of SEQ ID NOs: 37-42 and 123-191.

For example, fusion protein can be expressed under the control of a BclA promoter (e.g., SEQ ID NO: 149,150,175,189, or 190), a CotY promoter (e.g., SEQ ID NO: 41,42, or 181), an ExsY promoter (e.g., SEQ ID NO: 37,38, or 180), or a rhamnose promoter (e.g., SEQ ID NO: 185), or a promoter having a high degree of sequence identity to any of these promoters.

Thus, for example, the fusion protein can be expressed under the control of a promoter comprising a nucleic acid sequence having at least 80% identity with a nucleic acid sequence of any one of SEQ ID NOs: 37, 38, 41, 42, 149, 150, 175, 180, 181, 185, 189, or 190.

The fusion protein can be expressed under the control of a promoter comprising a nucleic acid sequence having at least 85% identity with a nucleic acid sequence of any one of SEQ ID NOs: 37, 38, 41, 42, 149, 150, 175, 180, 181, 185, 189, or 190.

The fusion protein can be expressed under the control of a promoter comprising a nucleic acid sequence having at least 90% identity with a nucleic acid sequence of any one of SEQ ID NOs: 37, 38, 41, 42, 149, 150, 175, 180, 181, 185, 189, or 190.

The fusion protein can be expressed under the control of a promoter comprising a nucleic acid sequence having at least 95% identity with a nucleic acid sequence of any one of SEQ ID NOs: 37, 38, 41, 42, 149, 150, 175, 180, 181, 185, 189, or 190.

The fusion protein can be expressed under the control of a promoter comprising a nucleic acid sequence having at least 98% identity with a nucleic acid sequence of any one of SEQ ID NOs: 37, 38, 41, 42, 149, 150, 175, 180, 181, 185, 189, or 190.

The fusion protein can be expressed under the control of a promoter comprising a nucleic acid sequence having at least 99% identity with a nucleic acid sequence of any one of SEQ ID NOs: 37, 38, 41, 42, 149, 150, 175, 180, 181, 185, 189, or 190.

The fusion protein can be expressed under the control of a promoter comprising a nucleic acid sequence having 100% identity with a nucleic acid sequence of any one of SEQ ID NOs: 37, 38, 41, 42, 149, 150, 175, 180, 181, 185, 189, or 190.

The fusion protein can be expressed under the control of a promoter comprising a sigma-K sporulation specific polymerase promoter sequence, wherein the sigma-K sporulation-specific polymerase promoter sequence or sequences have 100% identity with the corresponding nucleotides of any of SEQ ID NOs: 37-42 and 123-191.

The fusion proteins can be expressed under the control of a promoter that is native to the targeting sequence, exosporium protein, or exosporium protein fragment of the fusion protein. Thus, for example, where the targeting sequence is derived from BclA, the fusion protein can be expressed under the control of a native BclA promoter (e.g., SEQ ID NO: 149, 150, 175, 189 or 190).

Table 4 also provides illustrative minimal promoter sequences. The fusion proteins can be expressed under any of these minimal promoter sequences.

Furthermore, the fusion protein can be expressed under a portion of any of the promoters listed above in Table 4, so long as the portion of the promoter includes a sigma-K sporulation-specific polymerase promoter sequence. For example, the fusion protein can be expressed under a promoter region that comprises the first 25, 50, 100, 150, 200, 250, or 300 nucleotides upstream of the start codon, so long as that region comprises a sigma-K sporulation-specific polymerase promoter sequence.

IV. Mutations and Other Genetic Alterations to Recombinant Bacillus cereus Family Members that Allow for Collection of Free Exosporium and Exosporium Fragments Derived from Such Recombinant Bacillus cereus Family Members

As is described further hereinbelow, the recombinant Bacillus cereus family members that express fusion proteins comprising a protein or peptide of interest (e.g., a pectinase, including any of the pectate lyases or polygalacturonases described herein) and a targeting sequence, an exosporium protein, or an exosporium protein fragment that targets the fusion protein to the exosporium of the recombinant Bacillus cereus family member can be used for various purposes, including delivering the proteins or peptides of interest plants, seeds, a plant growth medium, or an area surrounding a seed or a plant (e.g., via soil drench, foliar application, or as a seed treatment). However, in some cases, the presence of the living microorganisms may not be desirable, and instead, it would be desirable to separate the living spore from the fusion proteins in the exosporium on the outside surface of the spore. For example, in some applications it will be desirable to increase enzyme activity without concern for spore integrity. In such situations, use of exosporium fragments that have been separated from the spores may be preferred over the use of living microorganisms having the enzyme on their exosporium.

In addition, for some uses, it may be desirable to reduce the density of the product. In such instances, it would be desirable to separate the dense spore from the exosporium (containing the fusion proteins). Furthermore, under some circumstances the presence of live spores would lead to potential for bacterial growth in a product, which would be undesirable for some applications.

Mutations or other genetic alterations (e.g., overexpression of a protein) can be introduced into the recombinant Bacillus cereus family members that allow free exosporium to be separated from spores of the recombinant Bacillus cereus family member. This separation process yields exosporium fragments that contain the fusion proteins but that are substantially free of the spores themselves. By “substantially free of spores” it is meant that once the free exosporium is separated from the spores, a preparation is obtained that contains less than 5% by volume of spores, preferably less than 3% by volume of spores, even more preferably less than 1% by volume of spores, and most preferably contains no spores or if spores are present, they are undetectable. These exosporium fragments can be used in place of the recombinant Bacillus cereus family members themselves in any of the formulations, plant seeds, and methods described herein.

Exosporium fragments derived from spores of a recombinant Bacillus cereus family member can be used in any of the formulations, plant seeds, and methods described herein. The recombinant Bacillus cereus family member expresses any of the fusion proteins described herein. The recombinant Bacillus cereus family member also comprises a mutation or expresses a protein, wherein the expression of the protein is increased as compared to the expression of the protein in a wild-type Bacillus cereus family member under the same conditions. The mutation or the increased expression of the protein results in Bacillus cereus family member spores having an exosporium that is easier to remove from the spore as compared to the exosporium of a wild-type spore.

The recombinant Bacillus cereus family member: (i) can comprise a mutation in a CotE gene; (ii) can express an ExsY protein, wherein the expression of the ExsY protein is increased as compared to the expression of the ExsY protein in a wild-type Bacillus cereus family member under the same conditions, and wherein the ExsY protein comprises a carboxy-terminal tag comprising a globular protein; (iii) can express a BclB protein, wherein the expression of the BclB protein is increased as compared to the expression of the BclB protein in a wild-type Bacillus cereus family member under the same conditions; (iv) can express a YjcB protein, wherein the expression of the YjcB protein is increased as compared to the expression of the YjcB protein in a wild-type Bacillus cereus family member under the same conditions; (v) can comprise a mutation in an ExsY gene; (vi) can comprise a mutation in a CotY gene; (vii) can comprise a mutation in an ExsA gene; or (viii) can comprise a mutation in a CotO gene.

The recombinant Bacillus cereus family member can comprise a mutation in the CotE gene, such as a knock-out of the CotE gene or a dominant negative form of the CotE gene. The mutation in the CotE gene can partially or completely inhibit the ability of CotE to attach the exosporium to the spore.

The recombinant Bacillus cereus family member can express an ExsY protein. The ExsY protein comprises a carboxy-terminal tag comprising a globular protein (e.g., a green fluorescent protein (GFP) or a variant thereof), and the expression of the ExsY protein is increased as compared to the expression of the ExsY protein in a wild-type Bacillus cereus family member under the same conditions. The globular protein can have a molecular weight of between 25 kDa and 100 kDa. Expression of the ExsY protein comprising the carboxy-terminal tag comprising a globular protein can inhibit binding of the ExsY protein to its targets in the exosporium.

The recombinant Bacillus cereus family member can express a BclB protein. Expression of the BclB protein can result in the formation of a fragile exosporium. The expression of the BclB protein can be increased as compared to the expression of the BclB protein in a wild-type Bacillus cereus family member under the same conditions.

The recombinant Bacillus cereus family member can express a YjcB protein. Expression of the YjcB protein can cause the exosporium to form in pieces rather than in a complete structure. The expression of the YjcB protein can be increased as compared to the expression of the YjcB protein in a wild-type Bacillus cereus family member under the same conditions.

The recombinant Bacillus cereus family member can comprise a mutation an ExsY gene, such as a knock-out of the ExsY gene. The mutation in the ExsY gene can partially or completely inhibit the ability of ExsY to complete the formation of the exosporium or attach the exosporium to the spore.

The recombinant Bacillus cereus family member can comprise a mutation a CotY gene, such as a knock-out of the CotY gene. The mutation in the CotY gene can result in the formation of a fragile exosporium.

The recombinant Bacillus cereus family member can comprise a mutation an ExsA gene, such as a knock-out of the ExsA gene. The mutation in the ExsA gene can result in the formation of a fragile exosporium.

The recombinant Bacillus cereus family member can comprise a mutation a CotO gene, such as a knock-out of the CotO gene or a dominant negative form of the CotO gene. The mutation in the CotO gene can cause the exosporium to form in strips.

For ease of reference, descriptions of illustrative sequences for CotE, ExsY, BclB, YjcB, CotY, ExsA, and CotO are provided in Table 5 below.

TABLE 5
Sequences of Proteins that Can be Mutated or Otherwise Genetically
Altered to Allow for Collection of Free Exosporium

| Protein | SEQ ID NO: |
|---|---|
| CotE, Bacillus cereus group | 192 |
| ExsY, Bacillus thuringiensis | 193 |
| BclB, variant 1, Bacillus anthracis Sterne | 194 |
| BclB, variant 2, Bacillus anthracis Sterne | 195 |
| YjcB, Variant 1, Bacillus cereus | 196 |
| YjcB, Variant 2, Bacillus cereus | 197 |
| CotY, Bacillus cereus | 198 |
| CotO, Bacillus anthracis | 199 |

Exosporium fragments can be prepared from any of these recombinant Bacillus cereus family members and used for various purposes as described further herein below. Where the recombinant Bacillus cereus family member expresses a fusion protein, the exosporium fragments will comprise the fusion proteins. Upon purification of the exosporium fragments that contain the fusion proteins from the spores, a cell-free protein preparation is obtained in which the fusion proteins are stabilized and supported through covalent bonds to the exosporium fragments.

To remove the exosporium from spores of the recombinant Bacillus cereus family members that have mutations or other genetic alterations that allow for collection of free exosporium, a suspension or fermentation broth of the spores can be subjected to centrifugation or filtration to produce fragments of exosporium that are separated from the spores. Where the recombinant Bacillus cereus family member expresses a fusion protein, the exosporium fragments will comprise the fusion protein.

A suspension or fermentation broth comprising the spores can be subjected to centrifugation, followed by collection of the supernatant. The supernatant comprises the fragments of the exosporium and is substantially free of spores.

Alternatively, a suspension or fermentation broth comprising the spores can be subjected to filtration, followed by collection of the filtrate. The filtrate comprises the fragments of the exosporium and is substantially free of spores.

The suspension or fermentation broth of spores can be agitated or mechanically disrupted prior to centrifugation or filtration.

The exosporium fragments can also be separated from the spores by gradient centrifugation, affinity purification, or by allowing the spores to settle out of the suspension or fermentation broth.

Due to the strong covalent bonds between the fusion proteins and the exosporium fragments, the fusion proteins become resistant to heat. The heat resistance of the fusion proteins bound to the exosporium fragments allows them to be used for applications that require heat-resistant proteins or enzymes.

Exosporium fragments derived from a recombinant Bacillus cereus family member are provided.

The exosporium fragments can be derived from any of the recombinant Bacillus cereus family members that comprise any of the mutations or other genetic alterations described herein that allow for collection of free exosporium.

The exosporium fragments can comprise any of the fusion proteins described above in Section I.

### V. Formulations

A formulation is provided. The formulation comprises any of the recombinant Bacillus cereus family members described herein. The formulation further comprises an agriculturally acceptable carrier.

Another formulation is provided. The formulation comprises exosporium fragments derived from any of the recombinant Bacillus cereus family members described herein. The formulation further comprises an agriculturally acceptable carrier.

### VI. Treated Seeds

A treated plant seed is provided. The plant seed can be treated with any of the recombinant Bacillus cereus family members described herein. The recombinant Bacillus cereus family member can express any of the fusion proteins described herein.

Another treated plant seed is provided. The plant seed can be treated with any of the exosporium fragments described herein. The exosporium fragments can be derived from any of the Bacillus cereus family members described herein. The exosporium fragments can comprise any of the fusion proteins described herein.

Yet another treated plant seed is provided. The plant seed can be treated with any of the formulations described herein.

In any of the treated plant seeds, the plant seed can be coated with the recombinant Bacillus cereus family member, the exosporium fragments, or the formulation.

The recombinant Bacillus cereus family members, exosporium fragments, or formulations can used as seed treatments, e.g., seed coatings or dressings. Seed coating or dressing formulations may be in the form of a liquid carrier formulation, a slurry formulation, or a powder formulation.

Seed coating or dressing formulations can be applied with conventional additives that are provided to make the seed treatment have sticky qualities to stick to and coat the seeds. Suitable additives comprise: talcs, graphites, gums, stabilizing polymers, coating polymers, finishing polymers, slip agents for seed flow and plantability, cosmetic agents, and cellulosic materials such as carboxymethyl cellulose and the like.

The seed treatments formulations can further comprise colorant agents and/or other additives.

The seed treatment formulations(s) may be applied to seeds in a suitable carrier such as water or a powder. The seeds can then be allowed to dry and planted in conventional fashion. The recombinant Bacillus cereus family members or exosporium fragments can be applied directly to the seed as a solution or in combination with other commercially available additives. For example, the recombinant Bacillus cereus family members or exosporium fragments can be applied in combination with seedling-acceptable carrier(s) (e.g., a liquid carrier or a solid carrier).

Solutions containing the recombinant Bacillus cereus family members or exosporium fragments can be sprayed or otherwise applied to the seed (e.g., in a seed slurry or a seed soak).

Solid or dry materials containing recombinant Bacillus cereus family members or exosporium fragments are also useful to promote effective seedling germination, growth, and protection during early seedling establishment.

The recombinant Bacillus cereus family members or exosporium fragments can be used with a solubilizing carrier such as water, a buffer (e.g., citrate or phosphate buffer), other treating agents (e.g., alcohol or another solvent), and/or any soluble agent.

In addition, small amounts of drying agent enhancers, such as lower alcohols, etc. can be used in seed coating formulations.

Surfactants, emulsifiers and preservatives can also be added at relatively low (e.g., about 0.5% w/v or less) levels in order to enhance the stability of the seed coating product.

Seeds can be treated using a variety of methods including, but not limited to, pouring, pumping, drizzling, or spraying an aqueous solution containing the recombinant Bacillus cereus family members or exosporium fragments on or over a seed; or spraying or applying the recombinant Bacillus cereus family members or exosporium fragments onto a layer of seeds either with or without the use of a conveyor system.

Mixing devices useful for seed treatment include but are not limited to tumblers, mixing basins, mixing drums, and fluid application devices that include basins or drums used to contain the seed while coating.

After seed treatment, the seed may be air-dried or a stream of dry air may be optionally used to aid in the drying of the seed coatings.

Seed treatments containing the recombinant Bacillus cereus family members or exosporium fragments can be applied using any commercially available seed treatment machinery or can also be applied using any acceptable non-commercial method(s) such as the use of syringes or any other seed treatment device.

VII. Methods for Stimulating Plant Growth and/or Promoting Plant Health

A method for stimulating plant growth and/or promoting plant health is provided. The method comprises applying a recombinant Bacillus cereus family member to a plant growth medium, a plant, a plant seed, or an area surrounding a plant or a plant seed. The recombinant Bacillus cereus family member can comprise any of the recombinant Bacillus cereus family members described herein. The recombinant Bacillus cereus family member can express any of the fusion proteins described herein.

Another method for stimulating plant growth and/or promoting plant health is provided. The method comprises applying exosporium fragments to a plant growth medium, a plant, a plant seed, or an area surrounding a plant or a plant seed. The exosporium fragments can comprise exosporium fragments derived from any of the recombinant Bacillus cereus family members described herein. The exosporium fragments can comprise any of the fusion proteins described herein.

Yet another method for stimulating plant growth and/or promoting plant health is provided. The method comprises applying a formulation to a plant growth medium, a plant, a plant seed, or an area surrounding a plant or a plant seed. The formulation can comprise any of the formulations described herein.

In any of the methods described herein, the method can further comprise inactivating the recombinant Bacillus cereus family member prior to applying the recombinant Bacillus cereus family member to the plant growth medium, the plant, the plant seed, or the area surrounding the plant or the plant seed.

In any of the methods described herein, the method can comprise applying the recombinant Bacillus cereus family member, the exosporium fragments, or the formulation to the plant growth medium.

In any of the methods described herein involving the use of a plant growth medium, the plant growth medium can comprise soil, water, an aqueous solution, sand, gravel, a polysaccharide, mulch, compost, peat moss, straw, logs, clay, soybean meal, yeast extract, or a combination thereof.

The plant growth medium can comprise a fertilizer.

Any of the methods described herein can further comprise supplementing the plant growth medium with a substrate for an enzyme. Suitable substrates include, but are not limited to a homogalacturonan, a pectin, a pectate, a polygalacturonate, an oligogalacturonate or a combination of any thereof.

In any of the methods described herein, the method can comprise applying the recombinant Bacillus cereus family member, the exosporium fragments, or the formulation to the plant.

For example, the method can comprise applying the recombinant Bacillus cereus family member, the exosporium fragments, or the formulation to roots of the plant.

Alternatively or in addition, the method can comprise applying the recombinant Bacillus cereus family member, the exosporium fragments, or the formulation foliarly.

In any of the methods described herein, the method can comprise applying the recombinant Bacillus cereus family member, the exosporium fragments, or the formulation to the plant seed.

Where the method comprises applying the recombinant Bacillus cereus family member, the exosporium fragments, or the formulation to the plant seed, applying the recombinant Bacillus cereus family member, the exosporium fragments, or the formulation to the plant seed can comprise: (a) applying recombinant Bacillus cereus family member, the exosporium fragments, or the formulation to the plant seed at the time of planting; or (b) coating the plant seed with the recombinant Bacillus cereus family member, the exosporium fragments, or the formulation.

In any of the methods described herein, plants grown in the presence of the recombinant Bacillus cereus family member, the exosporium fragments, or the formulation can exhibit increased growth as compared to plants grown in the absence of the enzyme or the microorganism, under the same conditions.

In any of the methods described herein, seeds to which the recombinant Bacillus cereus family member, the exosporium fragments, or the formulation has been applied can exhibit increased germination rates as compared to seeds to which the enzyme or microorganism has not been applied, under the same conditions.

In any of the methods described herein, plants grown in the presence of the recombinant Bacillus cereus family member, the exosporium fragments, or the formulation can exhibit increased nutrient uptake as compared to plants grown in the absence of the enzyme or the microorganism, under the same conditions.

In any of the methods described herein, plants grown in the presence of the recombinant Bacillus cereus family member, the exosporium fragments, or the formulation can exhibit decreased susceptibility to a pathogen as compared to plants grown in the absence of the enzyme or the microorganism, under the same conditions.

In any of the methods described herein, plants grown in the presence of the recombinant Bacillus cereus family member, the exosporium fragments, or the formulation can exhibit decreased susceptibility to an environmental stress (e.g., drought, flood, heat, freezing, salt, heavy metals, low pH, high pH, or a combination of any thereof) as compared to plants grown in the absence of the enzyme or the microorganism, under the same conditions.

In any of the methods described herein, plants grown in the presence of the recombinant Bacillus cereus family member, the exosporium fragments, or the formulation can exhibit increased nutrient content as compared to plants grown in the absence of the enzyme or the microorganism, under the same conditions.

The nutrient can comprise, for example, a polysaccharide, an oligosaccharide, a monosaccharide, a protein, phytic acid, a phosphatate, a phospholipid, or a combination of any thereof.

In any of the methods described herein, plants grown in the presence of the recombinant Bacillus cereus family member, the exosporium fragments, or the formulation exhibit can increased root nodulation as compared to plants grown in the absence of the enzyme or the microorganism, under the same conditions.

In any of the methods described herein, plants grown in the presence of the recombinant Bacillus cereus family member, the exosporium fragments, or the formulation can exhibit greater crop yield as compared to plants grown in the absence of the enzyme, or the microorganism, under the same conditions. In one embodiment, the recombinant Bacillus cereus family member of the present invention increases yield or total plant weight by at least about 0.5%, or by at least about 1%, or by at least about 2%, or by at least about 3%, or by at least about 5%, or by at least about 6%, or by at least about 7%, or by at least about 8%, or by at least about 9%, or by at least about 10%, or by at least about 11%, or by at least about 12% when compared to plants produced under the same conditions but without treatment by a recombinant Bacillus cereus family member. In another embodiment, the recombinant Bacillus cereus family member of the present invention improves some aspect of plant vigor, such as germination, by at least about 0.5%, or by at least about 1%, or by at least about 2%, or by at least about 3%, or by at least about 5%, or by at least about 6%, or by at least about 7%, or by at least about 8%, or by at least about 9%, or by at least about 10%, or by at least about 11%, or by at least about 12% when compared to plants produced under the same conditions but without treatment by a recombinant Bacillus cereus family member.

In any of the methods described herein, plants grown in the presence of the recombinant Bacillus cereus family member, the exosporium fragments, or the formulation can exhibit altered leaf senescence as compared to plants grown in the absence of the enzyme or the microorganism, under the same conditions.

### VIII. Carriers

As described above, the formulations described herein comprise an agriculturally acceptable carrier.

The agriculturally acceptable carrier can comprise a dispersant, a surfactant (e.g., a heavy petroleum oil, a heavy petroleum distillate, a polyol fatty acid ester, a polyethoxylated fatty acid ester, an aryl alkyl polyoxyethylene glycol, an alkyl amine acetate, an alkyl aryl sulfonate, a polyhydric alcohol, an alkyl phosphate, or a combination of any thereof), an additive (e.g., an oil, a gum, a resin, a clay, a polyoxyethylene glycol, a terpene, a viscid organic, a fatty acid ester, a sulfated alcohol, an alkyl sulfonate, a petroleum sulfonate, an alcohol sulfate, a sodium alkyl butane diamate, a polyester of sodium thiobutane dioate, a benzene acetonitrile derivative, a proteinaceous material, or a combination of any thereof), water, a thickener (a long chain alkylsulfonate of polyethylene glycol, a polyoxyethylene oleate, or a combination of any thereof), an anti-caking agent (e.g., sodium salt, a calcium carbonate, diatomaceous earth, or a combination of any thereof), a residue breakdown product, a composting formulation, a granular application, diatomaceous earth, an oil, a coloring agent, a stabilizer, a preservative, a polymer, a coating, or a combination of any thereof.

Where the agriculturally acceptable carrier comprises a surfactant, the surfactant can comprise a non-ionic surfactant.

Where the agriculturally acceptable carrier comprises an additive and the additive comprises a proteinaceous material, the proteinaceous material can comprise a milk product, wheat flour, soybean meal, blood, albumin, gelatin, alfalfa meal, yeast extract, or a combination of any thereof.

Where the agriculturally acceptable carrier comprises an anti-caking agent and the anti-caking agent comprises a sodium salt, the sodium salt can comprise a sodium salt of monomethyl naphthalene sulfonate, a sodium salt of dimethyl naphthalene sulfonate, a sodium sulfite, a sodium sulfate, or a combination of any thereof.

The agriculturally acceptable carrier can comprise vermiculite, charcoal, sugar factory carbonation press mud, rice husk, carboxymethyl cellulose, peat, perlite, fine sand, calcium carbonate, flour, alum, a starch, talc, polyvinyl pyrrolidone, or a combination of any thereof.

Any of the formulations described herein can comprise a seed coating formulation (e.g., an aqueous or oil-based solution for application to seeds or a powder or granular formulation for application to seeds), a liquid formulation for application to plants or to a plant growth medium (e.g., a concentrated formulation or a ready-to-use formulation), or a solid formulation for application to plants or to a plant growth medium (e.g., a granular formulation or a powder agent).

The agriculturally acceptable carrier may comprise a formulation ingredient. The formulation ingredient may be a wetting agent, extender, solvent, spontaneity promoter, emulsifier, dispersant, frost protectant, thickener, and/or an adjuvant. In one embodiment, the formulation ingredient is a wetting agent.

Compositions of the present invention may include formulation ingredients added to compositions of the present invention to improve recovery, efficacy, or physical properties and/or to aid in processing, packaging and administration. Such formulation ingredients may be added individually or in combination.

The formulation ingredients may be added to compositions comprising cells, cell-free preparations and/or exosporium fragments to improve efficacy, stability, and physical properties, usability and/or to facilitate processing, packaging and end-use application. Such formulation ingredients may include inerts, stabilization agents, preservatives, nutrients, or physical property modifying agents, which may be added individually or in combination. In some embodiments, the carriers may include liquid materials such as water, oil, and other organic or inorganic solvents and solid materials such as minerals, polymers, or polymer complexes derived biologically or by chemical synthesis. In some embodiments, the formulation ingredient is a binder, adjuvant, or adhesive that facilitates adherence of the composition to a plant part, such as leaves, seeds, or roots. See, for example, Taylor, A. G., et al., “Concepts and Technologies of Selected Seed Treatments,” Annu. Rev. Phytopathol., 28: 321-339 (1990). The stabilization agents may include anti-caking agents, anti-oxidation agents, anti-settling agents, antifoaming agents, desiccants, protectants or preservatives. The nutrients may include carbon, nitrogen, and phosphorus sources such as sugars, polysaccharides, oil, proteins, amino acids, fatty acids and phosphates. The physical property modifiers may include bulking agents, wetting agents, thickeners, pH modifiers, rheology modifiers, dispersants, adjuvants, surfactants, film-formers, hydrotropes, builders, antifreeze agents or colorants. In some embodiments, the composition comprising cells, cell-free preparation and/or exosporium fragments produced by fermentation of the recombinant Bacillus cereus family member may be used directly with or without water as the diluent without any other formulation preparation. In a particular embodiment, a wetting agent, or a dispersant, is added to a dried concentrate of the whole bruth resulting from the fermentation, such as a freeze-dried or spray-dried powder. A wetting agent increases the spreading and penetrating properties, or a dispersant increases the dispersibility and solubility of the active ingredient (once diluted) when it is applied to surfaces. Exemplary wetting agents are known to those of skill in the art and include sulfosuccinates and derivatives, such as MULTIWET™ MO-70R (Croda Inc., Edison, N.J.); siloxanes such as BREAK-THRU® (Evonik, Germany); nonionic compounds, such as ATLOX™ 4894 (Croda Inc., Edison, N.J.); alkyl polyglucosides, such as TERWET® 3001 (Huntsman International LLC, The Woodlands, Tex.); C12-C14 alcohol ethoxylate, such as TERGITOL® 15-S-15 (The Dow Chemical Company, Midland, Mich.); phosphate esters, such as RHODAFAC® BG-510 (Rhodia, Inc.); and alkyl ether carboxylates, such as EMULSOGEN™ LS (Clariant Corporation, North Carolina).

As described above, any of the formulations described herein can comprise an agrochemical.

### IX. Free Enzymes

Any of the enzymes described herein can also be used as free enzymes or as enzymes expressed in recombinant microorganisms.

### X. Plants

In any of the above methods relating to plants, the plant can be a dicotyledon, a monocotyledon, a gymnosperm, or an angiosperm.

Likewise, for any of the seeds described herein the seed can be a seed of a dicotyledon, a monocotyledon, a gymnosperm, or an angiosperm.

For example, where the plant is a dicotyledon or the seed is a seed of a dicotyledon, the dicotyledon can be selected from the group consisting of bean, pea, tomato, pepper, squash, alfalfa, almond, aniseseed, apple, apricot, arracha, artichoke, avocado, bambara groundnut, beet, bergamot, black pepper, black wattle, blackberry, blueberry, bitter orange, bok-choi, Brazil nut, breadfruit, broccoli, broad bean, Brussels sprouts, buckwheat, cabbage, camelina, Chinese cabbage, cacao, cantaloupe, caraway seeds, cardoon, carob, carrot, cashew nuts, cassava, castor bean, cauliflower, celeriac, celery, cherry, chestnut, chickpea, chicory, chili pepper, chrysanthemum, cinnamon, citron, citrus, clementine, clove, clover, coffee, cola nut, colza, corn, cotton, cottonseed, cowpea, crambe, cranberry, cress, cucumber, currant, custard apple, drumstick tree, earth pea, eggplant, endive, fennel, fenugreek, fig, filbert, flax, geranium, gooseberry, gourd, grape, grapefruit, guava, hemp, hempseed, henna, hop, horse bean, horseradish, indigo, jasmine, Jerusalem artichoke, jute, kale, kapok, kenaf, kiwi, kohlrabi, kumquat, lavender, lemon, lentil, lespedeza, lettuce, lime, liquorice, litchi, loquat, lupine, macadamia nut, mace, mandarin, mangel, mango, medlar, melon, mint, mulberry, mustard, nectarine, niger seed, nutmeg, okra, olive, opium, orange, papaya, parsnip, pea, peach, peanut, pear, pecan nut, persimmon, pigeon pea, pistachio nut, plantain, plum, pomegranate, pomelo, poppy seed, potato, sweet potato, prune, pumpkin, quebracho, quince, trees of the genus Cinchona, quinoa, radish, ramie, rapeseed, raspberry, rhea, rhubarb, rose, rubber, rutabaga, safflower, sainfoin, salsify, sapodilla, Satsuma, scorzonera, sesame, shea tree, soybean, spinach, squash, strawberry, sugar beet, sugarcane, sunflower, swede, sweet pepper, tangerine, tea, teff, tobacco, tomato, trefoil, tung tree, turnip, urena, vetch, walnut, watermelon, yerba mate, wintercress, shepherd's purse, garden cress, peppercress, watercress, pennycress, star anise, laurel, bay laurel, cassia, jamun, dill, tamarind, peppermint, oregano, rosemary, sage, soursop, pennywort, calophyllum, balsam pear, kukui nut, Tahitian chestnut, basil, huckleberry, hibiscus, passionfruit, star apple, sassafras, cactus, St. John's wort, loosestrife, hawthorn, cilantro, curry plant, kiwi, thyme, zucchini, ulluco, jicama, waterleaf, spiny monkey orange, yellow mombin, starfruit, amaranth, wasabi, Japanese pepper, yellow plum, mashua, Chinese toon, New Zealand spinach, bower spinach, ugu, tansy, chickweed, jocote, Malay apple, paracress, sowthistle, Chinese potato, horse parsley, hedge mustard, campion, agate, cassod tree, thistle, burnet, star gooseberry, saltwort, glasswort, sorrel, silver lace fern, collard greens, primrose, cowslip, purslane, knotgrass, terebinth, tree lettuce, wild betel, West African pepper, yerba santa, tarragon, parsley, chervil, land cress, burnet saxifrage, honeyherb, butterbur, shiso, water pepper, perilla, bitter bean, oca, kampong, Chinese celery, lemon basil, Thai basil, water mimosa, cicely, cabbage-tree, moringa, mauka, ostrich fern, rice paddy herb, yellow sawah lettuce, lovage, pepper grass, maca, bottle gourd, hyacinth bean, water spinach, catsear, fishwort, Okinawan spinach, lotus sweetjuice, gallant soldier, culantro, arugula, cardoon, caigua, mitsuba, chipilin, samphire, mampat, ebolo, ivy gourd, cabbage thistle, sea kale, chaya, huauzontle, Ethiopian mustard, magenta spreen, good king henry, epazole, lamb's quarters, centella plumed cockscomb, caper, rapini, napa cabbage, mizuna, Chinese savoy, kai-lan, mustard greens, Malabar spinach, chard, marshmallow, climbing wattle, China jute, paprika, annatto seed, spearmint, savory, marjoram, cumin, chamomile, lemon balm, allspice, bilberry, cherimoya, cloudberry, damson, pitaya, durian, elderberry, feijoa, jackfruit, jambul, jujube, physalis, purple mangosteen, rambutan, redcurrant, blackcurrant, salal berry, satsuma, ugli fruit, azuki bean, black bean, black-eyed pea, borlotti bean, common bean, green bean, kidney bean, lima bean, mung bean, navy bean, pinto bean, runner bean, mangetout, snap pea, sweet pea, broccoflower, calabrese, nettle, bell pepper, raddichio, daikon, white radish, skirret, tat soi, broccolini, black radish, burdock root, fava bean, broccoli raab, lablab, lupin, sterculia, velvet beans, winged beans, yam beans, mulga, ironweed, umbrella bush, tjuntjula, wakalpulka, witchetty bush, wiry wattle, chia, beech nut, candlenut, colocynth, mamoncillo, Maya nut, mongongo, ogbono nut, paradise nut, and cempedak.

Where the plant is a monocotyledon or the seed is a seed of a monocotyledon, the monocotyledon can be selected from the group consisting of corn, wheat, oat, rice, barley, millet, banana, onion, garlic, asparagus, ryegrass, millet, fonio, raishan, nipa grass, turmeric, saffron, galangal, chive, cardamom, date palm, pineapple, shallot, leek, scallion, water chestnut, ramp, Job's tears, bamboo, ragi, spotless watermeal, arrowleaf elephant ear, Tahitian spinach, abaca, areca, bajra, betel nut, broom millet, broom sorghum, citronella, coconut, cocoyam, maize, dasheen, durra, durum wheat, edo, fique, formio, ginger, orchard grass, esparto grass, Sudan grass, guinea corn, Manila hemp, henequen, hybrid maize, jowar, lemon grass, maguey, bulrush millet, finger millet, foxtail millet, Japanese millet, proso millet, New Zealand flax, oats, oil palm, palm palmyra, sago palm, redtop, sisal, sorghum, spelt wheat, sweet corn, sweet sorghum, sugarcane, taro, teff, timothy grass, triticale, vanilla, wheat, and yam.

Where the plant is a gymnosperm or the seed is a seed of a gymnosperm, the gymnosperm can be from a family selected from the group consisting of Araucariaceae, Boweniaceae, Brassicaceae, Cephalotaxaceae, Cupressaceae, Cycadaceae, Ephedraceae, Ginkgoaceae, Gnetaceae, Pinaceae, Podocarpaceae, Taxaceae, Taxodiaceae, Welwitschiaceae, and Zamiaceae.

When the plant is from the family Brassicaceae, the plant can comprise a plant of the genus Brassica. For example, the plant of the family Brassicaceae can comprise Brassica napus, Brassica rapa, Brassica juncea, Brassica hirta, Brassica oleracea, Raphanus sativus, Sinapus alba, or Lepidium sativum.

The plants and plant seeds described herein may include transgenic plants or plant seeds, such as transgenic cereals (wheat, rice), maize, soybean, potato, cotton, tobacco, oilseed rape and fruit plants (fruit of apples, pears, citrus fruits and grapes, including wine grapes). Preferred transgenic plants include corn, soybeans, potatoes, cotton, tobacco, sugar beet, sugarcane, and oilseed rape.

Suitable transgenic plants and seeds can be characterized by the plant's formation of toxins, especially from the Bacillus thuringiensis genetic material (e.g., by gene CryIA (a), CryIA (b), CryIA (c), CryIIA, CryIIIA, CryIIIB2, Cry9c, Cry2Ab, Cry3Bb, CryIF or a combination thereof). The formation of toxins in plants increases the plant's resistance to insects, arachnids, nematodes and slugs and snails (hereinafter referred to as “Bt plants”). Bt plants, for example, are commercially available under the tradename YIELD CARD® (for example maize, cotton, soybeans), KNOCKOUT® (for example maize), STARLINK® (for example maize), BOLLGARD® (cotton), NUCOTN® (cotton) and NEWLEAF® (potato) maize varieties, cotton varieties, soybean varieties and potato varieties. Herbicide tolerance plants include plants under the trade names ROUNDUP READY® (a glyphosate tolerance, such as corn, cotton, soybeans), CLEARFIELD® (for example maize), LIBERTY LINK® (tolerance with glufosinate, for example oilseed rape), IMI® (with imidazolinone tolerance) and STS® (tolerance to a sulfonylurea, such as maize).

Plant seeds as described herein can be genetically modified (e.g., any seed that results in a genetically modified plant or plant part that expresses herbicide tolerance, tolerance to environmental factors such as water stress, drought, viruses, and nitrogen production, or resistance to bacterial, fungi or insect toxins). Suitable genetically modified seeds include those of cole crops, vegetables, fruits, trees, fiber crops, oil crops, tuber crops, coffee, flowers, legume, cereals, as well as other plants of the monocotyledonous and dicotyledonous species. Preferably, the genetically modified seeds include peanut, tobacco, grasses, wheat, barley, rye, sorghum, rice, rapeseed, sugarbeet, sunflower, tomato, pepper, bean, lettuce, potato, and carrot. Most preferably, the genetically modified seeds include cotton, soybean, and corn (sweet, field, seed, or popcorn).

Particularly useful transgenic plants which may be treated according to the invention are plants containing transformation events, or a combination of transformation events, that are listed for example in the databases from various national or regional regulatory agencies (see for example http://gmoinfojrc.it/gmp_browse.aspx and http://www.agbios.com/dbase.php).

Having described the invention in detail, it will be apparent that modifications and variations are possible without departing from the scope of the invention defined in the appended claims.

### EXAMPLES

The following non-limiting examples are provided to further illustrate the present invention.

### Example 1. Construction of a Bacillus cereus Family Member Displaying Endopolygalacturonase

To construct a Bacillus cereus family member displaying the endopolygalacturonase of SEQ ID NO: 227, the pSUPER plasmid was generated through fusion of the pUC57 plasmid (containing an ampicillin resistance cassette and a ColE1 origin of replication) with the pBC16-1 plasmid from Bacillus cereus (containing a tetracycline resistance gene, repU replication gene and oriU origin of replication). Note that SEQ ID NO: 227 is the same as SEQ ID NO: 210, except that SEQ ID NO: 227 contains an additional cysteine at its carboxy terminus. This 5.8 kb plasmid can replicate in both E. coli and Bacillus spp. and can be selected by conferring resistance to β-lactam antibiotics in E. coli and resistance to tetracycline in Bacillus spp. The basal pSUPER plasmid was modified by insertion of a PCR-generated fragment that fused the BclA promoter (SEQ ID NO: 149), a start codon, amino acids 20-35 of BclA (amino acids 20-35 of SEQ ID NO: 1) and an alanine linker sequence in frame with SEQ ID NO: 227 resulting in a plasmid termed pSUPER-BclA 20-35-SEQ ID NO: 227. This construct was transformed into E. coli and plated on Lysogeny broth plates plus ampicillin (100 μg/mL) to obtain single colonies. Individual colonies were used to inoculate Lysogeny broth plus ampicillin and incubated overnight at 37° C., 300 rpm. Plasmids from resulting cultures were extracted using a commercial plasmid purification kit. DNA concentrations of these plasmid extracts were determined via spectrophotometry, and obtained plasmids subjected to analytical digests with appropriate combinations of restriction enzymes. The resulting digestion patterns were visualized by agarose gel electrophoresis to investigate plasmid size and presence of distinct plasmid features. Relevant sections, such as the SEQ ID NO: 227 expression cassette, of the purified pSUPER derivatives were further investigated by Sanger sequencing. Verified pSUPER plasmids were introduced by electroporation into Bacillus thuringiensis BT013A. Single transformed colonies were isolated by plating on nutrient broth plates containing tetracycline (10 μg/mL). Individual positive colonies were used to inoculate brain heart infusion broth containing tetracycline (10 μg/mL) and incubated overnight at 30° C., 300 rpm. Genomic DNA of resulting cultures was purified and relevant sections of the pSUPER plasmid were re-sequenced to confirm genetic purity of the cloned sequences. Verified colonies were grown overnight in brain heart infusion broth with 10 μg/mL tetracycline and induced to sporulate through incubation in a yeast extract-based media at 30° C. for 48 hours.

Bacillus thuringiensis BT013A was also grown in the same way as was the recombinant strain. The whole broth culture of BT013A was used as a control for the plant growth promotion and water stress studies described in the following examples.

Bacillus thuringiensis BT013A was deposited with the United States Department of Agriculture (USDA) Agricultural Research Service (ARS), having the address 1815 North University Street, Peoria, Ill. 61604, U.S.A., on Mar. 10, 2014, and assigned accession number NRRL B-50924. Bacillus thuringiensis BT013A is also known as Bacillus thuringiensis 4Q7.

### Example 2. Canola Plant Growth Promotion Studies

Clear trays (5″×5″×2½″) were overlaid with 5″×5″ VersaPak (Anchor Paper Company, St. Paul, Minn.) cushioning material. In each tray, fifty seeds of canola were placed using sterile forceps, and treated with 50 mL diluted whole broth, such that each tray received at least 2×10⁷ CFU of the BT013A wild type strain or the recombinant strain displaying the endopolygalacturonase cargo (the “EPG strain”), as described in Example 1. The untreated control trays received 50 mL of sterile water. A hundred seeds were assayed for each strain, and the untreated control in two trays each. The trays were placed in growth chamber racks set at 450 μmol m⁻² sec⁻¹ photosynthetic photon flux density (PPFD), 14-hours light (28° C.)/10-hours dark (18° C.) in a randomized order and rotated daily to minimize positional effects for 21 days. Watering was done daily beginning day seven until the end of the experiment. No fertilizer or growth amendments were used.

Canola seedlings were observed for plant growth promotion traits beginning five days after seed treatment. Photographs of seedlings in tray with a color and size calibrator were obtained using a camera (Nikon, Tokyo, Japan) for plant image analysis using the Easy Leaf Area software (Easlon, H. M., & Bloom, A. J. (2014). Easy Leaf Area: Automated digital image analysis for rapid and accurate measurement of leaf area. Applications in plant sciences, 2(7), 1400033) on days 5, 7, 14 and 21 after seed treatment. The seedlings treated with the whole broth culture of the EPG strain of Example 1 showed an increase of 15% in leaf area, as compared to seedlings treated with the wild type strain. Additionally, leaf area of seedlings treated with the whole broth culture of the EPG strain of Example 1 showed an increase of 25% when compared to untreated seedlings.

### Example 3. Corn and Soy Water Stress Studies

Clear trays (8″×8″×3″) were filled with sand to approximately an inch from the top, and hydrated with 150 mL water amended with approximately 238 ppm N (fertilizer concentration measured as parts per million of nitrogen) of Peters 20-20-20 General Purpose Fertilizer (The Scotts Company, Marysville, Ohio). Sixteen seeds of corn and soy were planted at 1″ depth in four rows of four seeds each. Each tray was subsequently drenched with 50 mL of the above-described whole broth samples, such that each tray received at least 2×10⁷ CFU of a whole broth culture of the BT013A wild type strain or the EPG strain. The untreated control trays received 50 mL of sterile water. The trays were placed in growth chamber racks set at 450 μmol m² sec⁻¹PPFD (photosynthetic photon flux density), 14-hours light (28° C.)/10-hours dark (18° C.) in a randomized order and rotated daily to minimize positional effects for fourteen days. Watering was done daily for eight days after planting, then withheld for two days to achieve water stress, and resumed until day fourteen after planting to enable plant recovery post-water limiting conditions.

Corn and soy seedlings were observed for plant growth promotion traits fourteen days after drenching the seeds. Seed germination was recorded five days after planting. The above-ground and root tissues were harvested, dried in an oven set at 75° C., and weighed after five days. The dry weight of corn seedlings treated with the whole broth culture of the EPG strain was 41% higher than seedlings treated with the wild type strain and 18% higher than untreated seedlings. The dry weight of soybean seedlings treated with the whole broth culture of the EPG strain was less than that of seedlings treated with the wild type strain but slightly higher than that of untreated seedlings.

### Example 4. Construction of a Bacillus cereus Family Member Displaying Endopolygalacturonase of SEQ ID NOs: 211-212 and Polygalacturonase of SEQ ID NOs: 219-220

pSUPER constructs encoding fusion proteins containing an endopolygalacturonase or a polygalacturonase sequence were prepared in the same manner as described above in Example 1 for SEQ ID NO: 227. Accordingly, each of the constructs contained the BclA promoter (SEQ ID NO: 149) fused to a start codon, a coding sequence for amino acids 20-35 of BclA (amino acids 20-35 of SEQ ID NO: 1) and an alanine linker in frame with a coding sequence for a polygalacturonase (SEQ ID NOs: 211, 212, 219, and 220).

Additionally and alternatively, a derivative plasmid of the pSUPER plasmids described above was created as follows. The pBC fragment (pBC16-1-derived section of pSUPER including BclA/polygalacturonase expression cassette) of the pSUPER plasmids described above was amplified by PCR and subsequently circularized by blunt-end ligation.

These tetracycline resistance-carrying pBC plasmid ligations were introduced by electroporation into Bacillus thuringiensis BT013A. Single transformed colonies were isolated by plating on nutrient broth plates containing tetracycline (10 μg/mL). Individual positive colonies were used to inoculate brain heart infusion broth containing tetracycline (10 μg/mL) and incubated overnight at 30° C., 300 rpm. Genomic DNA of resulting cultures was purified and relevant sections of the pBC plasmid were re-sequenced to confirm genetic purity of the cloned sequences and the correct ligation site. Verified colonies were grown overnight in brain heart infusion broth with 10 μg/mL tetracycline and induced to sporulate through incubation in a yeast extract-based media at 30° C. for 48 hours.

### Example 5. Construction and Purification of Exosporium Fragments from a Bacillus cereus Family Member Expressing Endopolygalacturonase

Knock-out (KO) Mutants: To make exsY knockout (KO) mutant strains of Bacillus thuringiensis BT013A, Bt013AexsYKO, the plasmid pKOKI shuttle and integration vector was constructed that contained the pUC57 backbone, which is able to replicate in E. coli, as well as the origin of replication and erythromycin resistance cassette from pE194. This construct is able to replicate in both E. coli and Bacillus spp. A construct was made that contained the 1 kb DNA region that corresponded to the upstream region of the exsY gene and a 1 kb region that corresponded to the downstream region of the gene exsY gene, both of which were PCR amplified from Bacillus thuringiensis BT013A. For each construct, the two 1 kb regions were then spliced together using homologous recombination with overlapping regions to each other and the pKOKI plasmid. This plasmid construct was verified by digestion and DNA sequencing. Clones were screened for erythromycin resistance.

Clones were passaged under high temperature (40° C.) in brain heart infusion broth. Individual colonies were toothpicked onto LB agar plates containing erythromycin 5 μg/mL, grown at 30° C., and screened for the presence of the pKOKI plasmid integrated into the chromosome by colony PCR. Colonies that had an integration event were continued through passaging to screen for single colonies that lost erythromycin resistance (signifying loss of the plasmid by recombination and removal of the exsY gene). Verified deletions were confirmed by PCR amplification and sequencing of the target region of the chromosome. Finally, each pBC plasmid containing the gene encoding for a polygalacturonase enzyme (described above in Example 4) was transformed into the exsY mutant strain of BT013A.

For each exsY mutant expressing the polygalacturonase of SEQ ID NO: 211-212 or 219-220 an overnight culture was grown in brain heart infusion media at 30° C., 300 rpm, in baffled flasks with antibiotic selection. One milliliter of this overnight culture was inoculated into a yeast extract-based media (50 mL) in a baffled flask and grown at 30° C. for 2 days. An aliquot of spores was removed, and the spores were agitated by vortexing. The spores were collected via centrifugation at 8,000×g for 10 minutes, and supernatant containing the exosporium fragments was filtered through a 0.22 μm filter to remove any residual spores. No spores were found in the filtrate.

The recombinant strains described in Examples 4 and 5 are described in Table 6, below.

TABLE 6

| Shortened Name of Recombinant | |
|---|---|
| Bacillus cereus Family Member | Plasmid |
| BT013A-pBC211 | pBC-BclA 20-35-SEQ ID NO: 211 |
| BT013A-pBC212 | pBC-BclA 20-35-SEQ ID NO: 212 |
| BT013A-pBC219 | pBC-BclA 20-35-SEQ ID NO: 219 |
| BT013A-pBC220 | pBC-BclA 20-35-SEQ ID NO: 220 |
| BT013AexsYKO-pBC211 | pBC-BclA 20-35-SEQ ID NO: 211 |
| BT013AexsYKO-pBC212 | pBC-BclA 20-35-SEQ ID NO: 212 |
| BT013AexsYKO-pBC219 | pBC-BclA 20-35-SEQ ID NO: 219 |
| BT013AexsYKO-pBC220 | pBC-BclA 20-35-SEQ ID NO: 220 |

### Example 6. Canola Plant Growth Promotion Studies with EPG and Polygalacturonase from Bacillus Species

Clear trays (5″×5″×2½″) were overlaid with 5″×5″ VersaPak (Anchor Paper Company, St. Paul, Minn.) cushioning material. In each tray, fifty seeds of canola were placed and treated with 50 mL diluted whole broth, such that each tray received (i) at least 1×10⁵ CFU of the BT013A wild type strain or (ii) at least 1×10⁵ CFU of the recombinant BT013A strain shown in Table 7, below or (iii) a volume of exosporium fragment preparation equivalent to the volume of the whole broth culture of the recombinant BT013AexsYKO strains shown in Table 7, below that contained at least 1×10⁵ CFU. (This volume was selected in order to obtain an amount of enzyme comparable to that in the whole broth samples used in (ii), as very little liquid is lost during the centrifugation and filtration processes that are used to separate exosporium fragments from cells, and it is assumed that whole broth cultures of BT013A-pBC2XX and BT013AexsYKO-pBC2XX have similar amounts of enzyme displayed on their exosporium.) The untreated control trays received 50 mL of sterile water. Two hundred seeds were assayed for each strain, and the untreated control in four trays each. The trays were placed in growth chamber racks set at 450 μmol m² sec⁻¹ photosynthetic photon flux density (PPFD), 14-hours light (28° C.)/10-hours dark (18° C.) in a randomized order and rotated daily to minimize positional effects for 14 days. No fertilizer or growth amendments were used. No additional watering was provided beyond what was given at the start of the assay.

Canola seedlings were observed for plant growth promotion traits beginning seven days after seed treatment. Photographs of seedlings in tray with a color and size calibrator were obtained using a camera (Nikon, Tokyo, Japan) for plant image analysis using the Easy Leaf Area software (Easlon, H. M., & Bloom, A. J. (2014). Easy Leaf Area: Automated digital image analysis for rapid and accurate measurement of leaf area. Applications in plant sciences, 2(7), 1400033) on days 7, 11 and 14 after seed treatment.

TABLE 7

| | Average Seedling Growth Gain | |
|---|---|---|
| | Over Bt013AWT Live Microbe | |
| | (%; n = 200) | |
| | Live Microbe | Strips |
| | BT013A- | BT013AexsYKO- |
| Treatment | pBC2XX* | pBC2XX* |
| Bacillus spp. EPG (SEQ ID NO: 212) | 16.059 | 12.703 |
| B. simplex 30N-5 EPG (SEQ ID NO: | 21.887 | 18.781 |
| 211) | | |
| B. safensis EPG (SEQ ID NO: 219) | 15.04 | 20.329 |
| B. altitudinis EPG (SEQ ID NO: 220) | 22.723 | 25.56 |

| | |
|---|---|
| Bare | 1.096 |
| (untreated control = naked seed | |
| drenched with sterile water) | |

*2XX corresponds to the SEQ ID NO shown in the “Treatment” column.

### Example 7. Construction of Bacillus cereus Family Members Displaying Pectate Lyase or Exo-Polygalacturonase

pSUPER constructs encoding fusion proteins containing a pectate lyase or an exo-polygalacturonase sequence were prepared as described above in Example 1 for SEQ ID NO: 227. Accordingly, each of the constructs contained the BclA promoter (SEQ ID NO: 149) fused to a start codon, a coding sequence for amino acids 20-35 of BclA (amino acids 20-35 of SEQ ID NO: 1) and an alanine linker in frame with a coding sequence for a pectate lyase (SEQ ID NOs: 222, 223, 224, 225, and 226) or exo-polygalacturonase (SEQ ID NO: 218).

The tetracycline-resistance carrying pBC sections of above described pSUPER constructs were transformed into wild type Bacillus thuringiensis BT013A as described in Example 4. Whole broth cultures of the expression strains that were based on the wildtype Bacillus thuringiensis BT013A strain were generated as described in Example 4. The tetracycline-resistance carrying pBC sections of above described pSUPER constructs were further transformed into the exsY knockout mutant of Bacillus thuringiensis BT013A from Example 5. Exosporium fragment preparations of expression strains that were based on the exsY KO mutant of Bacillus thuringiensis BT013A were generated as described in Example 5.

The recombinant strains described in Example 7 are described in Table 8, below.

TABLE 8

| Shortened Name of Recombinant | |
|---|---|
| Bacillus cereus Family Member | Plasmid |
| BT013A-pBC218 | pBC-BclA 20-35-SEQ ID NO: 218 |
| BT013A-pBC222 | pBC-BclA 20-35-SEQ ID NO: 222 |
| BT013A-pBC223 | pBC-BclA 20-35-SEQ ID NO: 223 |
| BT013A-pBC224 | pBC-BclA 20-35-SEQ ID NO: 224 |
| BT013A-pBC225 | pBC-BclA 20-35-SEQ ID NO: 225 |
| BT013A-pBC226 | pBC-BclA 20-35-SEQ ID NO: 226 |
| BT013AexsYKO-pBC218 | pBC-BclA 20-35-SEQ ID NO: 218 |
| BT013AexsYKO-pBC222 | pBC-BclA 20-35-SEQ ID NO: 222 |
| BT013AexsYKO-pBC223 | pBC-BclA 20-35-SEQ ID NO: 223 |
| BT013AexsYKO-pBC224 | pBC-BclA 20-35-SEQ ID NO: 224 |
| BT013AexsYKO-pBC225 | pBC-BclA 20-35-SEQ ID NO: 225 |
| BT013AexsYKO-pBC226 | pBC-BclA 20-35-SEQ ID NO: 226 |

### Example 8. Seed Treatment of Corn with Pectate Lyase and Exo-Polygalacturonase Resulted in Increased Plant Height

Pectate lyase or exo-polygalacturonase containing whole broth samples of BT013A-pBC218, BT013A-pBC222, BT013A-pBC223, BT013A-pBC224, BT013A-pBC225, BT013A-pBC226, and pectate lyase or exo-polygalacturonase containing exosporium fragment preparations of BT013AexsYKO-pBC218, BT013AexsYKO-pBC222, BT013AexsYKO-pBC223, BT013AexsYKO-pBC224, BT013AexsYKO-pBC225, BT013AexsYKO-pBC226, respectively, were prepared according to Example 7 (culture sporulation rates: approximately 99%; final spore concentrations: 1.1-1.7×10⁸ spores per mL) and applied to corn seed (Beck's corn hybrid 5765 YH) at a use rate of 0.72 μL per seed. Treated seed were investigated in replicated trials with three replicates per trial and 18 seed per treatment in each replicate. Coated seeds were sowed directly into 39.7 cm³ pots containing sandy loam topsoil at a depth of 2.54 cm², with two seeds per pot. After planting, 50 mL of room temperature water was added to each pot to initiate germination. The pots were kept in an artificial lighted growth room receiving approximately 300 μmol m⁻² s⁻¹ (light photons) for a 13/11 light/day cycle and a 21° C. day/15° C. night temperature range. All plants received the same watering and fertilizer regimes. Plant height was measured ten days after planting (DAP). Average plant height measurements were normalized to the average plant height of plants from seed that were treated with equivalent volumes of water instead of whole broth samples or exosporium fragment preparations within each replicate and subsequently averaged across all replicates of the trial. Obtained results are reported in Table 9 below. The p-values were obtained by utilizing a 2-tail t-test assuming equal variance across the samples.

TABLE 9
Effects of Pectate Lyase and Exo-Polygalacturonase on Plant
Height for Corn, Seed Treatment

| | Average Plant | | |
|---|---|---|---|
| | Height (cm) | | |
| | Normalized to | Standard | |
| | Control | Error | P- |
| Corn Treatment | (Seed Treatment) | Mean | value |
| BT013A-pBC218 | 100.7% | 2% | 0.92 |
| Exo-polygalacturonase | | | |
| ExoPG | | | |
| Bacillus licheniformis A4 | | | |
| BT013A-pBC222 | 106.0% | 2% | 0.06 |
| Pectate lyase PelC | | | |
| Bacillus subtilis 168 | | | |
| BT013A-pBC223 | 105.7% | 2% | 0.03 |
| Pectate lyase Pel-103 | | | |
| Bacillus sp. KSM-P103 | | | |
| BT013A-pBC224 | 106.4% | 3% | 0.03 |
| Pectate lyase Pel-22 | | | |
| Bacillus pumilus BS22 | | | |
| BT013A-pBC225 | 103.4% | 2% | 0.13 |
| Pectate lyase PelA | | | |
| Bacillus licheniformis A4 | | | |
| BT013A-pBC226 | 106.0% | 2% | 0.04 |
| Pectate lyase Pel | | | |
| Bacillus amyloliquefaciens | | | |
| subsp. plantarum UCMB511 | | | |
| BT013AexsYKO-pBC218 | 100.4% | 2% | 0.87 |
| Exo-polygalacturonase | | | |
| ExoPG | | | |
| Bacillus licheniformis A4 | | | |
| BT013AexsYKO-pBC222 | 103.3% | 2% | 0.43 |
| Pectate lyase PelC | | | |
| Bacillus subtilis 168 | | | |
| BT013AexsYKO-pBC223 | 113.9% | 2% | 0.00005 |
| Pectate lyase Pel-103 | | | |
| Bacillus sp. KSM-P103 | | | |
| BT013AexsYKO-pBC224 | 106.9% | 1% | 0.01 |
| Pectate lyase Pel-22 | | | |
| Bacillus pumilus BS22 | | | |
| BT013AexsYKO-pBC225 | 108.7% | 2% | 0.007 |
| Pectate lyase PelA | | | |
| Bacillus licheniformis A4 | | | |
| BT013AexsYKO-pBC226 | 111.0% | 2% | 0.02 |
| Pectate lyase Pel | | | |
| Bacillus amyloliquefaciens | | | |
| subsp. plantarum | | | |
| UCMB5113 | | | |

Treatment of corn seed with pectate lyases (BT013A-pBC222, BT013A-pBC223, BT013A-pBC224, BT013A-pBC225, BT013A-pBC226, BT013AexsYKO-pBC222, BT013AexsYKO-pBC223, BT013AexsYKO-pBC224, BT013AexsYKO-pBC225, BT013AexsYKO-pBC226) or exo-polygalacturonase (BT013A-pBC218, BT013AexsYKO-pBC218) applied at 0.72 μL per seed resulted in positive growth benefits toward V2 (second leaf collar stage, 10 DAP) corn plants. A pectate lyase Pel-103 containing exosporium fragment preparation of BT013AexsYKO-pBC223 significantly (p≤0.005) increased average plant height by +13.9% over water-treated control plants compared across the three-replicate trial. Whole broth samples containing the pectate lyases Pel-103, Pel-22 and Pel (BT013A-pBC223, BT013A-pBC224, BT013A-pBC226) significantly (p≤0.05) increased average plant height by +5.7%, +6.4%, +6.0%, respectively, over the water-treated control plants. Exosporium fragment preparations containing the pectate lyases Pel-22 and PelA, Pel (BT013AexsYKO-pBC224, BT013AexsYKO-pBC225, BT013AexsYKO-pBC226) led to significant (p<0.05) increases in average plant height by +6.9%, +8.7%, and +11%, over the water-treated control plants compared across the three replicates. Whole broth samples containing the pectate lyases PelC and PelA (BT013A-pBC222, BT013A-pBC225), and a pectate lyase PelC containing exosporium fragment preparation (BT013AexsYKO-pBC222) resulted in increased plant height by +6.0%, +3.4%, and +3.3%, respectively, over that of the water-treated control plants.

### Example 9: Effects of Soybean Seed Treatment with Pectate Lyases and Exo-Polygalacturonase on Germination, Plant Growth, Canopy Coverage and Biomass

Pectate lyase or exo-polygalacturonase containing whole broth samples of BT013A-pBC218, BT013A-pBC222, BT013A-pBC223, BT013A-pBC224, BT013A-pBC225, BT013A-pBC226, and pectate lyase or exo-polygalacturonase containing exosporium fragment preparations of BT013AexsYKO-pBC218, BT013AexsYKO-pBC222, BT013AexsYKO-pBC223, BT013AexsYKO-pBC224, BT013AexsYKO-pBC225, BT013AexsYKO-pBC226, respectively, were prepared according to Example 7 (culture sporulation rates: approximately 99%; final spore concentrations: 1.1-1.7×10⁸ spores per mL) and applied to soybean seed (Beck's soybean variety 366L4) at a use rate of 0.5 μL per seed. Control seeds were treated with an equivalent volume of water. Treated seed were investigated in replicated trials with three replicates per trial and 18 seed per treatment in each replicate. Coated seeds were sowed directly into 39.7 cm³ pots containing a sandy loam topsoil at a depth of 2.54 cm², with 2 seeds per pot. After planting, 50 mL of room temperature water was added to each pot to initiate germination. The pots were kept in an artificial lighted growth room receiving approximately 300 μmol m⁻² s⁻¹ (light photons) for a 13/11 light/day cycle and a 24° C. day/20° C. night temperature range. All plants received the same watering and fertilizer regimes. Emergence counts were recorded fourteen days after planting (Table 10). Emergence is defined as the percentage of all seedlings that emerged from the soil surface until fourteen days post planting. In addition, plant height (Table 11) and fractional green canopy cover (FGCC) (Table 12) were determined at VC (cotyledon stage) fourteen days post planting and compared to 14-day-old plants grown from seeds that received only a water treatment. FGCC is a diagnostic parameter used to estimate green canopy area. Canopeo (Matlab, Mathworks, Inc., Natick, Mass.) serves as a measurement tool for FGCC and is based on determining color ratios of red to green (R/G) and blue to green (B/G) to enable compensation for excess green index and the background. FGCC ranges from 0 (0% green canopy cover) to 1 (100% green canopy cover). The classification of green canopy is based on the following criteria:

$$\text{R} \text{/} \text{G} < {P}_{1} and \text{B} \text{/} \text{G} < {P}_{2} and 2 \text{G} \text{-} \text{R} \text{-} \text{B} > {P}_{3}$$

Where P₁ and P₂ are parameters that have a value near 1 to classify the pixels that are in the green wavelength band range (500-570 nm), and P₃ is a parameter that sets the minimum excess green vegetation and allows for background subtraction. Default parameters of Canopeo were used (P₁=0.95, P₂=0.95, and P₃=20).

Four to five weeks after planting, measurements of dry biomass were obtained and compared to control plants grown from water-treated seeds. Table 13 summarizes normalized biomass results per treatment across the 54-seed trial.

TABLE 10
Effects of Pectate Lyase on Germination for
Soybean, Seed Treatment

| Soybean Treatment | Percentage of Seeds Germinated |
|---|---|
| Water Control | 91% |
| BT013A-pBC223 | 100% |
| Pectate lyase Pel-103 | |
| Bacillus sp. KSM-P103 | |
| BT013A-pBC225 | 98% |
| Pectate lyase PelA | |
| Bacillus licheniformis A4 | |
| BT013AexsYKO-pBC223 | 96% |
| Pectate lyase Pel-103 | |
| Bacillus sp. KSM-P103 | |
| BT013AexsYKO-pBC225 | 93% |
| Pectate lyase PelA | |
| Bacillus licheniformis A4 | |

Treatment of soybean seed with pectate lyases (BT013A-pBC223, BT013A-pBC225, BT013AexsYKO-pBC223, BT013AexsYKO-pBC225) applied at 0.5 μL per seed improved germination percentage over plants that were grown from water-treated control seed. Whole broth samples containing the pectate lyases Pel-103 and PelA (BT013A-pBC223, BT013A-pBC225), respectively, increased germination by +9% and +7%, over that of water-treated control seed. Exosporium fragment preparations containing the pectate lyases Pa-103 and PelA (BT013AexsYKO-pBC223, BT013AexsYKO-pBC225) increased germination of soybean plants by +5%, and +2%, respectively, over that of water-treated control seed.

TABLE 11
Effects of Pectate Lyase and Exo-Polygalacturonase on Plant
Height for Soybean, Seed Treatment

| | Average Plant | | |
|---|---|---|---|
| | Height (cm) | | |
| | normalized | Standard | |
| | to control | Error | P- |
| Soybean Treatment | (Seed Treatment) | Mean | value |
| BT013AexsYKO-pBC218 | 104.6% | 3% | 0.32 |
| Exo-polygalacturonase ExoPG | | | |
| Bacillus licheniformis A4 | | | |
| BT013AexsYKO-pBC223 | 112.4% | 4% | 0.03 |
| Pectate lyase Pel-103 | | | |
| Bacillus sp. KSM-P103 | | | |
| BT013AexsYKO-pBC225 | 110.8% | 3% | 0.02 |
| Pectate lyase PelA | | | |
| Bacillus licheniformis A4 | | | |

Treatment of soybean seed with pectate lyases (BT013AexsYKO-pBC223, BT013AexsYKO-pBC225) or exo-polygalacturonase (BT013AexsYKO-pBC218) applied at 0.5 μL per seed resulted in positive growth benefits to VC (cotyledon stage; 14 DAP) soybean plants. Exosporium fragment preparations containing the pectate lyases Pel-103 and PelA (BT013AexsYKO-pBC223, BT013AexsYKO-pBC225) significantly (p<0.05) increased soybean plant height by +12.4% and +10.8% over that of plants grown from water-treated seed across three 18-seed replicates. An exosporium fragment preparation containing exo-polygalacturonase ExoPG (BT013AexsYKO-pBC218) showed increased growth with an average plant height +4.6% compared to the water-treated control plants.

TABLE 12
Effects of Pectate Lyase on FGCC for Soybean, Seed Treatment

| | Average Plant |
|---|---|
| | Canopy Coverage |
| | Normalized to Control |
| Soybean Treatment | (Seed Treatment) |
| BT013AexsYKO-pBC218 | 120.0% |
| Exo-polygalacturonase ExoPG | |
| Bacillus licheniformis A4 | |
| BT013AexsYKO-pBC223 | 152.2% |
| Pectate lyase Pel-103 | |
| Bacillus sp. KSM-P103 | |
| BT013AexsYKO-pBC224 | 100.2% |
| Pectate lyase Pel-22 | |
| Bacillus pumilus BS22 | |
| BT013AexsYKO-pBC225 | 131.3% |
| Pectate lyase PelA | |
| Bacillus licheniformis A4 | |
| BT013AexsYKO-pBC226 | 120.2% |
| Pectate lyase Pel, | |
| Bacillus amyloliquefaciens subsp. | |
| plantarum UCMB5113 | |

Treatment of soybean seed with pectate lyase (BT013AexsYKO-pBC223, BT013AexsYKO-pBC225, BT013AexsYKO-pBC226) or exo-polygalacturonase (BT013AexsYKO-pBC218) applied at 0.5 μL per seed resulted in positive effects for canopy coverage for VC (cotyledon stage; 14 DAP) soybean plants. Exosporium fragment preparations containing the pectate lyases Pel-103, PelA and Pel (BT013AexsYKO-pBC223, BT013AexsYKO-pBC225, BT013AexsYKO-pBC226) and the exo-polygalacturonase ExoPG (BT013AexsYKO-pBC218), respectively, increased average plant canopy coverage by +52.2%, +31.3%, +20.2%, and +20.0%, respectively, compared to plants grown from water-treated control seed across three 18-seed replicates

TABLE 13
Effects of Pectate Lyase on Dry Weight in Soybean, Seed Treatment

| | Average | Average | Average |
|---|---|---|---|
| | Dry Total | Dry Above Ground | Dry Root |
| | Biomass (g) | Biomass (g) | Biomass (g) |
| | Per Plant | Per Plant | Per Plant |
| | Normalized to | Normalized to | Normalized to |
| | Control | Control | Control |
| Soybean Treatment | (Seed Treatment) | (Seed Treatment) | (Seed Treatment) |
| BT013AexsYKO-pBC218 | 100% | 101.3% | 98.7% |
| Exo-polygalacturonase | | | |
| ExoPG | | | |
| Bacillus licheniformis A4 | | | |
| BT013AexsYKO-pBC223 | 104.6% | 99.7% | 114.0% |
| Pectate lyase Pel-103 | | | |
| Bacillus sp. KSM-P103 | | | |
| BT013AexsYKO-pBC224 | 115.7% | 123.2% | 102.6% |
| Pectate lyase Pel-22 | | | |
| Bacillus pumilus BS22 | | | |
| BT013AexsYKO-pBC225 | 108.8% | 102.2% | 121.3% |
| Pectate lyase PelA | | | |
| Bacillus licheniformis A4 | | | |
| BT013AexsYKO-pBC226 | 123.9% | 126.5% | 121.0% |
| Pectate lyase Pel | | | |
| Bacillus amyloliquefaciens | | | |
| subsp. plantarum | | | |
| UCMB5113 | | | |

Treatment of soybean seed with pectate lyase (BT013AexsYKO-pBC223, BT013AexsYKO-pBC224, BT013AexsYKO-pBC225, BT013AexsYKO-pBC226) as well as exo-polygalacturonase (BT013AexsYKO-pBC218) applied at 0.5 μL per seed resulted in positive effects in biomass in 4-5-week-old soybean plants in a 54-seed trial. An exosporium fragment preparation containing the pectate lyase Pel (BT013AexsYKO-pBC226) increased the average dry total biomass, average dry above ground biomass, and average dry root biomass per plant by +23.9%, +26.5%, and +21.0%, respectively, compared to plants grown from water-treated seeds. An exosporium fragment preparation containing the pectate lyase Pel-22 (BT013AexsYKO-pBC224) increased average dry total, dry above ground, and dry root biomass per plant by +15.7%, +23.2%, and +2.6%, respectively, compared to the control plants. An exosporium fragment preparation containing the pectate lyase Pel-103 (BT013AexsYKO-pBC223) resulted in an increase in average dry total and dry root biomass per plant by +4.6% and +14.0%, respectively, compared to the control plants. Pectate lyase Pel A provided in an exosporium fragment preparation (BT013AexsYKO-pBC225) to seed, increased average dry total biomass, dry above ground biomass, and dry root biomass per plant by +8.8%, +2.2%, and +21.3%, respectively, compared to the water-treated control plants. An exosporium fragment preparation containing exo-polygalacturonase ExoPG (BT013AexsYKO-pBC218) applied as a seed treatment increased the average dry above ground biomass by 1.3% compared to water-treated control plants.

In view of the above, it will be seen that the several objects of the invention are achieved and other advantageous results attained.

As various changes could be made in the above products, formulations, and methods without departing from the scope of the invention, it is intended that all matter contained in the above description shall be interpreted as illustrative and not in a limiting sense.

### EMBODIMENTS

For further illustration, additional non-limiting embodiments of the present disclosure are set forth below.

Embodiment 1 is a fusion protein comprising the targeting sequence, exosporium protein or exosporium protein fragment described in Column 2 and the enzyme described in Column 1 of Table 14, below.

TABLE 14
Fusion Proteins

| | Column 2 - Targeting Sequence, Exosporium Protein or |
|---|---|
| Column 1 - Enzyme | Exosporium Protein Fragment |
| An amino acid sequence | One of the targeting sequences, exosporium proteins or exosporium |
| having at least 95% | protein fragments numbered as 1-487 in paragraph [00180] or one |
| identity to SEQ ID NO: | of the targeting sequences, exosporium proteins or exosporium |
| 210 or 227 | protein fragments disclosed in paragraphs [00181]-[00215]. |
| An amino acid sequence | One of the targeting sequences, exosporium proteins or exosporium |
| having at least 85% | protein fragments numbered as 1-487 in paragraph [00180] or one |
| identity to SEQ ID NO: | of the targeting sequences, exosporium proteins or exosporium |
| 211 or 212 | protein fragments disclosed in paragraphs [00181]-[00215]. |
| An amino acid sequence | One of the targeting sequences, exosporium proteins or exosporium |
| having at least 95% | protein fragments numbered as 1-487 in paragraph [00180] or one |
| identity to SEQ ID NO: | of the targeting sequences, exosporium proteins or exosporium |
| 213 | protein fragments disclosed in paragraphs [00181]-[00215]. |
| An amino acid sequence | One of the targeting sequences, exosporium proteins or exosporium |
| having at least 90% | protein fragments numbered as 1-487 in paragraph [00180] or one |
| identity to SEQ ID NO: | of the targeting sequences, exosporium proteins or exosporium |
| 214 or 215 | protein fragments disclosed in paragraphs [00181]-[00215]. |
| An amino acid sequence | One of the targeting sequences, exosporium proteins or exosporium |
| having at least 95% | protein fragments numbered as 1-487 in paragraph [00180] or one |
| identity to SEQ ID NO: | of the targeting sequences, exosporium proteins or exosporium |
| 216 | protein fragments disclosed in paragraphs [00181]-[00215]. |
| An amino acid sequence | One of the targeting sequences, exosporium proteins or exosporium |
| having at least 95% | protein fragments numbered as 1-487 in paragraph [00180] or one |
| identity to SEQ ID NO: | of the targeting sequences, exosporium proteins or exosporium |
| 217 | protein fragments disclosed in paragraphs [00181]-[00215]. |
| An amino acid sequence | One of the targeting sequences, exosporium proteins or exosporium |
| having at least 95% | protein fragments numbered as 1-487 in paragraph [00180] or one |
| identity to SEQ ID NO: | of the targeting sequences, exosporium proteins or exosporium |
| 218 | protein fragments disclosed in paragraphs [00181]-[00215]. |
| An amino acid sequence | One of the targeting sequences, exosporium proteins or exosporium |
| having at least 90% | protein fragments numbered as 1-487 in paragraph [00180] or one |
| identity to SEQ ID NO: | of the targeting sequences, exosporium proteins or exosporium |
| 219 or 220 or 221 | protein fragments disclosed in paragraphs [00181]-[00215]. |

Embodiment 2 is any of the fusion proteins of Embodiment 1 with an amino acid linker between the targeting sequence, the exosporium protein, or the exosporium protein fragment and the enzyme.

Embodiment 3 is any of the fusion proteins of Embodiment 2, where the linker comprises a polyalanine linker, a polyglycine linker, or a linker comprising a mixture of both alanine and glycine residues.

Embodiment 4 is a recombinant Bacillus cereus family member that expresses any of the fusion proteins of Embodiments 1-3.

Embodiment 5 is the recombinant Bacillus cereus family member of Embodiment 4 where the recombinant Bacillus cereus family member comprises Bacillus thuringiensis Bt013A.

Embodiment 6 is the recombinant Bacillus cereus family member of Embodiment 4 having a mutation that results in Bacillus cereus family member spores having an exosporium that is easier to remove from the spore as compared to the exosproium of a wild-type spore. Specifically, the recombinant Bacillus cereus family member with the mutation can have one of the following mutations:
(i) a mutation in a CotE gene;
(ii) expresses an ExsY protein, wherein the expression of the ExsY protein is increased as compared to the expression of the ExsY protein in a wild-type Bacillus cereus family member under the same conditions, and wherein the ExsY protein comprises a carboxy-terminal tag comprising a globular protein;
(iii) expresses a BclB protein, wherein the expression of the BclB protein is increased as compared to the expression of the BclB protein in a wild-type Bacillus cereus family member under the same conditions;
(iv) expresses a YjcB protein, wherein the expression of the YjcB protein is increased as compared to the expression of the YjcB protein in a wild-type Bacillus cereus family member under the same conditions;
(v) comprises a mutation in an ExsY gene;
(vi) comprises a mutation in a CotY gene;
(vii) comprises a mutation in an ExsA gene; or
(viii) comprises a mutation in a CotO gene.

Embodiment 7 comprises the recombinant Bacillus cereus family member of Embodiment 6, having a mutation in an ExsY or CotY gene.

Embodiment 8 comprises exosporium fragments from the recombinant Bacillus cereus family member of any one of Embodiments 6 and 7.

Embodiment 9 comprises a whole broth culture of the recombinant Bacillus cereus family member of any one of Embodiments 4 and 5.

Embodiment 10 comprises a whole broth culture or other fermentation product of the recombinant Bacillus cereus family member of any one of Embodiments 6 and 7.

Embodiment 11 comprises exopsorium fragments from the whole broth culture of Embodiment 10.

Embodiment 12 comprises the recombinant Bacillus cereus family member of any one of Embodiments 4 and 5 and an agriculturally acceptable carrier.

Embodiment 13 comprises the whole broth culture or other fermentation product of Embodiment 9 and an agriculturally acceptable carrier.

Embodiment 14 comprises the exosporium fragments of Embodiment 8 or Embodiment 11 and an agriculturally acceptable carrier.

1. A fusion protein comprising:
a targeting sequence, exosporium protein, or exosporium protein fragment that targets the fusion protein to the exosporium of a recombinant Bacillus cereus family member; and
a polygalacturonase, the polygalacturonase:
comprising an endopolygalacturonase from Apergillus niger ATCC 9029;
comprising a Bacillus simplex endopolygalacturonase;
comprising a Bacillus safensis polygalacturonase;
comprising a Bacillus altitudinis polygalacturonase;
comprising a Bacillus licheniformis polygalacturonase;
comprising an amino acid sequence having at least 80%, at least 85%, at least 90%, at least 95%, at least 98%, at least 99%, or 100% identity to SEQ ID NO: 210;
consisting of an amino acid sequence having at least 80%, at least 85%, at least 90%, at least 95%, at least 98%, at least 99%, or 100% identity to any one of SEQ ID NO: 210 and 227;
comprising an amino acid sequence having at least 60%, at least 70%, at least 75%, at least 80%, at least 85%, at least 90%, at least 95%, at least 98%, at least 99%, or 100% identity to any one of SEQ ID NOs: 218-221;
consisting of an amino acid sequence having at least 60%, at least 70%, at least 75%, at least 80%, at least 85%, at least 90%, at least 95%, at least 98%, at least 99%, or 100% identity to any one of SEQ ID NOs: 218-221;
comprising an amino acid sequence having at least 70%, at least 75%, at least 80%, at least 85%, at least 90%, at least 95%, at least 98%, at least 99%, or 100% identity to SEQ ID NOs: 211 or 212;
consisting of an amino acid sequence having at least 70%, at least 75%, at least 80%, at least 85%, at least 90%, at least 95%, at least 98%, at least 99%, or 100% identity to SEQ ID NO: 211 or 212;
or a combination of any thereof.

2. The fusion protein of claim 1, wherein the targeting sequence comprises or consists of:
(a) an amino acid sequence having at least about 43% identity with amino acids 20-35 of SEQ ID NO: 1, wherein the identity with amino acids 25-35 is at least about 54%;
(b) amino acids 1-35 of SEQ ID NO: 1;
(c) amino acids 20-35 of SEQ ID NO: 1;
(d) SEQ ID NO: 1;
(e) SEQ ID NO: 96; or
(f) SEQ ID NO: 120.

3. The fusion protein of claim 1 wherein the targeting sequence comprises the sequence X₁-X₂-X₃-X₄-X₅-X₆-X₇-X₈-X₉-X₁₀-X₁₁-X₁₂-X₁₃-X₁₄-X₁₅-X₁₆, wherein:
X₁ is any amino acid or absent;
X₂ is phenylalanine (F), leucine (L), isoleucine (I), or methionine (M);
X₃ is any amino acid;
X₄ is proline (P) or serine (S);
X₅ is any amino acid;
X₆ is leucine (L), asparagine (N), serine (S), or isoleucine (I);
X₇ is valine (V) or isoleucine (I);
X₈ is glycine (G);
X₉ is proline (P);
X₁₀ is threonine (T) or proline (P);
X₁₁ is leucine (L) or phenylalanine (F);
X₁₂ is proline (P);
X₁₃ is any amino acid;
X₁₄ is any amino acid;
X₁₅ is proline (P), glutamine (Q), or threonine (T); and
X₁₆ is proline (P), threonine (T), or serine (S).

4. (canceled)

5. The fusion protein of claim 1, wherein the fusion protein further comprises an amino acid linker between the targeting sequence, the exosporium protein, or the exosporium protein fragment and the polygalacturonase.

6. (canceled)

7. The fusion protein of claim 1, wherein the polygalacturonase comprises or consists of an endopolygalacturonase from Aspergillus niger ATCC 9029.

8. The fusion protein of claim 1, wherein the polygalacturonase comprises or consists of an amino acid sequence having at least 85% sequence identity to SEQ ID NO: 211.

9. The fusion protein of claim 1, wherein the polygalacturonase comprises an amino acid sequence having at least 60%, at least 70%, at least 75%, at least 80%, at least 85%, at least 90%, at least 95%, at least 98%, at least 99%, or 100% identity to any one of SEQ ID NOs: 218-221.

10. The fusion protein of claim 1, wherein the polygalacturonase comprises an amino acid sequence having at least 90% sequence identity to any one of SEQ ID NOs: 219-221.

11. (canceled)

12. A fusion protein comprising:
a targeting sequence, exosporium protein, or exosporium protein fragment that targets the fusion protein to the exosporium of a recombinant Bacillus cereus family member; and
a pectate lyase, the pectate lyase:
comprising a Bacillus subtilis pectate lyase;
comprising a Bacillus safensis pectate lyase;
comprising a Bacillus pumilus pectate lyase;
comprising a Bacillus licheniformis pectate lyase;
comprising a Bacillus amyloliquefaciens pectate lyase;
comprising an amino acid sequence having at least 60%, at least 70%, at least 75%, at least 80%, at least 85%, at least 90%, at least 95%, at least 98%, at least 99%, or 100% identity to any one of SEQ ID NOs: 213-217 and 222-226;
consisting of an amino acid sequence having at least 60%, at least 70%, at least 75%, at least 80%, at least 85%, at least 90%, at least 95%, at least 98%, at least 99%, or 100% identity to any one of SEQ ID NOs: 213-217 and 222-226;
or a combination of any thereof.

13. The fusion protein of claim 12, wherein the targeting sequence comprises or consists of:
(a) an amino acid sequence having at least about 43% identity with amino acids 20-35 of SEQ ID NO: 1, wherein the identity with amino acids 25-35 is at least about 54%;
(b) amino acids 1-35 of SEQ ID NO: 1;
(c) amino acids 20-35 of SEQ ID NO: 1;
(d) SEQ ID NO: 1;
(e) SEQ ID NO: 96; or
(f) SEQ ID NO: 120.

14. The fusion protein of claim 12 wherein the targeting sequence comprises the sequence X₁-X₂-X₃-X₄-X₅-X₆-X₇-X₈-X₉-X₁₀-X₁₁-X₁₂-X₁₃-X₁₄-X₁₅-X₁₆, wherein:
X₁ is any amino acid or absent;
X₂ is phenylalanine (F), leucine (L), isoleucine (I), or methionine (M);
X₃ is any amino acid;
X₄ is proline (P) or serine (S);
X₅ is any amino acid;
X₆ is leucine (L), asparagine (N), serine (S), or isoleucine (I);
X₇ is valine (V) or isoleucine (I);
X₈ is glycine (G);
X₉ is proline (P);
X₁₀ is threonine (T) or proline (P);
X₁₁ is leucine (L) or phenylalanine (F);
X₁₂ is proline (P);
X₁₃ is any amino acid;
X₁₄ is any amino acid;
X₁₅ is proline (P), glutamine (Q), or threonine (T); and
X₁₆ is proline (P), threonine (T), or serine (S).

15. (canceled)

16. The fusion protein of claim 12, wherein the fusion protein further comprises an amino acid linker between the targeting sequence, the exosporium protein, or the exosporium protein fragment and the pectate lyase.

17. (canceled)

18. A fusion protein of claim 7, wherein the pectate lyase comprises or consists of any one of SEQ ID NOs: 213-217.

19. A recombinant Bacillus cereus family member that expresses a fusion protein of claim 1.

20. (canceled)

21. The recombinant Bacillus cereus family member of claim 19, wherein the recombinant Bacillus cereus family member comprises a mutation that results in Bacillus cereus family member spores having an exosporium that is easier to remove for the spore as compared to the exopsorium of a wild-type spore.

22. The recombinant Bacillus cereus family member of claim 21, wherein the recombinant Bacillus cereus family member comprises:
(i) a mutation in a CotE gene;
(ii) a mutation in an ExsY gene;
(iii) a mutation in a CotY gene;
(iv) a mutation in a ExsA gene;
(v) a mutation in a CotO gene.

23-25. (canceled)

26. Exosporium fragments derived from the recombinant Bacillus cereus family member of claim 21.

27-29. (canceled)

30. A method for stimulating plant growth and/or promoting plant health, comprising applying a recombinant Bacillus cereus family member of claim 19 to a plant growth medium, a plant, a plant seed, or an area surrounding a plant or a plant seed.

31. (canceled)

32. A method for stimulating plant growth and/or promoting plant health, comprising applying exosporium fragments of claim 26 to a plant growth medium, a plant, a plant seed, or an area surrounding a plant or a plant seed.

33-34. (canceled)
